SEQUENCE LISTING (12 sequences)

SEQ ID NO: 1 - DNA, 20 bp, Artificial Sequence
  primer
gctcggtgaccctggtcttt

SEQ ID NO: 2 - DNA, 20 bp, Artificial Sequence
  primer0
aatgtgaggcgggtggaact

SEQ ID NO: 3 - DNA, 21 bp, Artificial Sequence
  primer
aagaccttgagtagttgccca

SEQ ID NO: 4 - DNA, 21 bp, Artificial Sequence
  primer
tgctcgatcctacgtttgcag

SEQ ID NO: 5 - DNA, 20 bp, Artificial Sequence
  primer
accccagcatggaaacaaag

SEQ ID NO: 6 - DNA, 25 bp, Artificial Sequence
  primer
aagaatggtattgaagtcatctgtc

SEQ ID NO: 7 - DNA, 21 bp, Artificial Sequence
  primer
accccgtgaagaaaatcagaa

SEQ ID NO: 8 - DNA, 20 bp, Artificial Sequence
  primer
taacaggcggtcttggttgg

SEQ ID NO: 9 - DNA, 21 bp, Artificial Sequence
  primer
tggtggtcctgtttacagtgg

SEQ ID NO: 10 - DNA, 21 bp, Artificial Sequence
  primer
ttctgaagttcttcacggggt

SEQ ID NO: 11 - DNA, 24 bp, Artificial Sequence
  primer
cctacgaagactcaaattctcagc

SEQ ID NO: 12 - DNA, 19 bp, Artificial Sequence
  primer
gaggaccatggtggtgtcc

(19) United States
(12) Patent Application Publication
(10) Pub. No.: US 2019/0033325 A1
(43) Pub. Date: Jan. 31, 2019

(54) RENAL FUNCTION EVALUATING DEVICE, DEVICE FOR PREDICTING ONSET OF KIDNEY DISEASE COMPLICATIONS, AND PHOSPHOROUS INGESTION AMOUNT ESTIMATING DEVICE

(71) Applicant: ADVANCED TELECOMMUNICATIONS RESEARCH INSTITUTE INTERNATIONAL, Kyoto (JP)

(72) Inventor: Narutoku SATO, Kyoto (JP)

(73) Assignee: ADVANCED TELECOMMUNICATIONS RESEARCH INSTITUTE INTERNATIONAL, Kyoto (JP)

(21) Appl. No.: 16/072,708

(22) PCT Filed: Jan. 24, 2017
(86) PCT No.: PCT/JP2017/002406

§ 371 Date: Oct. 19, 2018

(30) Foreign Application Priority Data
Jan. 29, 2016 (JP) 2016-016166
Feb. 3, 2016 (JP) 2016-018926

(51) Int. Cl. G01N 33/68; C12Q 1/6883

(52) U.S. Cl. CPC G01N 33/6893; C12Q 1/6883; G01N 2800/347; C12Q 2600/118; C12Q 2600/106

(57) ABSTRACT

A device 1 comprises a measurement value obtaining unit 13 for obtaining a measurement value M2 relating to at least one protein selected from the group consisting of kidney function prediction markers contained in a specimen collected from a subject and/or a measurement value M3 of at least one mRNA selected from the group consisting of kidney function prediction markers contained in a specimen collected from the subject, and a kidney function evaluation unit 14 for evaluating kidney function based on the measurement value(s) obtained by the obtaining unit.

### TECHNICAL FIELD

The present invention relates to a device for evaluating kidney function, a method for evaluating kidney function, and a program that causes a computer to perform a kidney function evaluation function. The present invention also relates to a device for predicting onset of a complication(s) of kidney disease, a method for predicting onset of a complication(s) of kidney disease, and a program that causes a computer to perform a function for predicting onset of a complication(s) of kidney disease. The present invention further relates to a device for estimating phosphorus intake, a method for estimating phosphorus intake, and a program that causes a computer to perform a phosphorus intake prediction function.

### BACKGROUND ART

Diseases include those in a state that can be reversibly treated, and those in a state that cannot, i.e., those in an irreversible state. Early detection and treatment of abnormalities during a reversible state, or preventing such a state from occurring, is essential for health maintenance. Even in a reversible state, early detection of disease directly leads to milder treatment, a shorter treatment period, and better prognostic health. As in heart disease, brain disease, cancer, and diabetes, it is well known that abnormalities in one organ or tissue lead to a disease state in other organs (commonly called a “complication”). In such diseases, it is essential to prevent, at the earliest possible time, abnormalities in one organ or tissue from causing disease in other organs or tissue.

In all animals, including humans, each organ and tissue form a functional network, rather than serving as separate parts, and quality control at the individual level is achieved. Transport of endocrine factors, such as hormones, by the vascular network throughout the entire body and coordinated adjustment of organ functions by the neural network are typical examples of an “inter-organ cross-talk system,” and systematized as physiology or endocrinology.

Meanwhile, the number of end-stage kidney disease (ESKD) patients in need of dialysis or kidney transplant has been increasing worldwide. The number of ESKD patients increased from 430,000 to 1,065,000 over the decade from 1990 to 2000, and further increased to at least about 1,650,000 in 2008 (Non-patent Literature 1). Chronic kidney disease (CKD) progresses to ESKD. However, in kidneys, called the “silent organ,” even if kidney damage occurs, its condition is less likely to appear in clinical data etc. Thus, early detection of decreased kidney function before onset of chronic kidney disease is difficult.

### CITATION LIST

### Non-Patent Literature

NPL 1: Lysaght M J: J Am Soc Nephrol. 2002 January; 13 Suppl 1; S37-40.

### SUMMARY OF INVENTION

### Technical Problem

An object of the present invention is to provide a method for evaluating kidney function to detect decreased kidney function earlier than in conventional test methods; and a method for predicting onset of a complication(s) of kidney disease. Another object of the present invention is to provide a method for predicting phosphorus intake, and a method for determining an effect of dietary therapy for preventing a decrease in kidney function or suppressing progression of a decrease in kidney function. A further object of the present invention is to provide devices and programs for implementing the above methods.

### Solution to Problem

The present inventor conducted extensive research, and found that the expression of certain kidney function prediction markers increases in an animal model of kidney disease. The inventor also found that the expression of the kidney function prediction markers starts to increase earlier than the serum creatinine concentration, which is known to increase in kidney disease, increases. The present inventor further found that the kidney function prediction markers reflect phosphorus intake, which is not sufficiently evaluated in observation of the concentration of serum inorganic phosphorus.

The present invention has been accomplished based on these findings, and includes the following embodiments.

### I. Evaluation of Kidney Function

### I-1.

A device for evaluating kidney function of a subject, comprising the following computation means:

means for obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

means for evaluating the kidney function based on the measurement value(s) obtained by the obtaining means.

### I-2.

The device according to I-1, wherein the kidney function prediction marker is at least one member selected from the group consisting of proline-rich proteins (PRPs), defensins, and Hamp2.

I-3.

The device according to Item I-1 or I-2, wherein in the case where expression of the kidney function prediction marker(s) increases with a decrease in kidney function, the evaluation means compares the measurement value(s) with predetermined threshold(s), and determines that the subject has decreased kidney function when the measurement value(s) are higher than the threshold(s), or

in the case where expression of the kidney function prediction marker(s) decreases with a decrease in kidney function, the evaluation means compares the measurement value(s) with predetermined threshold(s), and determines that the subject has decreased kidney function when the measurement value(s) are lower than the threshold(s).

### I-4.

The device according to any one of Items I-1 to I-3, wherein when the specimen collected from the subject is a blood sample or a body fluid, the obtaining means obtains measurement values of the protein and the mRNA,

when the specimen collected from the subject is tissue, the obtaining means obtains a measurement value of the mRNA.

### I-5.

The device according to any one of Items I-1 to I-4, further comprising means for obtaining a measurement value of at least one kidney disease marker in the subject; and means for comparing the measurement value of the kidney disease marker obtained by the kidney disease marker value obtaining means with a threshold of a corresponding kidney disease marker, and identifying a subject in which the measurement value of the kidney disease marker is within a range of the threshold of the kidney disease marker.

### I-6.

The device according to any one of Items I-1 to I-5, wherein when the kidney function prediction marker is at least one member selected from the group consisting of PRPs, the measurement value relating to at least one protein selected from the group consisting of the kidney function prediction markers is a measurement value of proline.

### I-7.

The device according to any one of Items I-1 to I-6, wherein when the kidney function prediction marker is at least one member selected from the group consisting of PRPs, the specimen is at least one member selected from the group consisting of saliva and salivary glands.

### I-8.

The device according to any one of Items I-1 to I-5, wherein when the kidney function prediction marker is at least one member selected from the group consisting of defensins and Hamp2, the specimen is at least one member selected from the group consisting of adipose tissue, hair roots, skin, secretion from skin, and sweat.

### I-9.

A program that, when executed by a computer, causes the computer to carry out the following processing to evaluate a kidney function of a subject:

processing of obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

processing of evaluating the kidney function based on the measurement value(s) obtained in the obtaining processing.

### I-10.

The program according to I-9, wherein the kidney function prediction marker is at least one member selected from the group consisting of PRPs, defensins, and Hamp2.

### I-11.

The program according to Item I-9 or I-10, wherein in the evaluation processing, in the case where expression of the kidney function prediction marker(s) increases with a decrease in kidney function, the measurement value(s) are compared with predetermined threshold(s), and it is determined that the subject has decreased kidney function when the measurement value(s) are higher than the threshold(s), or

in the case where expression of the kidney function prediction marker(s) decreases with a decrease in kidney function, the measurement value(s) are compared with predetermined threshold(s), and it is determined that the subject has decreased kidney function when the measurement value(s) are lower than the threshold(s).

### I-12.

The program according to any one of Items I-9 to I-11, wherein when the specimen collected from the subject is a blood sample or a body fluid, measurement values of the protein and the mRNA are obtained in the obtaining processing,

when the specimen collected from the subject is tissue, a measurement value of the mRNA is obtained in the obtaining processing.

### I-13.

The program according to any one of Items I-9 to I-12, wherein the program further causes the computer to carry out processing of obtaining a measurement value of a kidney disease marker in the subject; and processing of comparing the measurement value of the kidney disease marker obtained in the kidney disease marker value obtaining processing with a threshold of a corresponding kidney disease marker and identifying a subject in which the measurement value of the kidney disease marker is within a range of the threshold of the kidney disease marker.

### I-14.

The program according to any one of Items I-9 to I-13, wherein when the kidney function prediction marker is at least one member selected from the group consisting of PRPs, the measurement value relating to at least one protein selected from the group consisting of the kidney function prediction markers is a measurement value of proline.

### I-15.

The program according to any one of Items I-9 to I-14, wherein when the kidney function prediction marker is at least one member selected from the group consisting of PRPs, the specimen is at least one member selected from the group consisting of saliva and salivary glands.

### I-16.

The program according to any one of Items I-9 to I-13, wherein when the kidney function prediction marker is at least one member selected from the group consisting of defensins and Hamp2, the specimen is at least one member selected from the group consisting of adipose tissue, hair roots, skin, secretion from skin, and sweat.

### I-17.

A method for supporting the evaluation of a kidney function of a subject, comprising the steps of:

obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

evaluating the kidney function based on the measurement value(s) obtained in the obtaining step.

### I-18.

The method according to Item I-17, wherein the kidney function prediction marker is at least one member selected from the group consisting of PRPs, defensins, and Hamp2.

### I-19.

The method according to Item I-17 or I-18, wherein in the evaluation step, in the case where expression of the kidney function prediction marker(s) increases with a decrease in kidney function, the measurement value(s) are compared with predetermined threshold(s), and it is determined that the subject has decreased kidney function when the measurement value(s) are higher than the threshold(s), or

in the case where expression of the kidney function prediction marker(s) decreases with a decrease in kidney function, the measurement value(s) are compared with predetermined threshold(s), and it is determined that the subject has decreased kidney function when the measurement value(s) are lower than the threshold(s).

### I-20.

The method according to any one of Items I-17 to I-19, wherein when the specimen collected from the subject is a blood sample or a body fluid, measurement values of the protein and the mRNA are obtained in the obtaining step,

when the specimen collected from the subject is tissue, a measurement value of the mRNA is obtained in the obtaining step.

### I-21.

The method according to any one of Items I-16 to I-20, further comprising, before the obtaining step, the steps of:

obtaining a measurement value of at least one kidney disease marker in the subject; and

comparing the measurement value of the kidney disease marker obtained in the kidney disease marker value obtaining step with a threshold of a corresponding kidney disease marker, and identifying a subject in which the measurement value of the kidney disease marker is within a range of the threshold of the kidney disease marker.

### I-22.

The method according to any one of Items I-17 to I-21, wherein when the kidney function prediction marker is at least one member selected from the group consisting of PRPs, the measurement value relating to at least one protein selected from the group consisting of the kidney function prediction markers is a measurement value of proline.

### I-23.

The method according to any one of Items I-17 to I-22, wherein when the kidney function prediction marker is at least one member selected from the group consisting of PRPs, the specimen is at least one member selected from the group consisting of saliva and salivary glands.

### I-24.

The method according to any one of Items I-17 to I-21, wherein when the kidney function prediction marker is at least one member selected from the group consisting of defensins and Hamp2, the specimen is at least one member selected from the group consisting of adipose tissue, hair roots, skin, secretion from skin, and sweat.

### I-25.

A test reagent for use in the method according to any one of Items I-17 to I-21, I-23, and I-24, comprising at least one antibody selected from the group consisting of anti-kidney function prediction marker antibodies, or at least one nucleic acid selected from the group consisting of nucleic acids for kidney function prediction marker mRNA detection.

### II. Estimation of Phosphorus Intake

### II-1.

A device for estimating phosphorus intake in a subject, comprising the following computation means:

means for obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

means for estimating the phosphorus intake in the subject based on the measurement value(s) obtained by the obtaining means.

### II-2.

The device according to Item II-1, wherein the kidney function prediction marker is at least one member selected from the group consisting of PRPs, defensins, and Hamp2.

### II-3.

The device according to Item II-1 or II-2, wherein in the case where expression of the kidney function prediction marker(s) increases with an increase in phosphorus intake, the estimation means compares the measurement value(s) with predetermined threshold(s), and determines that the phosphorus intake in the subject is high when the measurement value(s) are higher than the threshold(s), or

in the case where expression of the kidney function prediction marker(s) decreases with an increase in phosphorus intake, the estimation means compares the measurement value(s) with predetermined threshold(s), and determines that the phosphorus intake in the subject is high when the measurement value(s) are lower than the threshold(s).

### II-4.

The device according to any one of Items II-1 to II-3, further comprising prediction means for determining that the subject is at risk of a future decrease in kidney function when the phosphorus intake in the subject is determined to be high.

### II-5.

The device according to any one of Items II-1 to II-4, wherein when the specimen collected from the subject is a blood sample or a body fluid, the obtaining means obtains measurement values of the protein and the mRNA,

when the specimen collected from the subject is tissue, the obtaining means obtains a measurement value of the mRNA.

### II-6.

The device according to any one of Items II-1 to II-5, further comprising, before the obtaining means obtains the measurement value(s), the following means for:

obtaining a measurement value of at least one kidney disease marker in the subject; and

comparing the measurement value of the kidney disease marker obtained by the kidney disease marker value obtaining means with a threshold of a corresponding kidney disease marker, and identifying a subject in which the measurement value of the kidney disease marker is within a range of the threshold of the kidney disease marker.

### II-7.

The device according to any one of Items II-1 to II-6, wherein when the kidney function prediction marker is at least one member selected from the group consisting of PRPs, the measurement value relating to at least one protein selected from the group consisting of the kidney function prediction markers is a measurement value of proline.

### II-8.

The device according to any one of Items II-1 to II-7, wherein when the kidney function prediction marker is at least one member selected from the group consisting of PRPs, the specimen is at least one member selected from the group consisting of saliva and salivary glands.

### II-9.

The device according to any one of Items II-1 to II-6, wherein when the kidney function prediction marker is at least one member selected from the group consisting of defensins and Hamp2, the specimen is at least one member selected from the group consisting of adipose tissue, hair roots, skin, secretion from skin, and sweat.

### II-10.

A program that, when executed by a computer, causes the computer to carry out the following processing to estimate phosphorus intake in a subject:

processing of obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

processing of estimating the phosphorus intake in the subject based on the measurement value(s) obtained in the obtaining processing.

### II-11.

The program according to Item II-10, wherein the kidney function prediction marker is at least one member selected from the group consisting of PRPs, defensins, and Hamp2.

### II-12.

The program according to Item II-10 or II-11, wherein in the estimation processing, in the case where expression of the kidney function prediction marker(s) increases with an increase in phosphorus intake, the measurement value(s) are compared with predetermined threshold(s), and it is determined that the phosphorus intake in the subject is high when the measurement value(s) are higher than the threshold(s), or

in the case where expression of the kidney function prediction marker(s) decreases with an increase in phosphorus intake, the measurement value(s) are compared with predetermined threshold(s), and it is determined that the phosphorus intake in the subject is high when the measurement value(s) are lower than the threshold(s).

### II-13.

The program according to any one of II-10 to II-12, wherein the program further causes the computer to carry out processing of determining that the subject is at risk of a future decrease in kidney function when the phosphorus intake in the subject is determined to be high.

### II-14.

The program according to any one of Items II-10 to II-13, wherein when the specimen collected from the subject is a blood sample or a body fluid, measurement values of the protein and the mRNA are obtained in the obtaining processing,

when the specimen collected from the subject is tissue, a measurement value of the mRNA is obtained in the obtaining processing.

### II-15.

The program according to any one of Items II-10 to II-14, wherein before the obtaining processing, the program further causes the computer to carry out processing of obtaining a measurement value of at least one kidney disease marker in the subject; and processing of comparing the measurement value of the kidney disease marker obtained in the kidney disease marker value obtaining processing with a threshold of a corresponding kidney disease marker and identifying a subject in which the measurement value of the kidney disease marker is within a range of the threshold of the kidney disease marker.

### II-16.

The program according to any one of Items II-10 to II-15, wherein when the kidney function prediction marker is at least one member selected from the group consisting of PRPs, the measurement value relating to at least one protein selected from the group consisting of the kidney function prediction markers is a measurement value of proline.

### II-17.

The program according to any one of Items II-10 to II-16, wherein when the kidney function prediction marker is at least one member selected from the group consisting of PRPs, the specimen is at least one member selected from the group consisting of saliva and salivary glands.

### II-18.

The program according to any one of Items II-10 to II-15, wherein when the kidney function prediction marker is at least one member selected from the group consisting of defensins and Hamp2, the specimen is at least one member selected from the group consisting of adipose tissue, hair roots, skin, secretion from skin, and sweat.

### II-19.

A method for supporting the estimation of phosphorus intake in a subject, comprising the steps of:

obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

estimating the phosphorus intake in the subject based on the measurement value(s) obtained in the obtaining step.

### II-20.

The method according to Item II-19, wherein the kidney function prediction marker is at least one member selected from the group consisting of PRPs, defensins, and Hamp2.

### II-21.

The method according to Item II-19 or II-20, wherein in the estimation step, in the case where expression of the kidney function prediction marker(s) increases with an increase in phosphorus intake, the measurement value(s) are compared with predetermined threshold(s), and it is determined that the phosphorus intake in the subject is high when the measurement value(s) are higher than the threshold(s), or

in the case where expression of the kidney function prediction marker(s) decreases with an increase in phosphorus intake, the measurement value(s) are compared with predetermined threshold(s), and it is determined that the phosphorus intake in the subject is high when the measurement value(s) are lower than the threshold(s).

### II-22.

The method according to any one of Items II-19 to II-21, further comprising a prediction step of determining that the subject is at risk of a future decrease in kidney function when the phosphorus intake in the subject is determined to be high.

### II-23.

The method according to any one of Items II-19 to II-22, wherein when the specimen collected from the subject is a blood sample or a body fluid, measurement values of the protein and the mRNA are obtained in the obtaining step,

when the specimen collected from the subject is tissue, a measurement value of the mRNA is obtained in the obtaining step.

### II-24.

The method according to any one of Items II-19 to II-23, further comprising, before the obtaining step, the steps of:

obtaining a measurement value of at least one kidney disease marker in the subject; and

comparing the measurement value of the kidney disease marker obtained in the kidney disease marker value obtaining step with a threshold of a corresponding kidney disease marker and identifying a subject in which the measurement value of the kidney disease marker is within a range of the threshold of the kidney disease marker.

### II-25.

The method according to any one of Items II-19 to II-24, wherein when the kidney function prediction marker is at least one member selected from the group consisting of PRPs, the measurement value relating to at least one protein selected from the group consisting of the kidney function prediction markers is a measurement value of proline.

### II-26.

The method according to any one of Items II-19 to II-25, wherein when the kidney function prediction marker is at least one member selected from the group consisting of PRPs, the specimen is at least one member selected from the group consisting of saliva and salivary glands.

### II-27.

The method according to any one of Items II-19 to II-24, wherein when the kidney function prediction marker is at least one member selected from the group consisting of defensins and Hamp2, the specimen is at least one member selected from the group consisting of adipose tissue, hair roots, skin, secretion from skin, and sweat.

### II-28.

A test reagent for use in the method according to any one of Items II-19 to II-24, 11-26, and II-27, comprising an antibody against kidney function prediction marker or a nucleic acid for kidney function prediction marker mRNA detection.

### III. Prediction of Possibility of Developing Complication (1)

### III-1.

A device for predicting a possibility of developing a complication(s) associated with kidney disease in a subject in the future, comprising the following computation means:

means for obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

means for predicting the possibility of developing a complication(s) associated with kidney disease in the future based on the measurement value(s) obtained by the obtaining means.

### III-2.

The device according to Item III-1, wherein the kidney function prediction marker is at least one member selected from the group consisting of PRPs, defensins, and Hamp2.

### III-3.

The device according to Item III-1 or III-2, wherein in the case where expression of the kidney function prediction marker(s) increases with a decrease in kidney function, the prediction means compares the measurement value(s) with predetermined threshold(s), and determines that the subject has a possibility of developing a complication(s) associated with kidney disease in the future when the measurement value(s) are higher than the threshold(s), or

in the case where expression of the kidney function prediction marker(s) decreases with a decrease in kidney function, the prediction means compares the measurement value(s) with predetermined threshold(s), and determines that the subject has a possibility of developing a complication(s) associated with kidney disease in the future when the measurement value(s) are lower than the threshold(s). III-4.

The device according to any one of Items III-1 to III-3, wherein the complication(s) are at least one member selected from the group consisting of urine concentrating ability disorders, azotemia, water/electrolyte abnormalities, metabolic acidosis, renal anemia, and secondary hyperparathyroidism.

### III-5.

The device according to any one of Items III-1 to III-4, wherein when the specimen collected from the subject is a blood sample or a body fluid, the obtaining means obtains measurement values of the protein and the mRNA,

when the specimen collected from the subject is tissue, the obtaining means obtains a measurement value of the mRNA.

### III-6.

The device according to any one of Items III-1 to III-5, further comprising, before the obtaining means obtains the measurement value(s), the following means for:

obtaining a measurement value of at least one kidney disease marker in the subject; and

comparing the measurement value of the kidney disease marker obtained by the kidney disease marker value obtaining means with a threshold of a corresponding kidney disease marker, and identifying a subject in which the measurement value of the kidney disease marker is within a range of the threshold of the kidney disease marker.

### III-7.

The device according to any one of Items III-1 to III-6, wherein the subject is a subject diagnosed with chronic kidney disease GFR category G1 or chronic kidney disease GFR category G2.

### III-8.

The device according to any one of Items III-1 to III-7, wherein when the kidney function prediction marker is at least one member selected from the group consisting of PRPs, the measurement value relating to at least one protein selected from the group consisting of the kidney function prediction markers is a measurement value of proline.

### III-9.

The device according to any one of Items III-1 to III-8, wherein when the kidney function prediction marker is at least one member selected from the group consisting of PRPs, the specimen is at least one member selected from the group consisting of saliva and salivary glands.

### III-10.

The device according to any one of Items III-1 to III-7, wherein when the kidney function prediction marker is at least one member selected from the group consisting of defensins and Hamp2, the specimen is at least one member selected from the group consisting of adipose tissue, hair roots, skin, secretion from skin, and sweat.

### III-11.

A program that, when executed by a computer, causes the computer to carry out the following processing to predict a possibility of developing a complication(s) associated with kidney disease in a subject in the future:

processing of obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

processing of predicting the possibility of developing a complication(s) associated with kidney disease in the future based on the measurement value(s) obtained in the obtaining processing.

### III-12.

The program according to Item III-11, wherein the kidney function prediction marker is at least one member selected from the group consisting of PRPs, defensins, and Hamp2.

### III-13.

The program according to Item III-11 or III-12, wherein in the prediction processing, in the case where expression of the kidney function prediction marker(s) increases with a decrease in kidney function, the measurement value(s) are compared with predetermined threshold(s), and it is determined that the subject has a possibility of developing a complication(s) associated with kidney disease in the future when the measurement value(s) are higher than the threshold(s), or

in the case where expression of the kidney function prediction marker(s) decreases with a decrease in kidney function, the measurement value(s) are compared with predetermined threshold(s), and it is determined that the subject has a possibility of developing a complication(s) associated with kidney disease in the future when the measurement value(s) are lower than the threshold(s).

### III-14.

The program according to any one of Items III-11 to III-13, wherein the complication(s) are at least one member selected from the group consisting of urine concentrating ability disorders, azotemia, water/electrolyte abnormalities, metabolic acidosis, renal anemia, and secondary hyperparathyroidism.

### III-15.

The program according to any one of Items III-11 to III-14, wherein when the specimen collected from the subject is a blood sample or a body fluid, measurement values of the protein and the mRNA are obtained in the obtaining processing,

when the specimen collected from the subject is tissue, a measurement value of the mRNA is obtained in the obtaining processing.

### III-16.

The program according to any one of Items III-11 to III-15, wherein before the obtaining processing, the program further causes the computer to carry out processing of obtaining a measurement value of at least one kidney disease marker in the subject; and processing of comparing the measurement value of the kidney disease marker obtained in the kidney disease marker value obtaining processing with a threshold of a corresponding kidney disease marker and identifying a subject in which the measurement value of the kidney disease marker is within a range of the threshold of the kidney disease marker.

### III-17.

The program according to any one of Items III-11 to III-16, wherein the subject is a subject diagnosed with chronic kidney disease GFR category G1 or chronic kidney disease GFR category G2.

### III-18.

The program according to any one of Items III-11 to III-17, wherein when the kidney function prediction marker is at least one member selected from the group consisting of PRPs, the measurement value relating to at least one protein selected from the group consisting of the kidney function prediction markers is a measurement value of proline.

### III-19.

The program according to any one of Items III-11 to III-18, wherein when the kidney function prediction marker is at least one member selected from the group consisting of PRPs, the specimen is at least one member selected from the group consisting of saliva and salivary glands.

### III-20.

The program according to any one of Items III-11 to III-17, wherein when the kidney function prediction marker is at least one member selected from the group consisting of defensins and Hamp2, the specimen is at least one member selected from the group consisting of adipose tissue, hair roots, skin, secretion from skin, and sweat.

### III-21.

A method for supporting the prediction of a possibility of developing a complication(s) associated with kidney disease in a subject in the future, comprising the steps of:

obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

predicting the possibility of developing a complication(s) associated with kidney disease in the future based on the measurement value(s) obtained in the obtaining step.

### III-22.

The method according to Item II-21, wherein the kidney function prediction marker is at least one member selected from the group consisting of PRPs, defensins, and Hamp2.

### III-23.

The method according to Item III-21 or III-22, wherein in the prediction step, in the case where expression of the kidney function prediction marker(s) increases with a decrease in kidney function, the measurement value(s) are compared with predetermined threshold(s), and it is determined that the subject has a possibility of developing a complication(s) associated with kidney disease in the future when the measurement value(s) are higher than the threshold(s), or

in the case where expression of the kidney function prediction marker(s) decreases with a decrease in kidney function, the measurement value(s) are compared with predetermined threshold(s), and it is determined that the subject has a possibility of developing a complication(s) associated with kidney disease in the future when the measurement value(s) are lower than the threshold(s).

### III-24.

The method according to any one of Items III-21 to III-23, wherein the complication(s) are at least one member selected from the group consisting of urine concentrating ability disorders, azotemia, water/electrolyte abnormalities, metabolic acidosis, renal anemia, and secondary hyperparathyroidism.

### III-25.

The method according to any one of Items III-21 to III-24, wherein when the specimen collected from the subject is a blood sample or a body fluid, measurement values of the protein and the mRNA are obtained in the obtaining step,

when the specimen collected from the subject is tissue, a measurement value of the mRNA is obtained in the obtaining step.

### III-26.

The method according to any one of Items III-21 to III-25, further comprising, before the obtaining step, the steps of:

obtaining a measurement value of at least one kidney disease marker in the subject; and

comparing the measurement value of the kidney disease marker obtained in the kidney disease marker value obtaining step with a threshold of a corresponding kidney disease marker, and identifying a subject in which the measurement value of the kidney disease marker is within a range of the threshold of the kidney disease marker.

### III-27.

The method according to any one of Items III-21 to III-26, wherein the subject is a subject diagnosed with chronic kidney disease GFR category G1 or chronic kidney disease GFR category G2.

### III-28.

The method according to any one of Items III-21 to III-27, wherein when the kidney function prediction marker is at least one member selected from the group consisting of PRPs, the measurement value relating to at least one protein selected from the group consisting of the kidney function prediction markers is a measurement value of proline.

### III-29.

The method according to any one of Items III-21 to III-28, wherein when the kidney function prediction marker is at least one member selected from the group consisting of PRPs, the specimen is at least one member selected from the group consisting of saliva and salivary glands.

### III-30.

The method according to any one of Items III-21 to III-27, wherein when the kidney function prediction marker is at least one member selected from the group consisting of defensins and Hamp2, the specimen is at least one member selected from the group consisting of adipose tissue, hair roots, skin, secretion from skin, and sweat.

### III-31.

A test reagent for use in the method according to any one of Items III-19 to III-27, III-29, and III-30, comprising an antibody against kidney function prediction marker or a nucleic acid for kidney function prediction marker mRNA detection.

### IV. Prediction of Possibility of Developing Complication (2)

### IV-1.

A device for predicting a possibility of developing a complication(s) associated with kidney disease in a subject in the future, comprising the following computation means:

first obtaining means for obtaining a first measurement value relating to at least one protein selected from the group consisting of kidney function prediction markers contained in a specimen collected earlier from the subject, and/or a first measurement value of at least one mRNA selected from group consisting of kidney function prediction markers contained in a specimen collected earlier from the subject;

second obtaining means for obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

means for predicting the possibility of developing a complication(s) associated with kidney disease in the future based on the first measurement value(s) obtained by the first obtaining means and the second measurement value(s) obtained by the second obtaining means.

### IV-2.

The device according to Item IV-1, wherein the kidney function prediction marker is at least one member selected from the group consisting of PRPs, defensins, and Hamp2.

### IV-3.

The device according to Item IV-1 or IV-2, wherein in the case where expression of the kidney function prediction marker(s) increases with a decrease in kidney function, the prediction means compares the first measurement value(s) with the second measurement value(s), and determines that the subject has a possibility of developing a complication(s) associated with kidney disease in the future when the second measurement value(s) are higher than the first measurement value(s), or

in the case where expression of the kidney function prediction marker(s) decreases with a decrease in kidney function, the prediction means compares the first measurement value(s) with the second measurement value(s), and determines that the subject has a possibility of developing a complication(s) associated with kidney disease in the future when the second measurement value(s) are lower than the first measurement value(s).

### IV-4.

The device according to any one of Items IV-1 to IV-3, wherein the complication(s) are at least one member selected from the group consisting of urine concentrating ability disorders, azotemia, water/electrolyte abnormalities, metabolic acidosis, renal anemia, and secondary hyperparathyroidism.

### IV-5.

The device according to any one of Items IV-1 to IV-4, wherein when the specimen collected from the subject is a blood sample or a body fluid, the first obtaining means obtains first measurement values of the protein and the mRNA, and the second obtaining means obtains second measurement values of the protein and the mRNA,

when the specimen collected from the subject is tissue, the first obtaining means obtains a first measurement value of the mRNA, and the second obtaining means obtains a second measurement value of the mRNA.

### IV-6.

The device according to any one of Items IV-1 to IV-5, further comprising, before the first and second obtaining means respectively obtain the first and second measurement values, the following means for:

obtaining a measurement value of at least one kidney disease marker in the subject; and

comparing the measurement value of the kidney disease marker obtained by the kidney disease marker value obtaining means with a threshold of a corresponding kidney disease marker, and identifying a subject in which the measurement value of the kidney disease marker is within a range of the threshold of the kidney disease marker.

### IV-7.

The device according to any one of Items IV-1 to IV-6, wherein the subject is a subject diagnosed with chronic kidney disease GFR category G1 or chronic kidney disease GFR category G2.

### IV-8.

The device according to any one of Items IV-1 to IV-7, wherein when the kidney function prediction marker is at least one member selected from the group consisting of PRPs, the measurement value relating to at least one protein selected from the group consisting of the kidney function prediction markers is a measurement value of proline.

### IV-9.

The device according to any one of Items IV-1 to IV-8, wherein when the kidney function prediction marker is at least one member selected from the group consisting of PRPs, the specimen is at least one member selected from the group consisting of saliva and salivary glands.

### IV-10.

The device according to any one of Items IV-1 to IV-7, wherein when the kidney function prediction marker is at least one member selected from the group consisting of defensins and Hamp2, the specimen is at least one member selected from the group consisting of adipose tissue, hair roots, skin, secretion from skin, and sweat.

### IV-11.

A program that, when executed by a computer, causes the computer to carry out the following processing to predict a possibility of developing a complication(s) associated with kidney disease in a subject in the future:

first obtaining processing of obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

second obtaining processing of obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

processing of predicting the possibility of developing a complication(s) associated with kidney disease in the future based on the first measurement value(s) obtained in the first obtaining processing and the second measurement value(s) obtained in the second obtaining processing.

### IV-12.

The program according to Item IV-11, wherein the kidney function prediction marker is at least one member selected from the group consisting of PRPs, defensins, and Hamp2.

### IV-13.

The program according to Item IV-11 or IV-12, wherein in the prediction processing, in the case where expression of the kidney function prediction marker(s) increases with a decrease in kidney function, the first measurement value(s) are compared with the second measurement value(s) and it is determined that the subject has a possibility of developing a complication(s) associated with kidney disease in the future when the second measurement value(s) are higher than the first measurement value(s), or

in the case where expression of the kidney function prediction marker(s) decreases with a decrease in kidney function, the first measurement value(s) are compared with the second measurement value(s) and it is determined that the subject has a possibility of developing a complication(s) associated with kidney disease in the future when the second measurement value(s) are lower than the first measurement value(s).

### IV-14.

The program according to any one of Items IV-11 to IV-13, wherein the complication(s) are at least one member selected from the group consisting of urine concentrating ability disorders, azotemia, water/electrolyte abnormalities, metabolic acidosis, renal anemia, and secondary hyperparathyroidism.

### IV-15.

The program according to any one of Items IV-11 to IV-14, wherein when the specimen collected from the subject is a blood sample or a body fluid, first measurement values of the protein and the mRNA are obtained in the first obtaining processing, and second measurement values of the protein and the mRNA are obtained in the second obtaining processing,

when the specimen collected from the subject is tissue, a first measurement value of the mRNA is obtained in the first obtaining processing, and a second measurement value of the mRNA is obtained in the second obtaining processing.

### IV-16.

The program according to any one of Items IV-11 to IV-15, wherein before the first and second obtaining processing, the program further causes the computer to carry out processing of obtaining a measurement value of at least one kidney disease marker in the subject; and processing of comparing the measurement value of the kidney disease marker obtained in the kidney disease marker value obtaining processing with a threshold of a corresponding kidney disease marker and identifying a subject in which the measurement value of the kidney disease marker is within a range of the threshold of the kidney disease marker.

### IV-17.

The program according to any one of Items IV-11 to IV-16, wherein the subject is a subject diagnosed with chronic kidney disease GFR category G1 or chronic kidney disease GFR category G2.

### IV-18.

The program according to any one of Items IV-11 to IV-17, wherein when the kidney function prediction marker is at least one member selected from the group consisting of PRPs, the measurement value relating to at least one protein selected from the group consisting of the kidney function prediction markers is a measurement value of proline.

### IV-19.

The program according to any one of Items IV-11 to IV-18, wherein when the kidney function prediction marker is at least one member selected from the group consisting of PRPs, the specimen is at least one member selected from the group consisting of saliva and salivary glands.

### IV-20.

The program according to any one of Items IV-11 to IV-17, wherein when the kidney function prediction marker is at least one member selected from the group consisting of defensins and Hamp2, the specimen is at least one member selected from the group consisting of adipose tissue, hair roots, skin, secretion from skin, and sweat.

### IV-21.

A method for supporting the prediction of a possibility of developing a complication(s) associated with kidney disease in a subject in the future, comprising the following steps:

a first obtaining step of obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

a second obtaining step of obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

a step of predicting the possibility of developing a complication(s) associated with kidney disease in the future based on the first measurement value(s) obtained in the first obtaining step and the second measurement value(s) obtained in the second obtaining step.

### IV-22.

The method according to Item IV-21, wherein the kidney function prediction marker is at least one member selected from the group consisting of PRPs, defensins, and Hamp2.

### IV-23.

The method according to Item IV-21 or IV-22, wherein in the prediction step, in the case where expression of the kidney function prediction marker(s) increases with a decrease in kidney function, the first measurement value(s) are compared with the second measurement value(s), and it is determined that the subject has a possibility of developing a complication(s) associated with kidney disease in the future when the second measurement value(s) are higher than the first measurement value(s), or

in the case where expression of the kidney function prediction marker(s) decreases with a decrease in kidney function, the first measurement value(s) are compared with the second measurement value(s), and it is determined that the subject has a possibility of developing a complication(s) associated with kidney disease in the future when the second measurement value(s) are lower than the first measurement value(s).

### IV-24.

The method according to any one of Items IV-21 to IV-23, wherein the complication(s) are at least one member selected from the group consisting of urine concentrating ability disorders, azotemia, water/electrolyte abnormalities, metabolic acidosis, renal anemia, and secondary hyperparathyroidism.

### IV-25.

The method according to any one of Items IV-21 to IV-24, wherein when the specimen collected from the subject is a blood sample or a body fluid, first measurement values of the protein and the mRNA are obtained in the first obtaining step, and second measurement values of the protein and the mRNA are obtained in the second obtaining step,

when the specimen collected from the subject is tissue, a first measurement value of the mRNA is obtained in the first obtaining step, and a second measurement value of the mRNA is obtained in the second obtaining step.

### IV-26.

The method according to any one of Items IV-21 to IV-25, further comprising, before the first and second obtaining steps, the steps of:

obtaining a measurement value of at least one kidney disease marker in the subject; and

comparing the measurement value of the kidney disease marker obtained in the kidney disease marker value obtaining step with a threshold of a corresponding kidney disease marker, and identifying a subject in which the measurement value of the kidney disease marker is within a range of the threshold of the kidney disease marker.

### IV-27.

The method according to any one of Items IV-21 to IV-26, wherein the subject is a subject diagnosed with chronic kidney disease GFR category G1 or chronic kidney disease GFR category G2.

### IV-28.

The method according to any one of Items IV-21 to IV-27, wherein when the kidney function prediction marker is at least one member selected from the group consisting of PRPs, the measurement value relating to at least one protein selected from the group consisting of the kidney function prediction markers is a measurement value of proline.

### IV-29.

The method according to any one of Items IV-21 to IV-28, wherein when the kidney function prediction marker is at least one member selected from the group consisting of PRPs, the specimen is at least one member selected from the group consisting of saliva and salivary glands.

### IV-30.

The method according to any one of Items IV-21 to IV-29, wherein when the kidney function prediction marker is at least one member selected from the group consisting of defensins and Hamp2, the specimen is at least one member selected from the group consisting of adipose tissue, hair roots, skin, secretion from skin, and sweat.

### V. Determination of Effect of Dietary Therapy

### V-1.

A device for determining an effect of dietary therapy in a subject, comprising the following computation means:

first obtaining means for obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

second obtaining means for obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

means for determining the effect of dietary therapy based on the first measurement value(s) obtained by the first obtaining means and the second measurement value(s) obtained by the second obtaining means.

### V-2.

The device according to Item V-1, wherein the kidney function prediction marker is at least one member selected from the group consisting of PRPs, defensins, and Hamp2.

### V-3.

The device according to Item V-1 or V-2, wherein in the case where expression of the kidney function prediction marker(s) decreases with a decrease in phosphorus intake, the determination means compares the first measurement value(s) with the second measurement value(s), and determines that the dietary therapy is effective when the second measurement value(s) are lower than the first measurement value(s), or

in the case where expression of the kidney function prediction marker(s) increases with a decrease in phosphorus intake, the determination means compares the first measurement value(s) with the second measurement value(s), and determines that the dietary therapy is effective when the second measurement value(s) are higher than the first measurement value(s).

### V-4.

The device according to any one of Items V-1 to V-3, wherein when the specimen collected from the subject is a blood sample or a body fluid, the first obtaining means obtains first measurement values of the protein and the mRNA, and the second obtaining means obtains second measurement values of the protein and the mRNA,

when the specimen collected from the subject is tissue, the first obtaining means obtains a first measurement value of the mRNA, and the second obtaining means obtains a second measurement value of the mRNA.

### V-5.

The device according to any one of Items V-1 to V-4, further comprising, before the first and second obtaining means respectively obtain the first and second measurement values, the following means for:

obtaining a measurement value of at least one kidney disease marker in the subject; and

comparing the measurement value of the kidney disease marker obtained by the kidney disease marker value obtaining means with a threshold of a corresponding kidney disease marker, and identifying a subject in which the measurement value of the kidney disease marker is within a range of the threshold of the kidney disease marker.

### V-6.

The device according to any one of Items V-1 to V-5, wherein the subject is a subject diagnosed with chronic kidney disease GFR category G1 or chronic kidney disease GFR category G2.

### V-7.

The device according to any one of Items V-1 to V-6, wherein when the kidney function prediction marker is at least one member selected from the group consisting of PRPs, the measurement value relating to at least one protein selected from the group consisting of the kidney function prediction markers is a measurement value of proline.

### V-8.

The device according to any one of Items V-1 to V-7, wherein when the kidney function prediction marker is at least one member selected from the group consisting of PRPs, the specimen is at least one member selected from the group consisting of saliva and salivary glands.

### V-9.

The device according to any one of Items V-1 to V-6, wherein when the kidney function prediction marker is at least one member selected from the group consisting of defensins and Hamp2, the specimen is at least one member selected from the group consisting of adipose tissue, hair roots, skin, secretion from skin, and sweat.

### V-10.

A program that, when executed by a computer, causes the computer to carry out the following processing to determine an effect of dietary therapy in a subject:

first obtaining processing of obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

second obtaining processing of obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

processing of determining the effect of dietary therapy based on the first measurement value(s) obtained in the first obtaining processing and the second measurement value(s) obtained in the second obtaining processing.

### V-11.

The program according to Item V-10, wherein the kidney function prediction marker is at least one member selected from the group consisting of PRPs, defensins, and Hamp2.

### V-12.

The program according to Item V-10 or V-11, wherein in the determination processing, in the case where expression of the kidney function prediction marker(s) decreases with a decrease in phosphorus intake, the first measurement value(s) are compared with the second measurement value(s), and it is determined that the dietary therapy is effective when the second measurement value(s) are lower than the first measurement value(s), or

in the case where expression of the kidney function prediction marker(s) increases with a decrease in phosphorus intake, the first measurement value(s) are compared with the second measurement value(s), and it is determined that the dietary therapy is effective when the second measurement value(s) are higher than the first measurement value(s).

### V-13.

The program according to any one of Items V-10 to V-12, wherein when the specimen collected from the subject is a blood sample or a body fluid, first measurement values of the protein and the mRNA are obtained in the first obtaining processing, and second measurement values of the protein and the mRNA are obtained in the second obtaining processing,

when the specimen collected from the subject is tissue, a first measurement value of the mRNA is obtained in the first obtaining processing, and a second measurement value of the mRNA is obtained in the second obtaining processing.

### V-14.

The program according to any one of Items V-10 to V-13, wherein before the first and second obtaining processing, the program further causes the computer to carry out processing of obtaining a measurement value of at least one kidney disease marker in the subject; and processing of comparing the measurement value of the kidney disease marker obtained in the kidney disease marker value obtaining processing with a threshold of a corresponding kidney disease marker and identifying a subject in which the measurement value of the kidney disease marker is within a range of the threshold of the kidney disease marker.

### V-15.

The program according to any one of Items V-10 to V-14, wherein the subject is a subject diagnosed with chronic kidney disease GFR category G1 or chronic kidney disease GFR category G2.

### V-16.

The program according to any one of Items V-10 to V-15, wherein when the kidney function prediction marker is at least one member selected from the group consisting of PRPs, the measurement value relating to at least one protein selected from the group consisting of the kidney function prediction markers is a measurement value of proline.

### V-17.

The program according to any one of Items V-10 to V-16, wherein when the kidney function prediction marker is at least one member selected from the group consisting of PRPs, the specimen is at least one member selected from the group consisting of saliva and salivary glands.

### V-18.

The program according to any one of Items V-10 to V-15, wherein when the kidney function prediction marker is at least one member selected from the group consisting of defensins and Hamp2, the specimen is at least one member selected from the group consisting of adipose tissue, hair roots, skin, secretion from skin, and sweat.

### V-19.

A method for supporting the determination of an effect of dietary therapy in a subject, comprising the following steps:

a first obtaining step of obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

a second obtaining step of obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

a step of determining the effect of dietary therapy based on the first measurement value(s) obtained in the first obtaining step and the second measurement value(s) obtained in the second obtaining step.

### V-20.

The method according to Item V-19, wherein the kidney function prediction marker is at least one member selected from the group consisting of PRPs, defensins, and Hamp2.

### V-21.

The method according to Item V-19 or V-20, wherein in the determination step, in the case where expression of the kidney function prediction marker(s) decreases with a decrease in phosphorus intake, the first measurement value(s) are compared with the second measurement value(s), and it is determined that the dietary therapy is effective when the second measurement value(s) are lower than the first measurement value(s), or

in the case where expression of the kidney function prediction marker(s) increases with a decrease in phosphorus intake, the first measurement value(s) are compared with the second measurement value(s), and it is determined that the dietary therapy is effective when the second measurement value(s) are higher than the first measurement value(s).

### V-22.

The method according to any one of Items V-19 to V-21, wherein when the specimen collected from the subject is a blood sample or a body fluid, first measurement values of the protein and the mRNA are obtained in the first obtaining step, and second measurement values of the protein and the mRNA are obtained in the second obtaining step,

when the specimen collected from the subject is tissue, a first measurement value of the mRNA is obtained in the first obtaining step, and a second measurement value of the mRNA is obtained in the second obtaining step.

### V-23.

The method according to any one of Items V-19 to V-22, further comprising, before the first and second obtaining steps, the steps of:

obtaining a measurement value of at least one kidney disease marker in the subject; and

comparing the measurement value of the kidney disease marker obtained in the kidney disease marker value obtaining step with a threshold of a corresponding kidney disease marker, and identifying a subject in which the measurement value of the kidney disease marker is within a range of the threshold of the kidney disease marker.

### V-24.

The method according to any one of Items V-19 to V-23, wherein the subject is a subject diagnosed with chronic kidney disease GFR category G1 or chronic kidney disease GFR category G2.

### V-25.

The method according to any one of Items V-19 to V-24, wherein when the kidney function prediction marker is at least one member selected from the group consisting of PRPs, the measurement value relating to at least one protein selected from the group consisting of the kidney function prediction markers is a measurement value of proline.

### V-26.

The method according to any one of Items V-19 to V-25, wherein when the kidney function prediction marker is at least one member selected from the group consisting of PRPs, the specimen is at least one member selected from the group consisting of saliva and salivary glands.

### V-27.

The method according to any one of Items V-19 to V-24, wherein when the kidney function prediction marker is at least one member selected from the group consisting of defensins and Hamp2, the specimen is at least one member selected from the group consisting of adipose tissue, hair roots, skin, secretion from skin, and sweat.

### V-28.

A test reagent for use in the method according to any one of Items V-19 to V-24, V-26, and V-27, comprising an antibody against kidney function prediction marker or a nucleic acid for kidney function prediction marker mRNA detection.

### Advantageous Effects of Invention

The present invention makes it possible to detect decreased kidney function. The present invention also makes it possible to predict onset of a complication(s) of kidney disease. The present invention further makes it possible to determine phosphorus intake and an effect of dietary therapy for preventing a decrease in kidney function or suppressing progression of a decrease in kidney function.

### BRIEF DESCRIPTION OF DRAWINGS

FIG. 1 is an overview of a system 100 according to one embodiment of the present invention.

FIG. 2 is a block diagram illustrating a hardware configuration of the system 100 according to the one embodiment of the present invention.

FIG. 3 is a block diagram illustrating functions of a computing device 1 according to a first embodiment of the present invention.

FIG. 4 is a flow chart illustrating a flow of data processing performed by the computing device 1 according to the first embodiment of the present invention to carry out a kidney function evaluation method.

FIG. 5 is a block diagram illustrating functions of a computing device 2 according to a second embodiment of the present invention.

FIG. 6 is a flow chart illustrating a flow of data processing performed by the computing device 2 according to the second embodiment of the present invention to carry out a phosphorus intake estimation method.

FIG. 7 is a block diagram illustrating functions of a computing device 3 according to a third embodiment of the present invention.

FIG. 8 is a flow chart illustrating a flow of data processing performed by the computing device 3 according to the third embodiment of the present invention to carry out a method for predicting the possibility of developing a complication(s).

FIG. 9 is a block diagram to illustrate functions of a computing device 4 according to a fourth embodiment of the present invention.

FIG. 10 is a flow chart illustrating a flow of data processing performed by the computing device 4 according to the fourth embodiment of the present invention to carry out a method for predicting the possibility of developing a complication(s).

FIG. 11 is a block diagram to illustrate functions of a computing device 5 according to a fifth embodiment of the present invention.

FIG. 12 is a flow chart illustrating a flow of data processing performed by the computing device 5 according to the fifth embodiment of the present invention to carry out a method for determining an effect of dietary therapy.

FIG. 13 is an overview illustrating an example of a test kit.

FIG. 14 is a list of RNAs in mice that can be detected by, for example, RNA-Seq. In FIG. 14, “Line No” indicates a line number in the list, “Gene Name” indicates a gene name registered with the U.S. National Center for Biotechnology Information (NCBI), and “Reference Seq. ID” indicates a reference sequence ID number registered with NCBI. “Chromosome Locus” indicates a chromosome locus registered in mm10.

FIG. 15: RNAs examined for their expression levels were classified as follows. RNAs in which CKD/Sham is more than 1 or less than 1 were classified as group 2, RNAs in which CKD/Sham is more than 1.5 or less than 0.67 were classified as group 3, RNAs in which CKD/Sham is more than 2 or less than 0.5 were classified as group 4, and RNAs in which CKD/Sham is more than 5 or less than 0.2 were classified as group 5. In FIG. 15, “Line No.” indicates a line number in the list, “Groups” indicates a group number of each of the groups classified based on the CKD/Sham values, “Gene Name” indicates a gene name registered with NCBI, “Human Gene ID” indicates a human gene number registered with NCBI that corresponds to the gene name, and “Updated” indicates the date of update to the Human Gene ID in NCBI. In “Sub-Group,” “V-1” indicates RNAs, among the RNAs of group 5, in which CKD/Sham is more than 5, and “V-2” indicates RNAs, among the RNAs of group 5, in which CKD/Sham is less than 0.2. “IV-1” indicates RNAs, among the RNAs of group 4, in which CKD/Sham is more than 2 and that are not included in group 5, and “IV-2” indicates RNAs, among the RNAs of group 4, in which CKD/Sham is less than 0.5 and that are not included in group 5. “III-1” indicates RNAs, among the RNAs of group 3, in which CKD/Sham is more than 1.5 and that are not included in group 4 or group 5, and “III-2” indicates RNAs, among the RNAs of group 3, in which CKD/Sham is less than 0.67 and that are not included in group 4 or group 5. “II-1” indicates RNAs, among the RNAs of group 2, in which CKD/Sham is more than 1 and that are not included in any of groups 3 to 5, and “II-2” indicates RNAs, among the RNAs of group 2, in which CKD/Sham is less than 1 and that are not included in any of groups 3 to 5. RNAs with no group number are a group in which CKD/Sham is 1.

FIG. 16 shows the expression levels of genes in which Sham>1 and CKD/Sham>5, genes in which Sham<1 and CKD/Sham >10, and genes in which Sham>10 and CKD/Sham <0.3 at the early and middle stages.

FIG. 17 shows the expression levels of PRPs in salivary gland in a group that ingested a diet particularly high in phosphorus (High Pi), and a group that ingested a diet particularly low in phosphorus (Low Pi). A: Prb1, B: Prh1, C: Prp2, and D: Prpmp5.

FIG. 18 shows the ratios of the concentrations of hPRB1 in saliva on the final day of a test (Day 7) divided by the concentrations of hPRB1 in saliva one day before the start of the test (Day −1) in group A and group B (hPRB1 Day 7/Day −1 ratios). “Phosphorus intake ratio” is a ratio of the total phosphorus intake for 7 days after the start of the high-phosphorus diet (or normal diet) ingestion test divided by the total phosphorus intake for 7 days before the start of the high-phosphorus diet (or normal diet) ingestion test.

FIG. 19 shows the concentrations of hPRB1 in saliva in subjects diagnosed with chronic kidney disease, or diagnosed as having multiple myeloma and being at risk for kidney disease (Patients); and healthy subjects (Control Subjects).

FIG. 20 shows the concentrations of proline in saliva in a group that ingested a high-phosphorus diet (group A: High Pi), and a group that ingested a normal diet (group B: Low Pi).

### DESCRIPTION OF EMBODIMENTS

### 1. Explanation of Terms

First, terms used in the present specification, claims, and abstract are explained. Unless otherwise stated, terms used in the present specification, claims, and abstract are in accordance with the definitions in this section.

“Kidney disease” as used herein refers to any disease of the kidney, and is not particularly limited as long as there is some impairment in the kidney functionally or physically. Specific examples include pyelonephritis, glomerulonephritis, and like acute nephritis, diabetic nephropathy, kidney glomerular fibrosis, and like chronic nephritis, nephrotic syndrome, kidney tumors, acute renal failure, chronic renal failure, and the like. Kidney disease is preferably a disease that causes decreased kidney function.

“Decreased kidney function” as used herein refers to, in the case of humans, a condition in which, for example, at least one kidney disease marker (preferably other than urinary proteins) shown in Tables 1-1 to 1-4 below, which are generally measured in clinical examination, falls outside a threshold range. More preferably, the kidney disease marker is at least one member selected from the group consisting of serum urea nitrogen, serum creatinine, serum inorganic phosphorus, fibrinogen in urine, creatinine clearance, 24-hour creatinine clearance estimated glomerular filtration rate (eGFR), urea clearance, inulin clearance, sodium thiosulfate clearance, renal plasma flow, filtration fraction, fractional excretion of sodium, fractional excretion of lithium, phenolsulfonphthalein test, concentration test, dilution test, free water clearance, free water reabsorption, maximal tubular excretory capacity, maximal tubular reabsorption capacity, rate of phosphate reabsorption, β2-microglobulin, and α1-microglobulin.

TABLE 1-1

| | | | | Measurement |
|---|---|---|---|---|
| | Item | Threshold | Unit | method |
| Serum | Total protein | 6.7 to 7.8 | g/dl | Biuret method |
| | Albumin | 3.8 to 5.3 | mg/dl | BCG method |
| | Urea | 8 to 20 | mg/dl | Urease-GLDH |
| | nitrogen | | | method |
| | Creatinine | Male: 0.6 to 1.0 | mg/dl | Enzymatic |
| | | Female: 0.4 to 0.8 | | method |
| | Uric acid | Male: 3 to 7.7 | mg/dl | Uricase-POD |
| | | Female: 2 to 7.7 | | method |
| | Ammonia | 12 to 66 | μg/dl | GLDH method |
| | Sodium | 136 to 145 | mEq/l | ISE |
| | Potassium | 3.4 to 4.5 | mEq/l | ISE |
| | Chlorine | 100 to 108 | mEq/l | ISE |
| | Total | 8.6 to 10.1 | mg/dl | OCPC method |
| | calcium | | | |
| | Magnesium | 1.8 to 2.3 | mg/dl | Enzymatic |
| | | | | method |
| | Inorganic | Adult: 2.2 to 4.1 | mg/dl | Enzymatic |
| | phosphorus | Child: 4.0 to 7.0 | | method |
| | Copper | 71 to 132 | μg/dl | Chelate |
| | | | | colorimetric |
| | | | | method |
| | Amylase | 40 to 126 | IU/l | JSCC |
| | | | | standardization |
| | | | | corresponding |
| | | | | method |
| | FGF23 | Full-length assay | pg/ml | ELISA |
| | | threshold: 10 to 50 | | |
| | | C-terminal assay | RU/ml | |
| | | threshold: 150 | | |
| Whole | Red blood | Male: 414 to 563 | ×10⁴/μl | Electrical |
| blood | cell count | Female: 373 to 495 | | resistance- |
| | | | | type automatic |
| | | | | blood cell |
| | | | | counter |
| | Hemoglobin | Male: 12.9 to 17.4 | g/dl | Oxyhemoglobin |
| | | Female: 10.7 to 15.3 | | method |
| | Pyruvic | 0.30 to 0.94 | mg/dl | Enzymatic |
| | acid | | | method |

TABLE 1-2

| | Item | Threshold | Unit | Measurement method |
|---|---|---|---|---|
| Arterial blood | O₂ saturation SₐO₂ | 94 to 99 | % | |
| gas analysis/ | O₂ partial pressure PₐO₂ | 80 to 100 | Torr | |
| acid-base | CO₂ partial pressure PₐCO₂ | 35 to 45 | Torr | |
| equilibrium | pH | 7.35 to 7.45 | | |
| | HCO₃— | 22 to 26 | mEq/l | |
| | Base excess (BE) | −2.2 to +2.2 | mEq/l | |
| | Buffer base (BB) | 46 to 52 | mEq/l | |
| | Standard bicarbonate (SB) | 21 to 25 | mEq/l | |
| Urine | Urinary output | 600 to 1,600 | ml/day | |
| | Specific gravity (spot urine) | 1,006 to 1,030 | | |
| | pH | 4.5 to 7.5 | | |
| | Urinary protein | 20 to 120 | mg/day | Pyrogallol red-Mo |
| | | | | coloring method |
| | Albumin | 5.7 ± 2.6 | mg/day | |
| | Glucose | 2 to 20 | mg/dl | |
| Urinary | Red blood cell count | <5 | /400x field | |
| sediment | Leukocyte count | <5 | | |
| | Epithelial cell count | Less than 1 (excluding | | |
| | | squamous epithelium) | | |
| | Cast count | <1 | | |

TABLE 1-3

| | Item | Threshold | Unit | Measurement method |
|---|---|---|---|---|
| Kidney | Creatinine clearance (Ccr) | 70 to 130 | ml/min | |
| function | 24-Hour creatinine clearance | Male: 62 to 108 | ml/min | |
| | | Female: 57 to 78 | | |
| | Glomerular filtration rate (GFR) | Male: 129 ± 26 | ml/min | |
| | | Female: 97 ± 13 | | |
| | Urea clearance | Maximum clearance: 62 to 77 | ml/min | |
| | | Standard clearance: 45 to 55 | | |
| | Inulin clearance (GFR) | Male: 72 to 176 | ml/min/1.73 m² | |
| | | Female: 81 to 137 | ml/min/1.73 m² | |
| | Sodium thiosulfate clearance | Male: 90 to 138 | ml/min | |
| | | Female: 86 to 120 | | |
| | Renal plasma flow (RPF) | 350 to 650 | ml/min | C_{PAII} |
| | Filtration fraction (FF) | 0.18 to 0.22 | | GRF/RPF |
| | Fractional excretion of sodium | 1≤ | % | |
| | Fractional excretion of lithium | 20 to 30 | % | |
| | Phenolsulfonphthalein (PSP) test | ≤100 | mOsm/kg | |
| | | 15 min value: ≥25 | % | |
| | | 120 min value: ≥55 | % | |
| | Concentration test | ≥1.025 | (Specific gravity) | Fishberg |
| | Dilution test | ≤1.006 | (Specific gravity) | Fishberg |
| | Free water clearance | At the time of water | ml/min | |
| | | diuresis: 13 to 15 | | |
| | Free water reabsorption | At the time of | ml/min | |
| | | concentration: 1.5 to 2.0 | | |
| | Maximal tubular excretory capacity | 81 ± 11 | mg/min/1.48 m³ | T_{mPAII} |
| | Maximal tubular reabsorption capacity | 340 ± 18 | mg/min/1.48 m³ | T_{mPAII} |
| | | 80 to 96 | % | % TRP |
| | | Serum: 0.8 to 0.2 | mg/l | LPIA |
| | β₂-Microglobulin | Urine: 11 to 253 | μg/day | LPIA |
| | | (30 to 340) | (μg/l) | |
| | α₁-Microglobulin | Serum: 10 to 30 | mg/l | EIA |
| | | Urine: 1.8 ± 0.9 | mg/l | EIA |

TABLE 1-4

| | | | | Measurement |
|---|---|---|---|---|
| | Item | Threshold | Unit | method |
| In blood | Fibrinogen* | 200 to 400 | mg/dl | Thrombin time |
| | | | | method |
| | Serum C3 | 86 to 160 | mg/dl | LTIA method** |
| | Serum C4 | 17 to 45 | mg/dl | LTIA method |
| Urine | Fibrinogen | Below detection limit | | ELISA method |
| | C3 | Below detection limit | | LTIA method |
| | C4 | Below detection limit | | LTIA method |

*In plasma (anticoagulant is citric acid salt)
**LTIA method: latex turbidimetry

The kidney disease markers described above can be measured according to known methods described in, for example, Kanai's Manual of Clinical Laboratory Medicine, Revised 32nd Edition (edited by Masamitsu Kanai; Kanehara & Co., Ltd.).

“Chronic kidney disease” as used herein refers to, when the subject is a human, a condition in which kidney damage (for example, urine abnormalities such as proteinuria including microalbuminuria, abnormal urinary sediment, imaging abnormalities such as a single kidney and polycystic kidney disease, decreased kidney function such as increased serum creatinine, electrolyte abnormalities such as hypokalemia due to tubular damage, abnormalities in histopathological examination such as renal biopsy), or decreased kidney function, i.e., an estimated GFR (glomerular filtration rate) of less than 60 mL/min/1.73 m², persists for 3 months or more, according to the Clinical Practice Guidebook for Diagnosis and Treatment of Chronic Kidney Disease 2012 (edited by the Japanese Society of Nephrology).

Here, the estimated GFR (eGFR) can be calculated using the estimation formulas (eGFRcreat) from a serum creatinine value shown in Table 2 below. The estimation formulas (eGFRcys) based on serum cystatin C can be applied to those who have extremely low muscle mass, such as lower-extremity amputees.

TABLE 2

| Male | eGFRcreat (mL/min/1.73 m²) = 194 × Cr^{-1.094} × age^{-0.287} |
|---|---|
| | eGFRcys (mL/min/1.73 m²) = 104 × Cys-C^{-1.019} × 0.996^{age}) − 8 |
| Female | eGFRcreat (mL/min/1.73 m²) = 194 × Cr^{-1.094} × age^{-0.287} × |
| | 0.739 |
| | eGFRcys (mL/min/1.73 m²) = (104 × Cys-C^{-1.019} × 0.996^{age} × |
| | 0.929) − 8 |

*This evaluation of kidney function is performed for persons aged 18 or older.

For example, in the case in which protein is used as an index, when the results of a urine test 3 months or more prior and a recent urine test show that the subject has a persistent urinary protein level of 0.15 g/gCr or more, such a condition can be diagnosed as chronic kidney disease. When the subject has diabetes and the results of an albuminuria test 3 months or more prior and a recent albuminuria test show that the subject has a persistent urinary albumin level of 30 mg/gCr or more, such a condition can be diagnosed as chronic kidney disease.

For children, a threshold of serum creatinine (Cr) can be determined by using an enzymatic method for Japanese children, and used to evaluate children with kidney function abnormalities. For example, the eGFR in % for children aged 2 or older but 11 or younger can be represented by equation 1 below.

eGFR (%)=(0.3×body height (m)/serum Cr value in subject)×100 Equation 1

In the case of non-human mammals, such as cats and dogs, it can be predicted whether a non-human mammal has chronic kidney disease from, for example, average daily water intake or urine specific gravity.

The severity of chronic kidney disease can be determined based on, for example, Table 3 below in the case of humans (Table 3 is Table 2 in the Clinical Practice Guidebook for Diagnosis and Treatment of Chronic Kidney Disease, 2012).

“Individual” as used herein is not particularly limited, and includes humans and non-human mammals. Examples of non-human mammals include bovines, horses, sheep, goats, pigs, dogs, cats, rabbits, monkeys, and the like. Humans, cats, and dogs are preferable. There is no limitation on the age or sex of the individual.

“Subject” may be an individual with a history of decreased kidney function or other kidney disease, or may be an individual with no history of decreased kidney function or other kidney disease. The subject may be an individual with symptoms, such as polyuria, thirst, increased water intake, excessive gastric juice, vomiting, bloody urine, and general malaise; or may be an individual without symptoms. Further, the subject also includes a subject suspected of having kidney damage or chronic kidney disease according to a known diagnostic method, such as a medical interview, a urine test, a biochemical test of blood, kidney diagnostic imaging, or a renal biopsy.

“Specimen” as used herein includes cells, tissue (adrenal glands, aorta, brain, lungs, pancreas, hypophysis, skin, skull, skeletal muscle, spleen, testes, thyroid gland, kidneys, large intestine, eyeballs, heart, liver, salivary glands, thymus, adipose tissue, stomach, jejunum, ileum, and the like), body fluids (sweat, secretion from skin, lacrimal fluid, saliva, spinal fluid, ascites fluid, and pleural effusion), urine, blood samples, and the like, derived from a living organism. As specimens, kidneys, adipose tissue, skin, hair roots, salivary glands (parotid glands, submandibular glands, and sublingual glands, and preferably parotid glands), sweat, secretion from skin, lacrimal fluid, saliva, urine, and blood samples are preferable, and kidneys, saliva, parotid glands, adipose tissue, hair roots, skin, secretion from skin, and sweat are more preferable.

Moreover, when the kidney function prediction markers described later are PRPs, it is preferable to use saliva or a salivary gland as a specimen. When the kidney function prediction markers are defensins and Hamp2, adipose tissue, hair roots, skin, secretion from skin, and sweat are preferable as specimens.

In particular, skin is preferable as a specimen for obtaining the measurement value of Defb8; stomach, skeletal muscle, or testes are preferable as a specimen for obtaining the measurement value of Defa24; adipose tissue is preferable as a specimen for obtaining the measurement values of Defb1, Defb10, Defb12, Defb14, Defb15, Defb18, Defb19, Defb2, Defb20, Defb21, Defb22, Defb23, Defb25, Defb26, Defb28, Defb29, Defb30, Defb35, Defb37, Defb39, Defb41, Defb42, Defb43, Defb45, Defb47, and Defb48; skull is preferable as a specimen for obtaining the measurement value of Oscar; saliva, salivary glands, or parotid glands are preferable as a specimen for obtaining the measurement values of Prb1, Prh1, Prp2, and Prpmp5; skull, kidneys, or heart is preferable as a specimen for obtaining the measurement value of Spp1; kidneys, salivary glands, preferably parotid glands, are preferable as a specimen for obtaining the measurement value of Dnase1; aorta is preferable as a specimen for obtaining the measurement value of Slc7a8; thyroid gland is preferable as a specimen for obtaining the measurement value of Anpep; kidneys or liver is preferable as a specimen for obtaining the measurement value of Slco1a1; adrenal glands, aorta, lungs, hypophysis, skin, skull, skeletal muscle, spleen, thyroid gland, kidneys, heart, or adipose tissue is preferable as a specimen for obtaining the measurement value of Aplnr.

In the section “2. Method for obtaining each measurement value” described later, when a measurement value relating to a kidney function prediction marker protein is obtained, the specimen is preferably a blood sample or a body fluid. In the same section, when a measurement value of a kidney function prediction marker mRNA is obtained, the specimen is preferably tissue, a blood sample, or a body fluid.

“Blood sample” as used herein refers to blood (whole blood) collected from a subject, or serum or plasma prepared from the blood. The blood sample is preferably serum or plasma, and more preferably serum. The type of anticoagulant used for collecting plasma is not particularly limited. The type of blood sample of a subject used for measurement and the type of the blood sample used for determining a predetermined threshold may be the same or different, and are preferably the same. When plasma is used as a blood sample, it is preferable that plasma for determining a predetermined threshold is prepared from blood collected using the same anticoagulant as used for plasma of the subject.

Further, the specimen may be a fresh specimen, or may be a preserved specimen. When the specimen is preserved, it can be preserved in a room-temperature environment, a refrigerated environment, or a frozen environment; and cryopreservation is preferable.

The complication of chronic kidney disease in the present invention is not particularly limited, and includes any complication that can develop in chronic kidney disease. The complication of chronic kidney disease is preferably at least one member selected from the group consisting of urine concentrating ability disorders (including polyuria and urine of low specific gravity), azotemia (including high blood urea nitrogen, hypercreatininemia, hyperuricemia, and uremia), water/electrolyte abnormalities (including fluid overload and hyperkalemia), metabolic acidosis, renal anemia, and secondary hyperparathyroidism (including renal osteopathy).

“Phosphorus intake” as used herein refers to the amount of phosphorus taken from foods, drinks, etc. “Phosphorus” is not particularly limited, and is preferably inorganic phosphorus. According to “Dietary Reference Intakes for Japanese (2015)” (Ministry of Health, Labour and Welfare), the dietary reference intake for phosphorus is, in the case of men, 1,000 mg/day at 18 to 70 years of age, 1,200 mg/day at 12 to 17 years of age, 1,100 mg/day at 10 to 11 years of age, 1,100 mg/day at 8 to 9 years of age, and 900 mg/day at 6 to 7 years of age. In the case of women, the dietary reference intake for phosphorus is 800 mg/day at 18 to 70 years of age, 900 mg/day at 15 to 17 years of age, 1,100 mg/day at 12 to 14 years of age, 1,000 mg/day at 10 to 11 years of age, and 900 mg/day at 6 to 9 years of age. The tolerable upper intake level of dietary reference intake for phosphorus is 3,000 mg/day in both adult men and women. Thus, high “phosphorus intake” means 1,500 mg/day or more, preferably 3,000 mg/day or more.

The dietary therapy in the present invention is not particularly limited. Preferable examples include dietary therapies intended to limit protein intake, limit salt intake, limit potassium intake, limit fluid intake, etc.

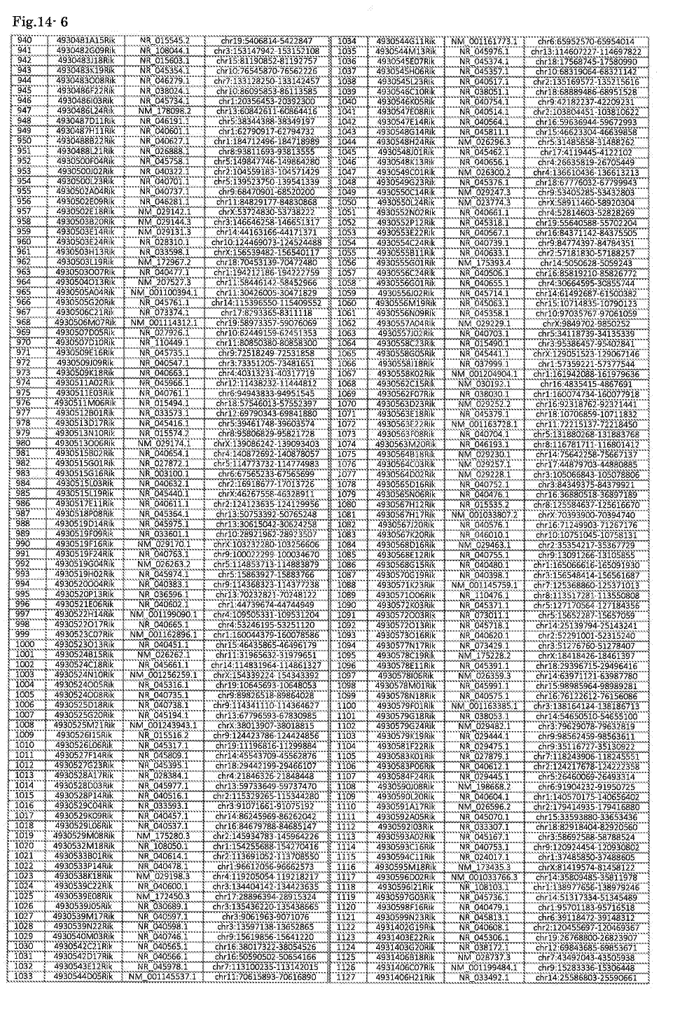

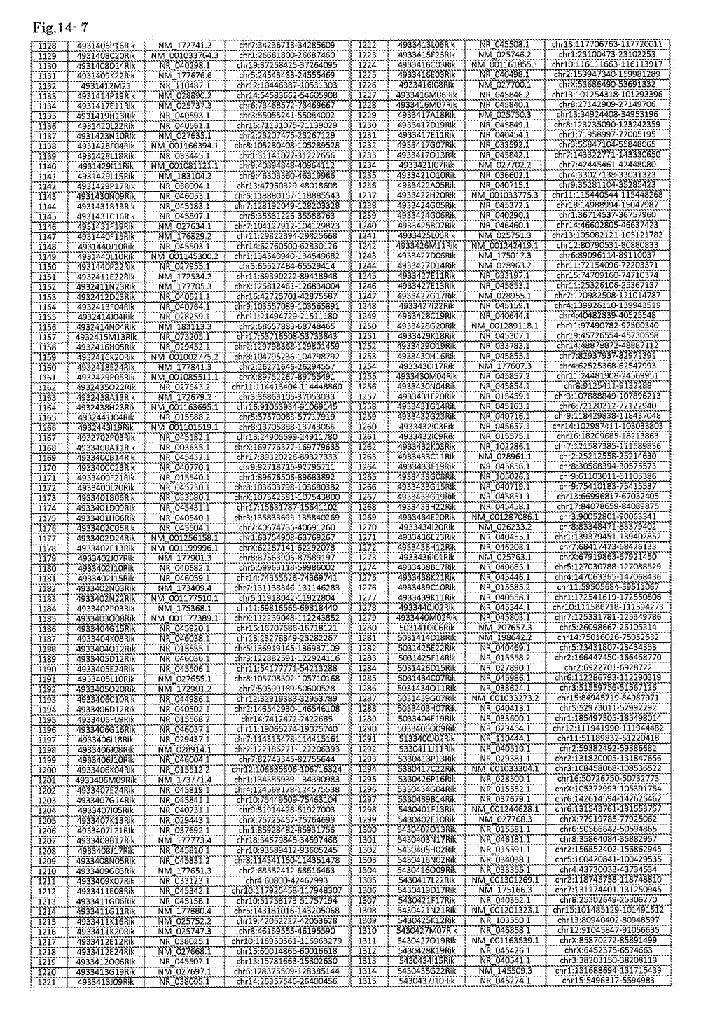

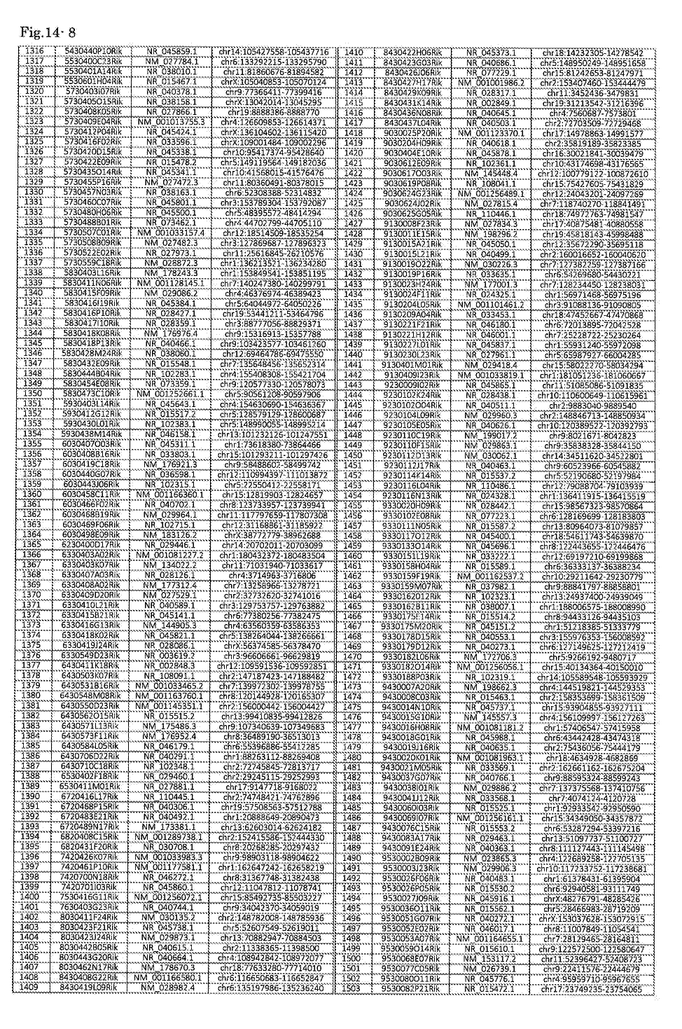

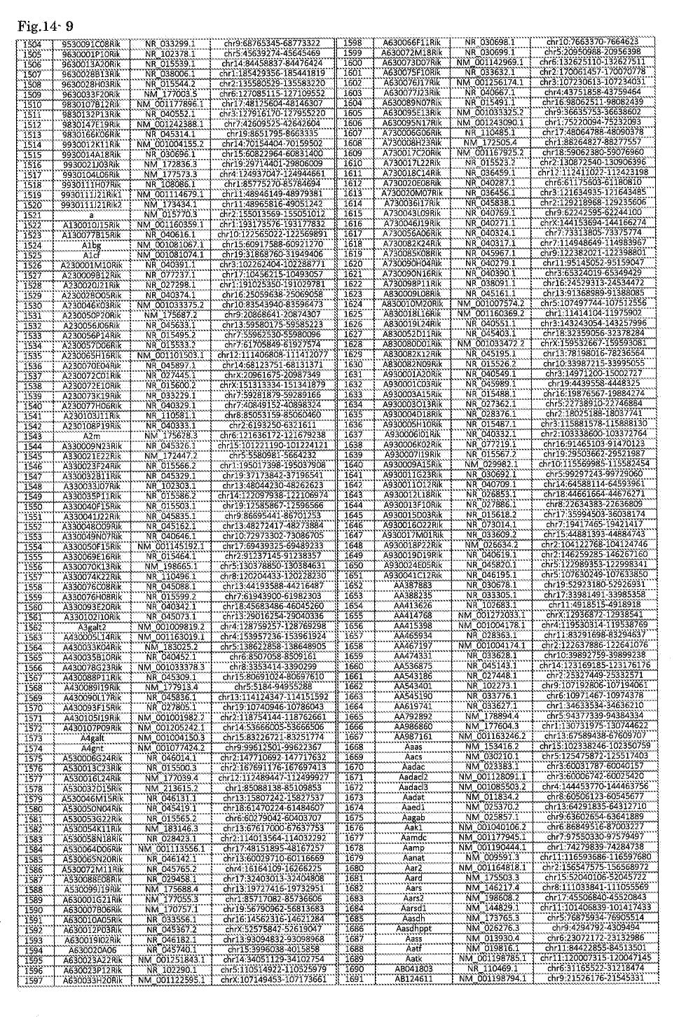

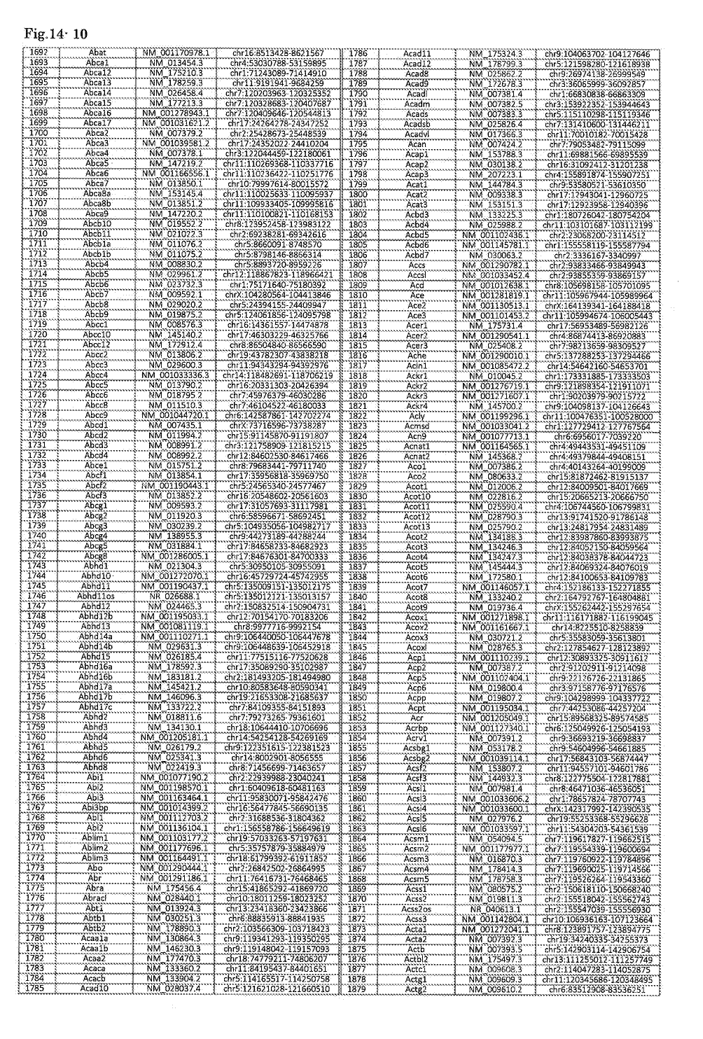

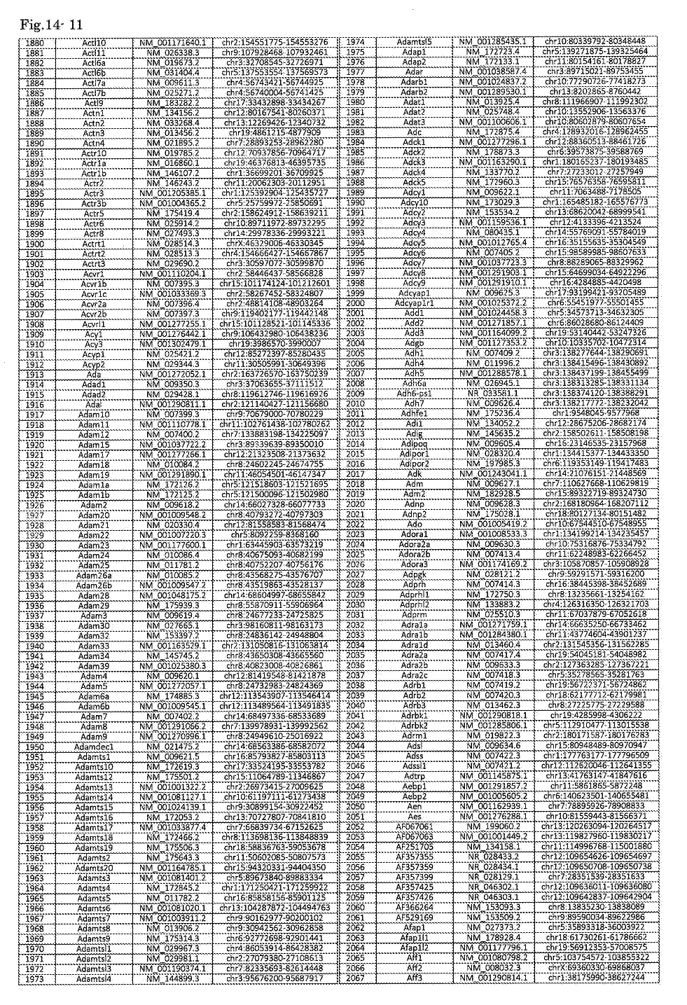

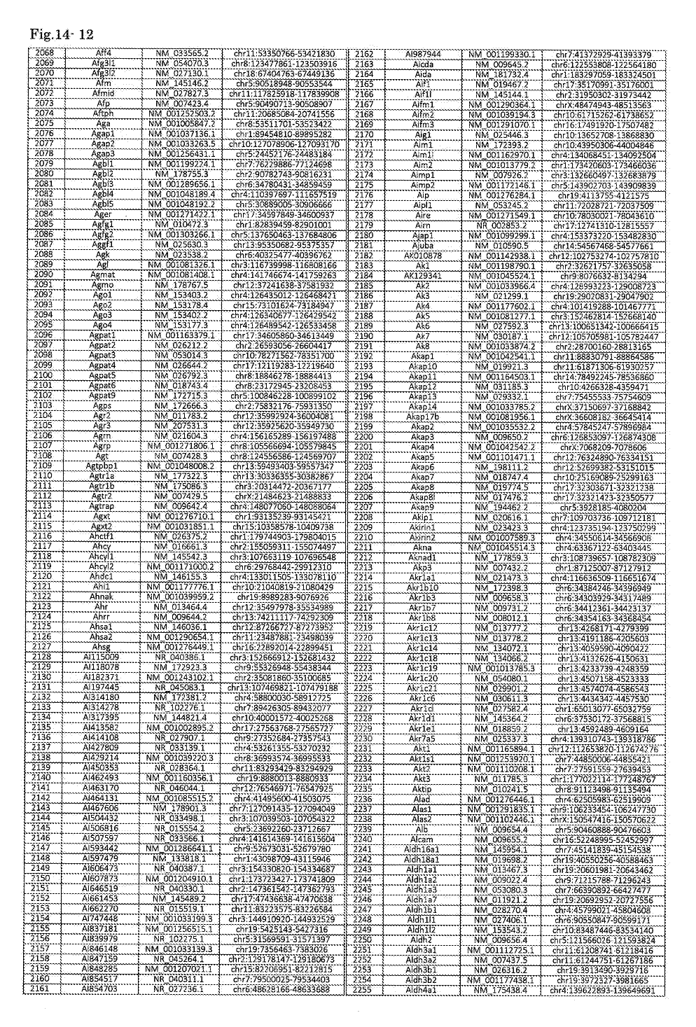

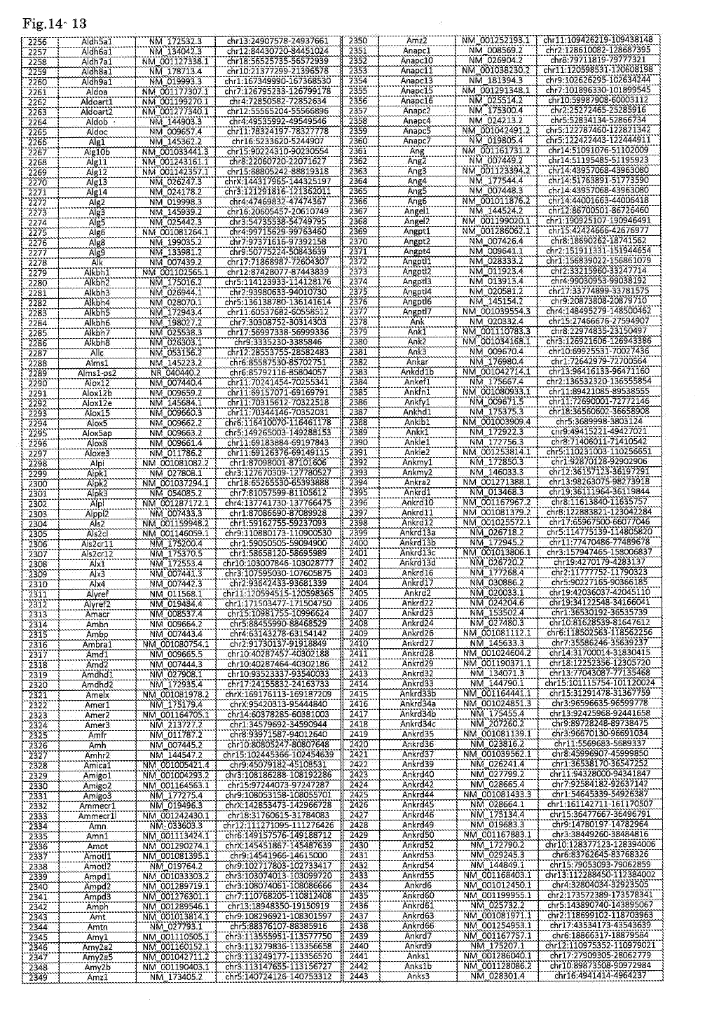

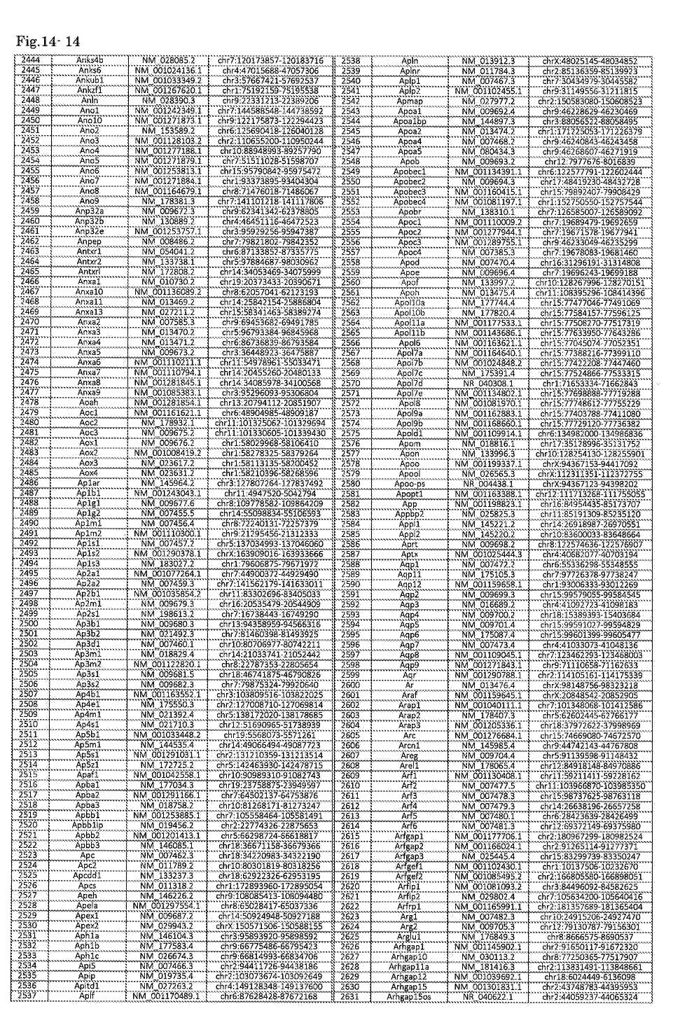

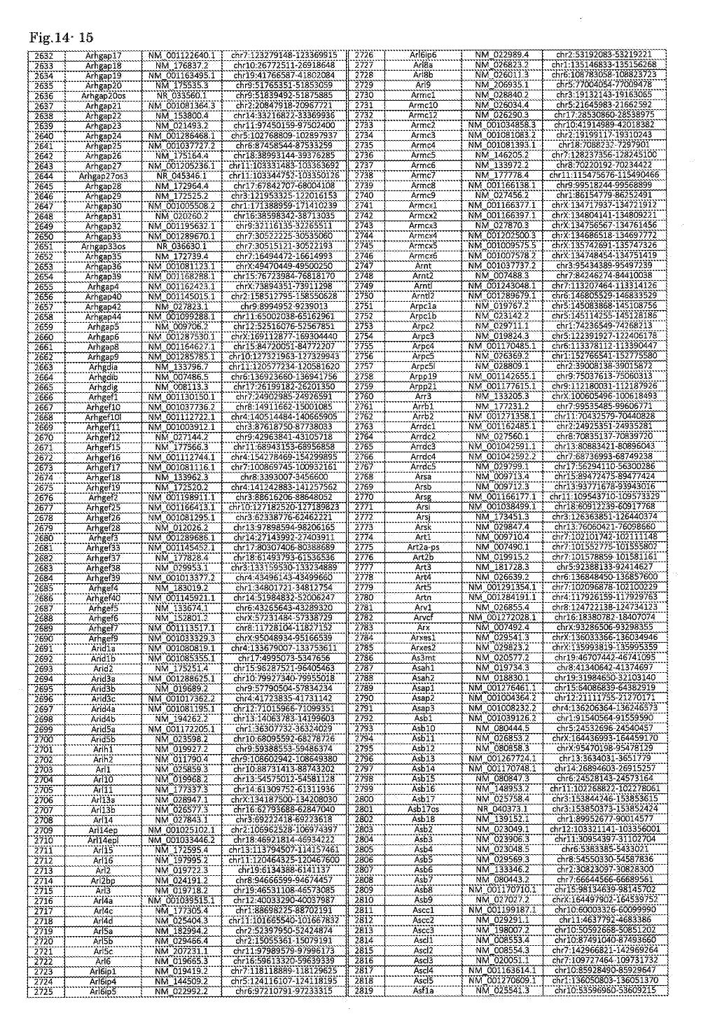

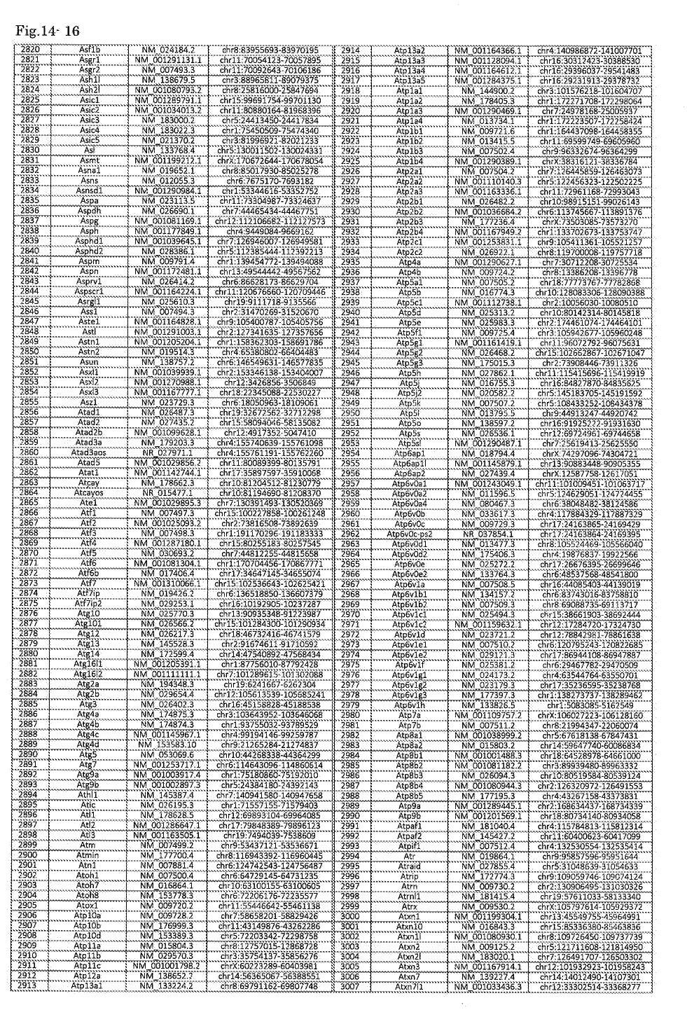

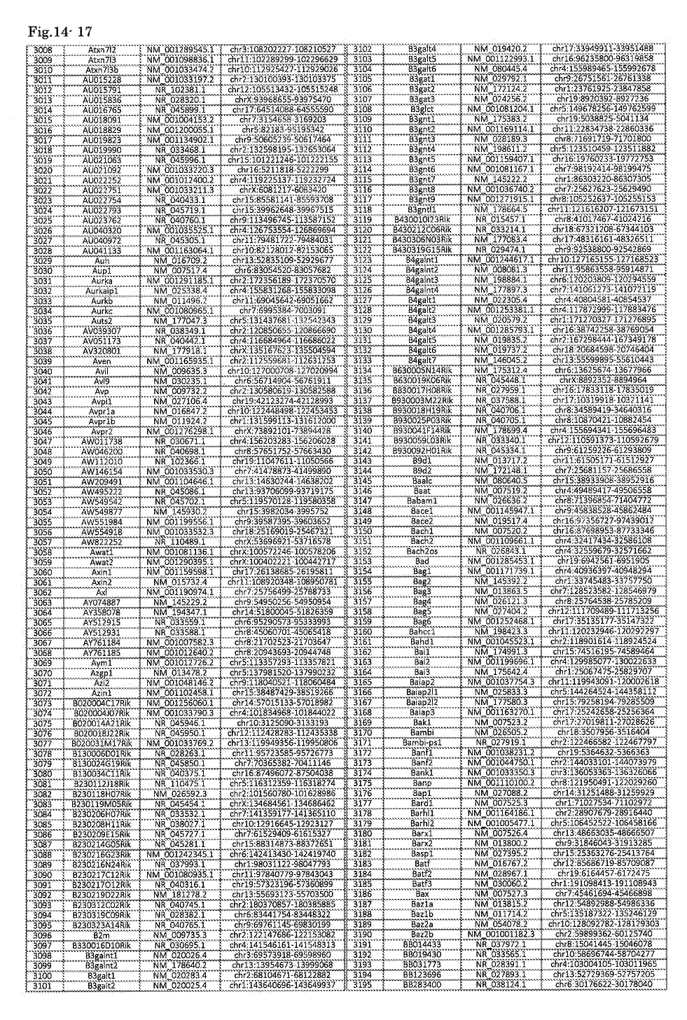

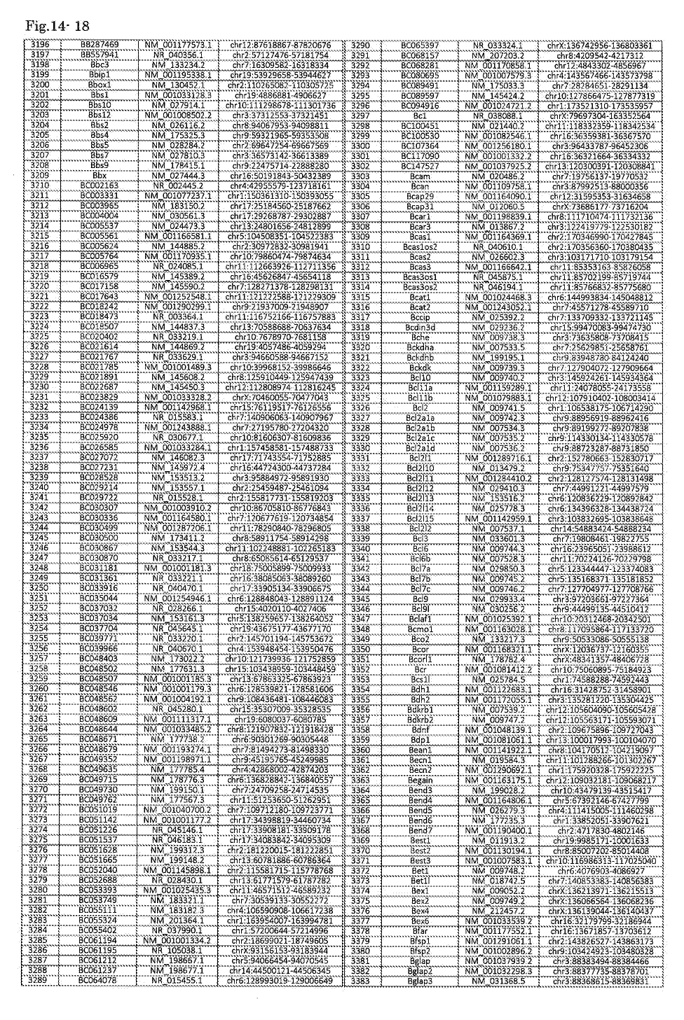

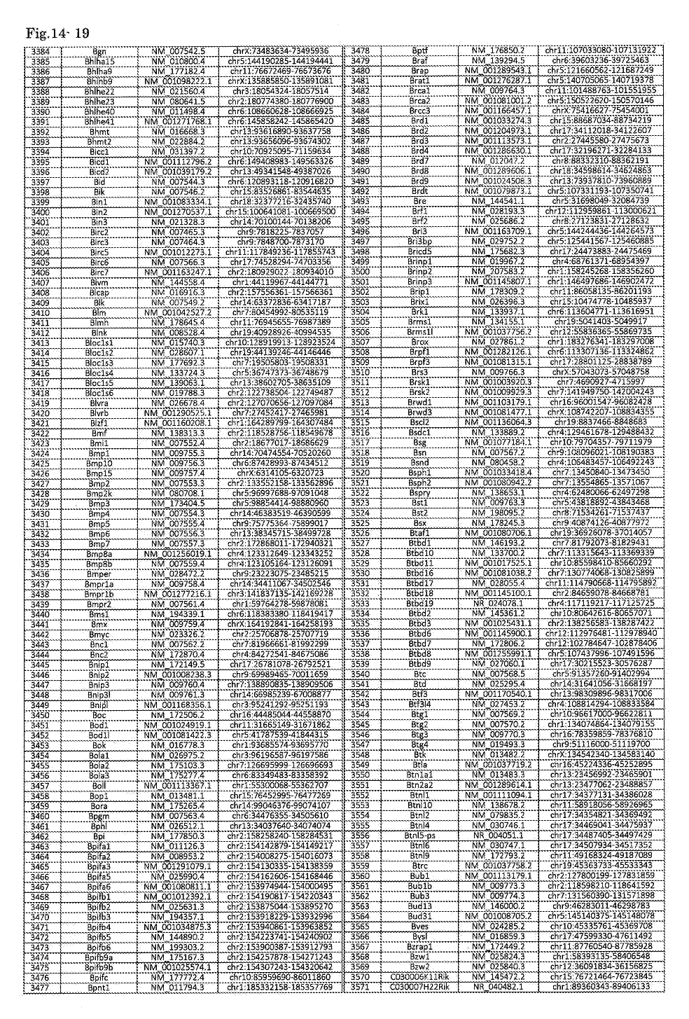

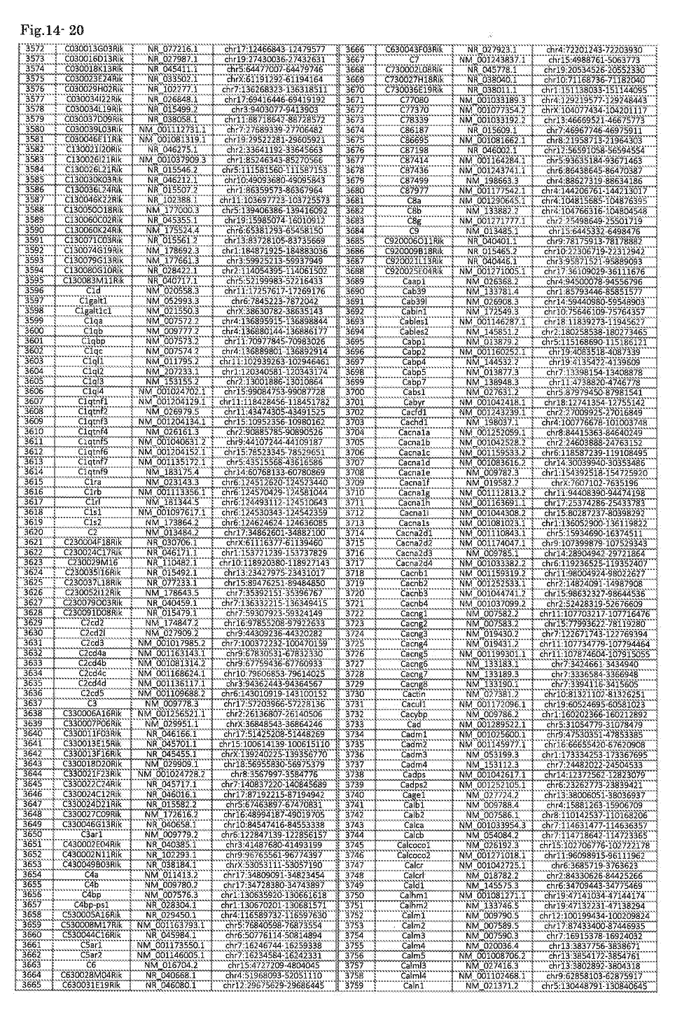

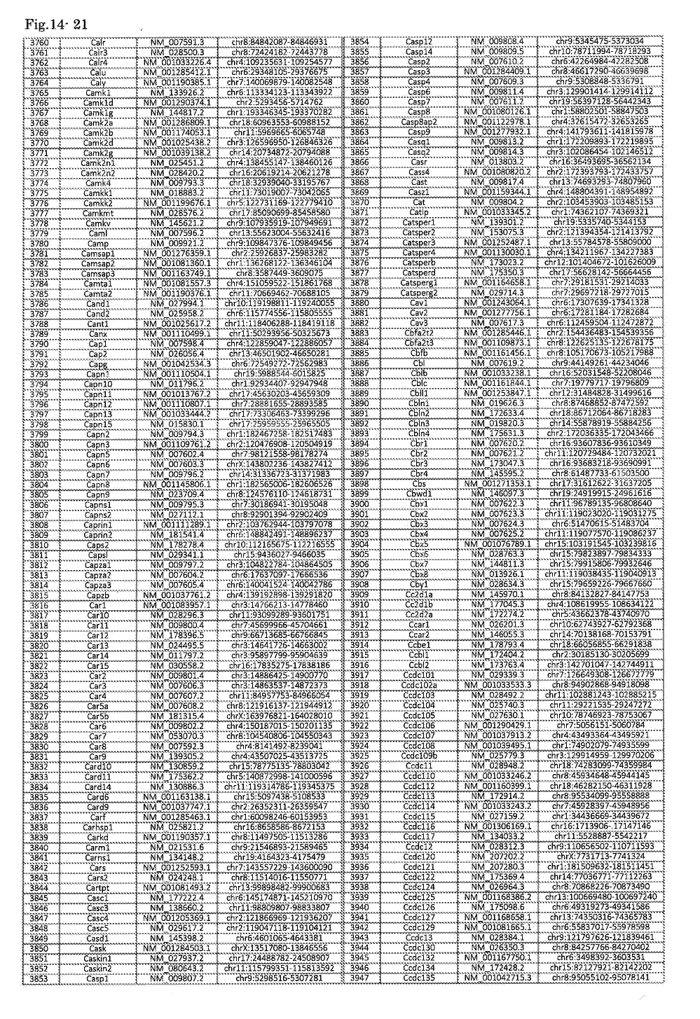

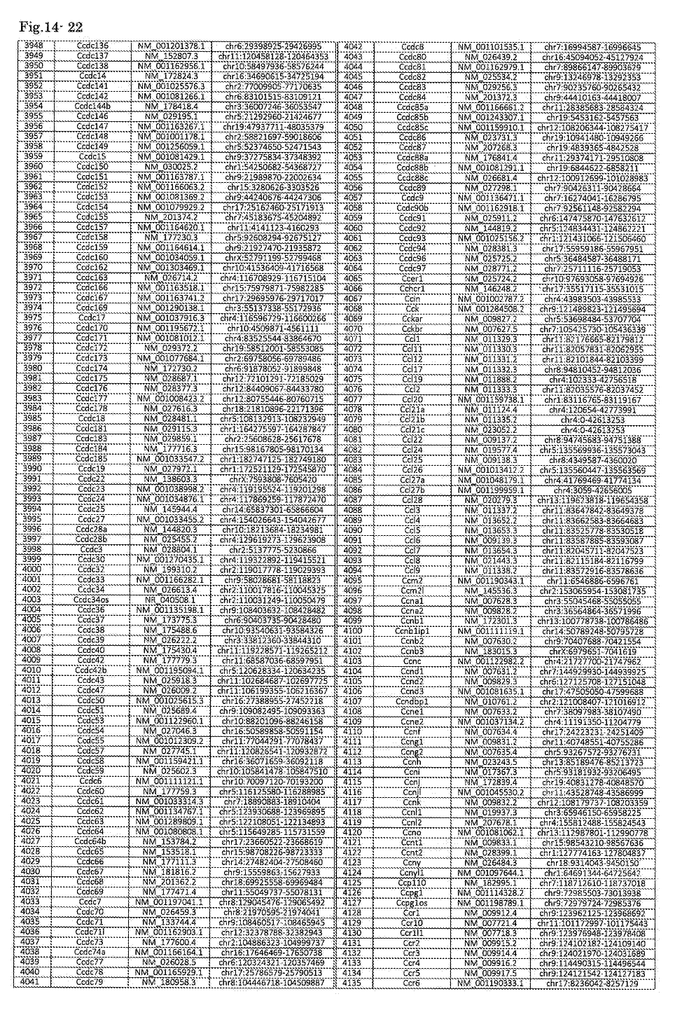

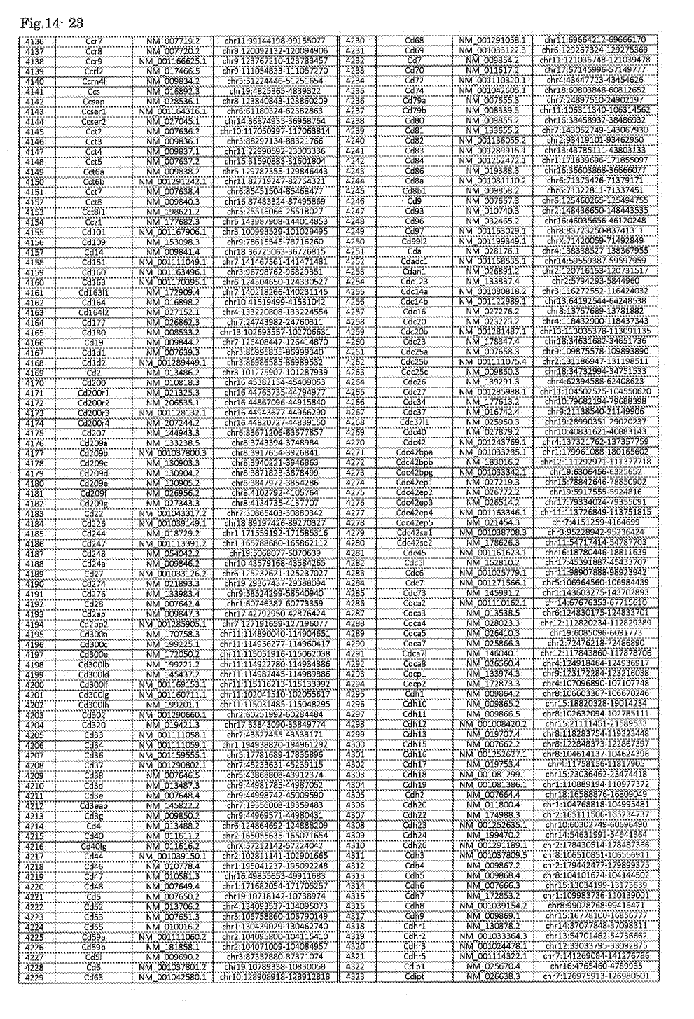

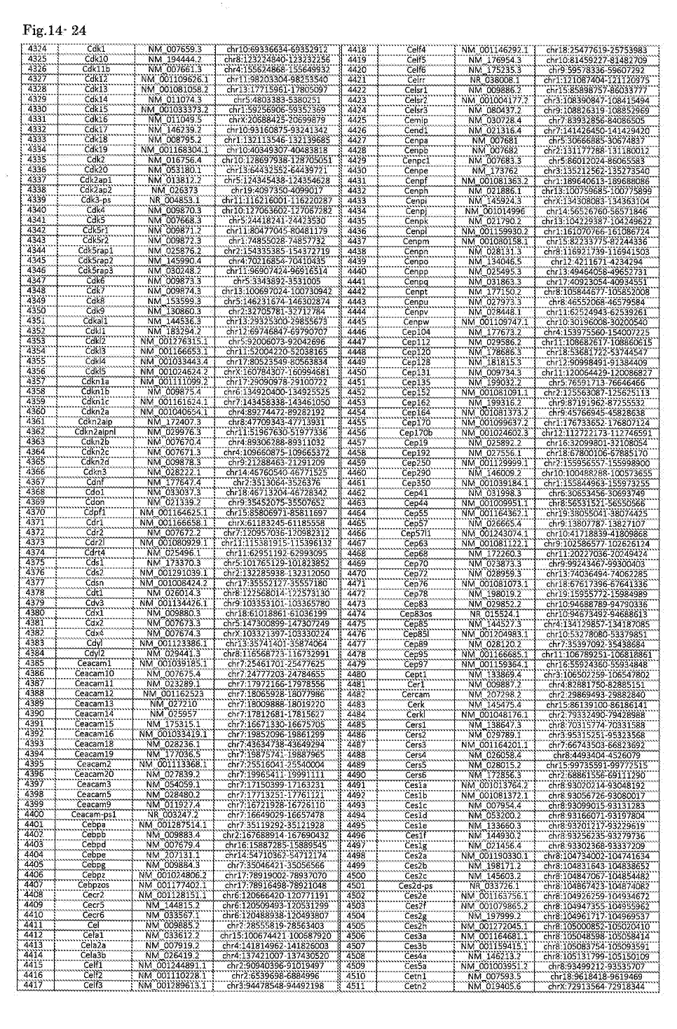

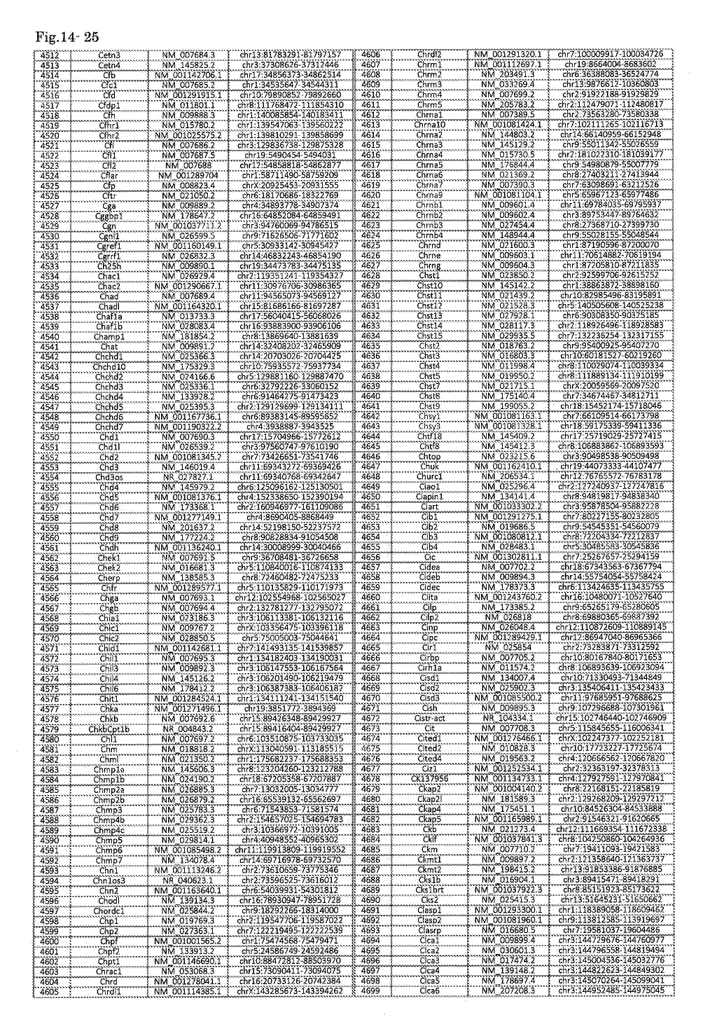

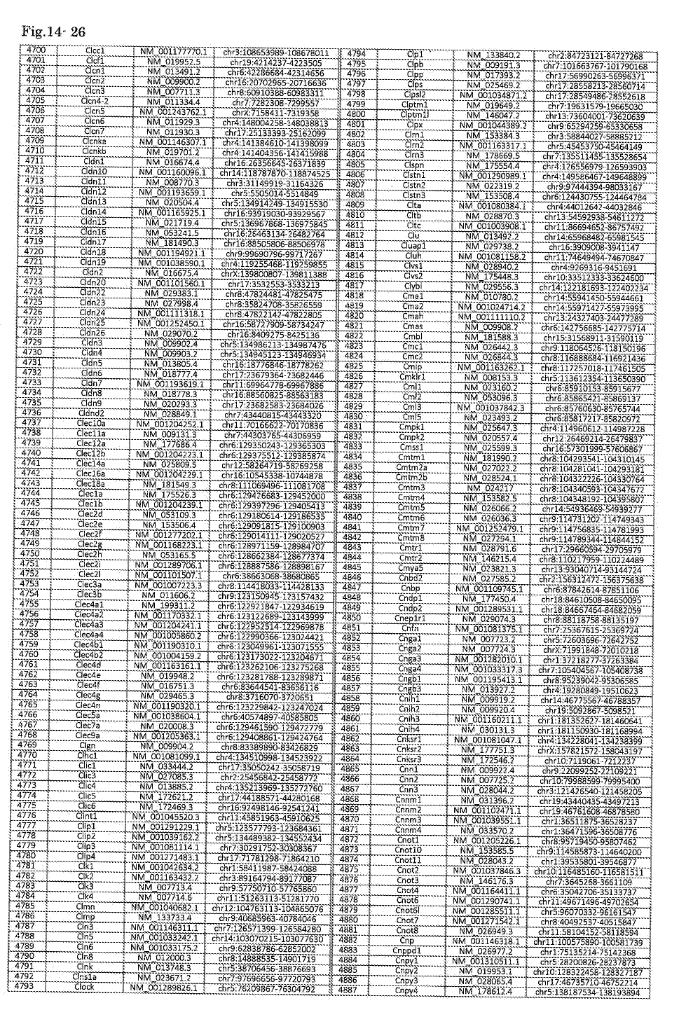

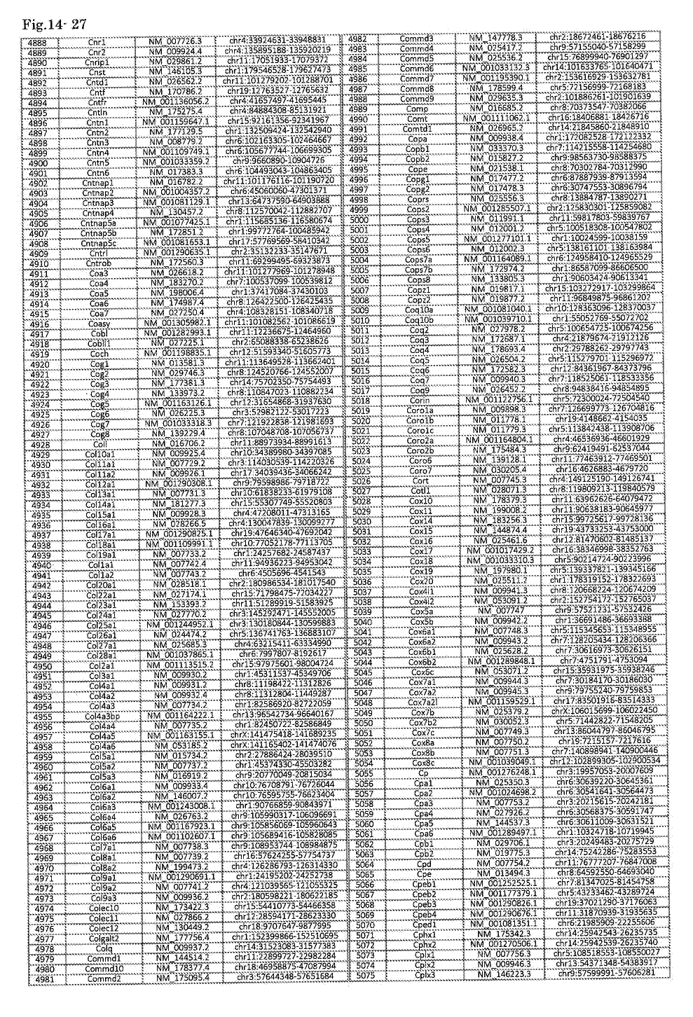

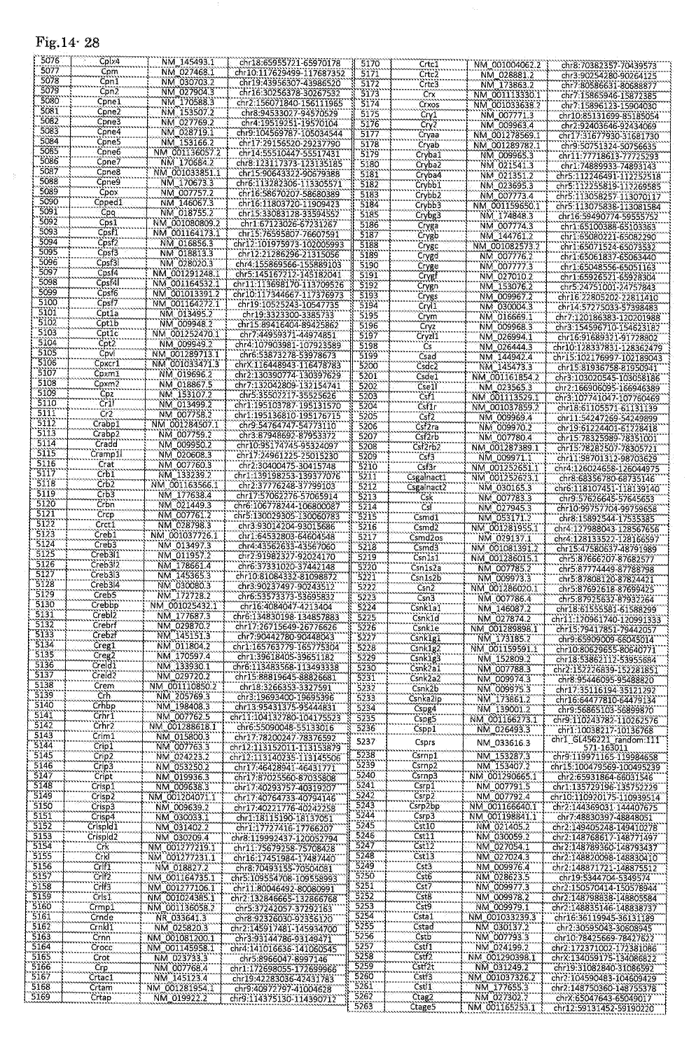

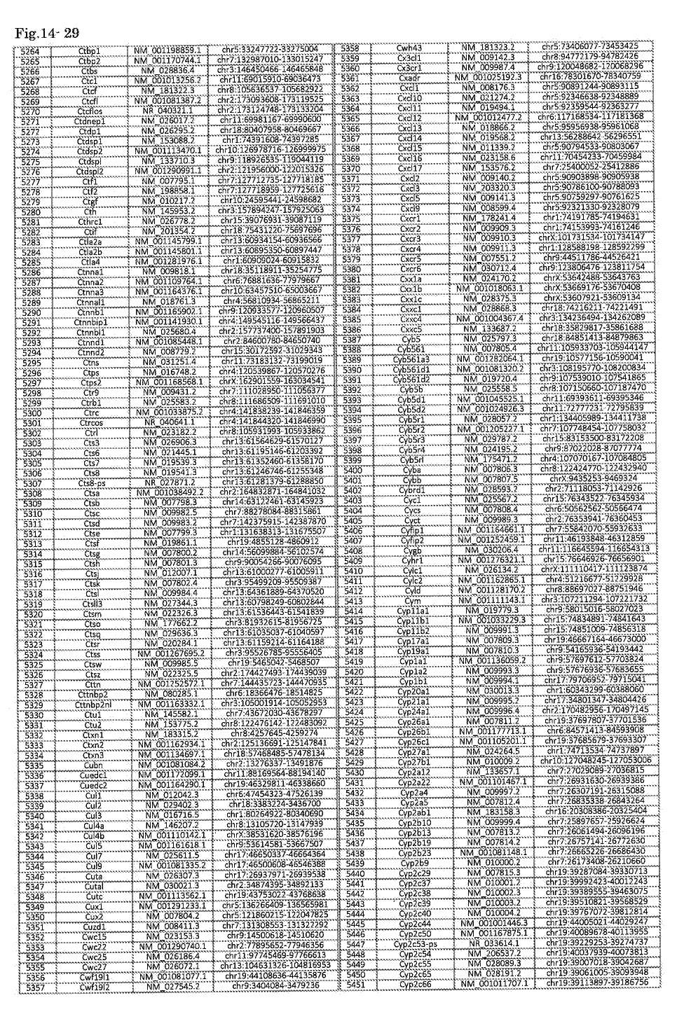

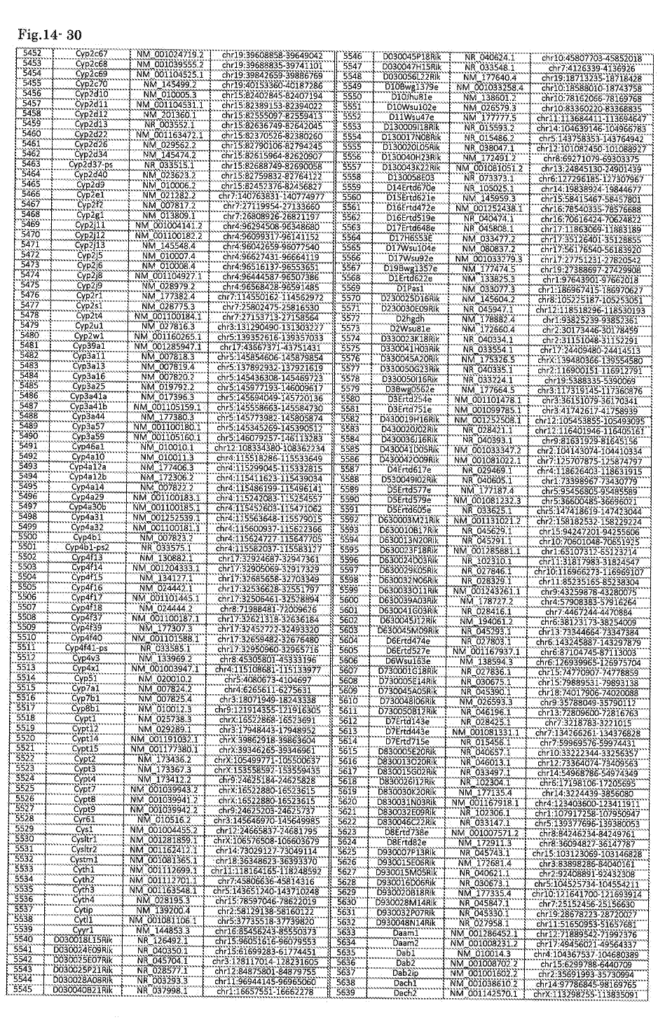

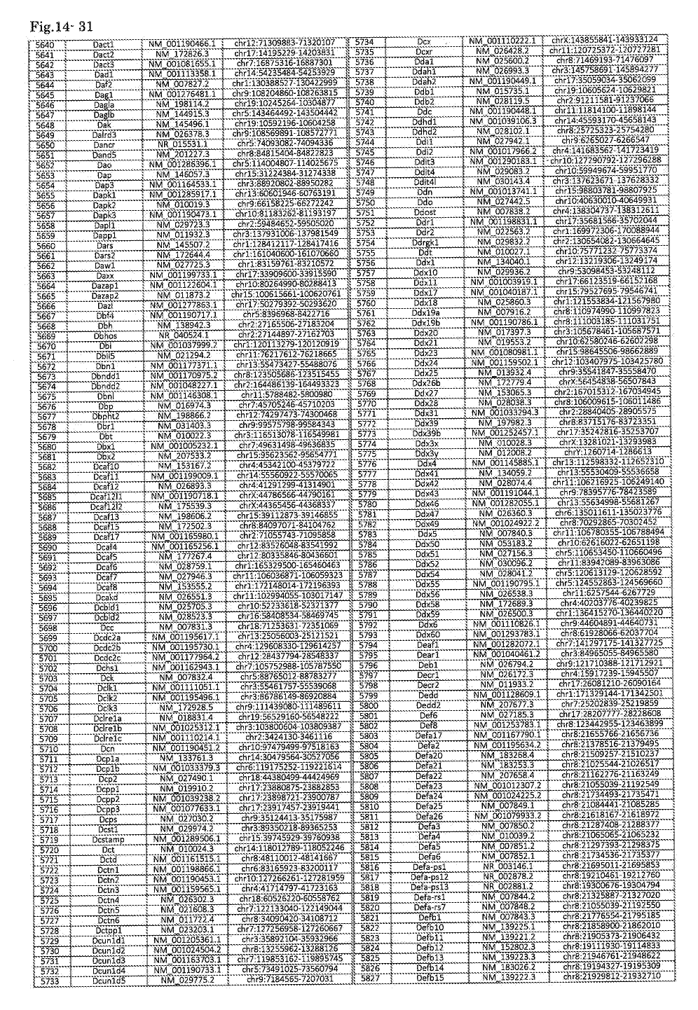

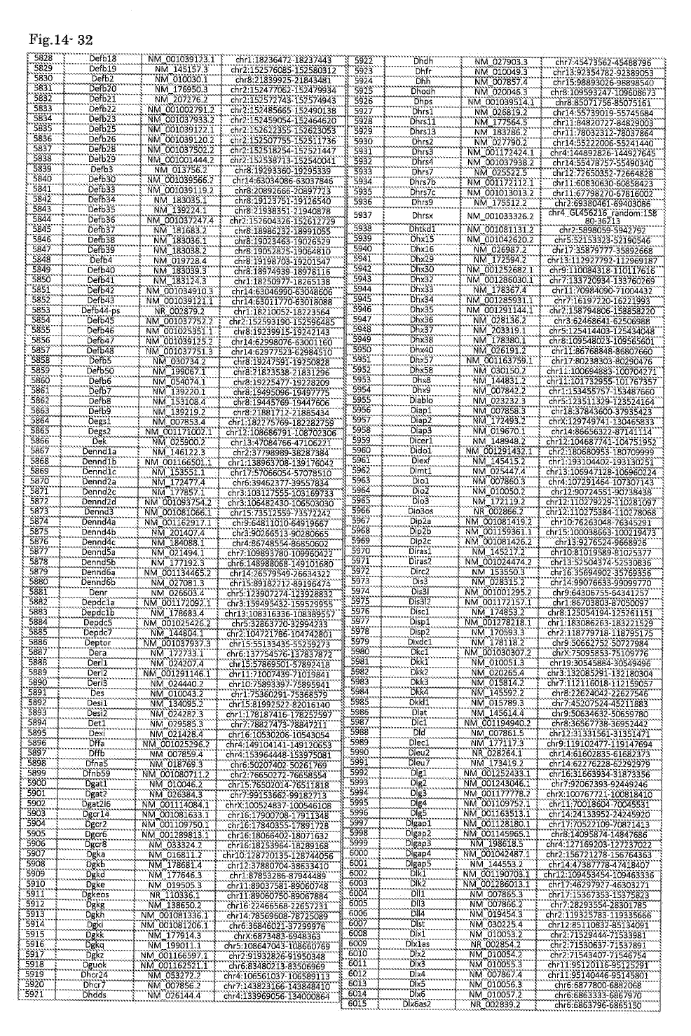

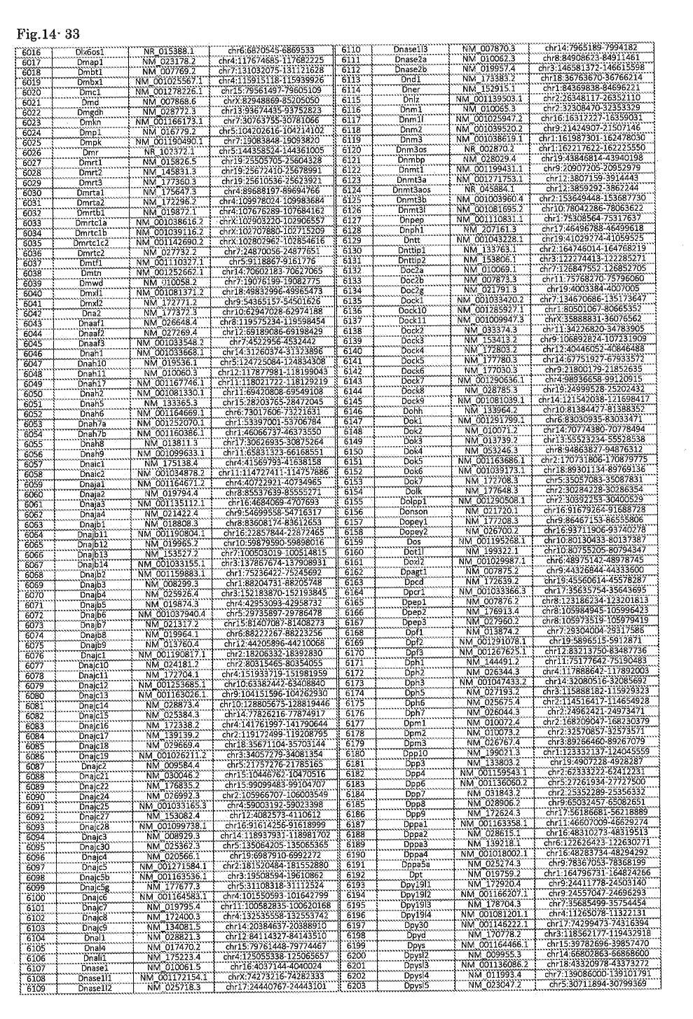

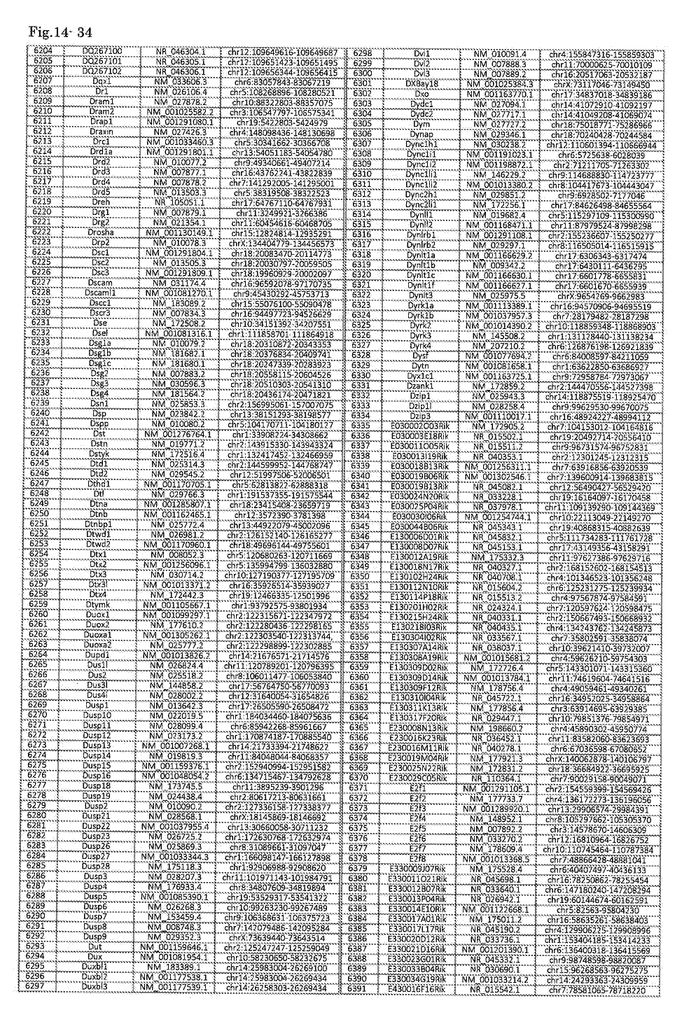

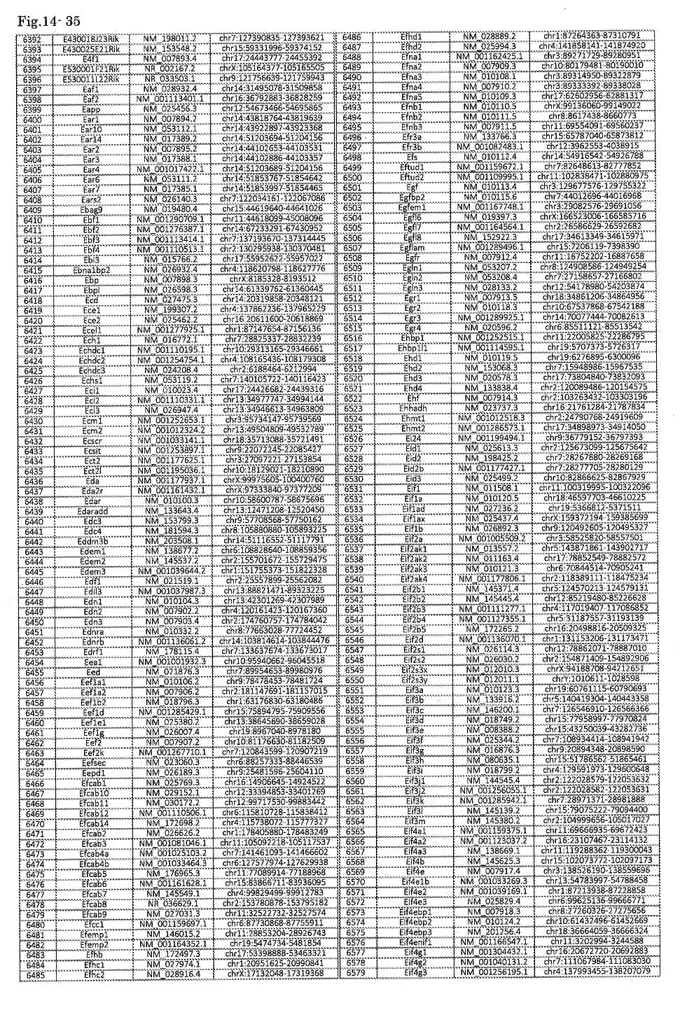

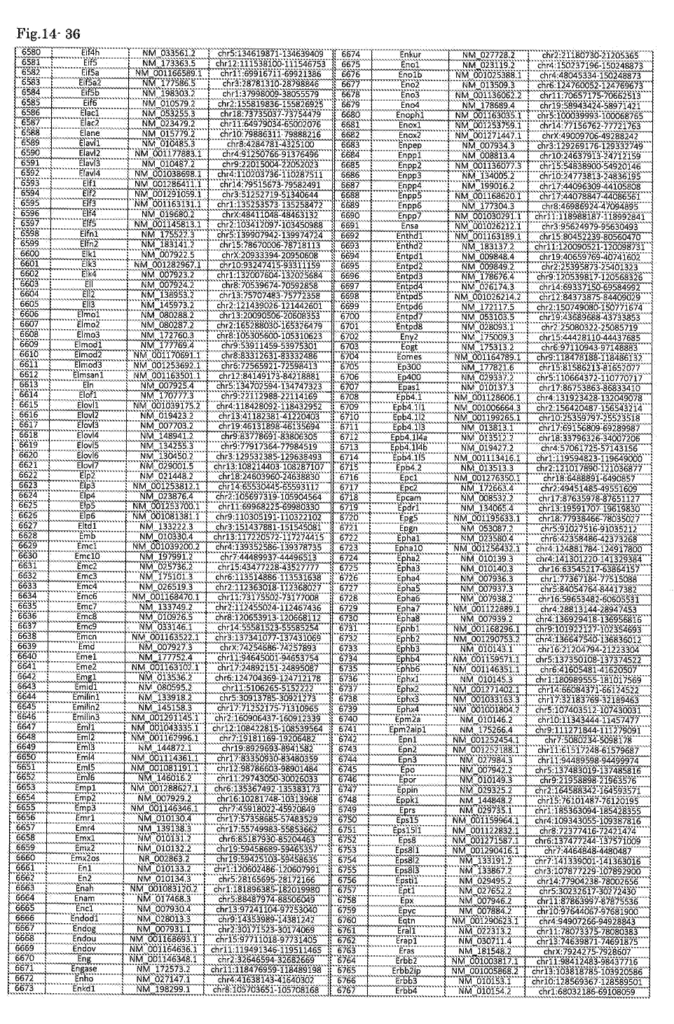

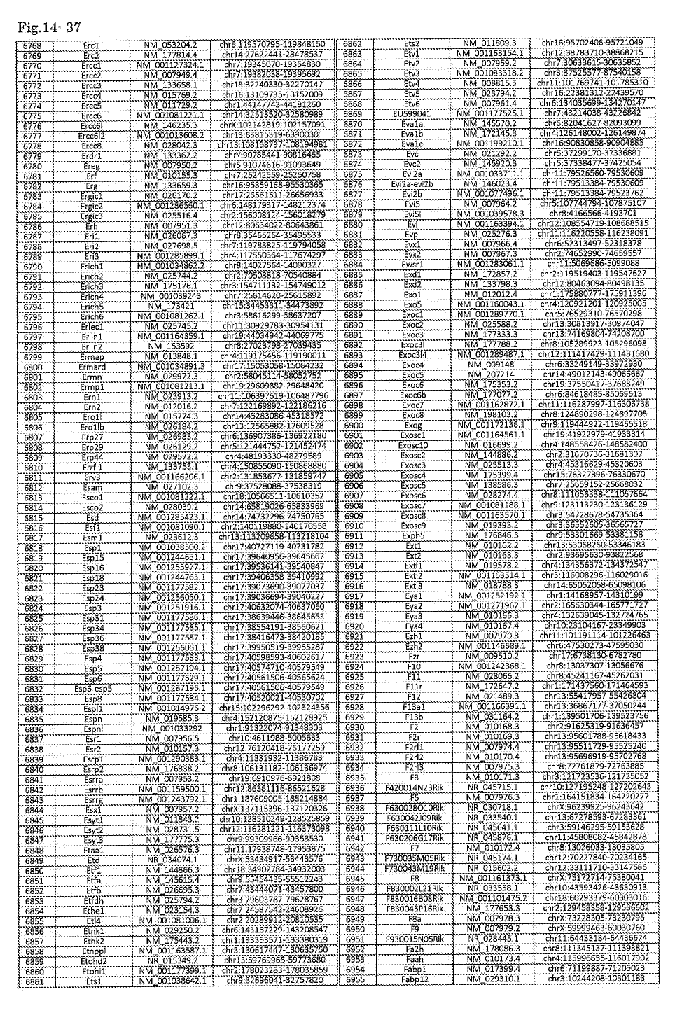

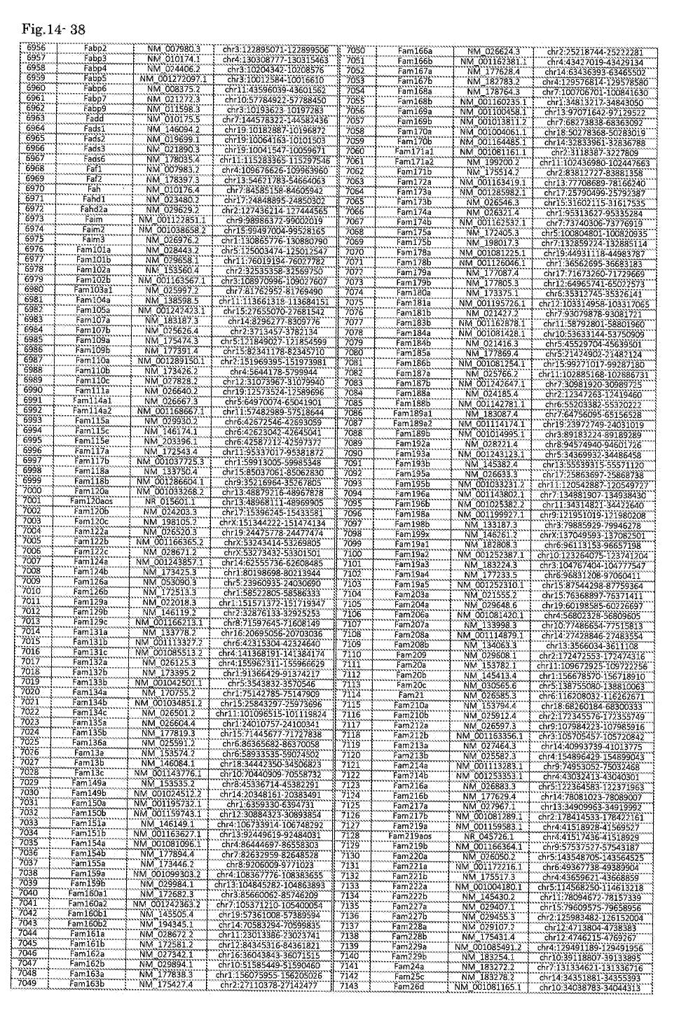

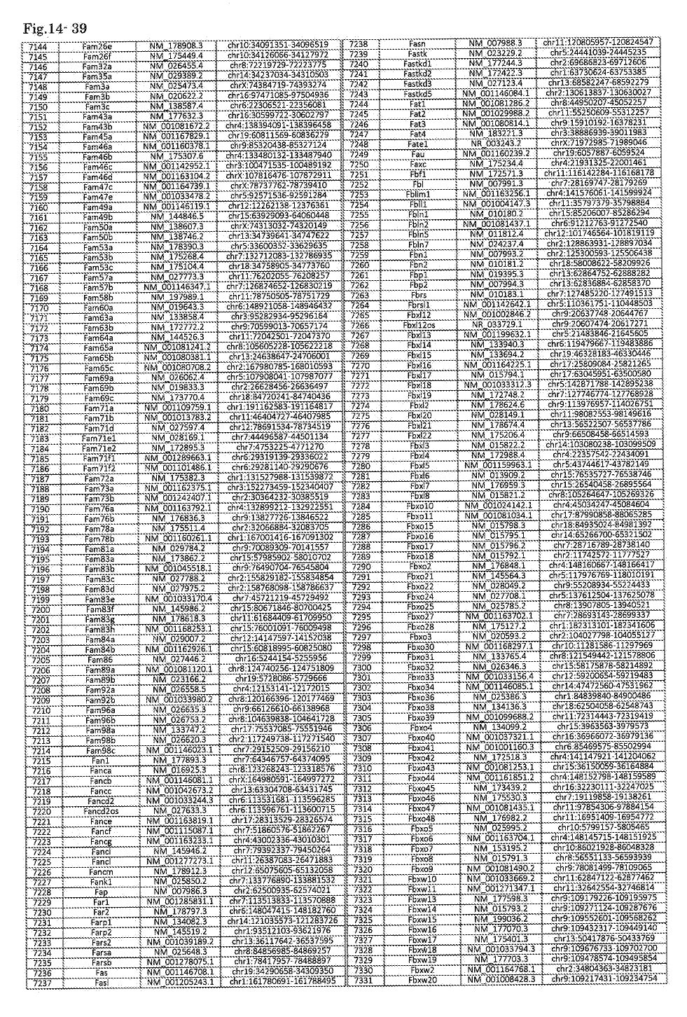

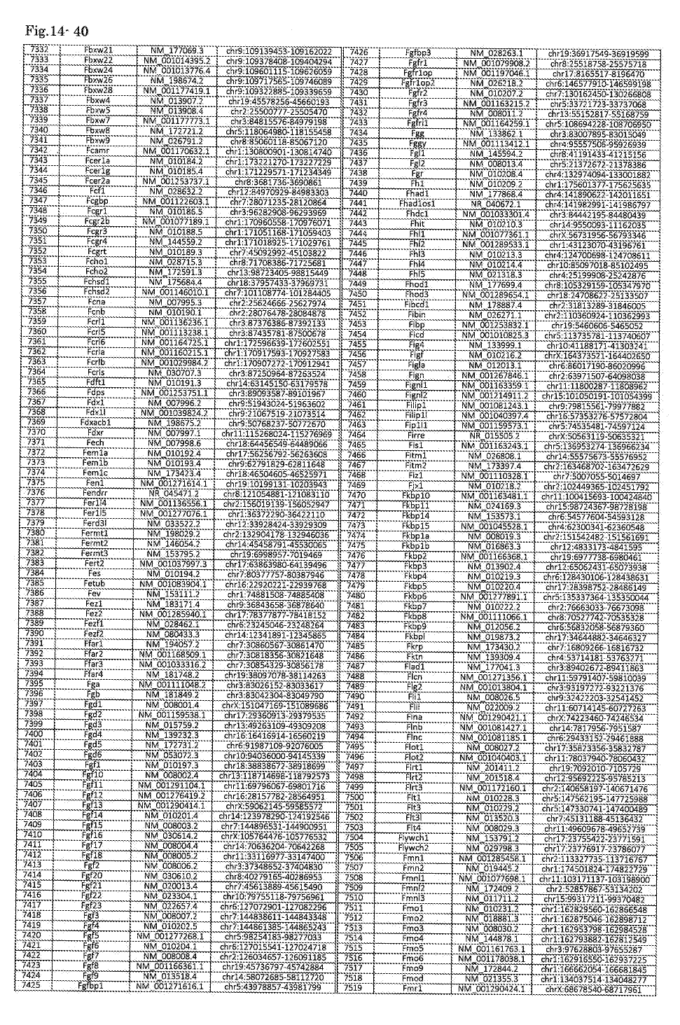

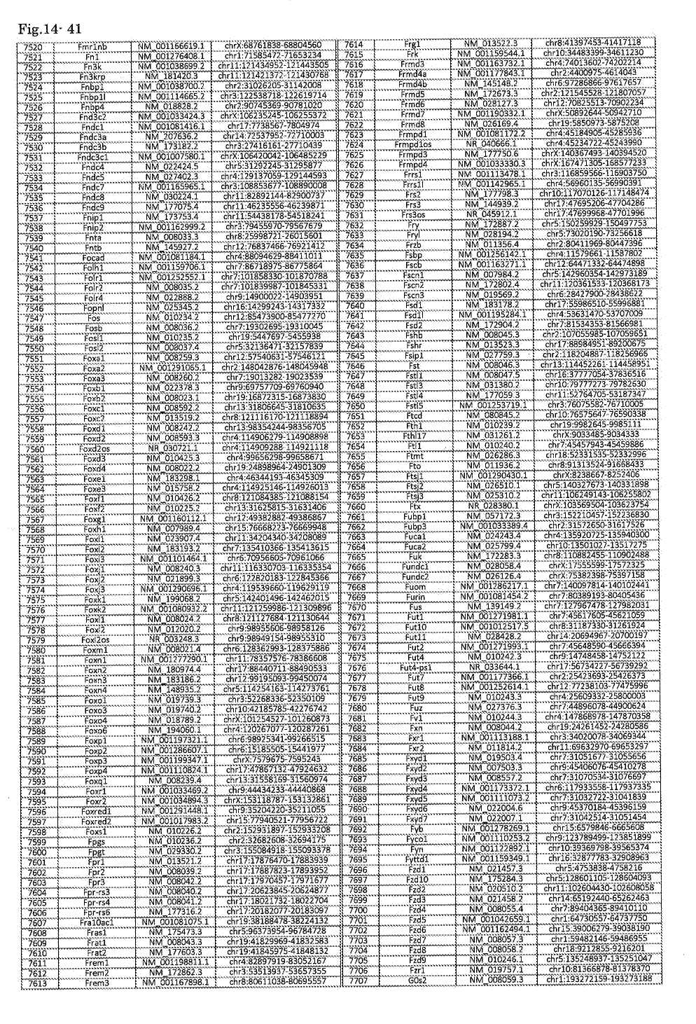

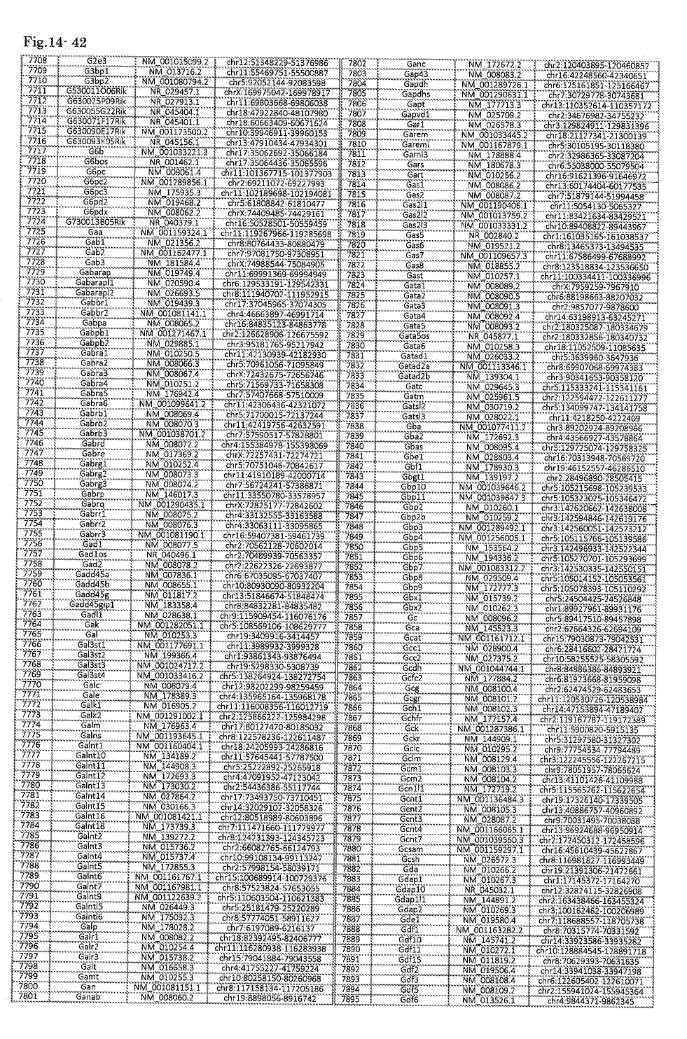

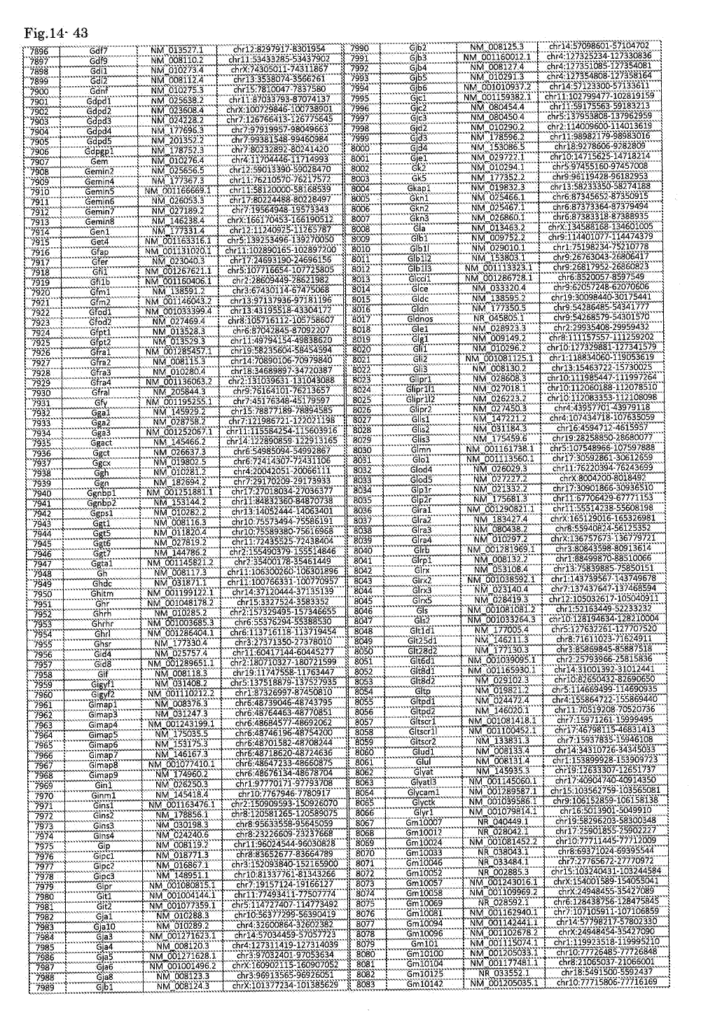

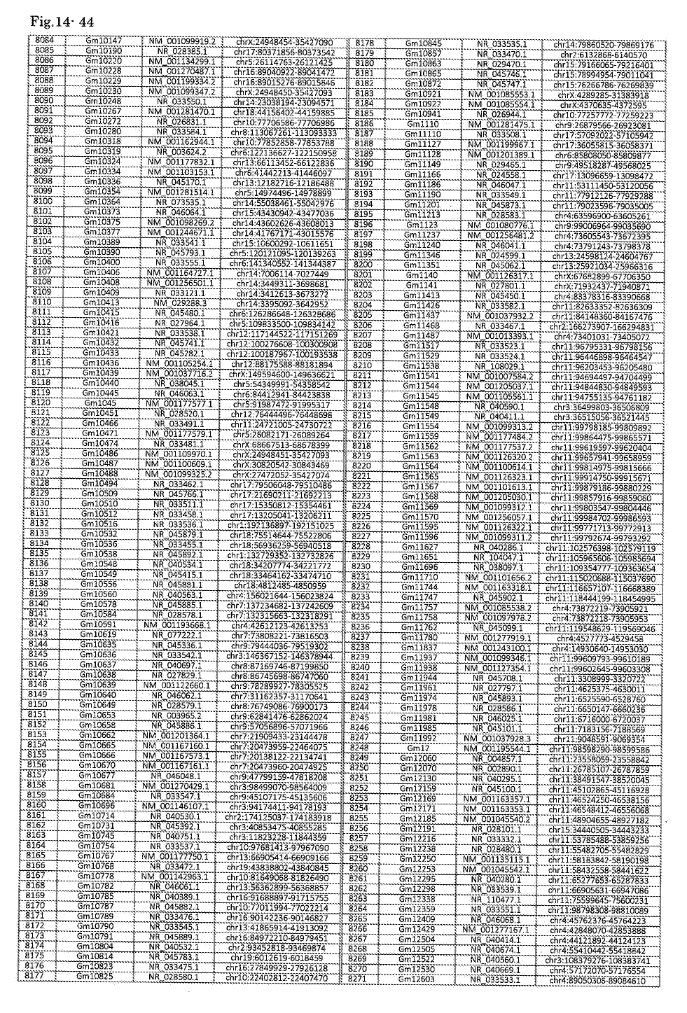

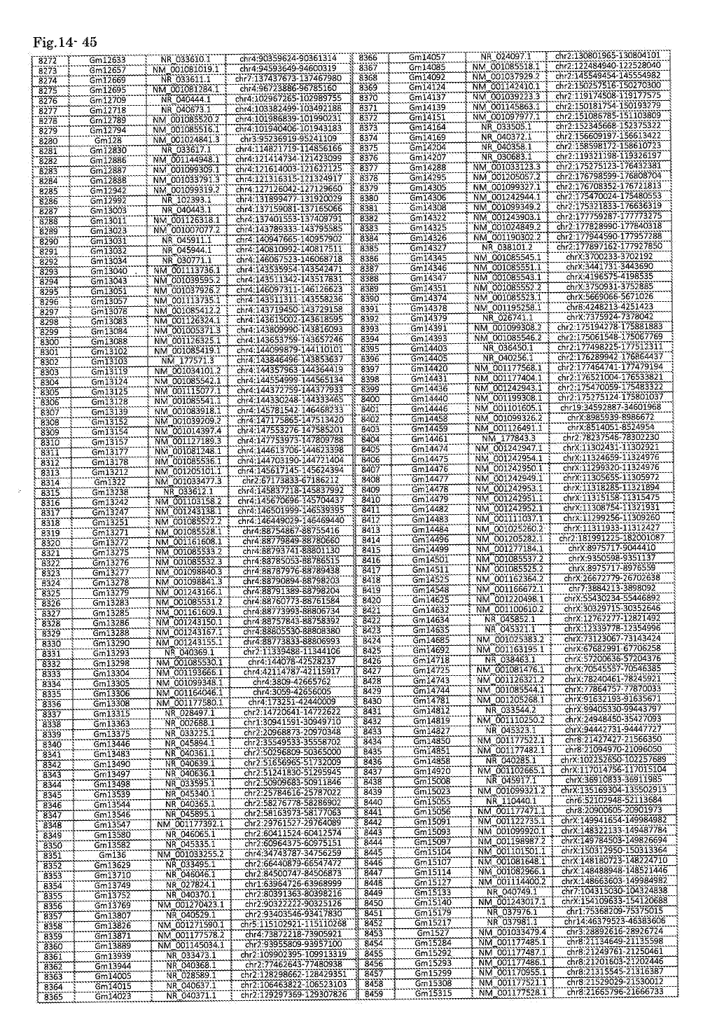

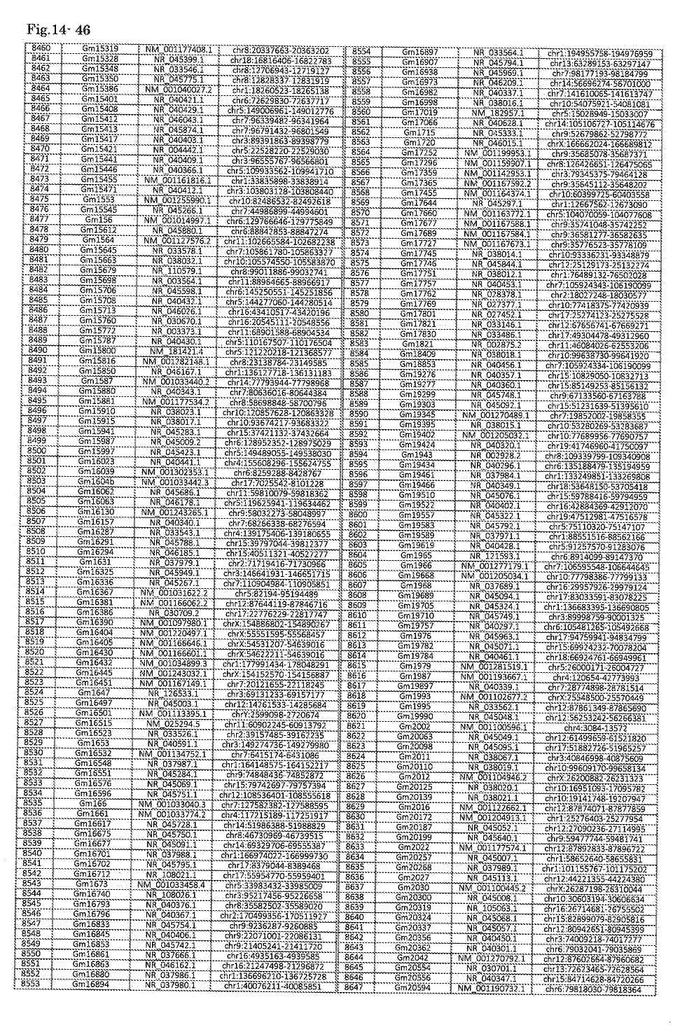

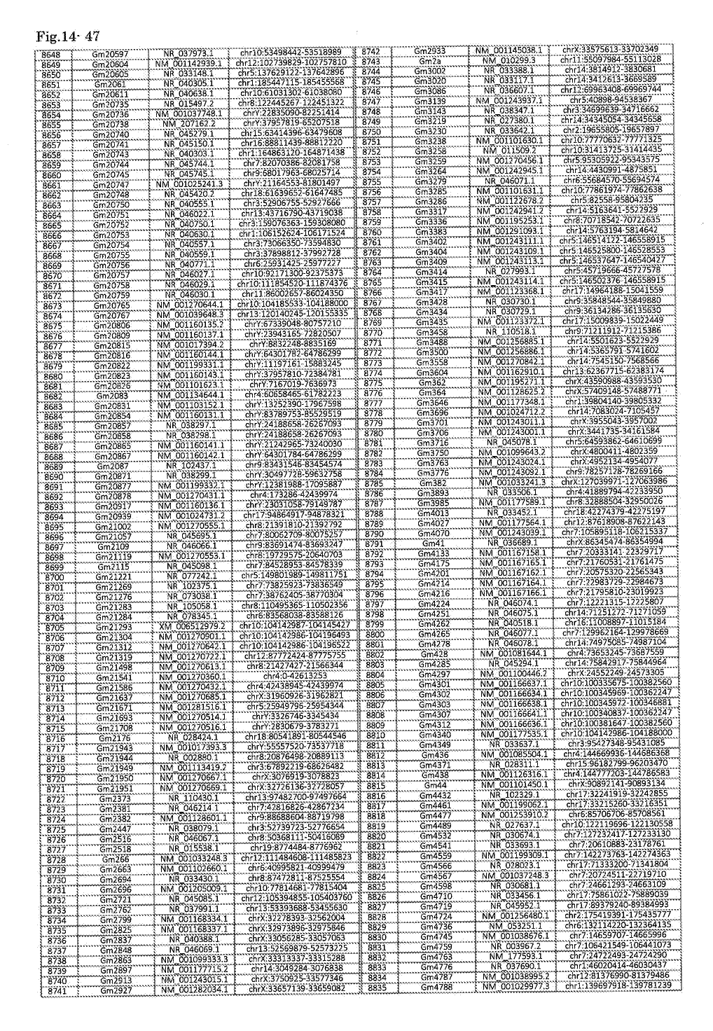

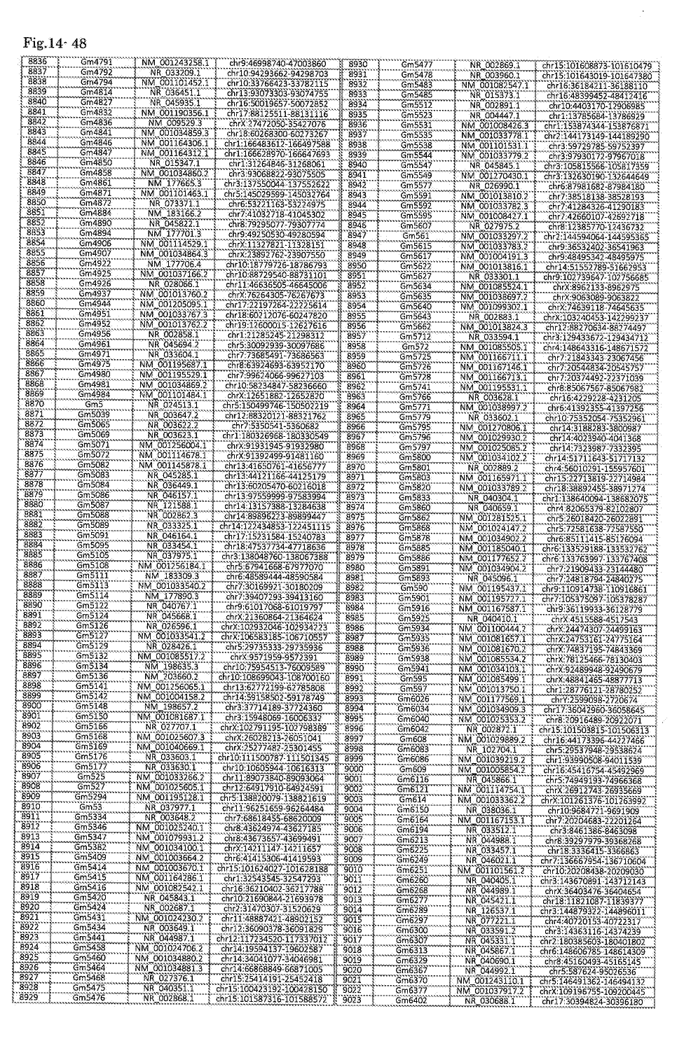

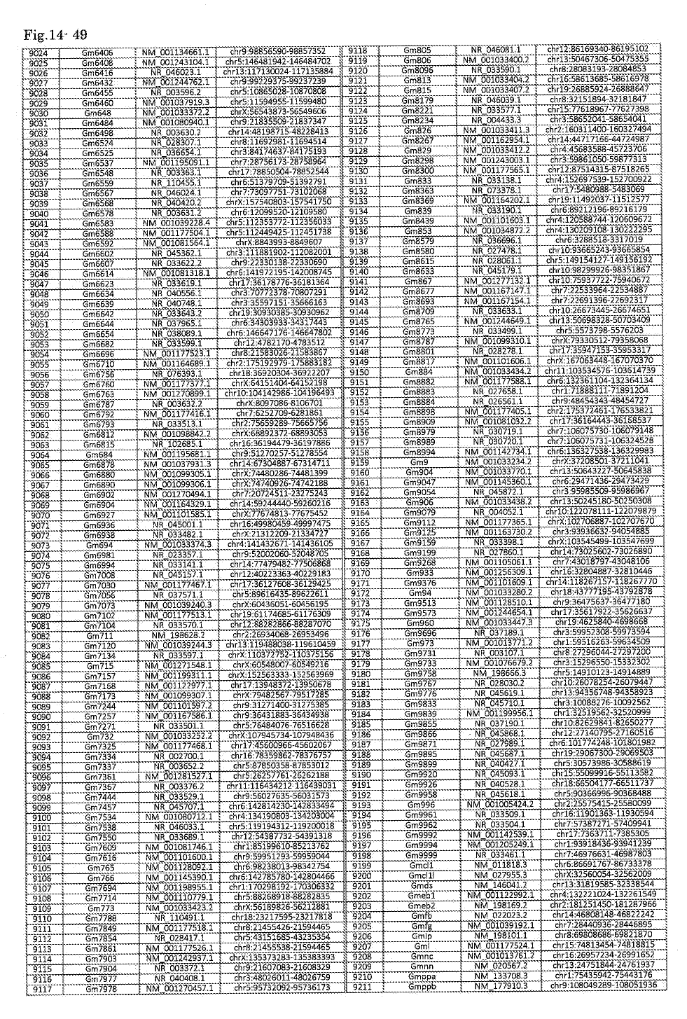

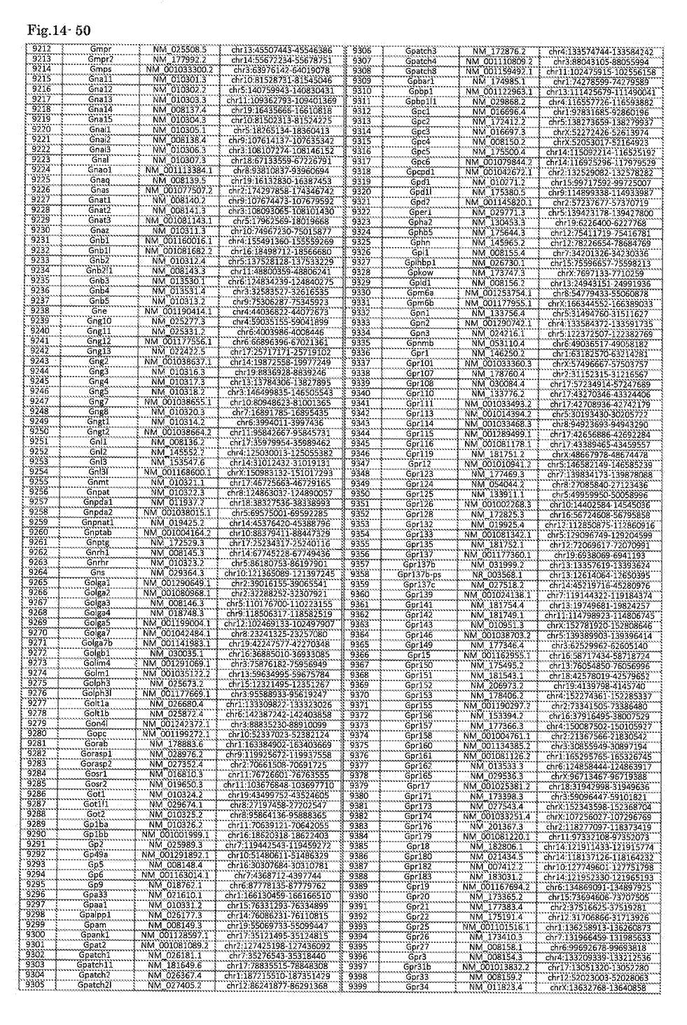

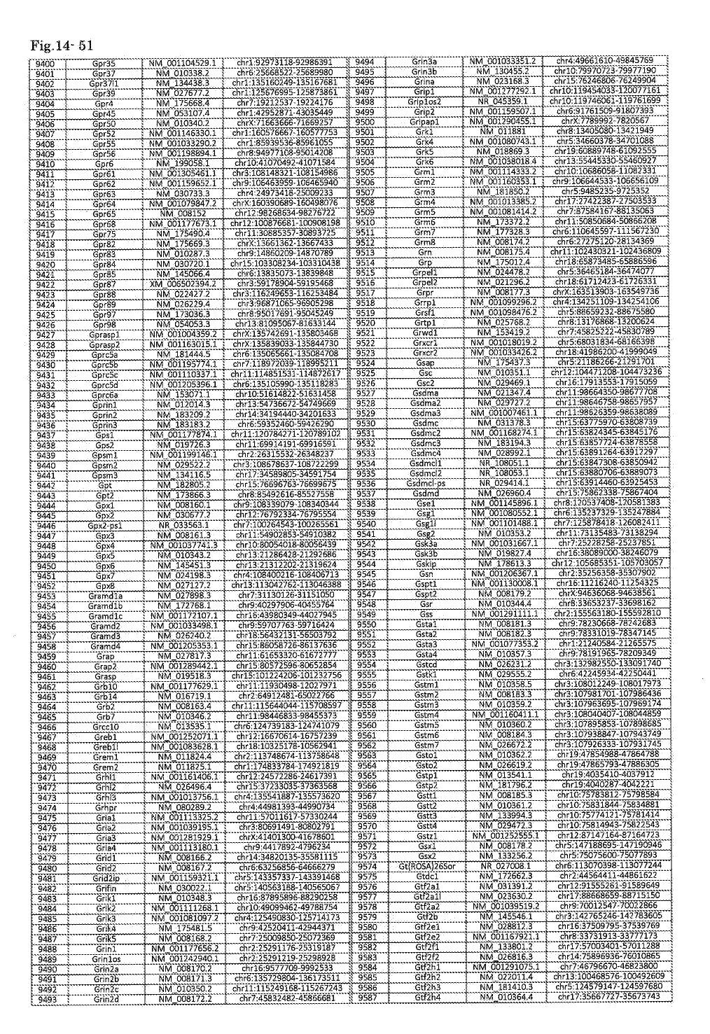

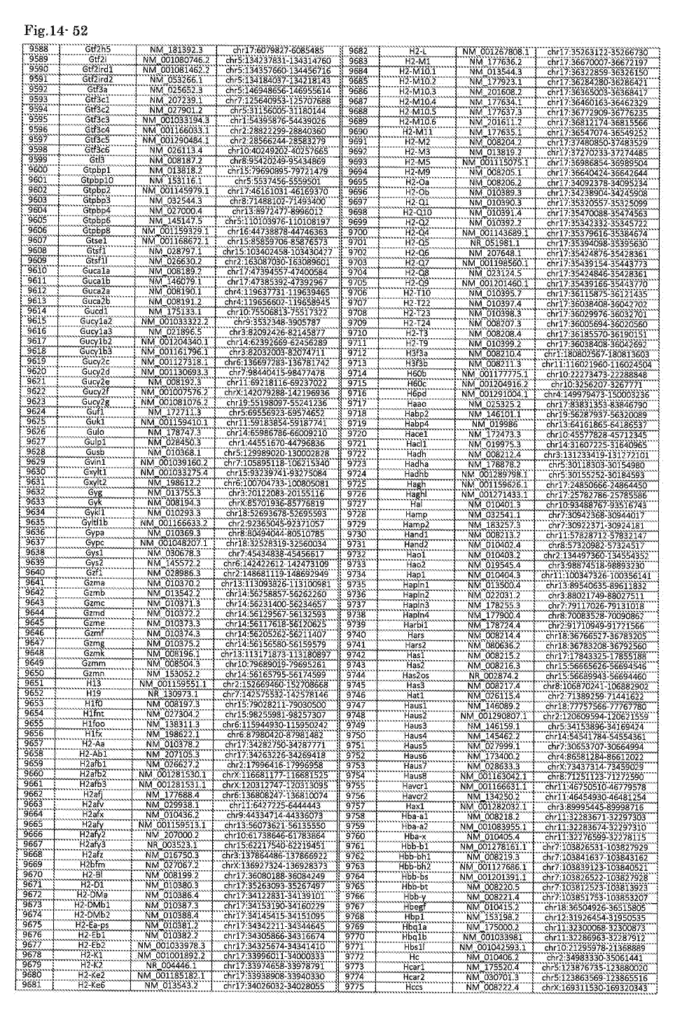

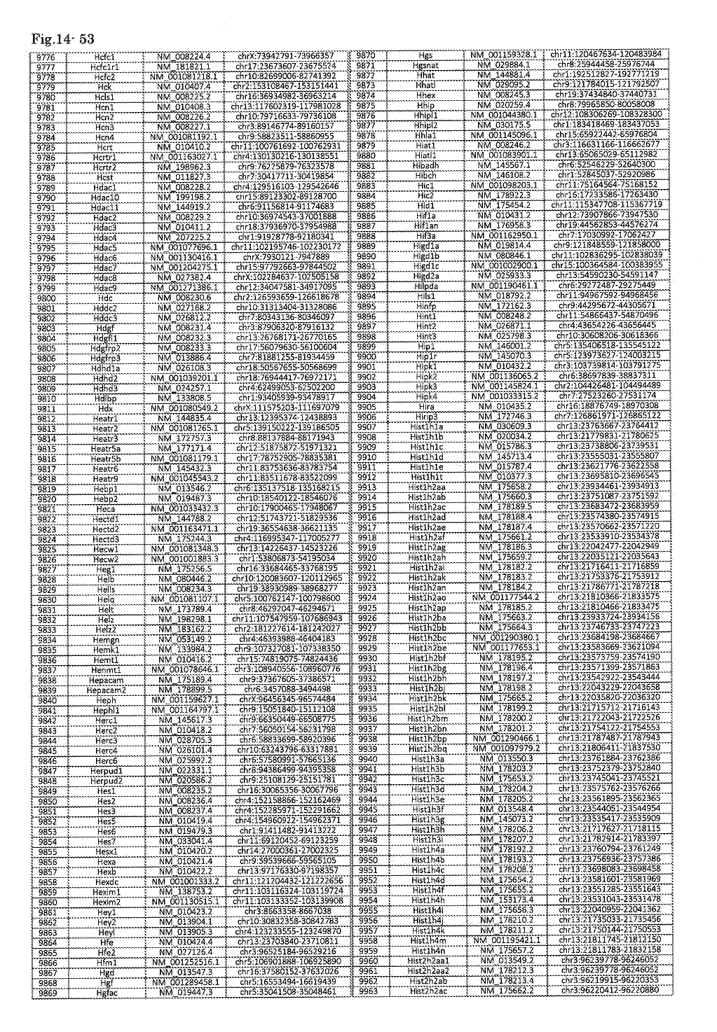

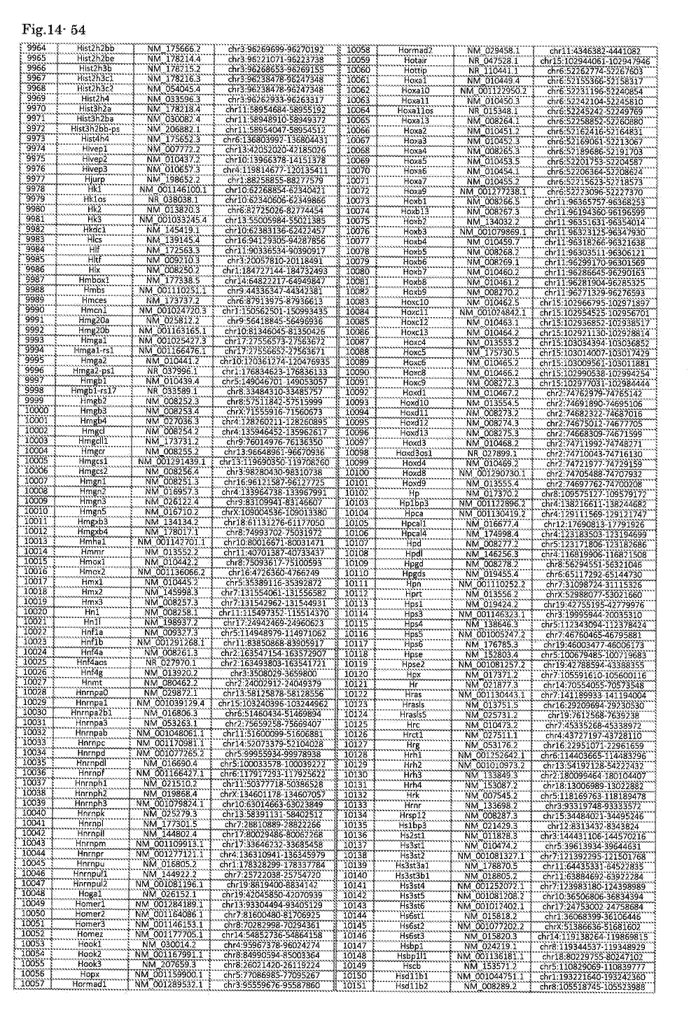

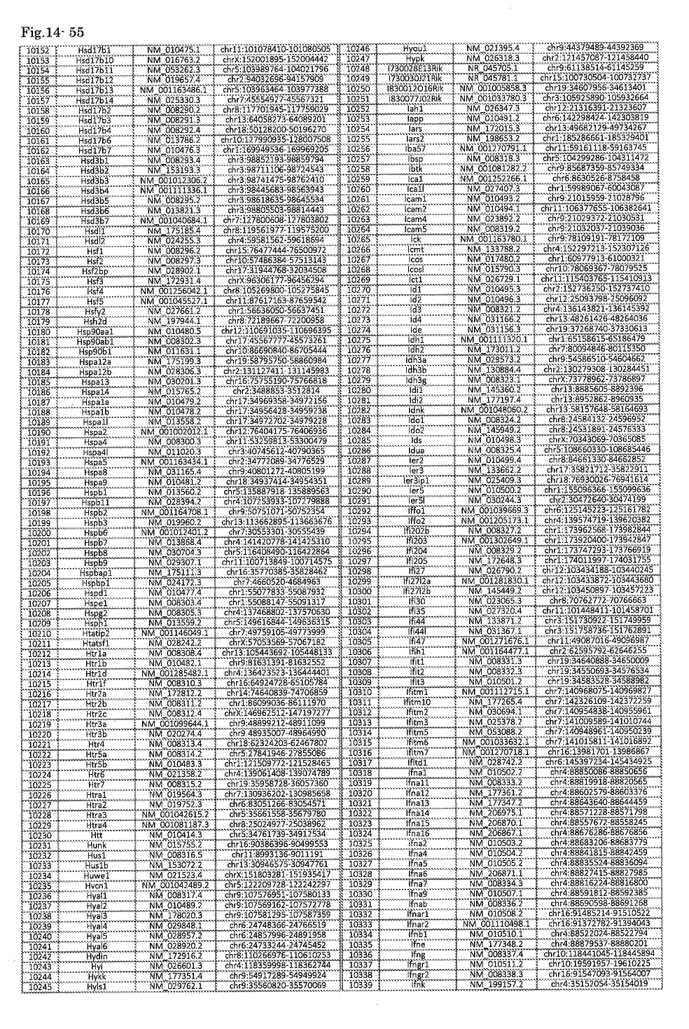

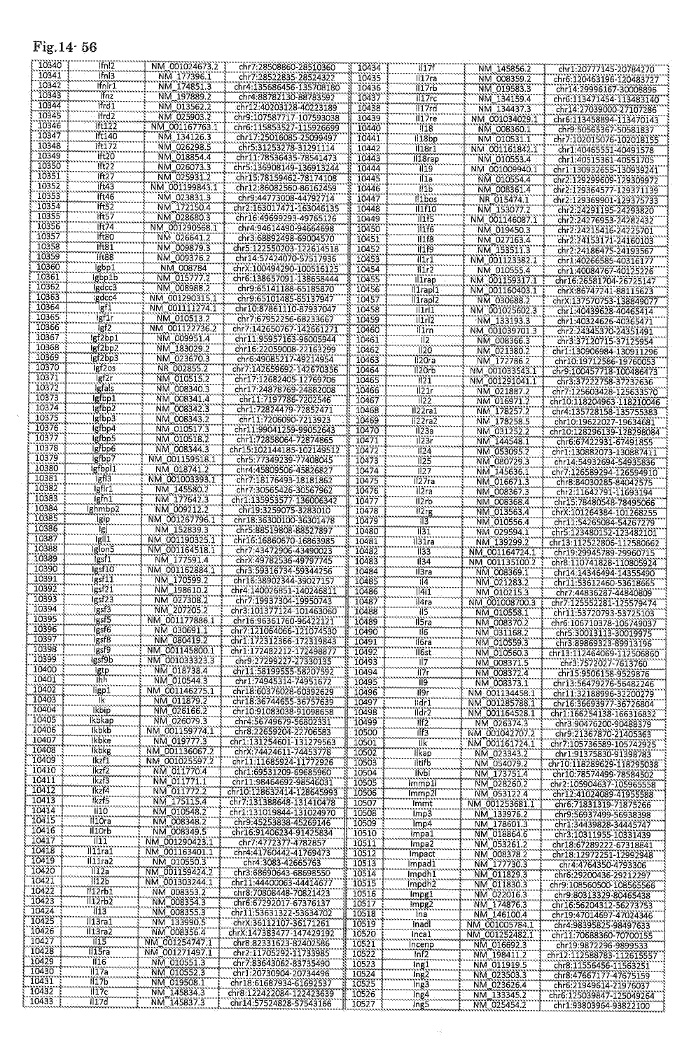

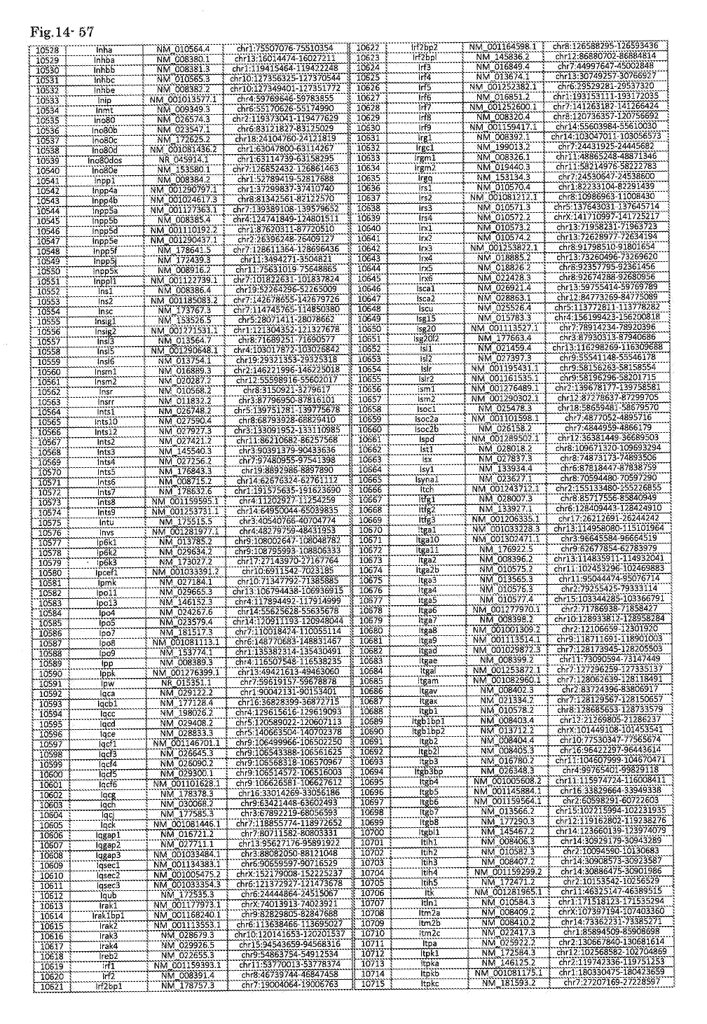

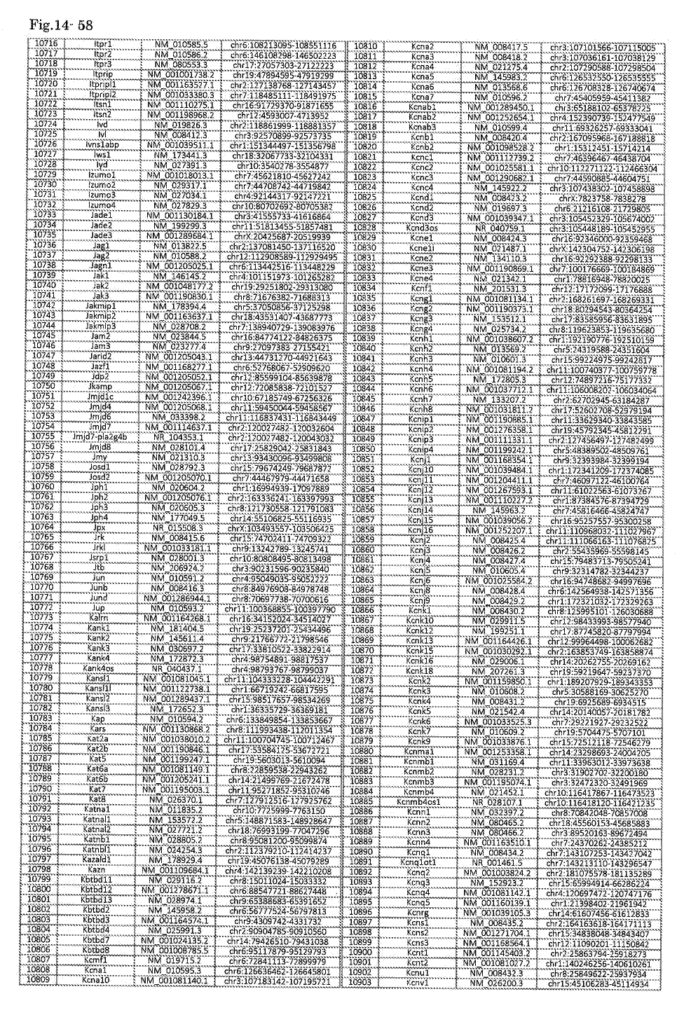

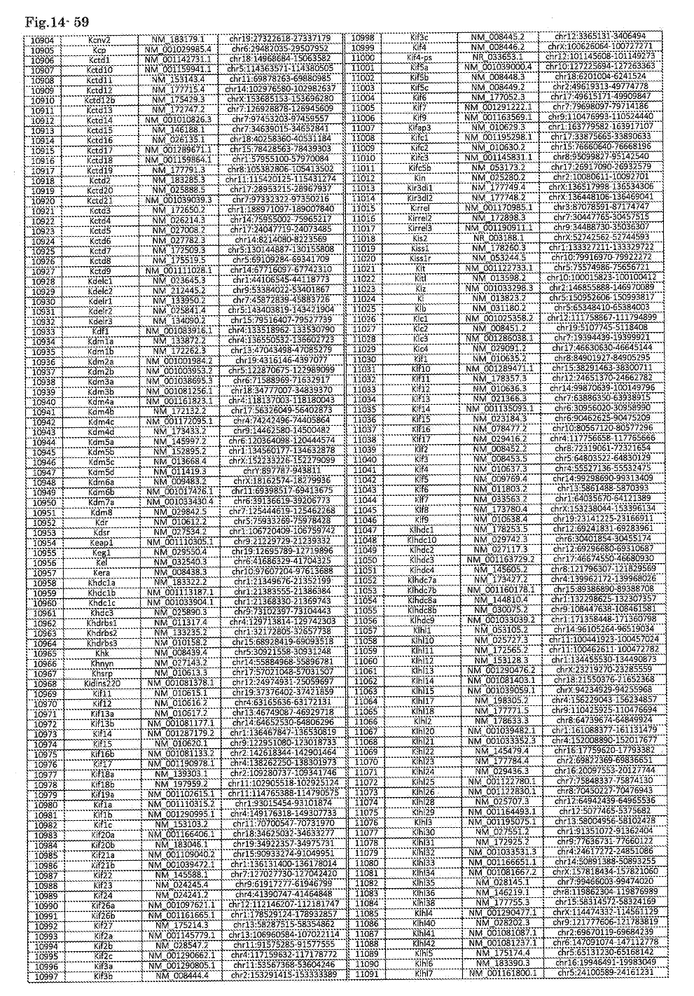

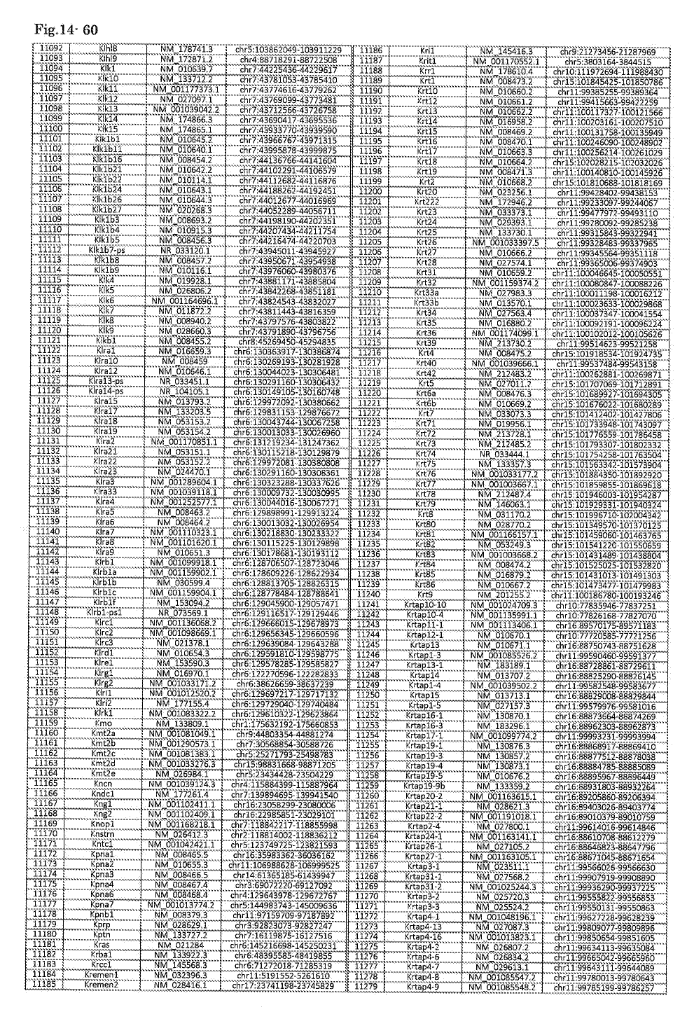

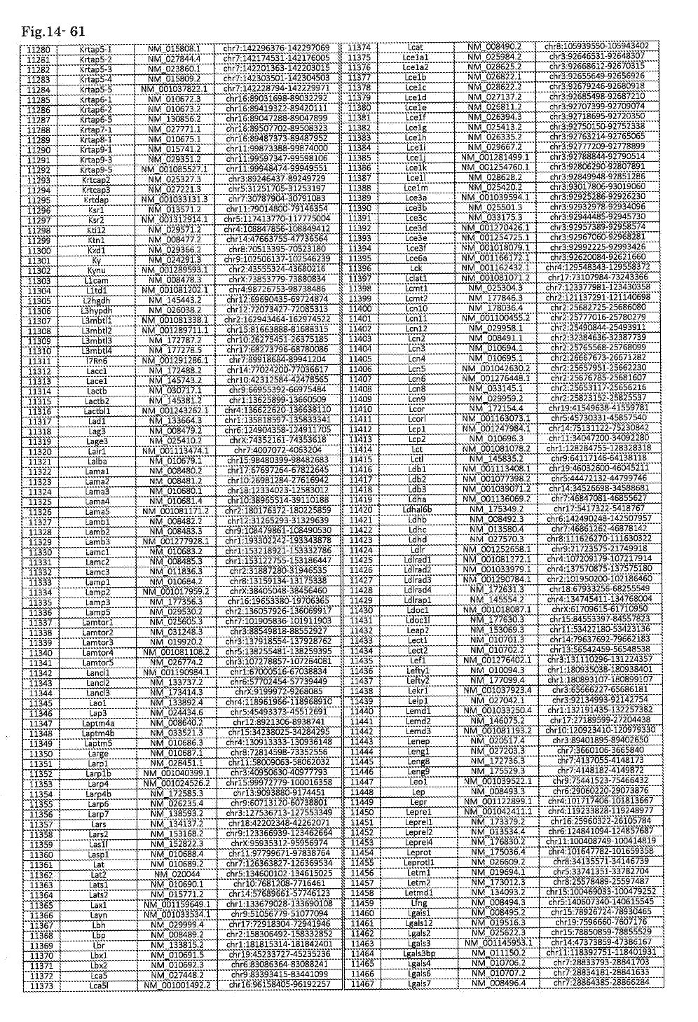

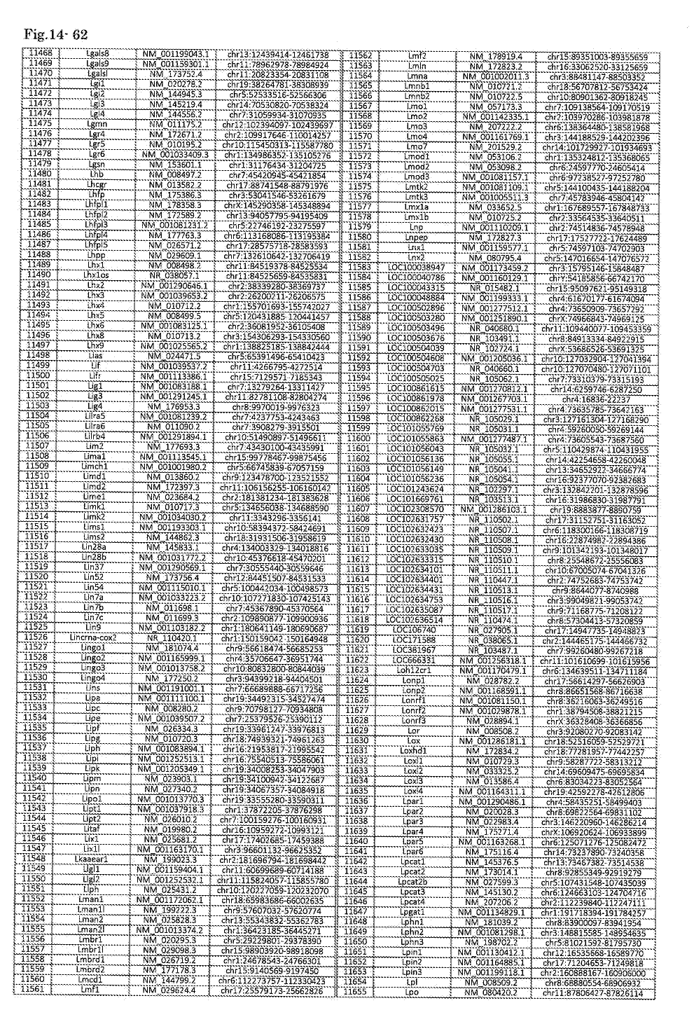

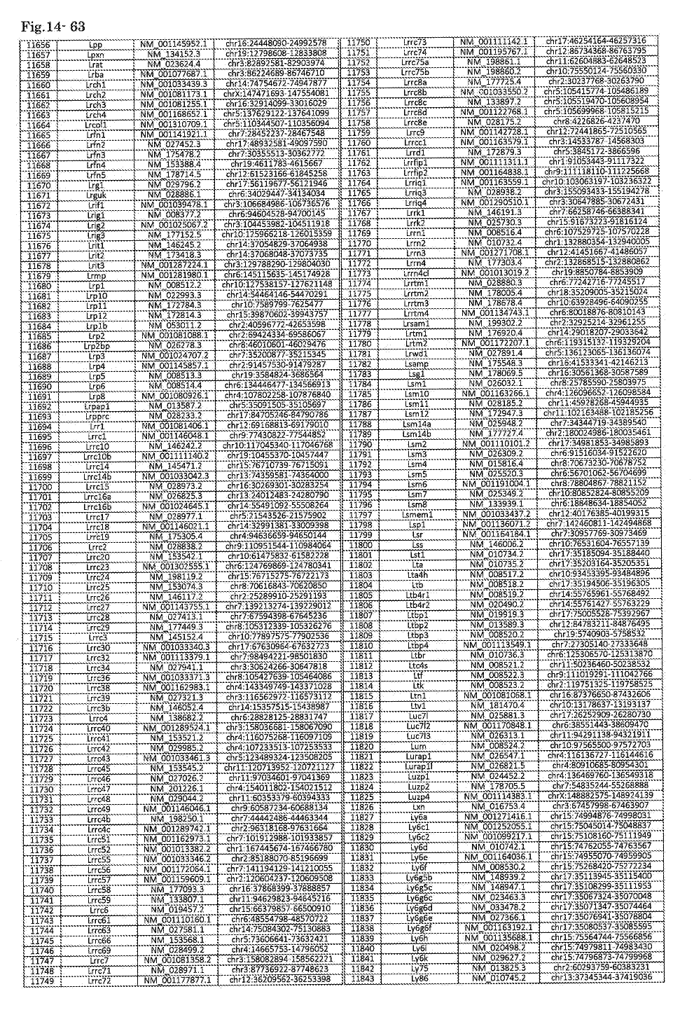

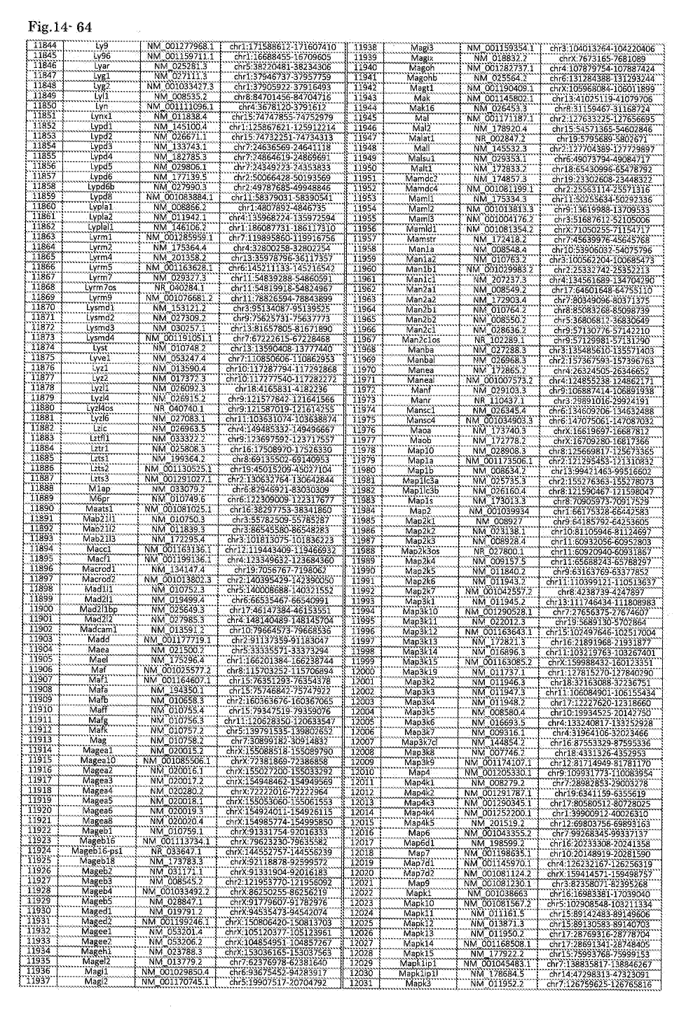

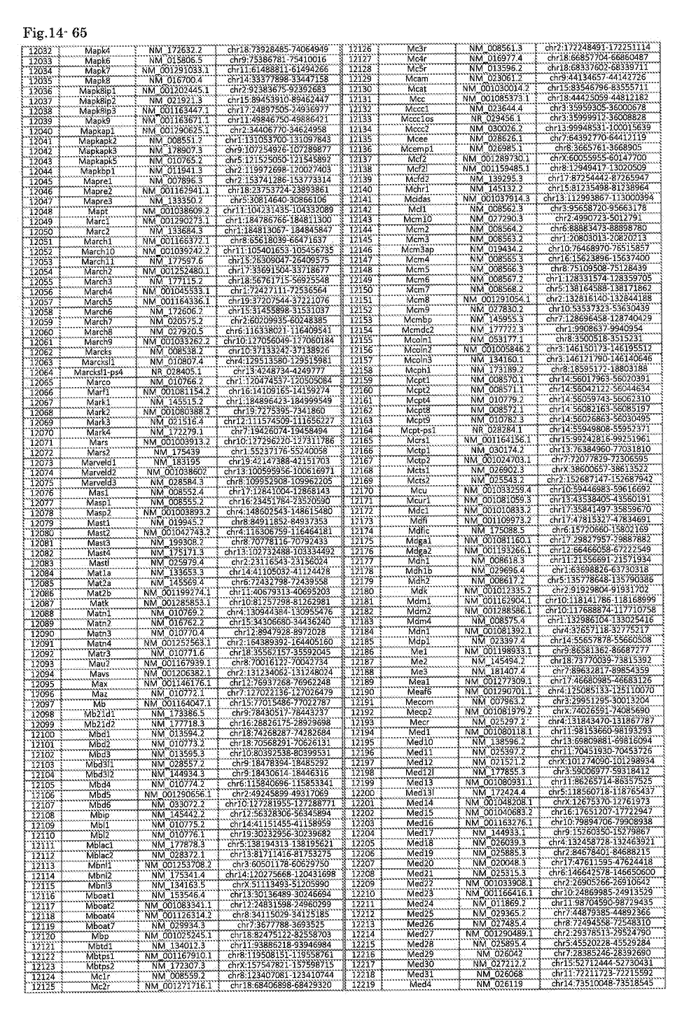

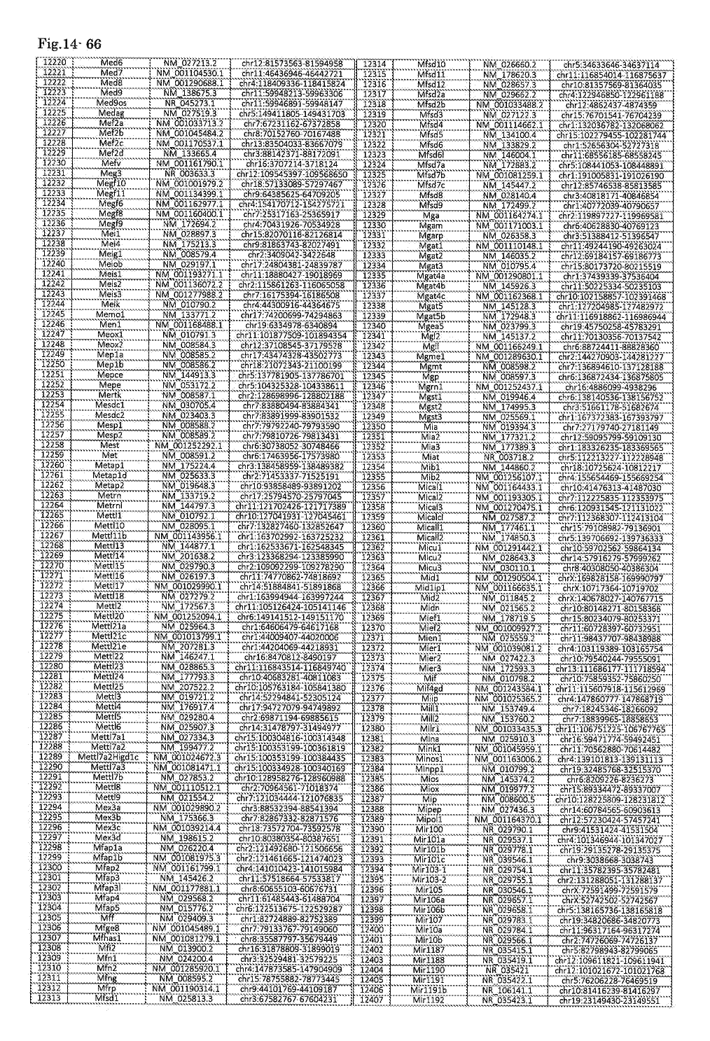

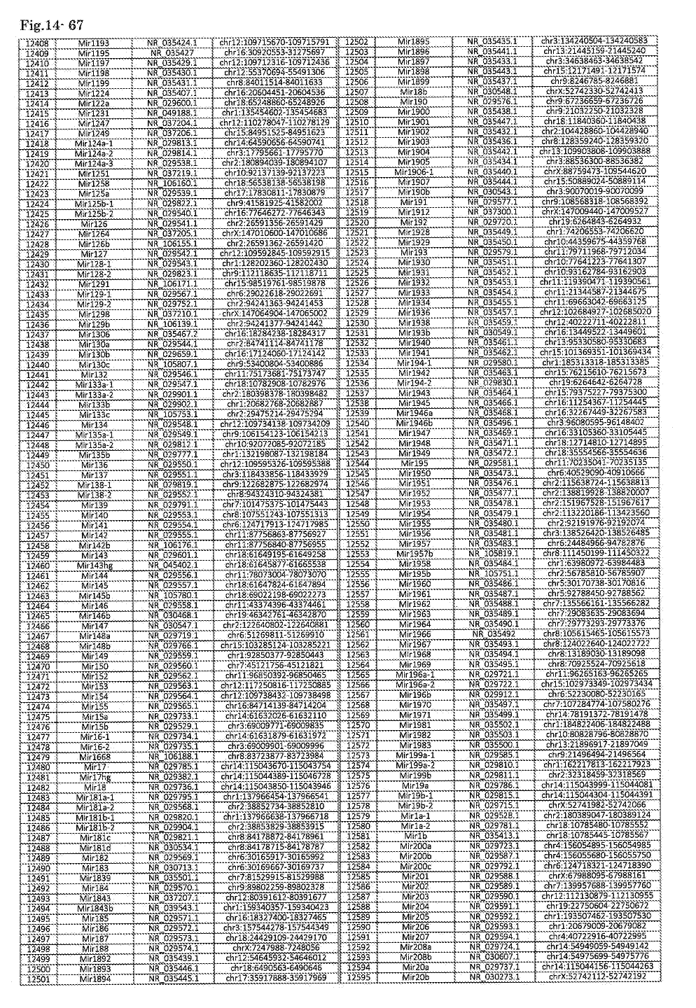

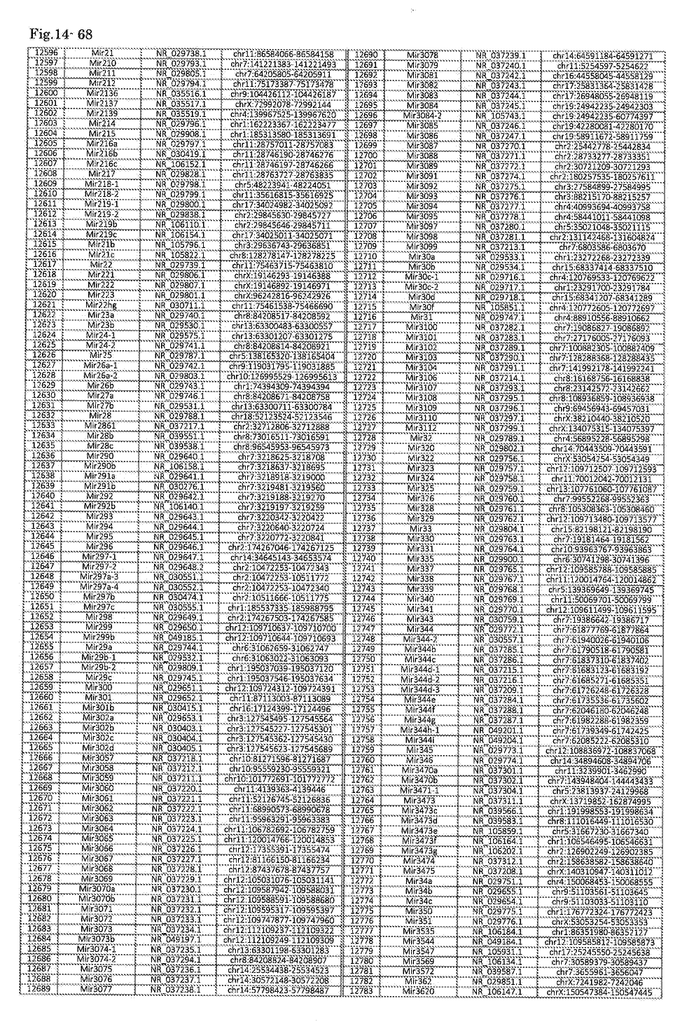

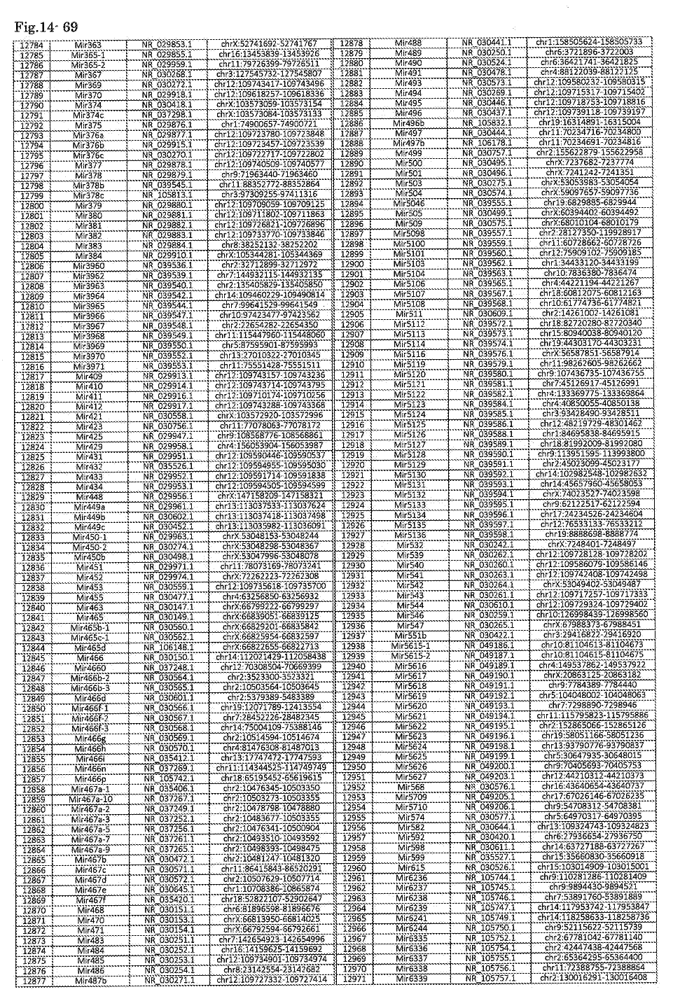

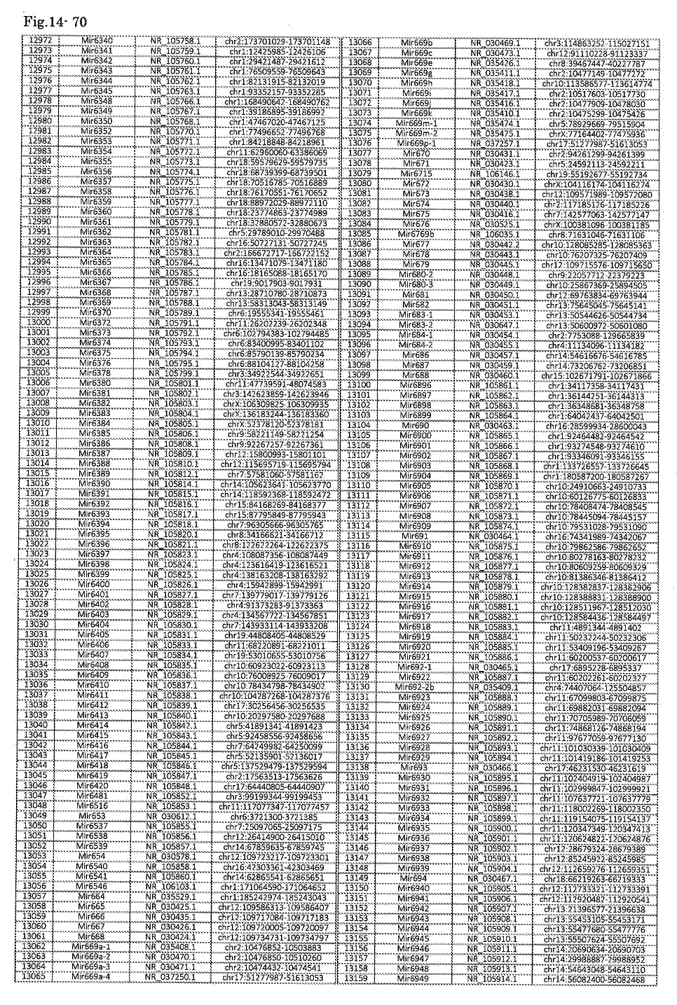

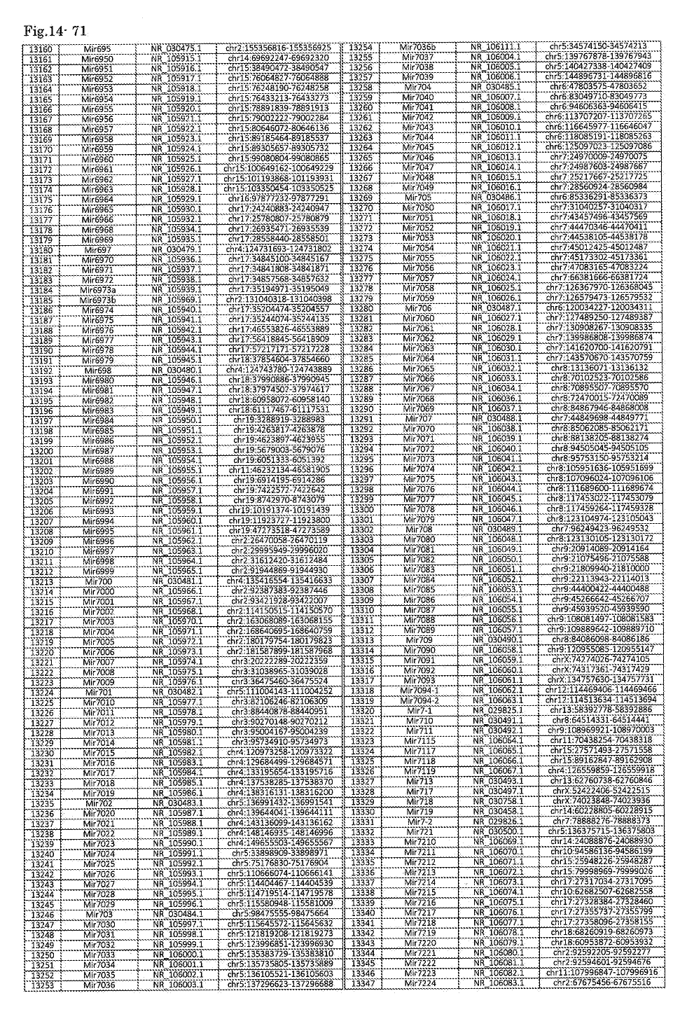

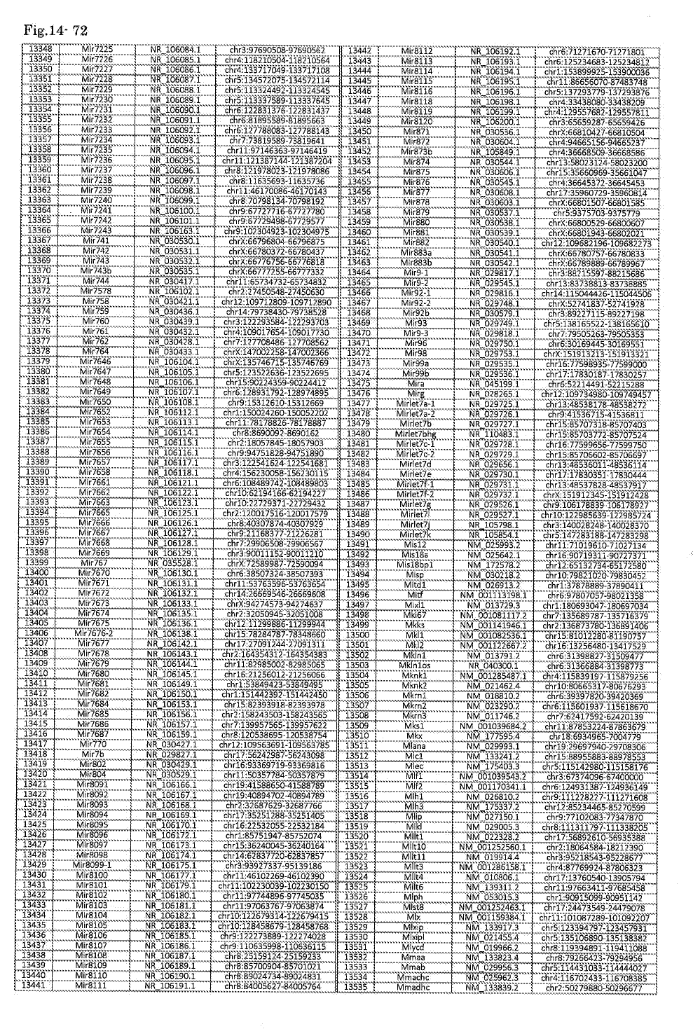

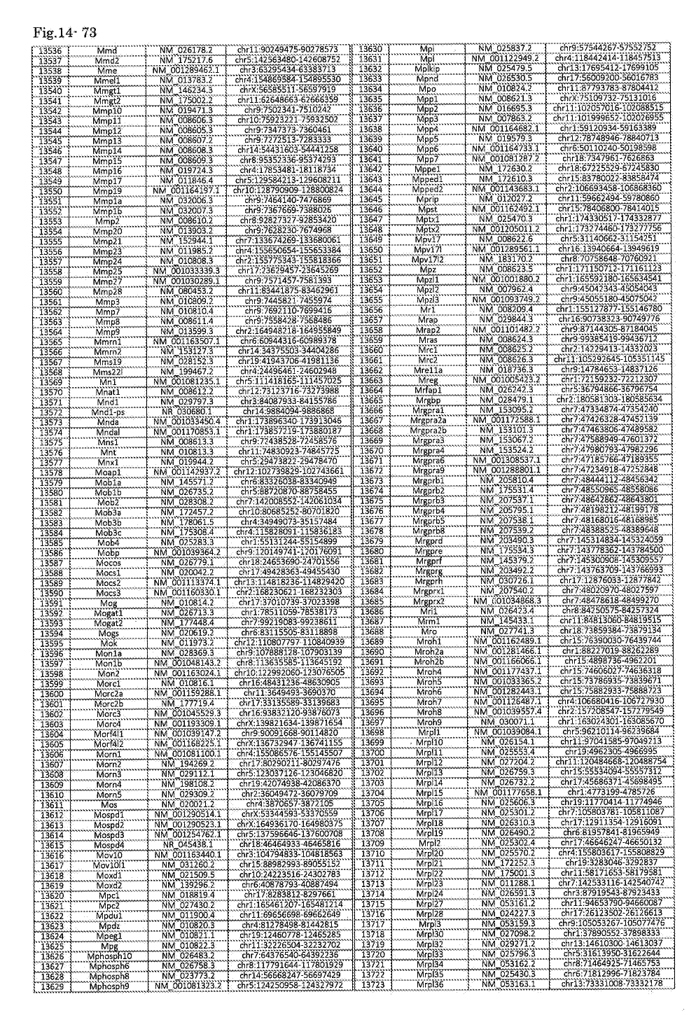

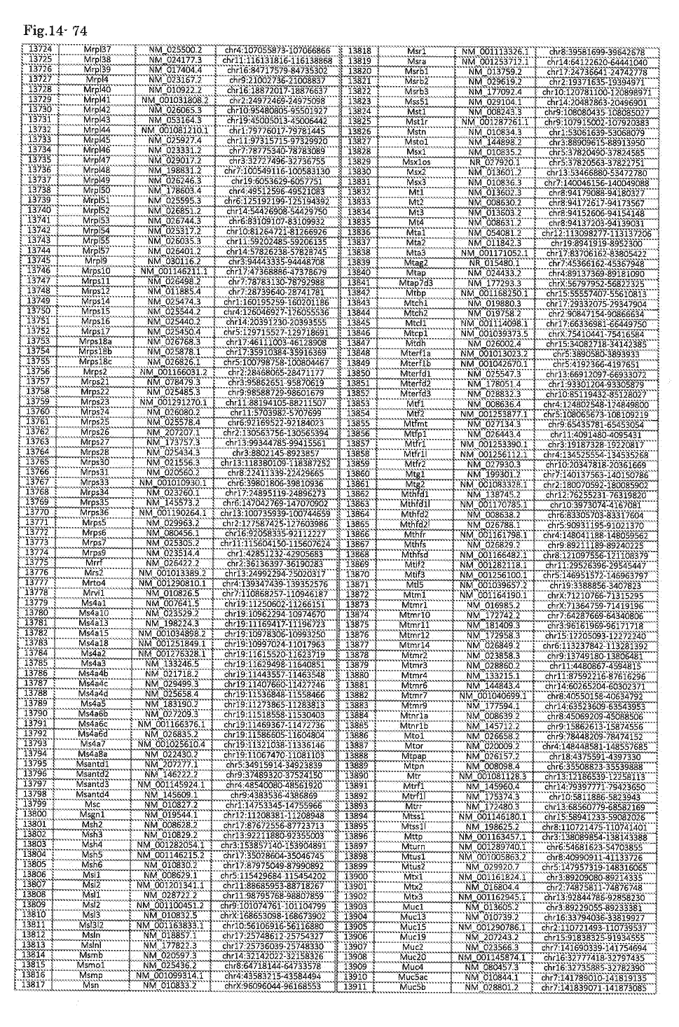

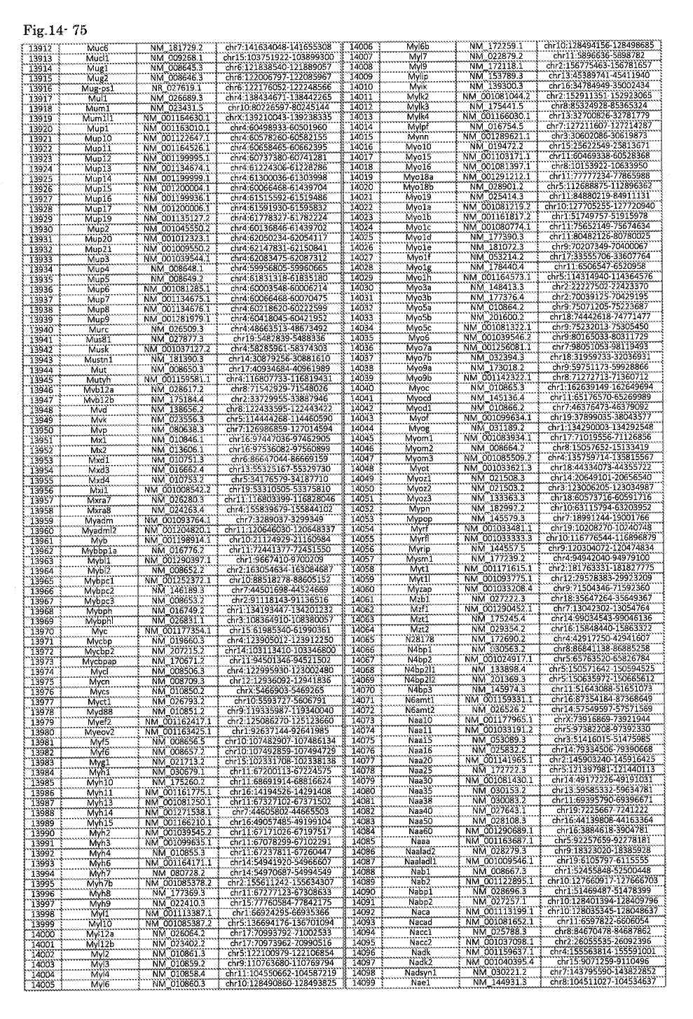

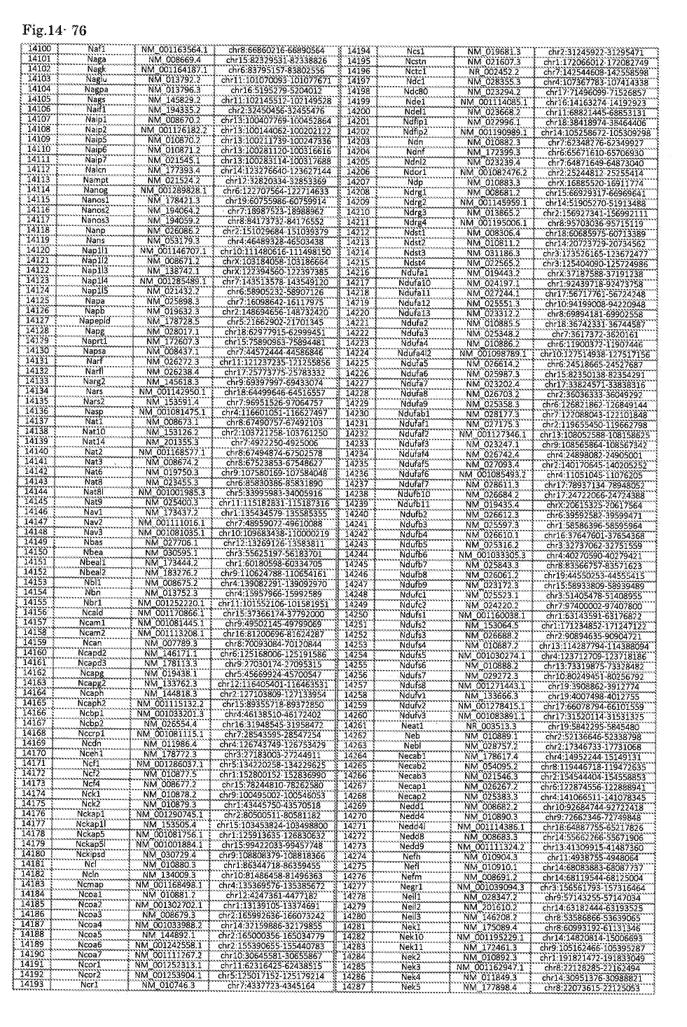

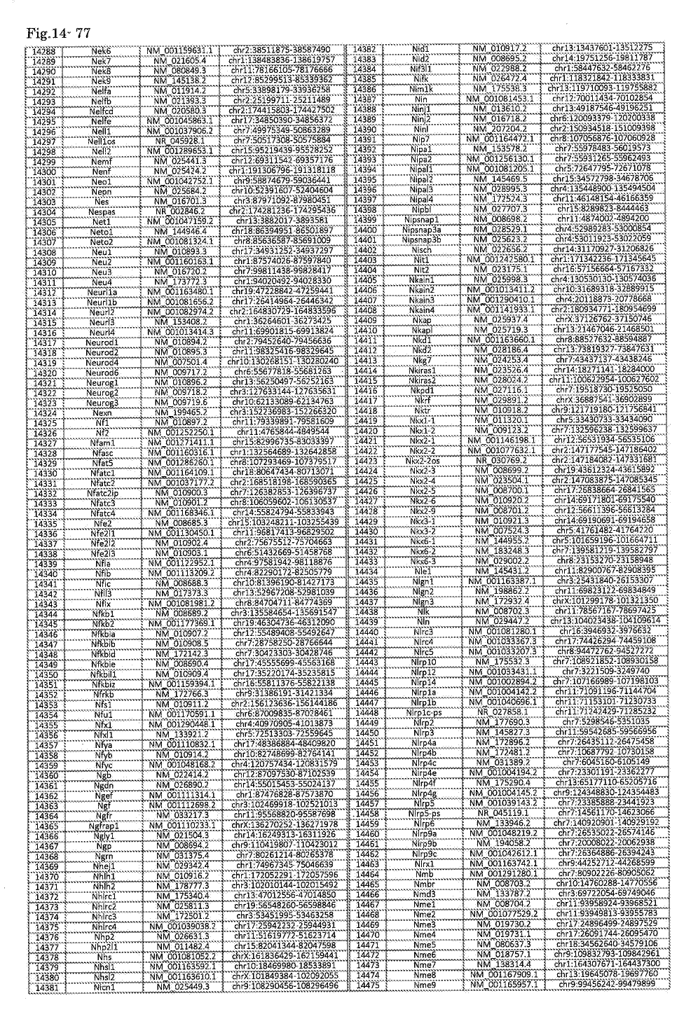

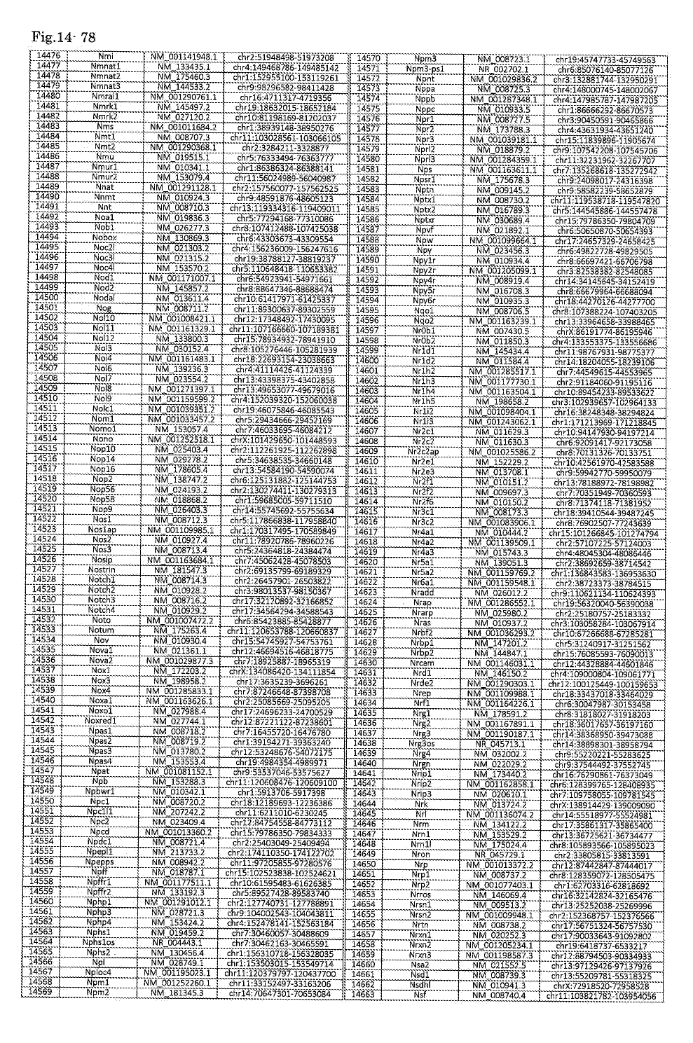

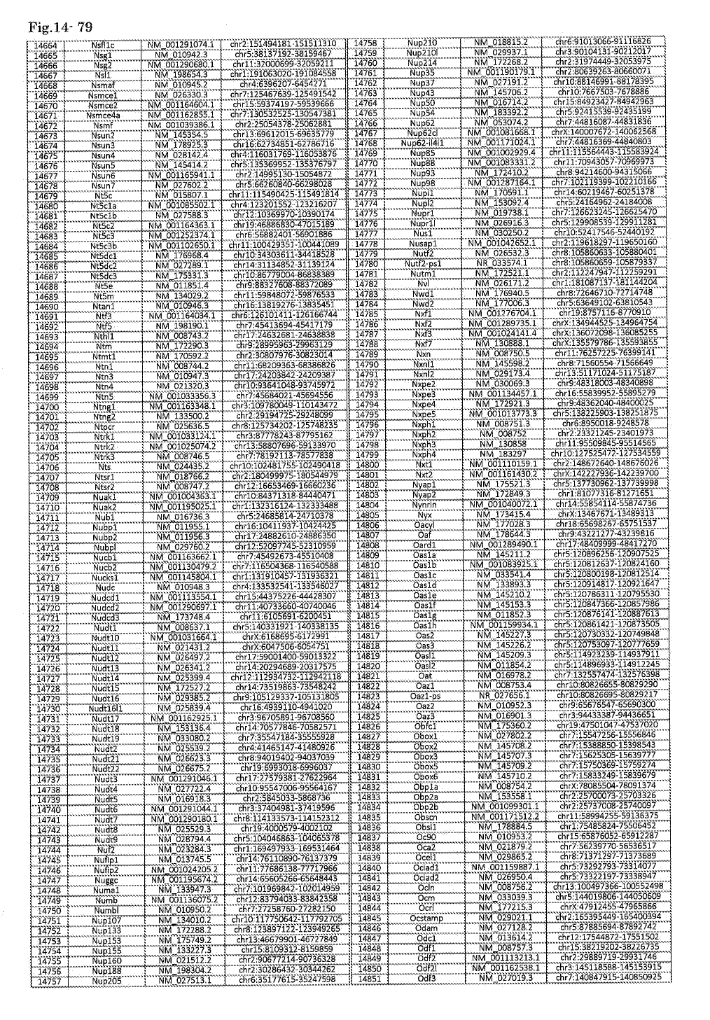

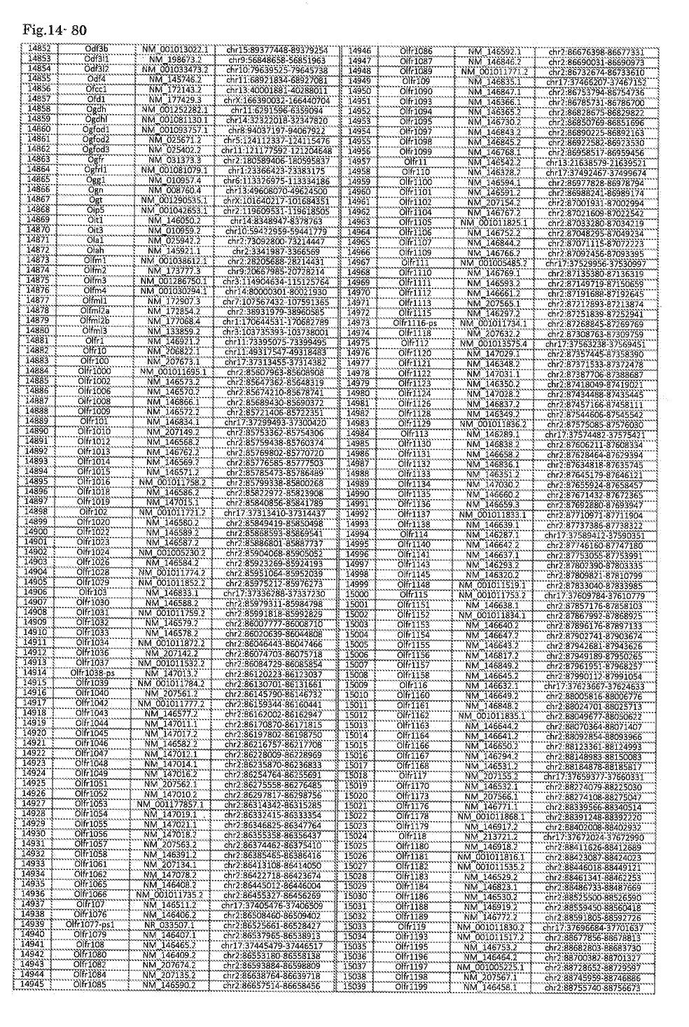

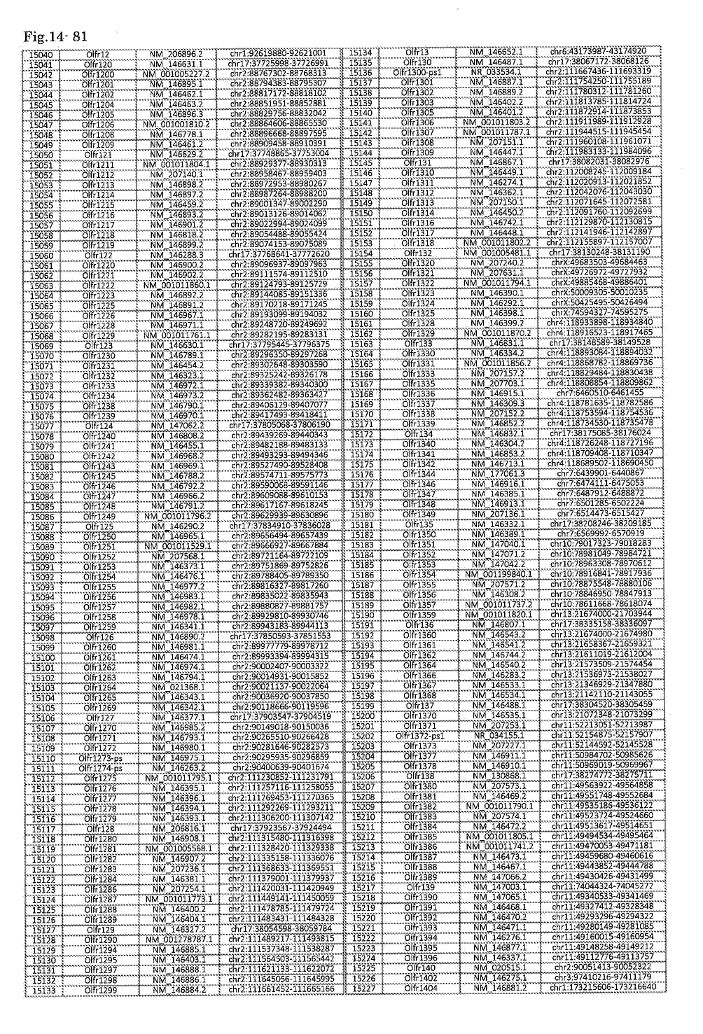

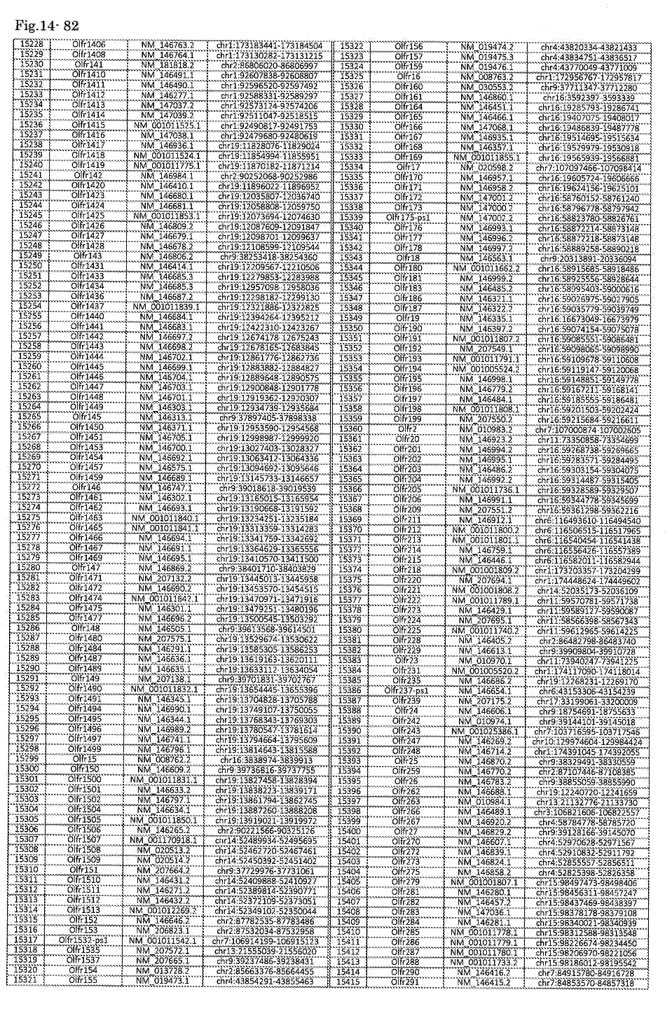

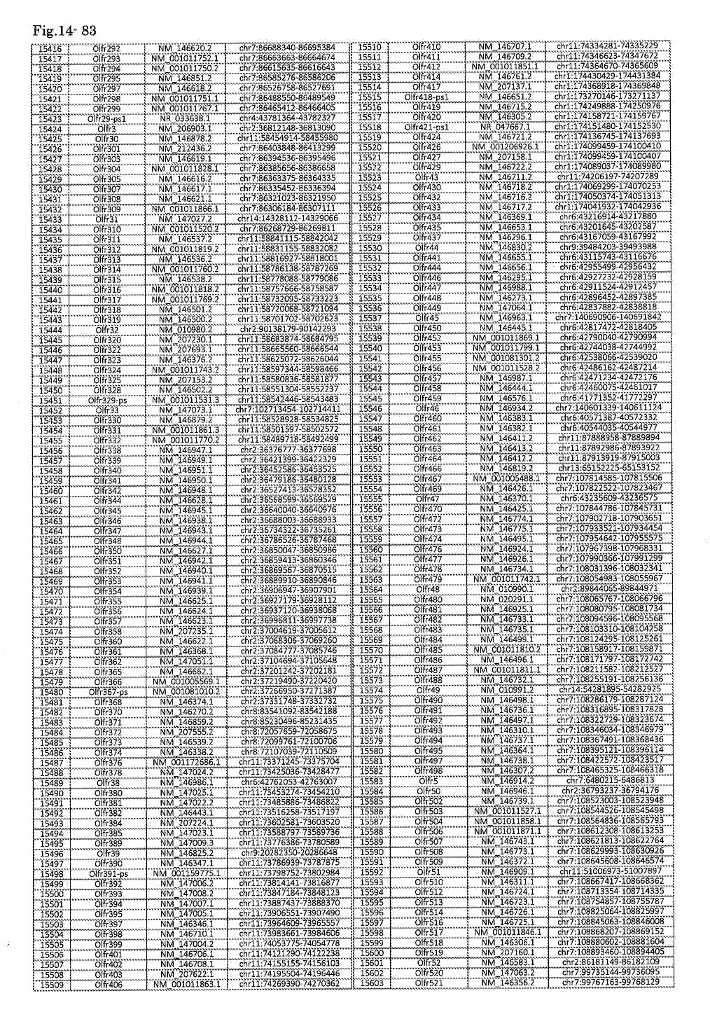

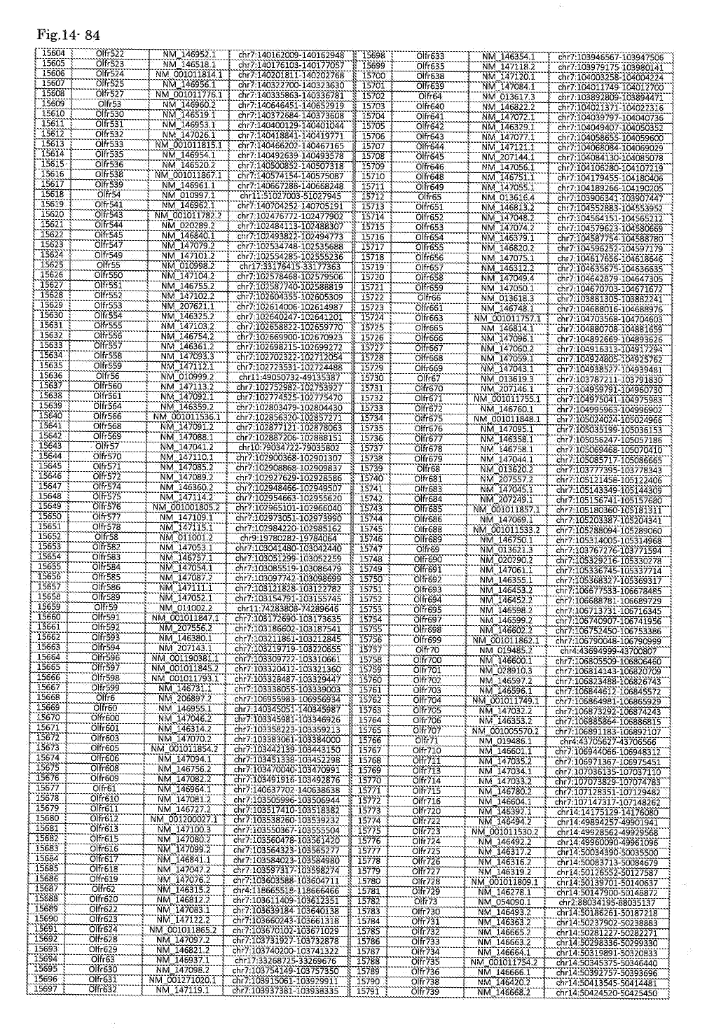

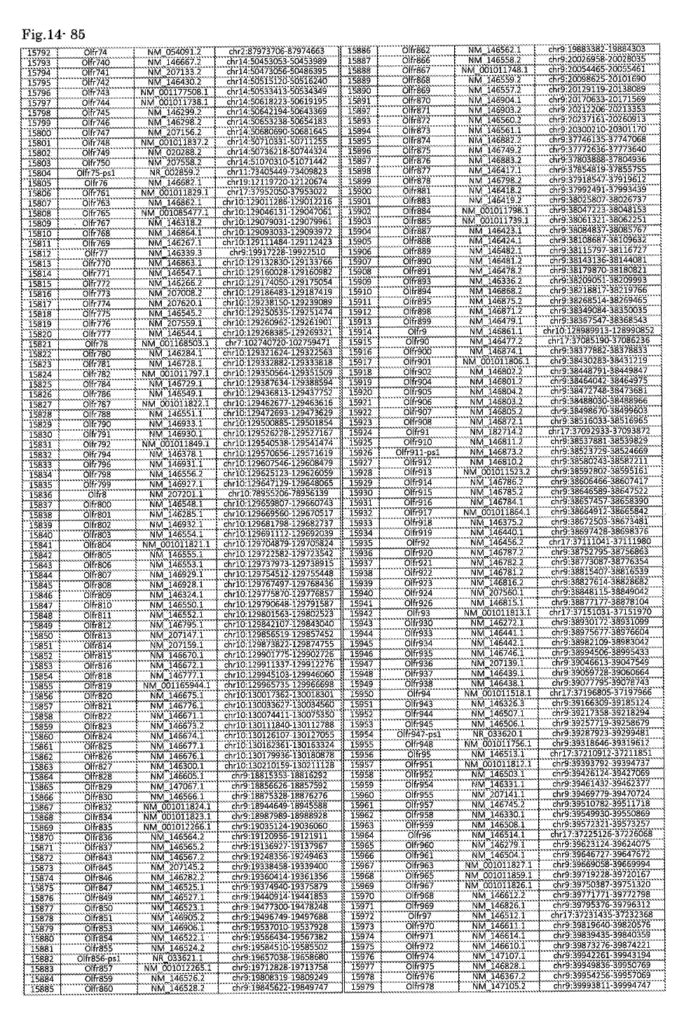

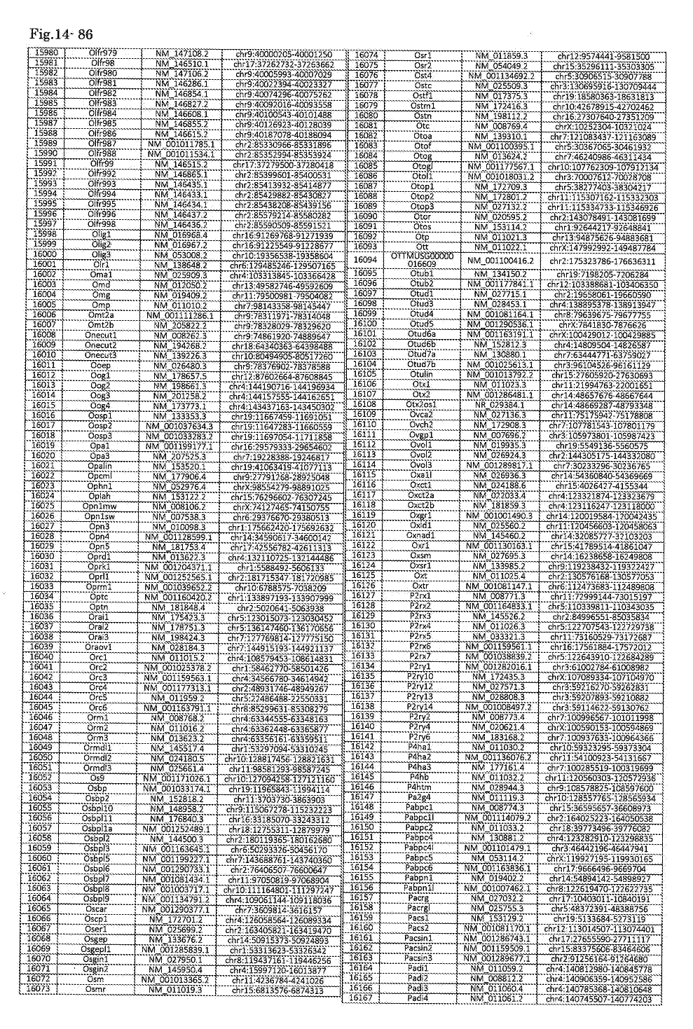

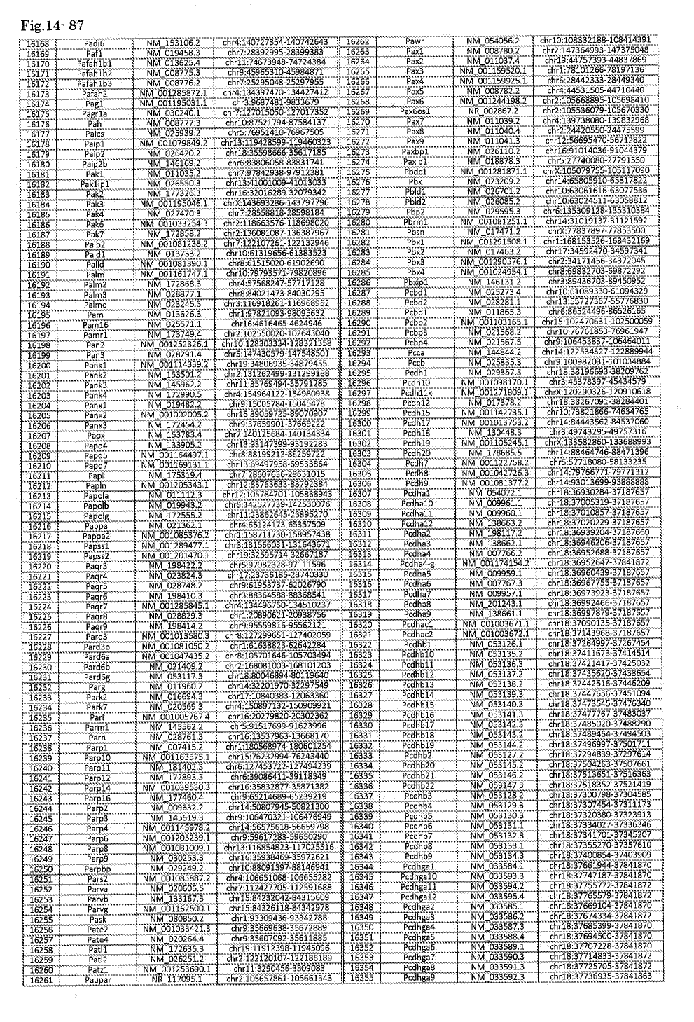

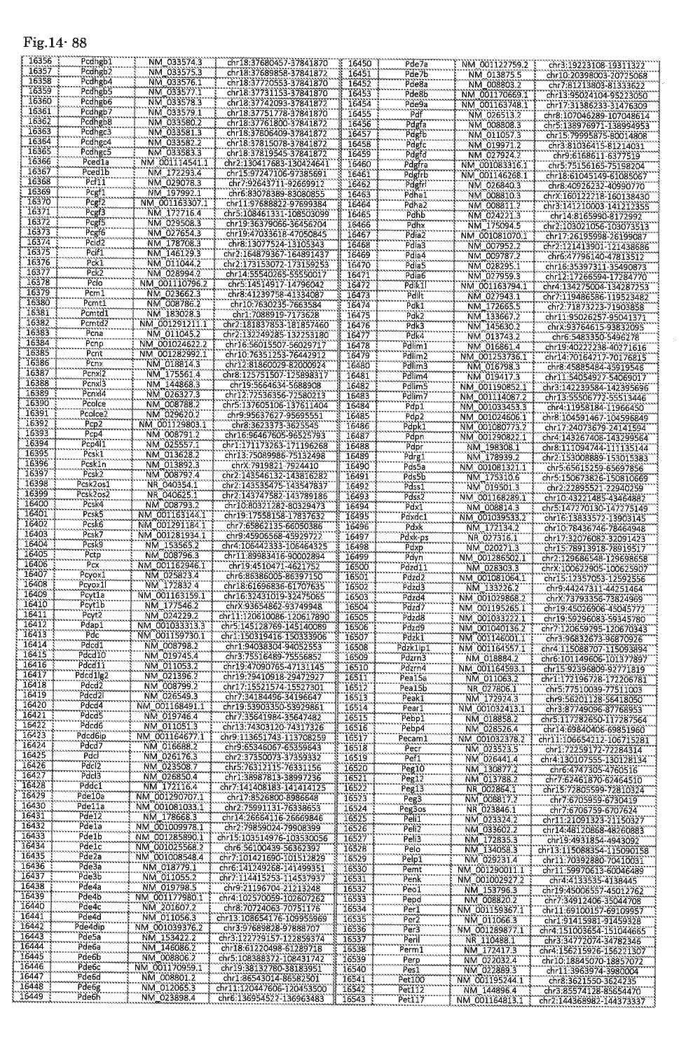

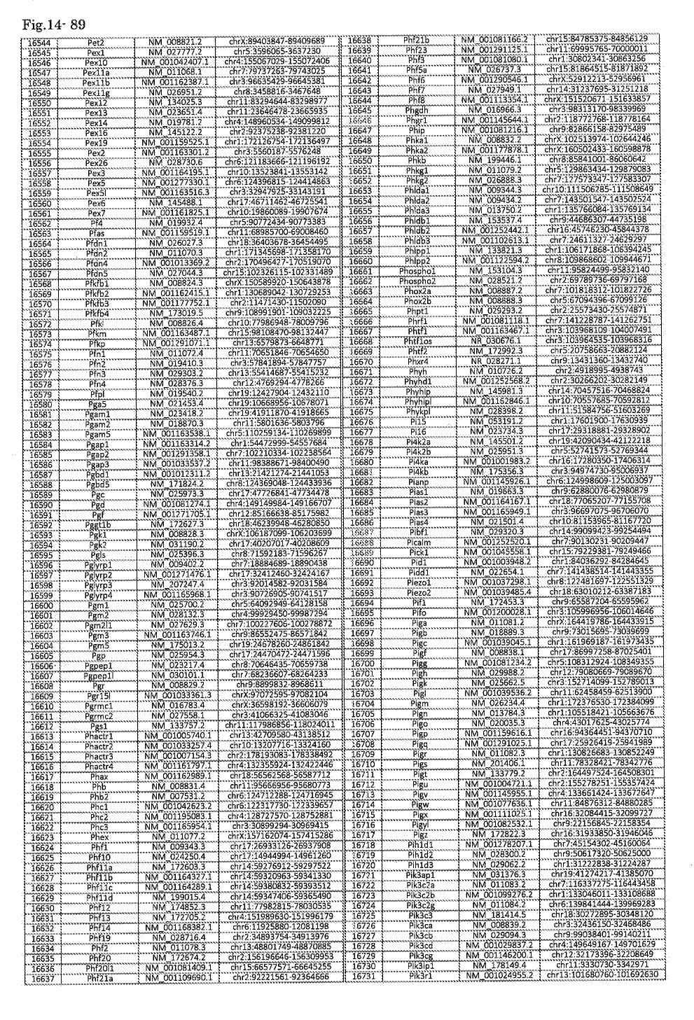

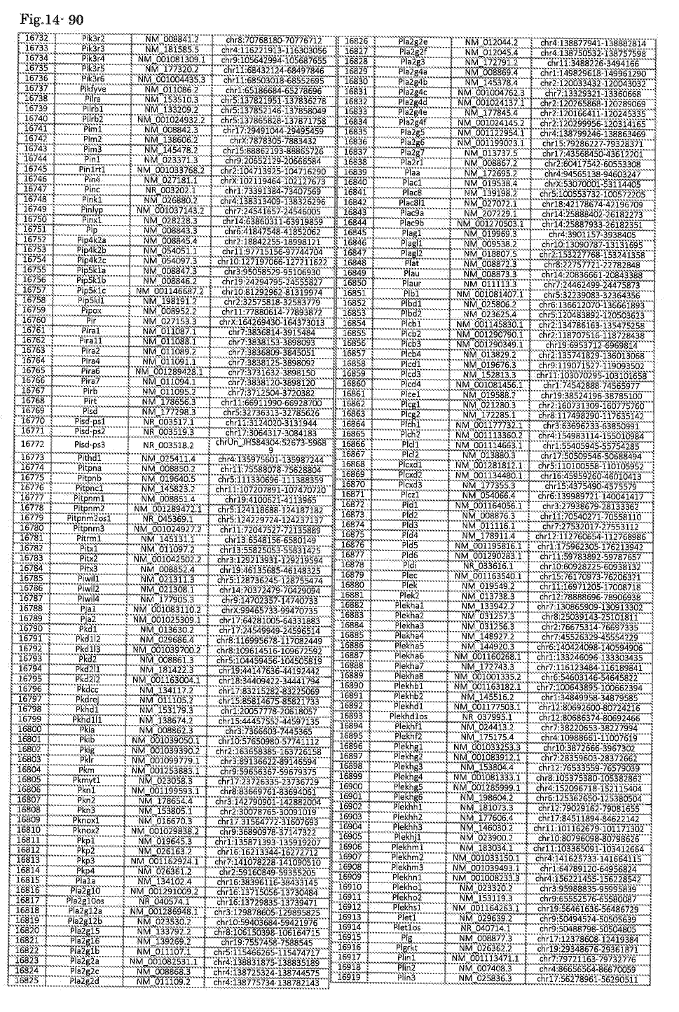

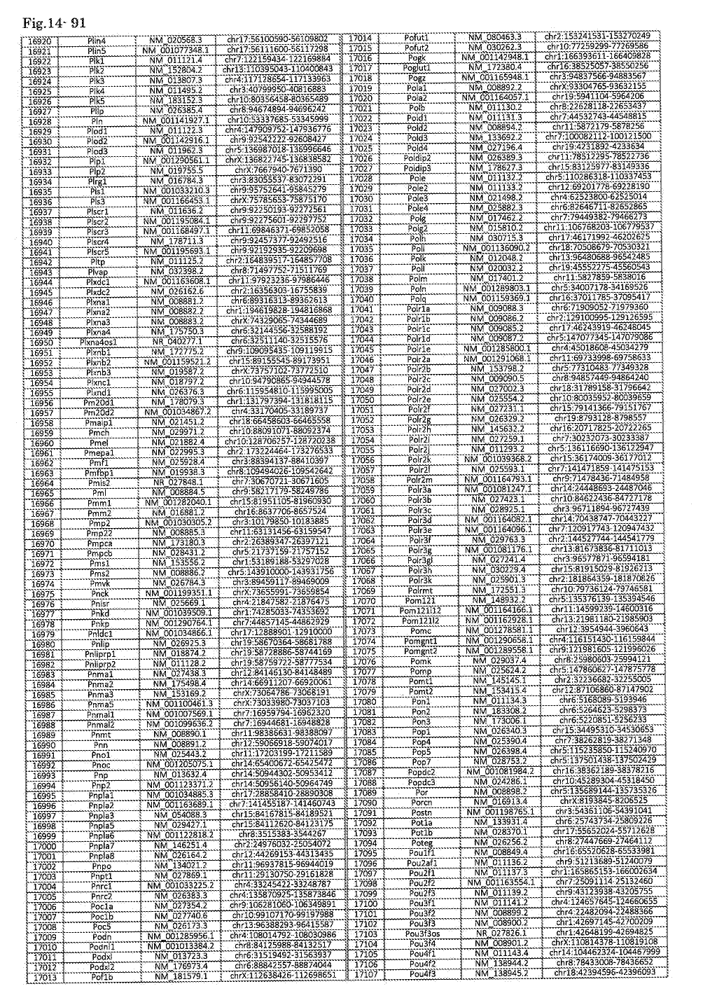

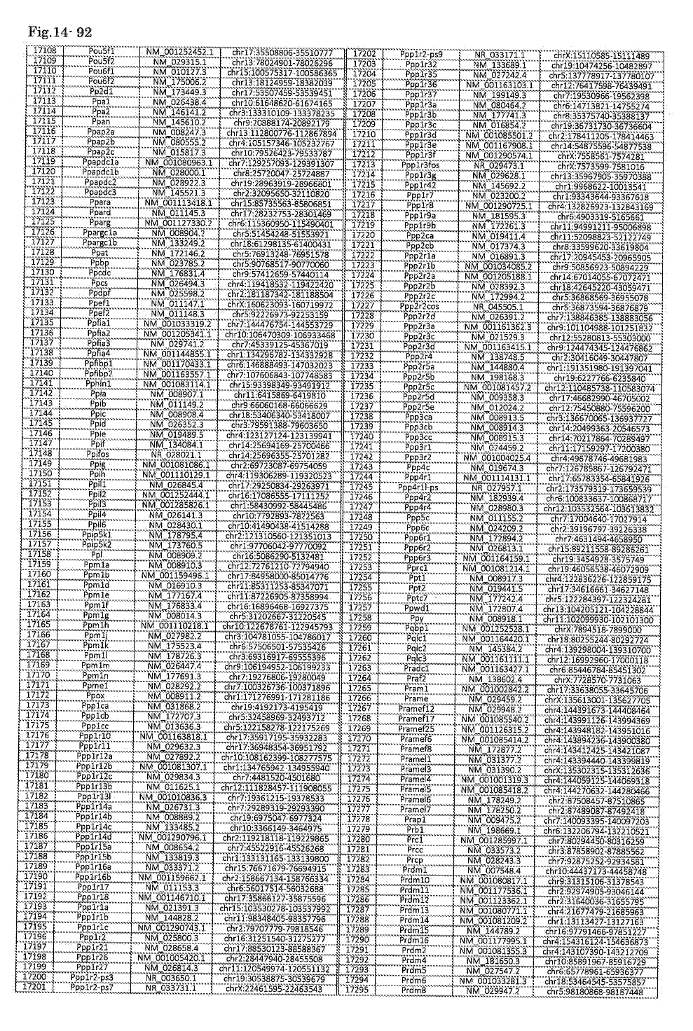

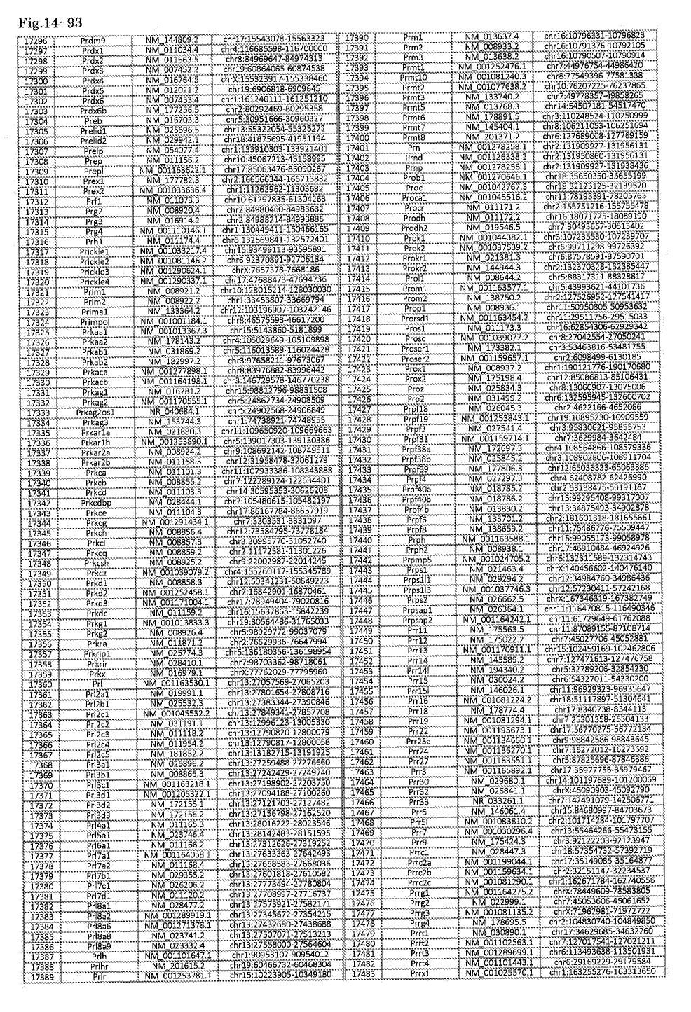

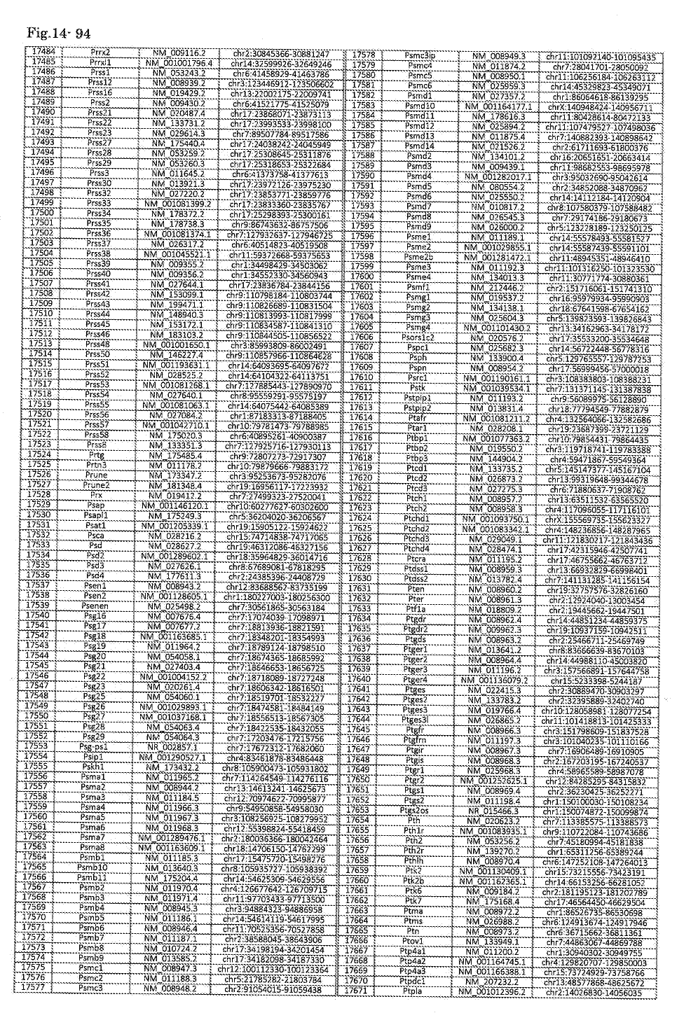

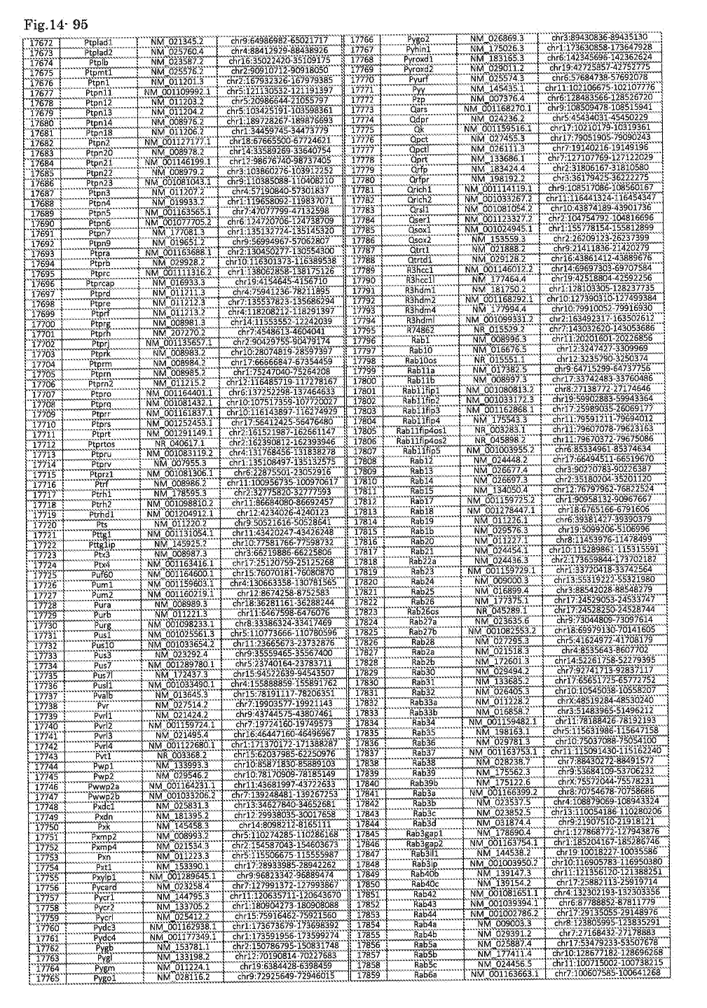

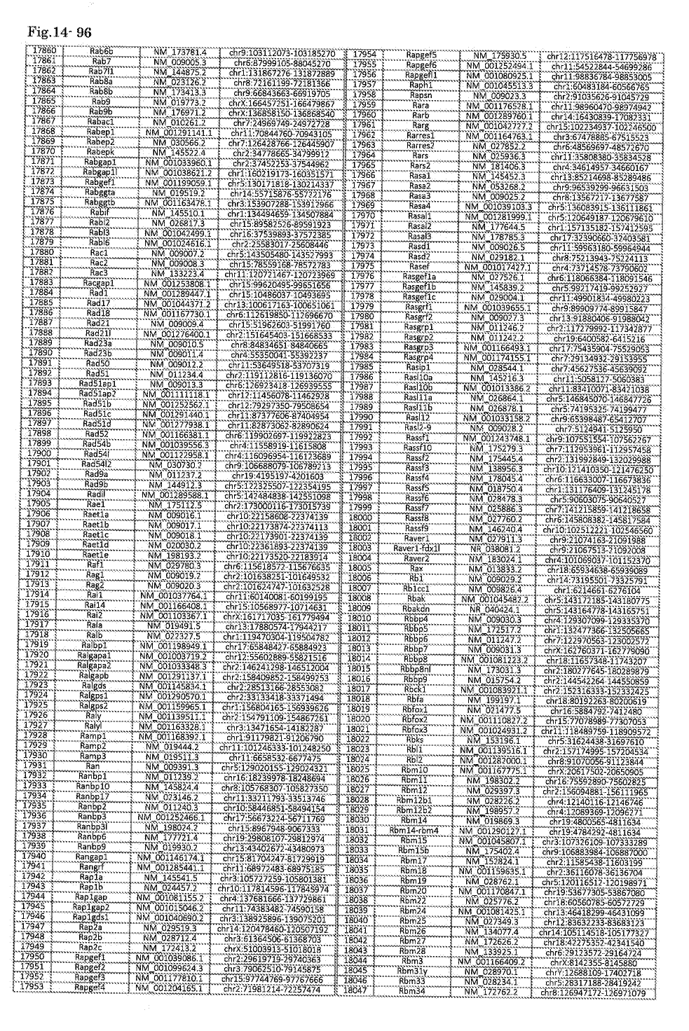

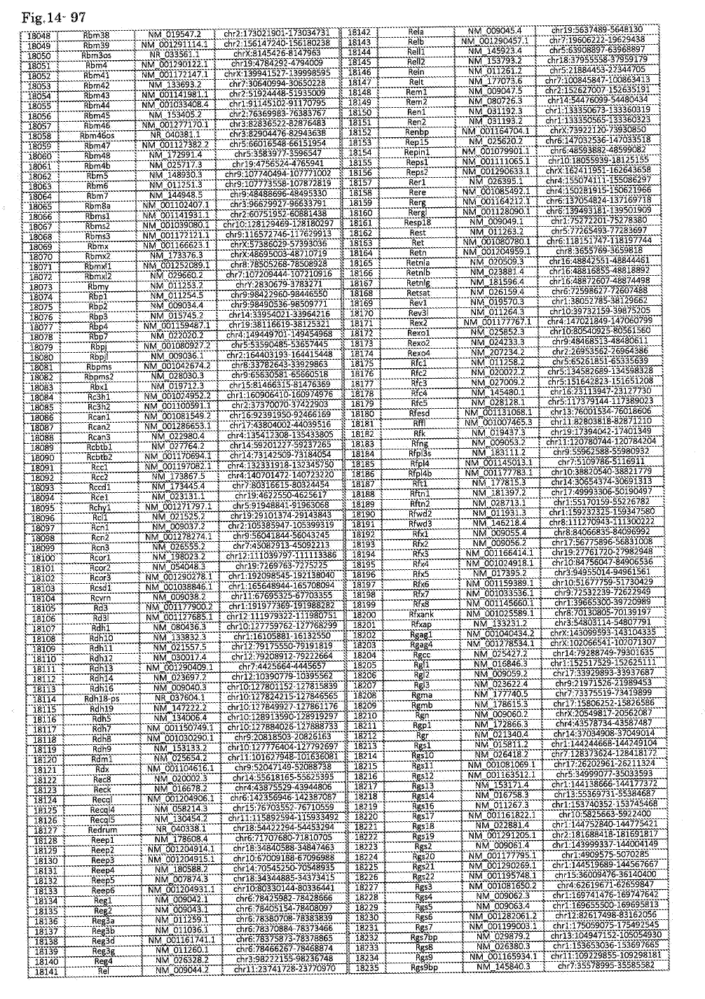

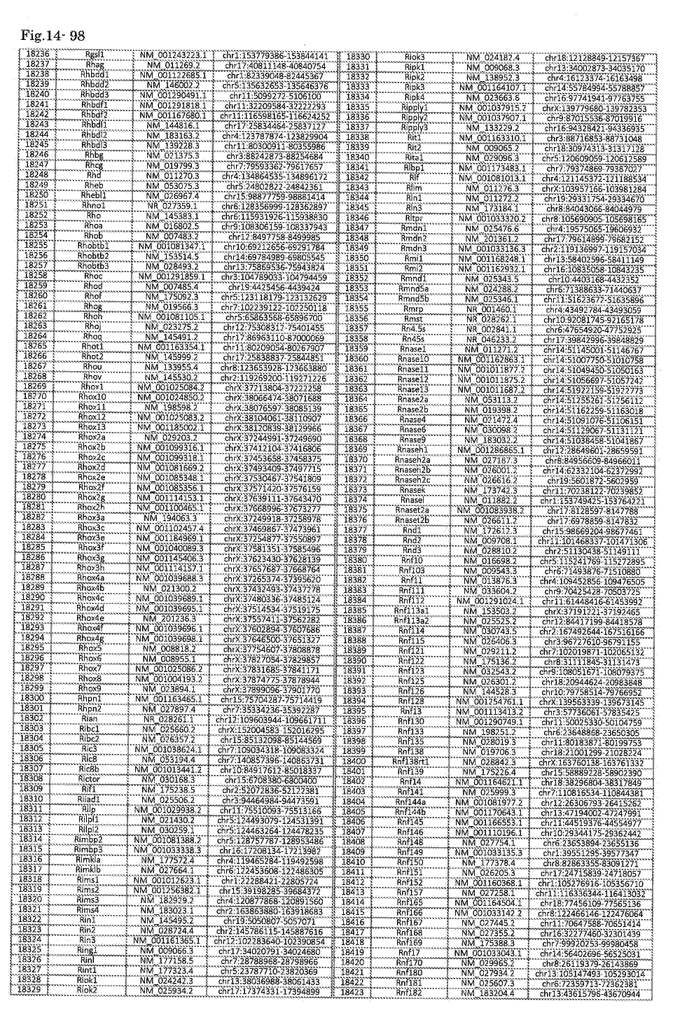

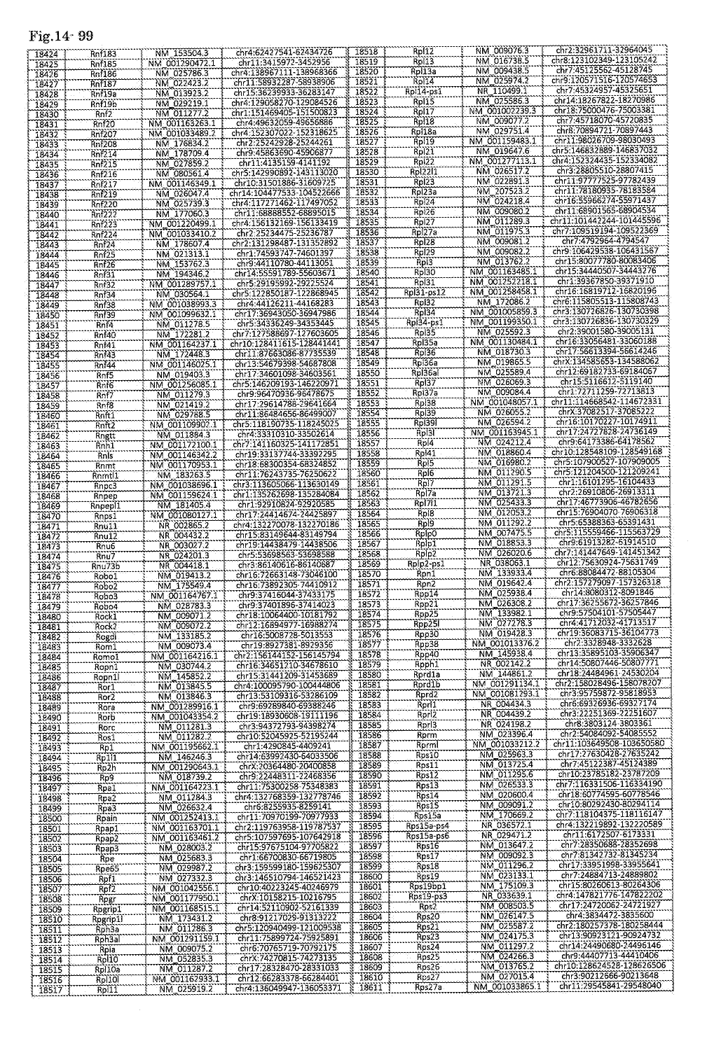

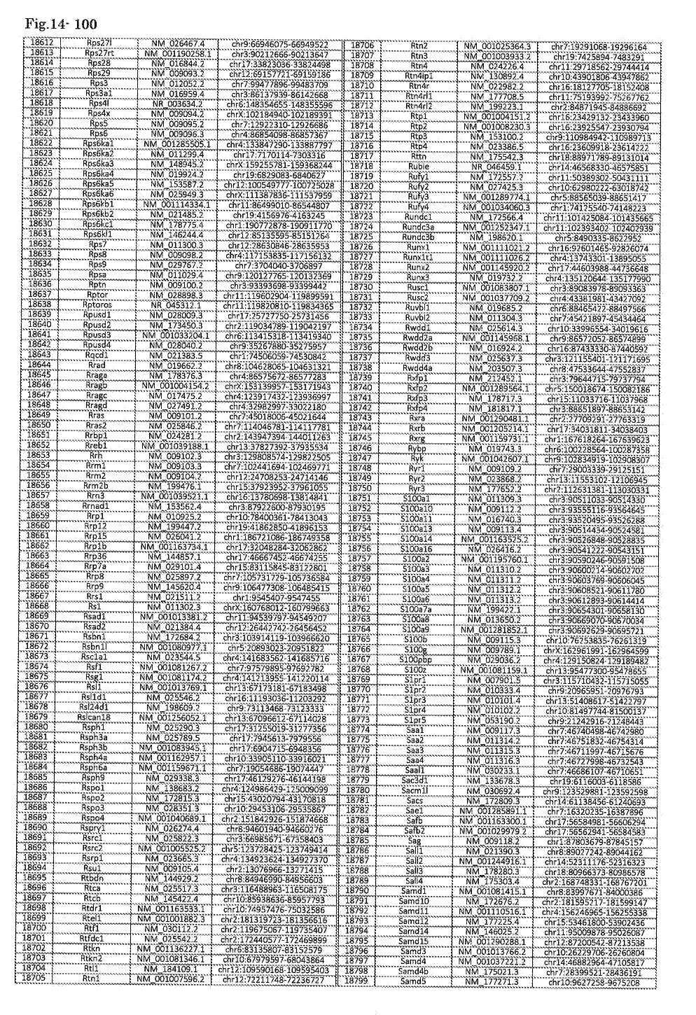

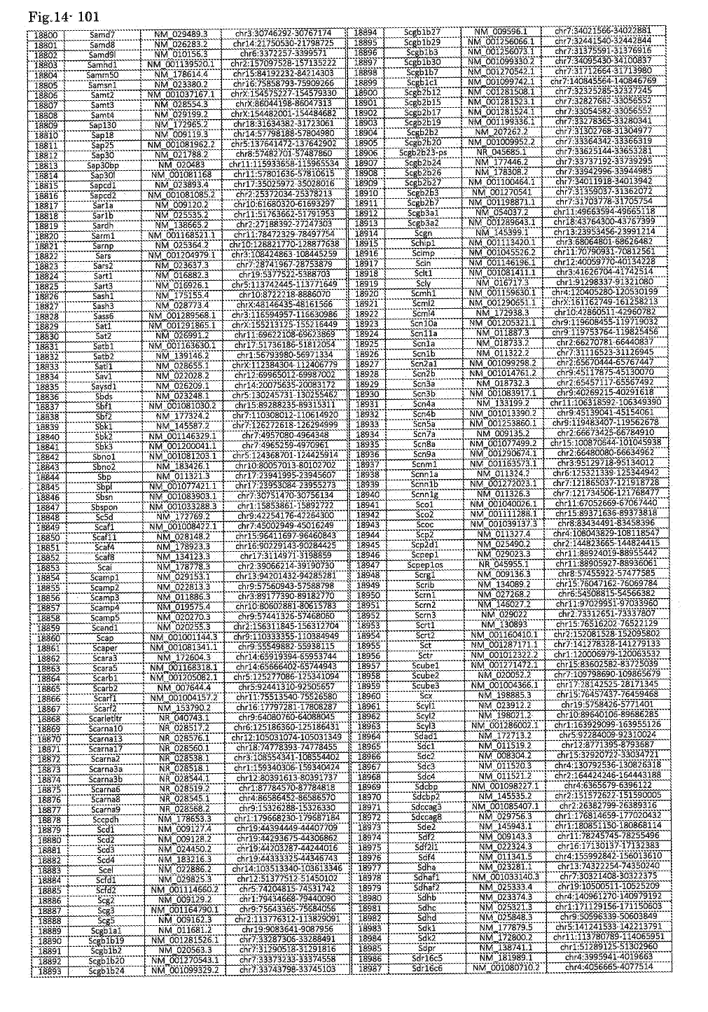

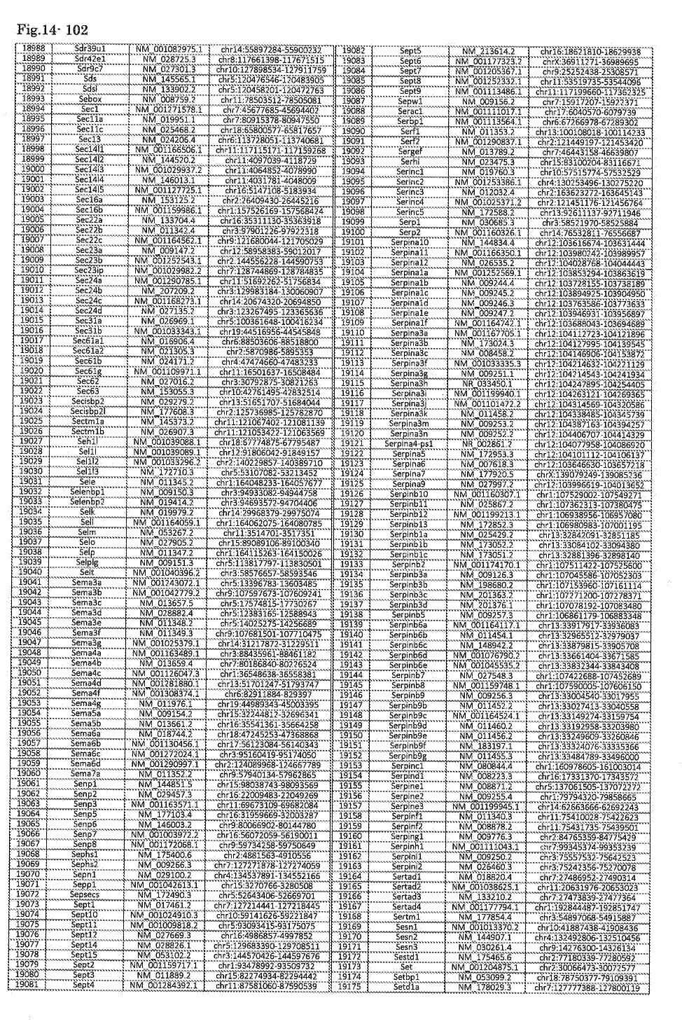

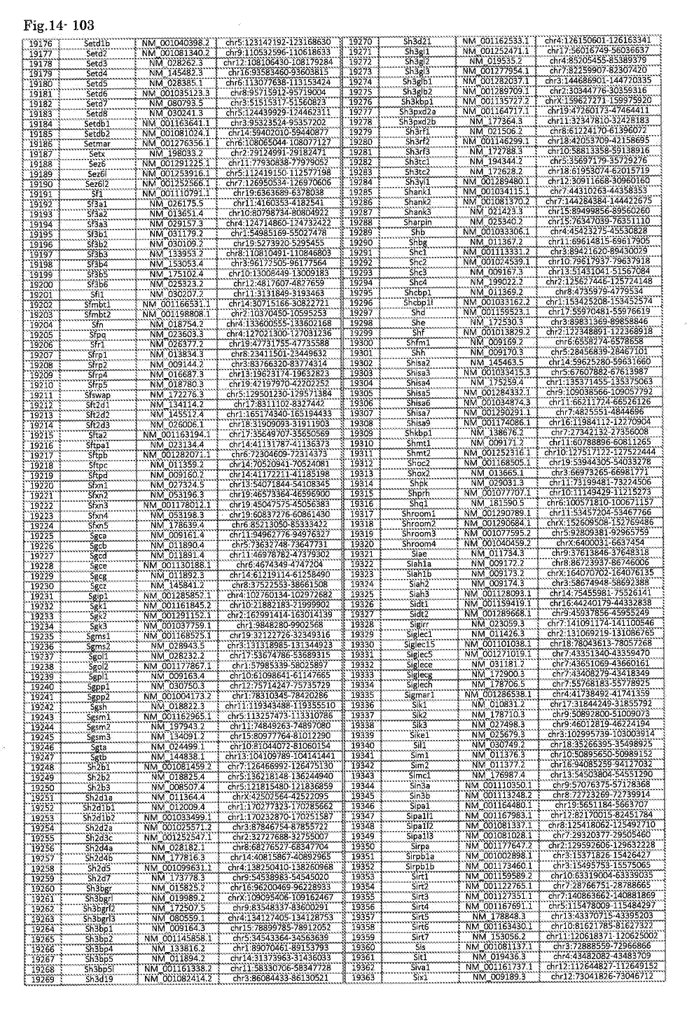

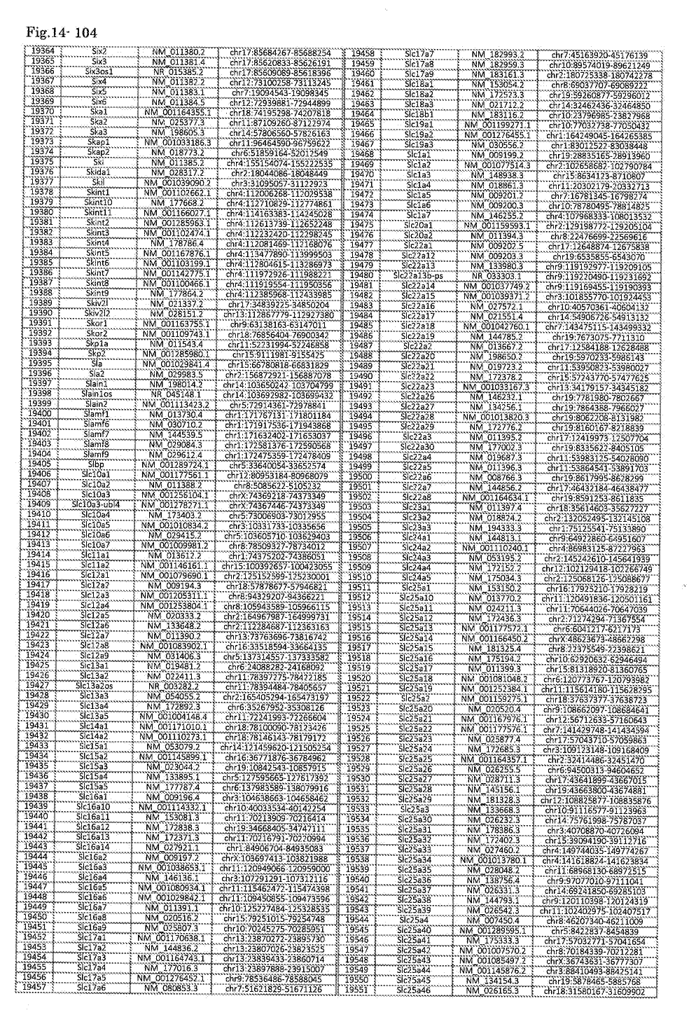

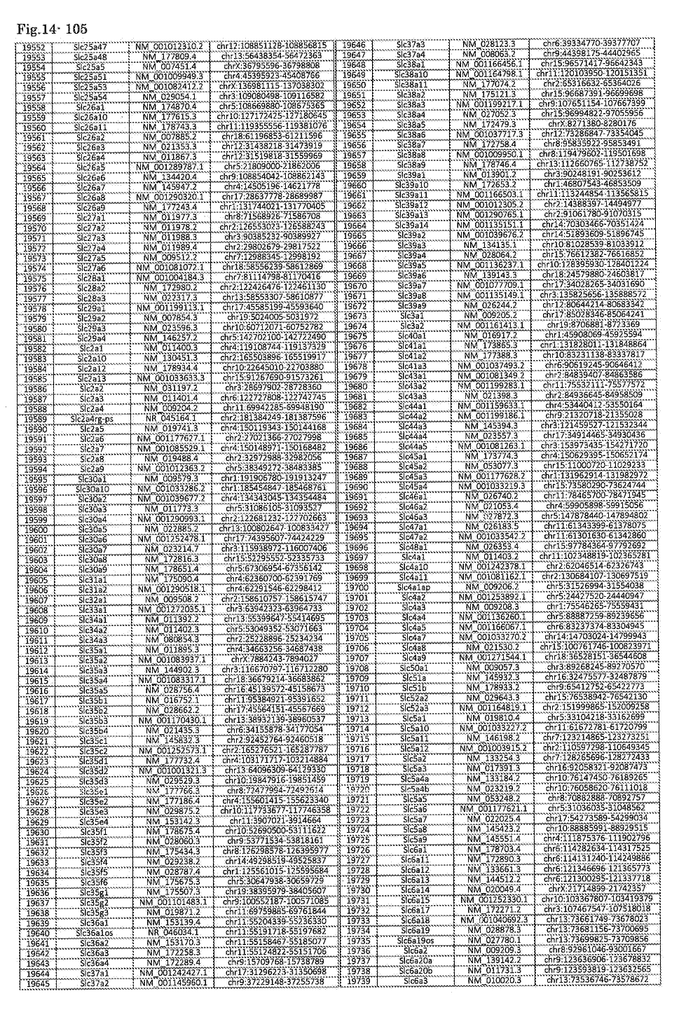

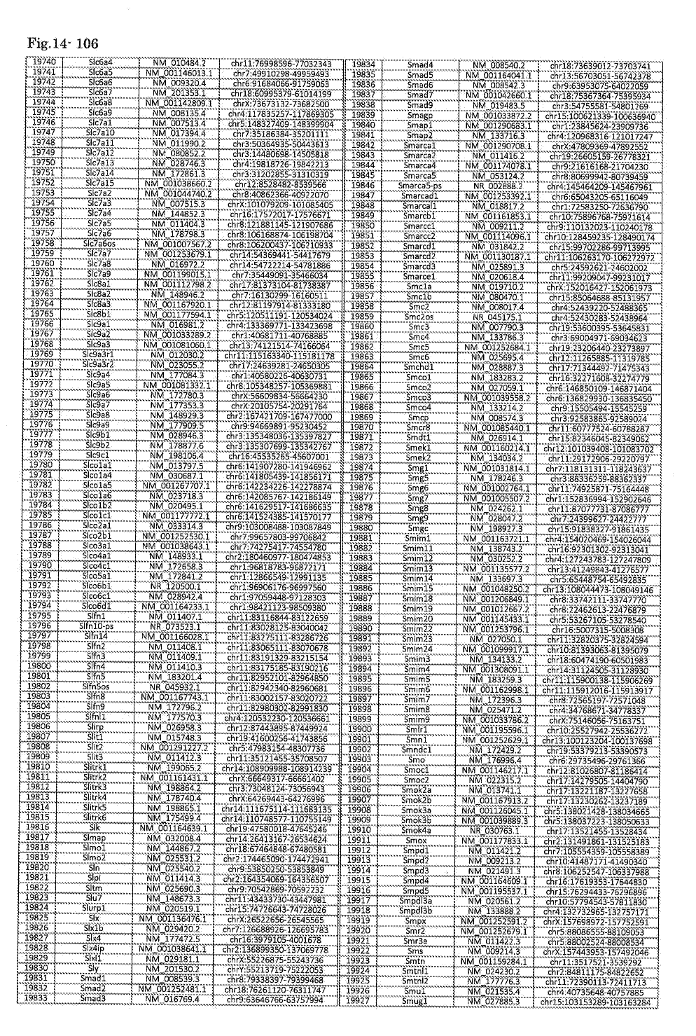

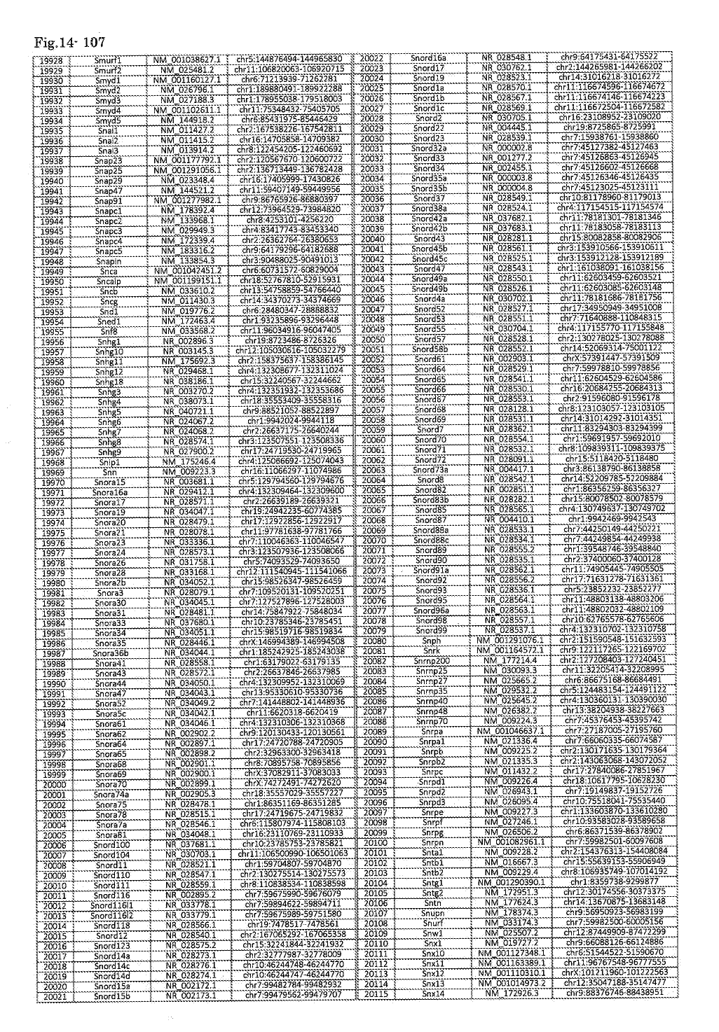

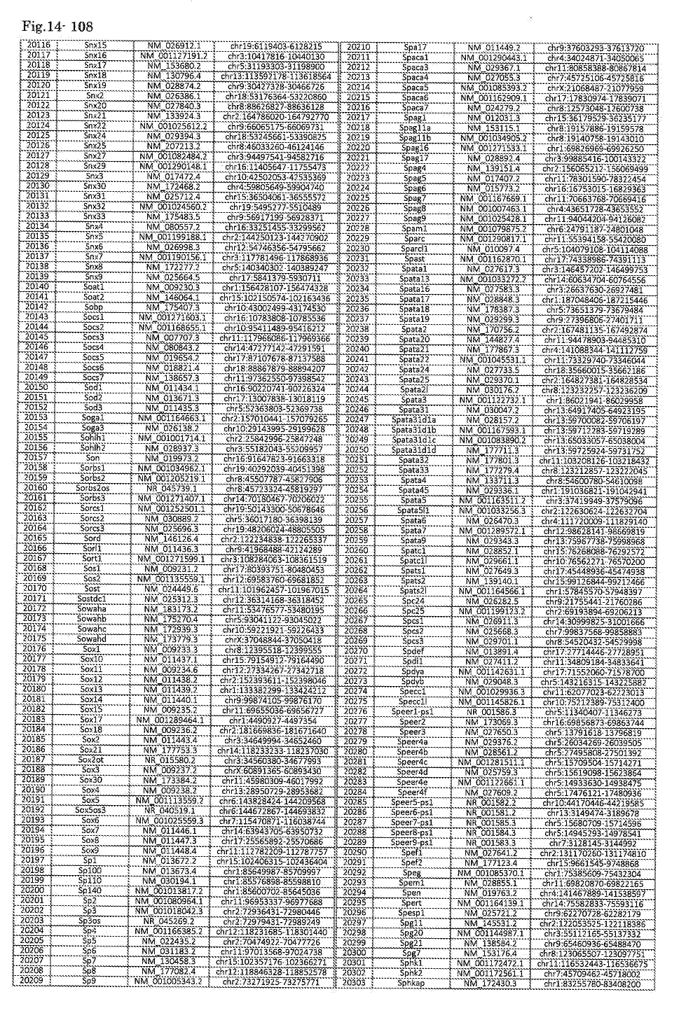

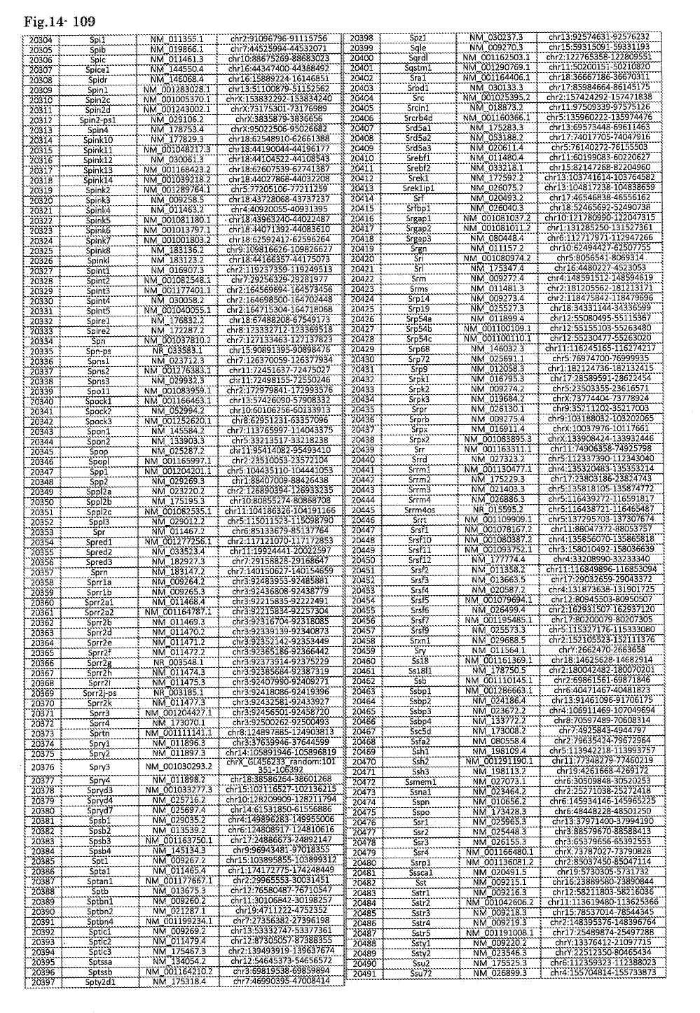

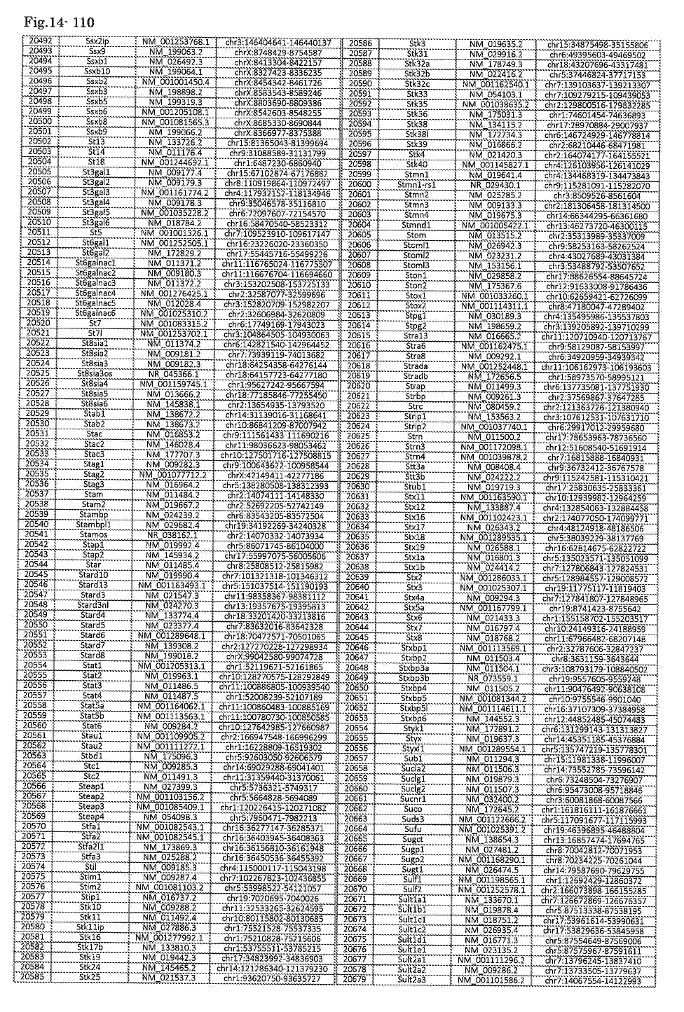

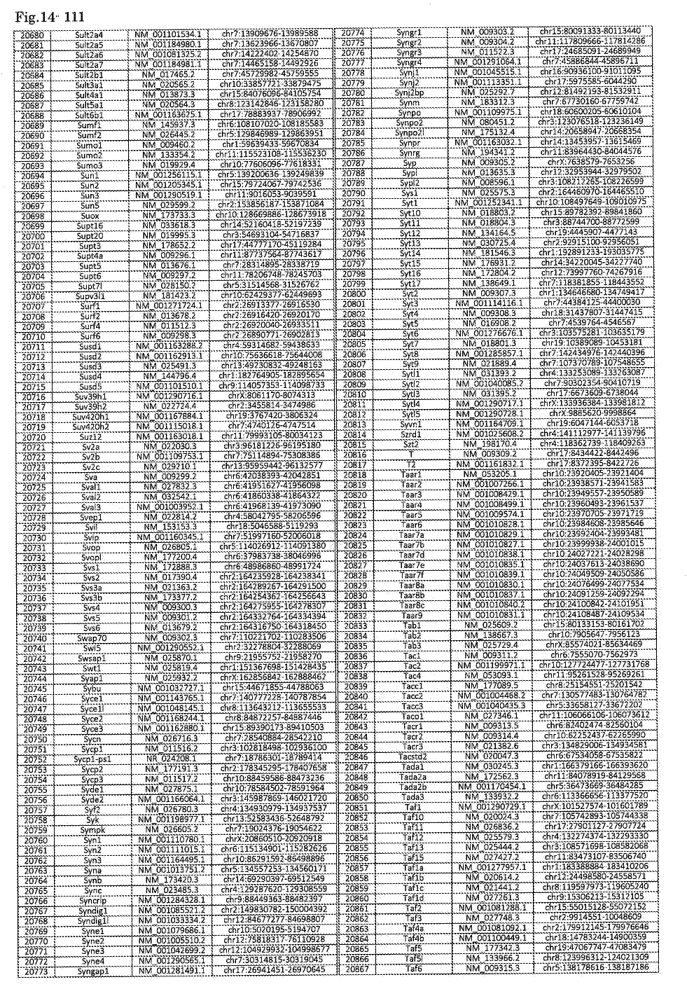

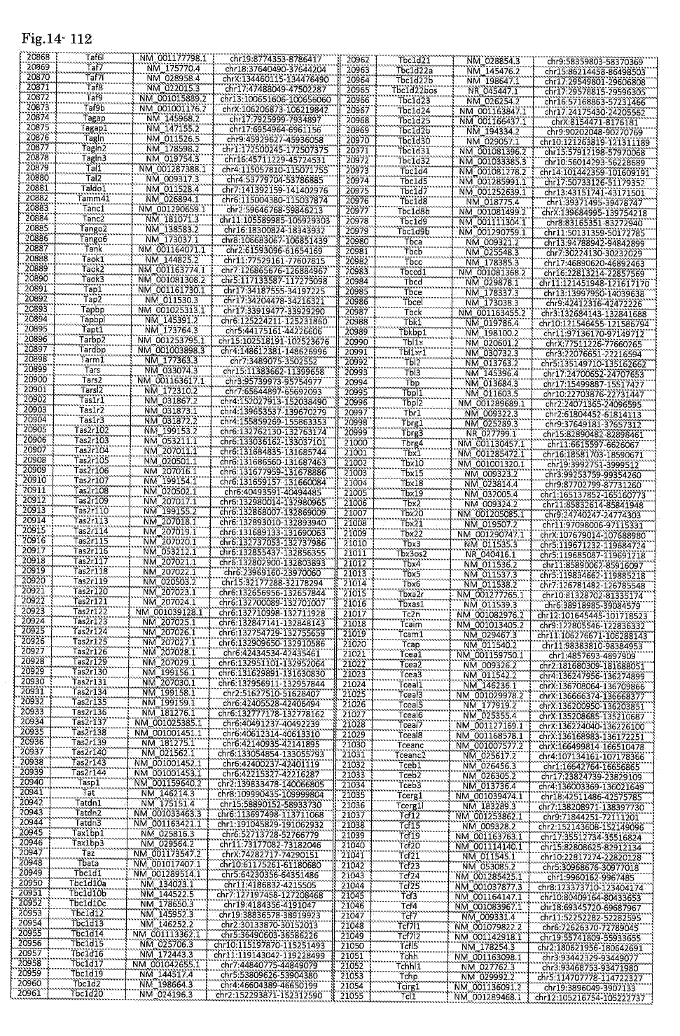

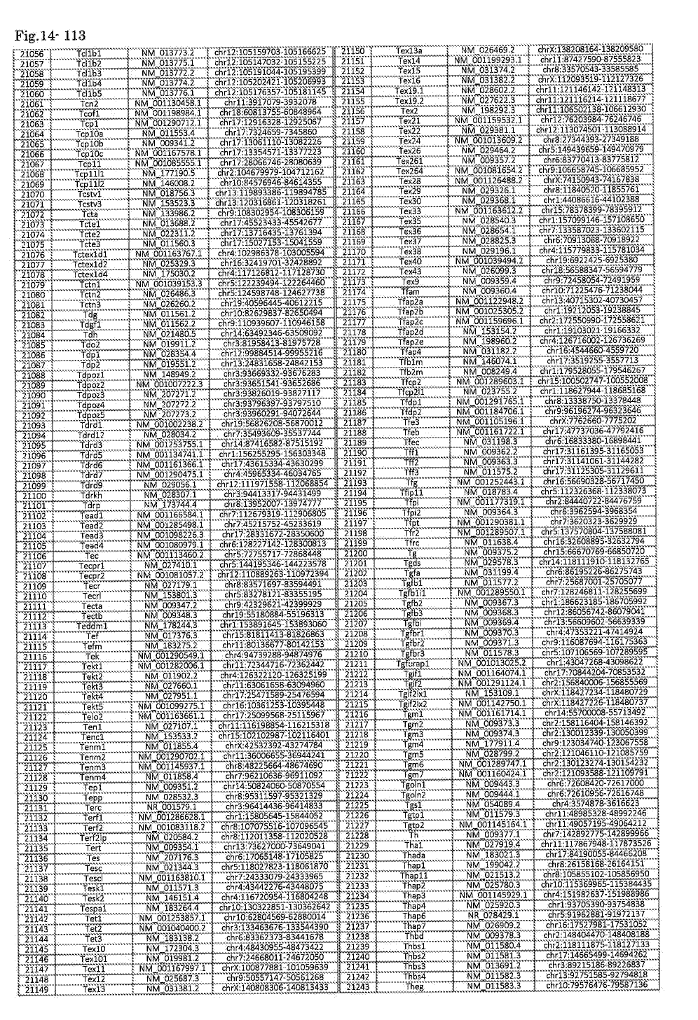

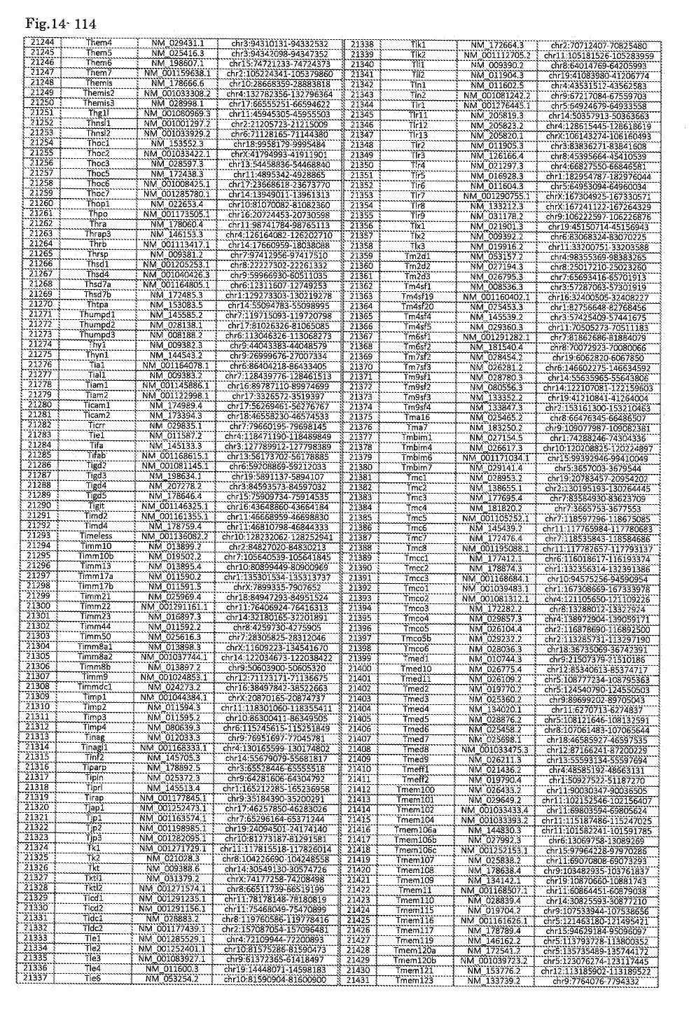

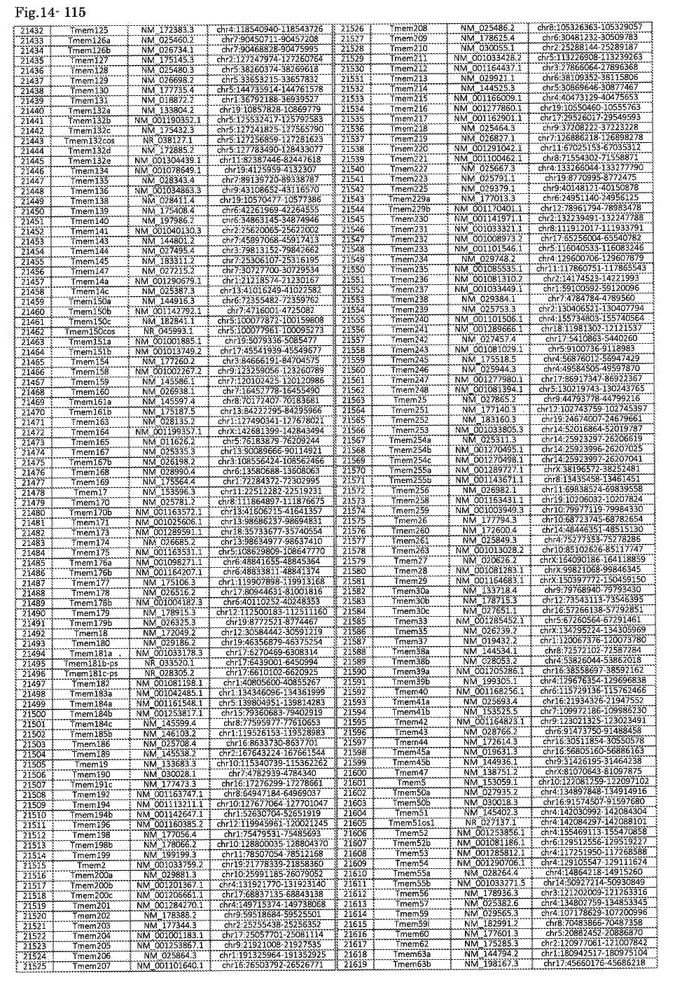

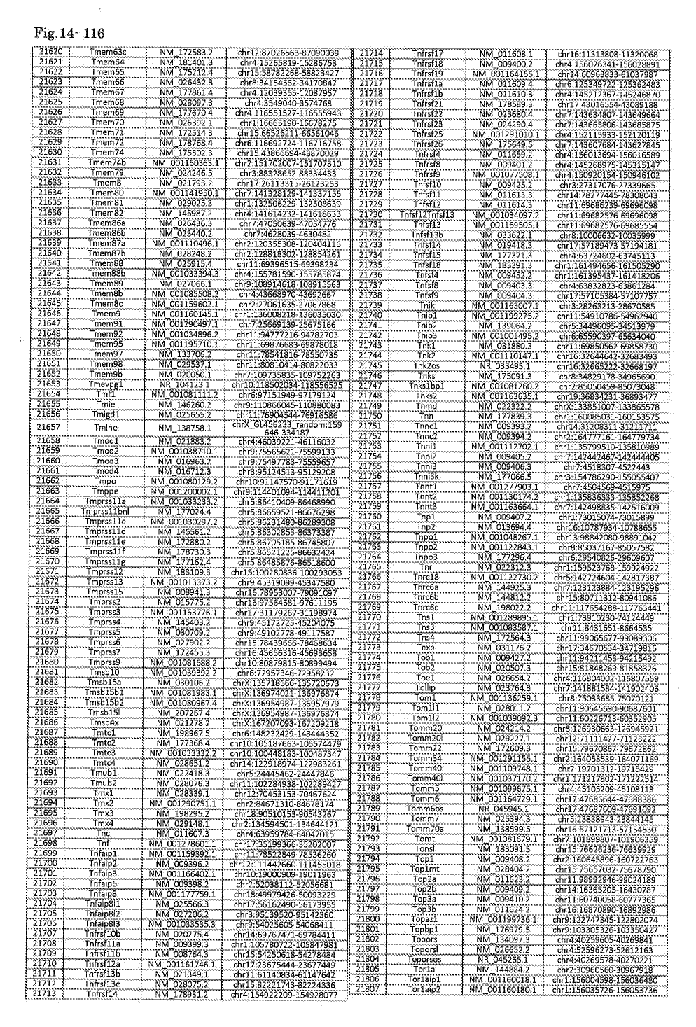

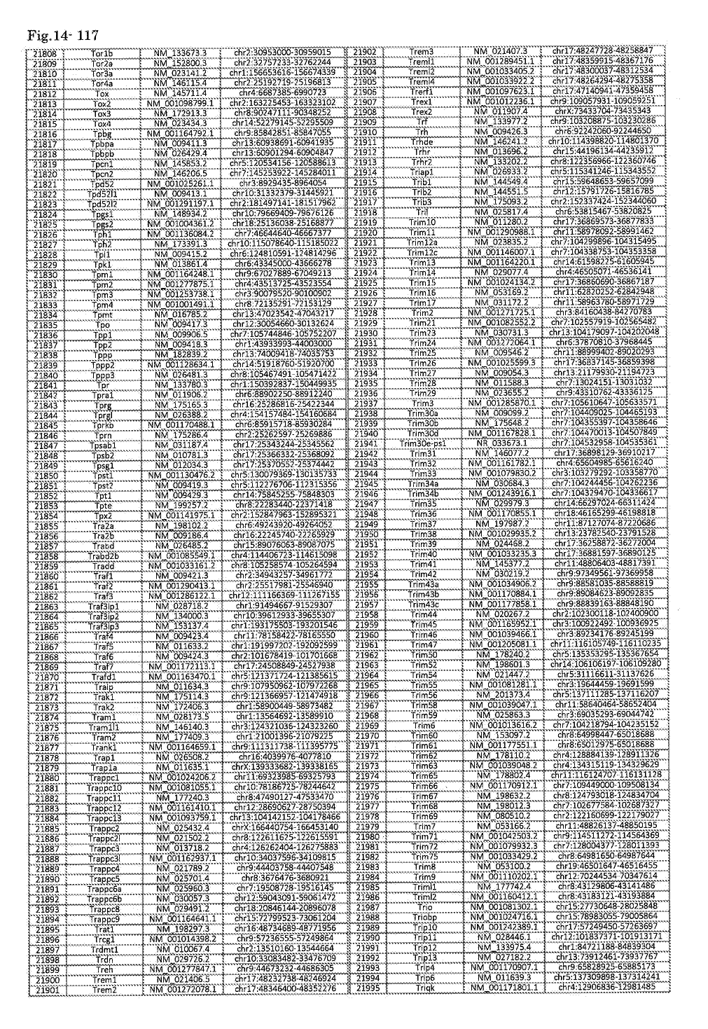

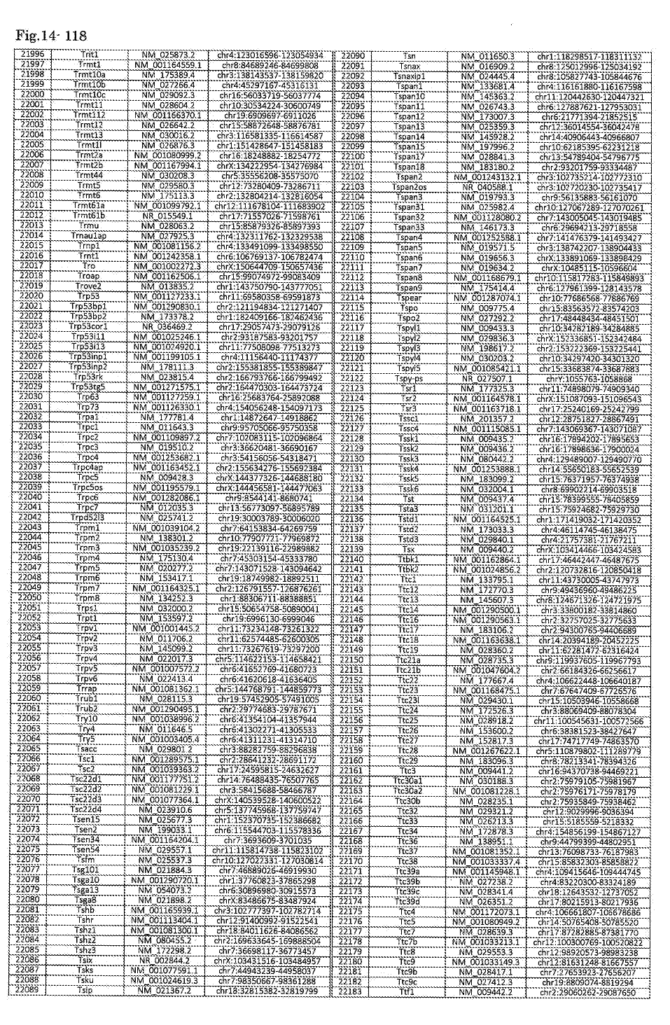

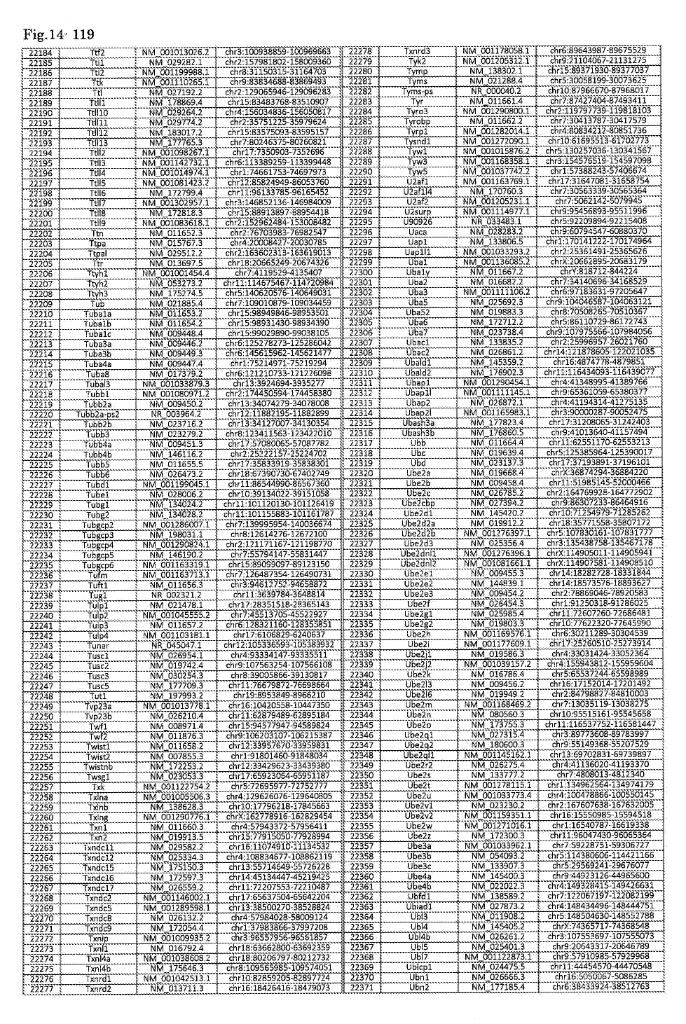

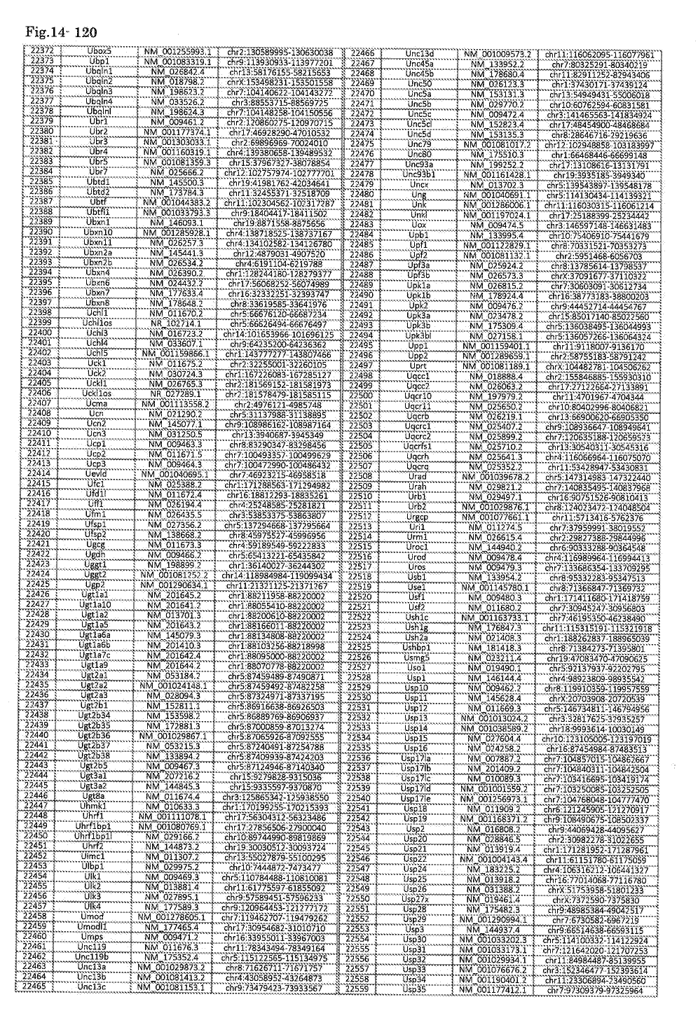

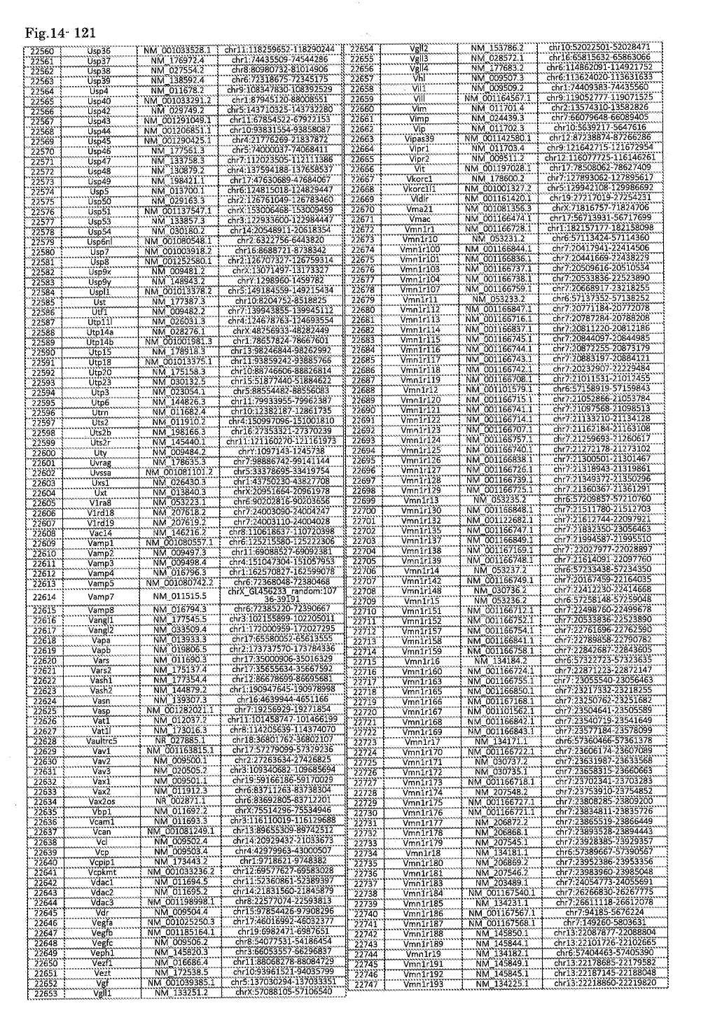

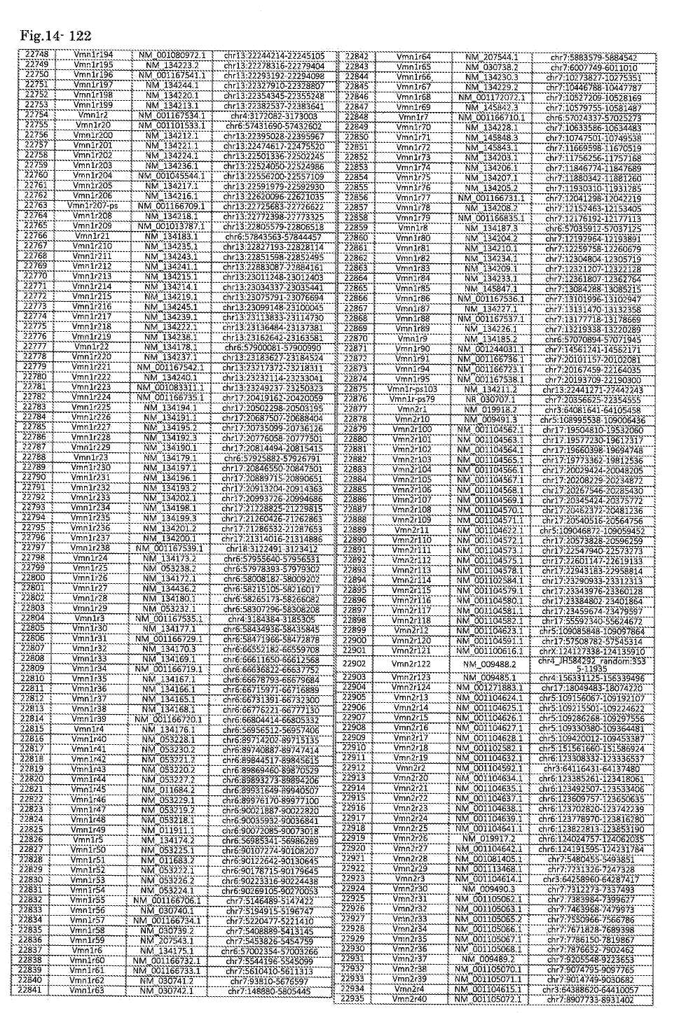

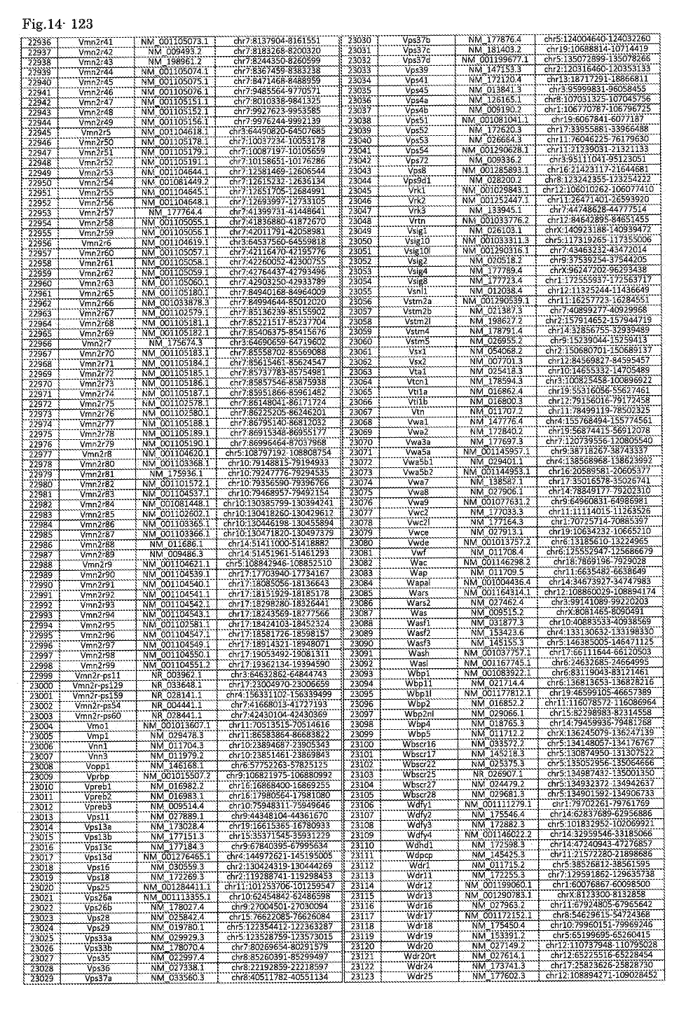

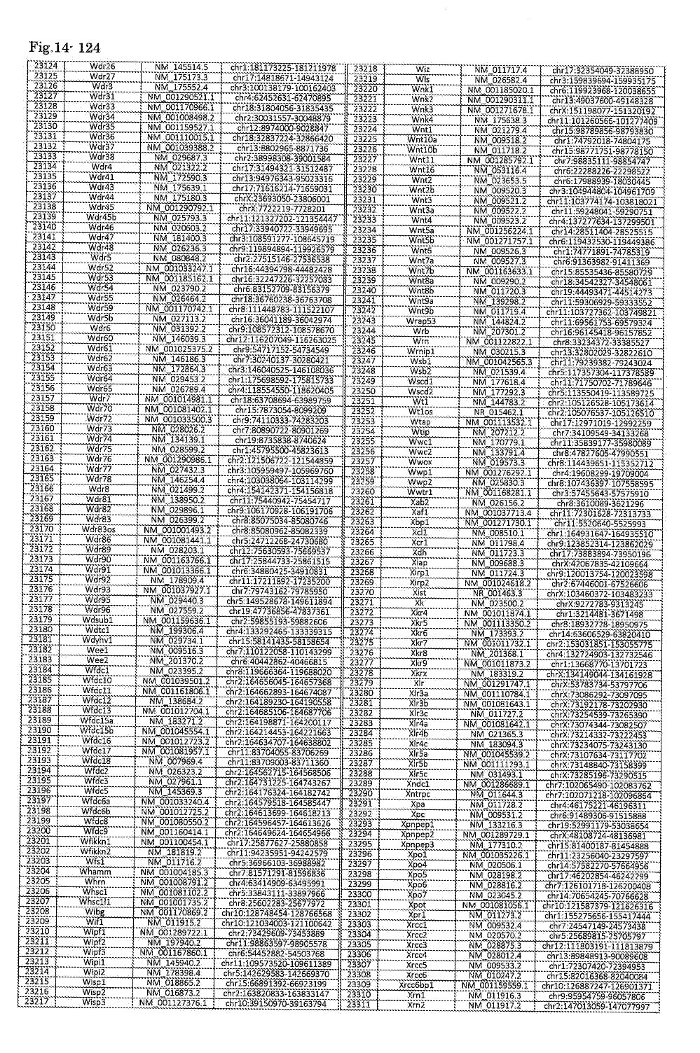

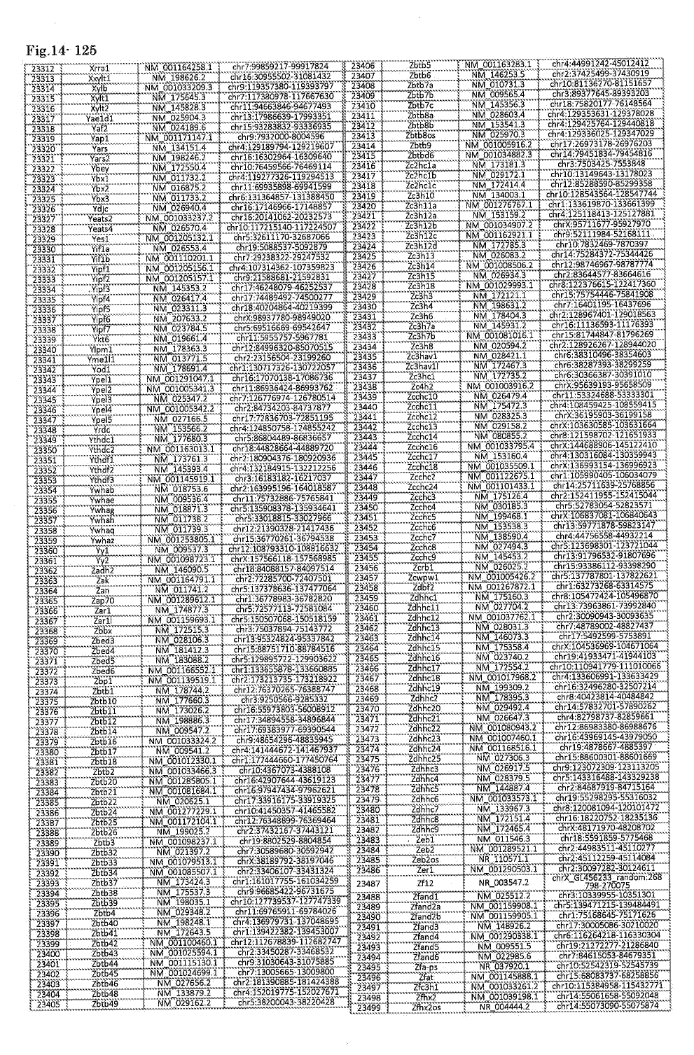

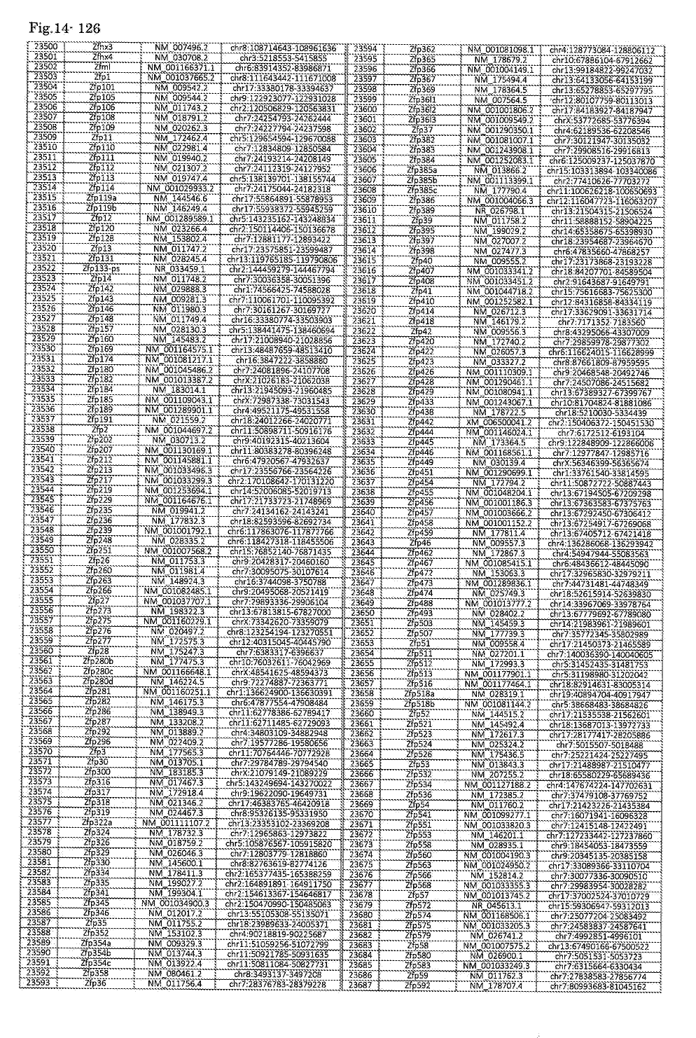

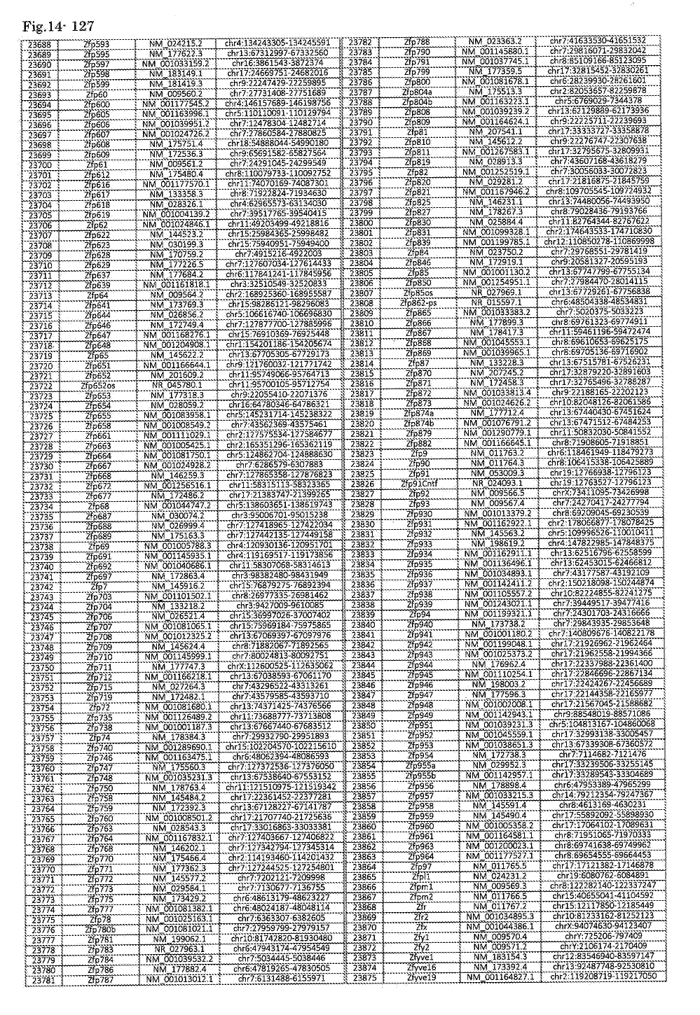

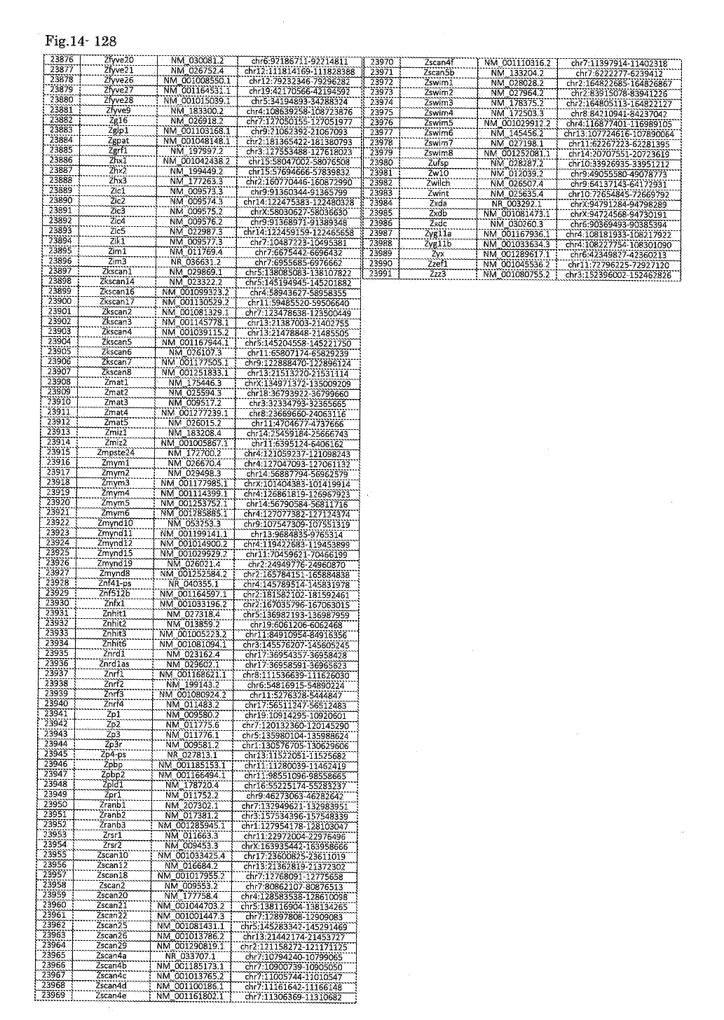

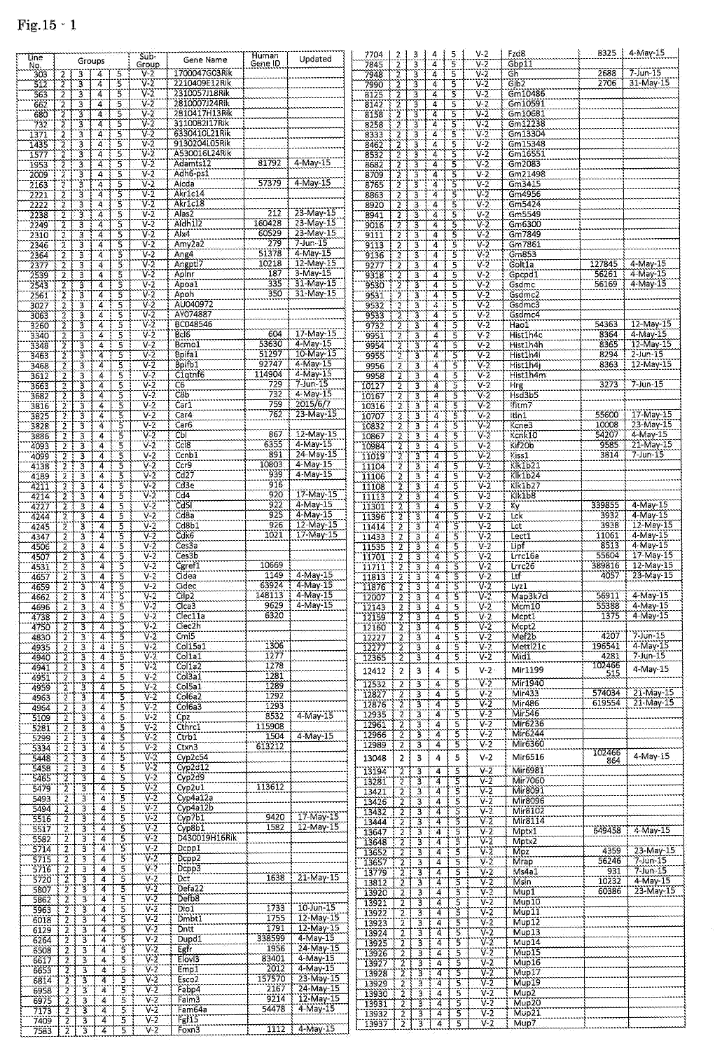

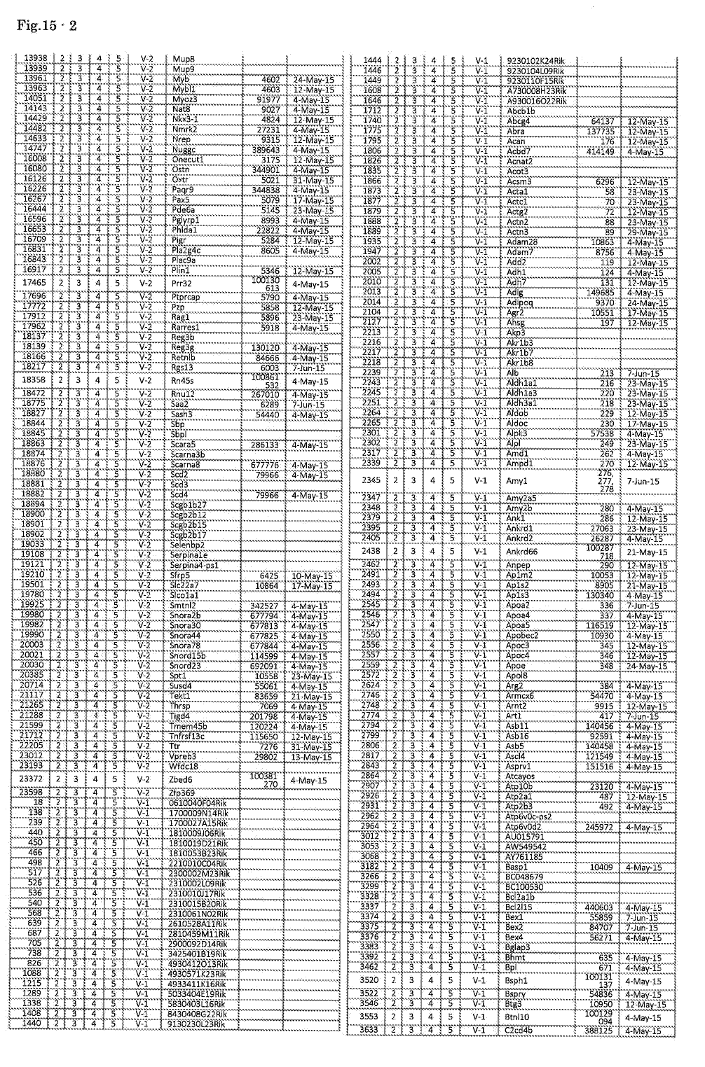

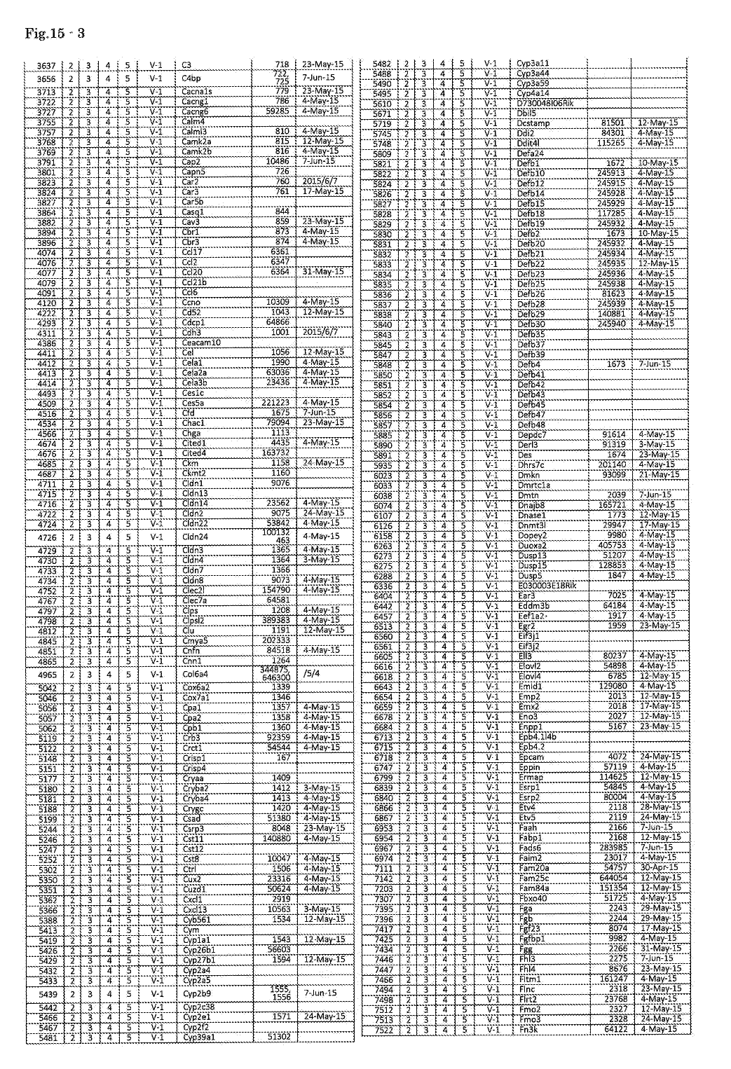

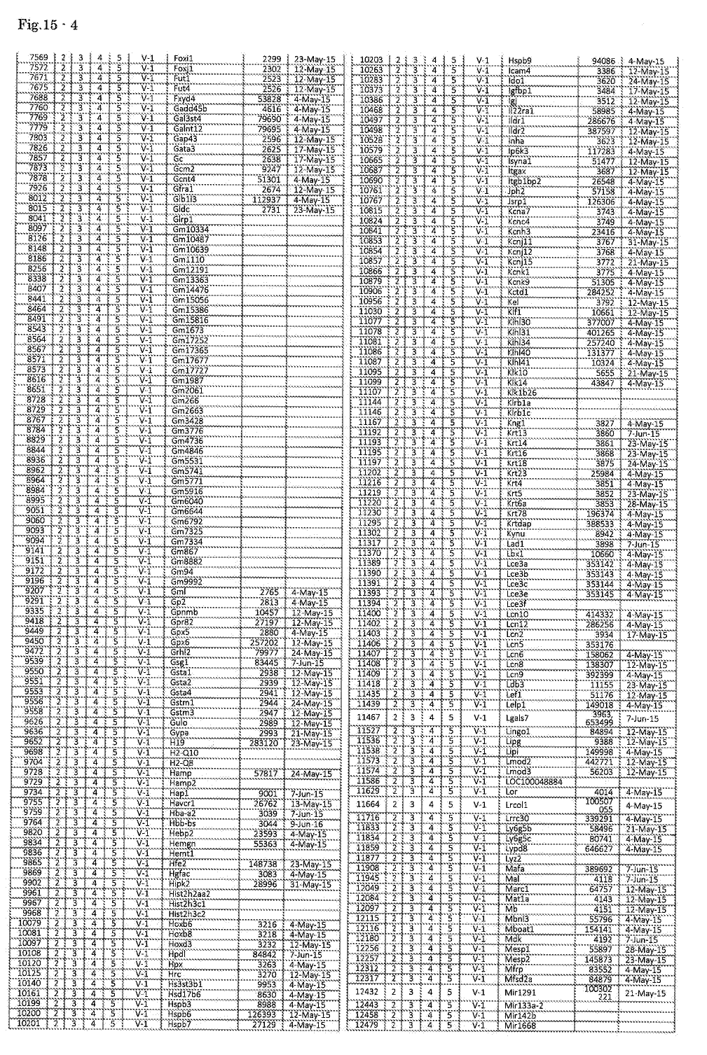

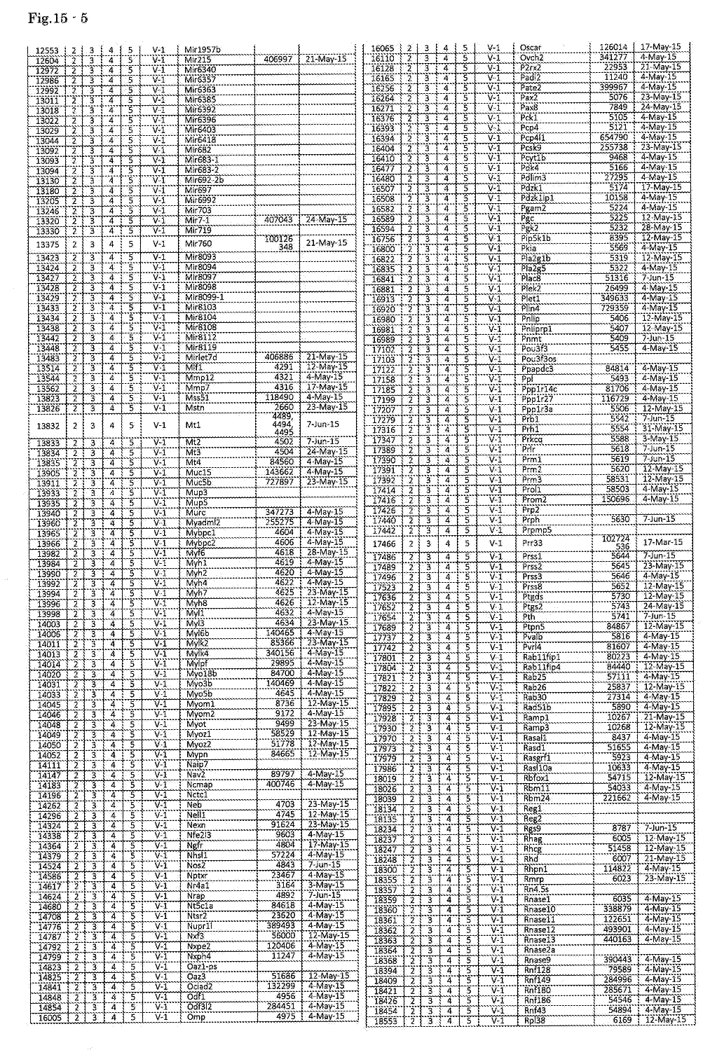

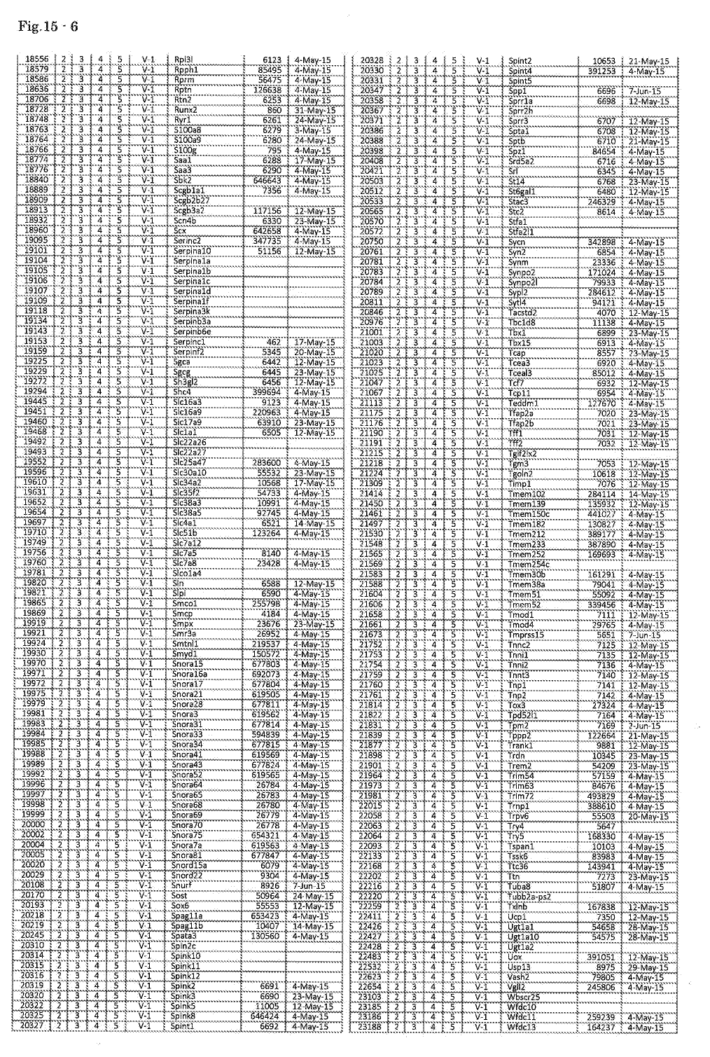

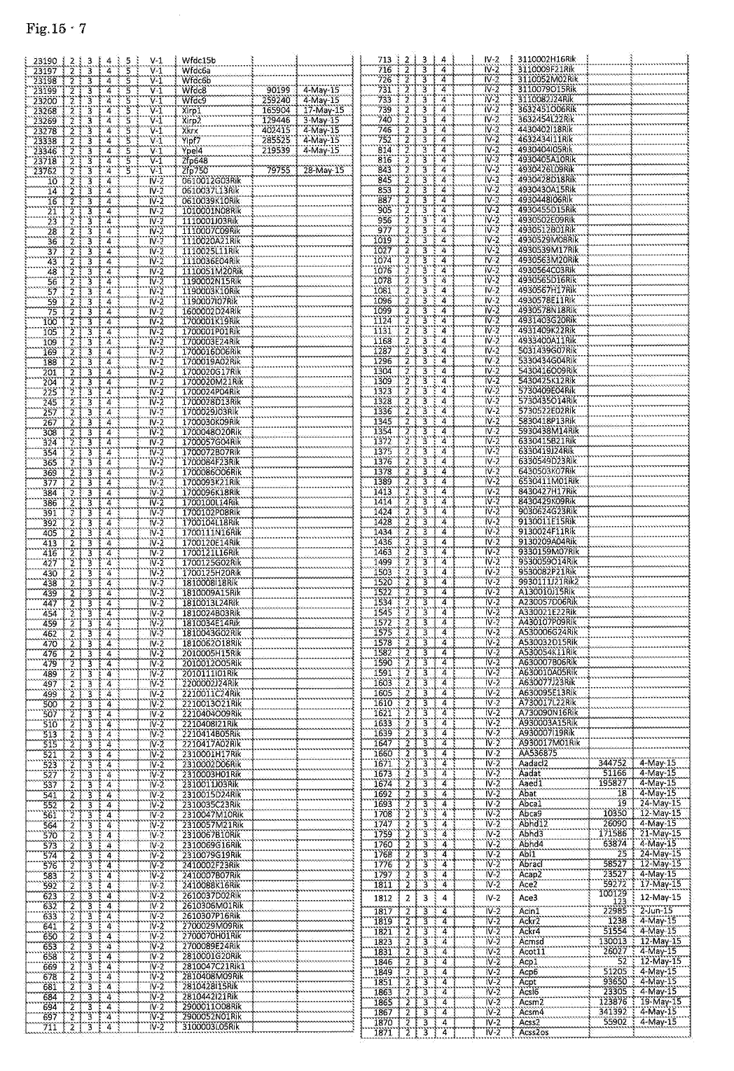

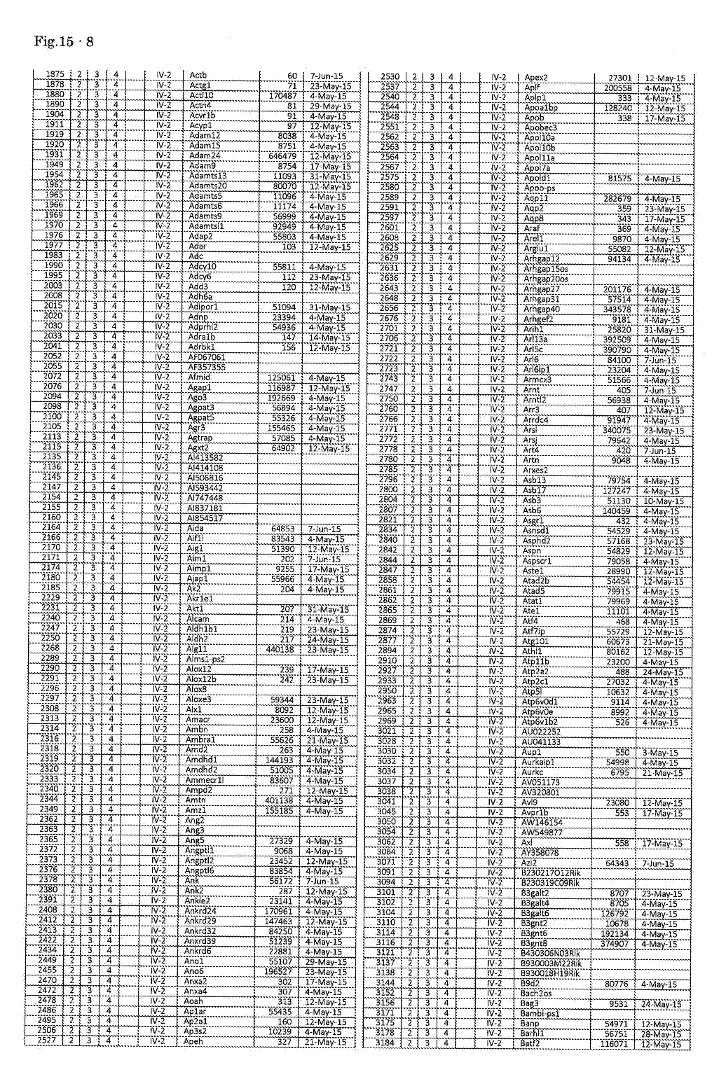

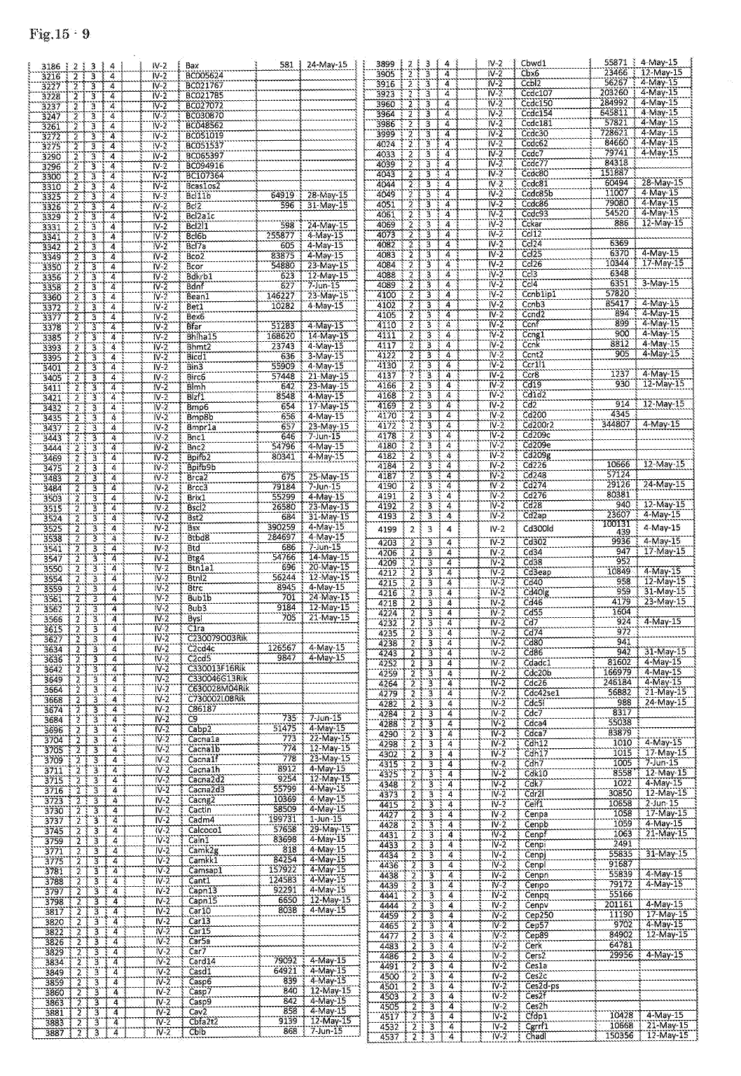

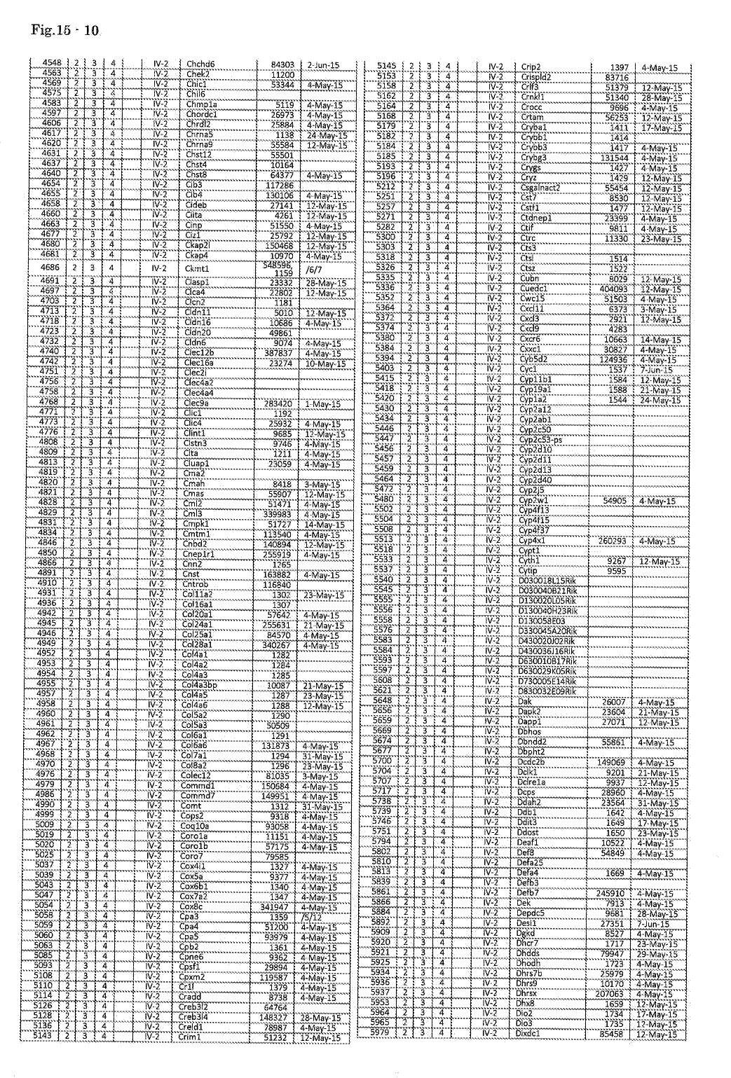

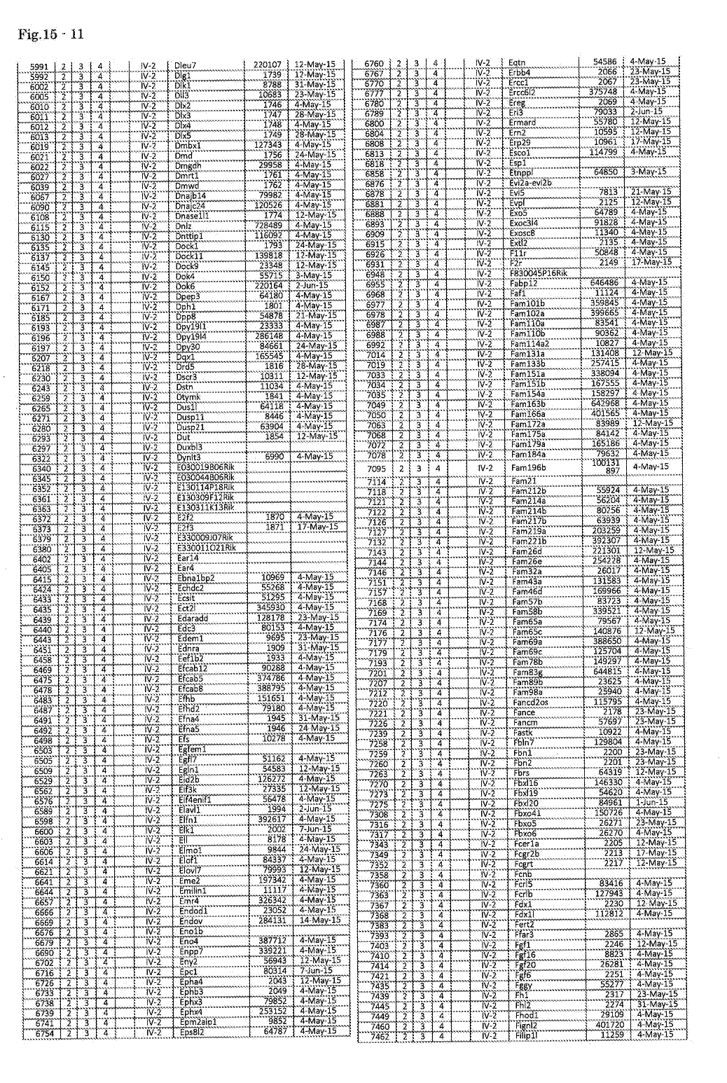

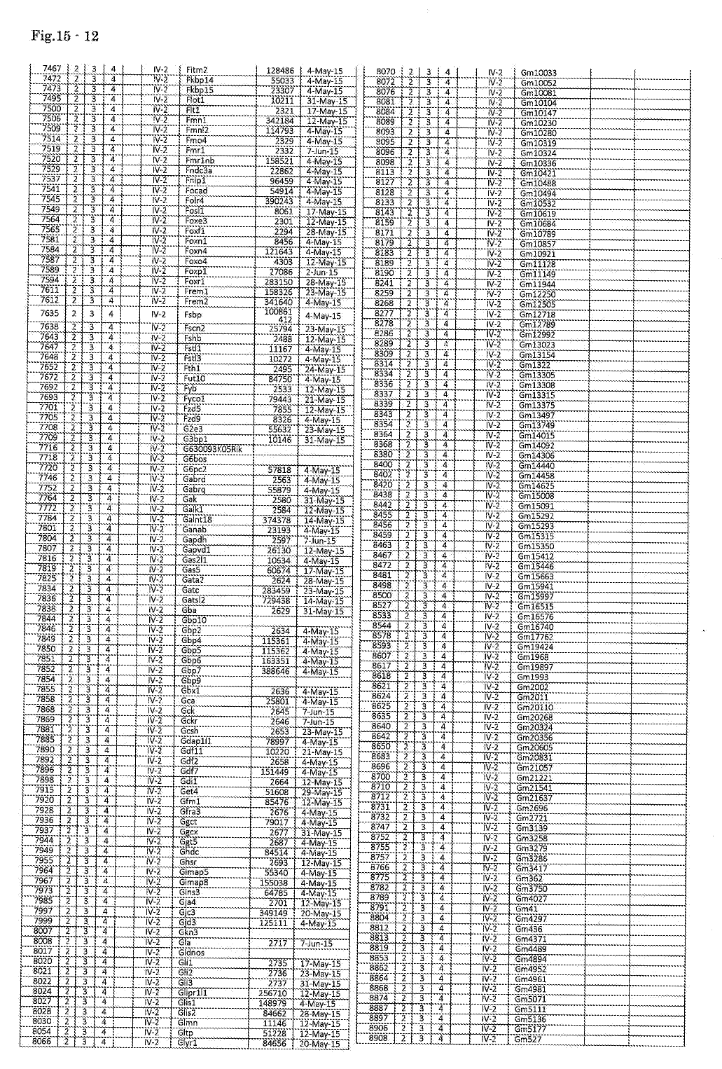

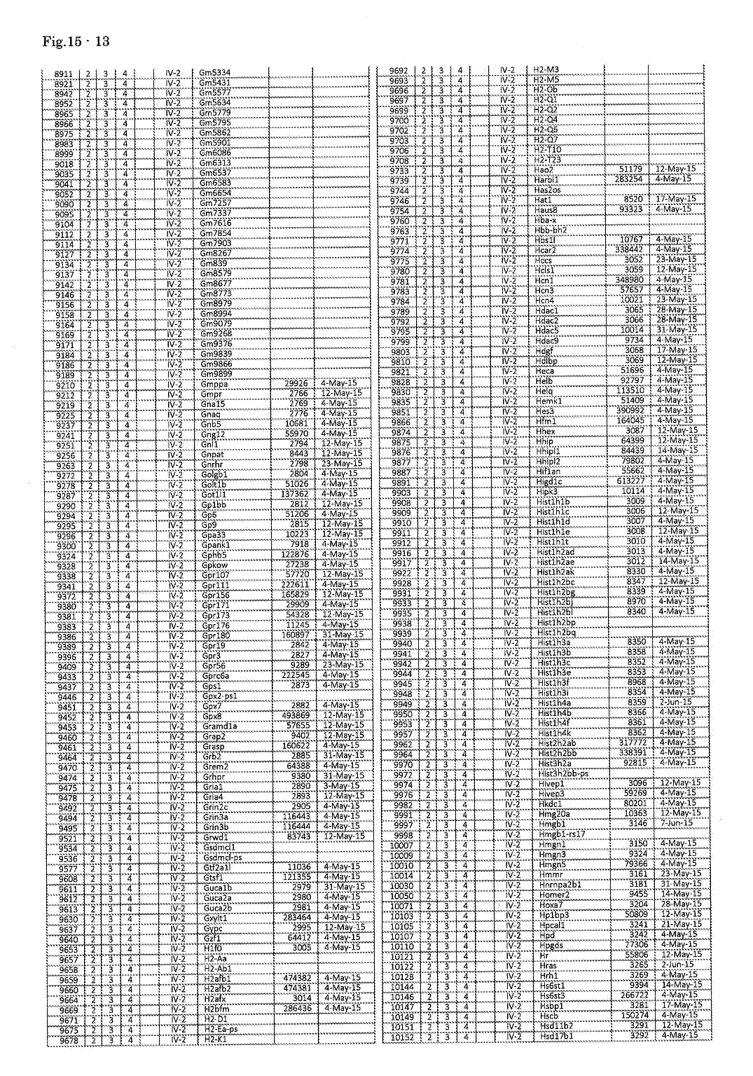

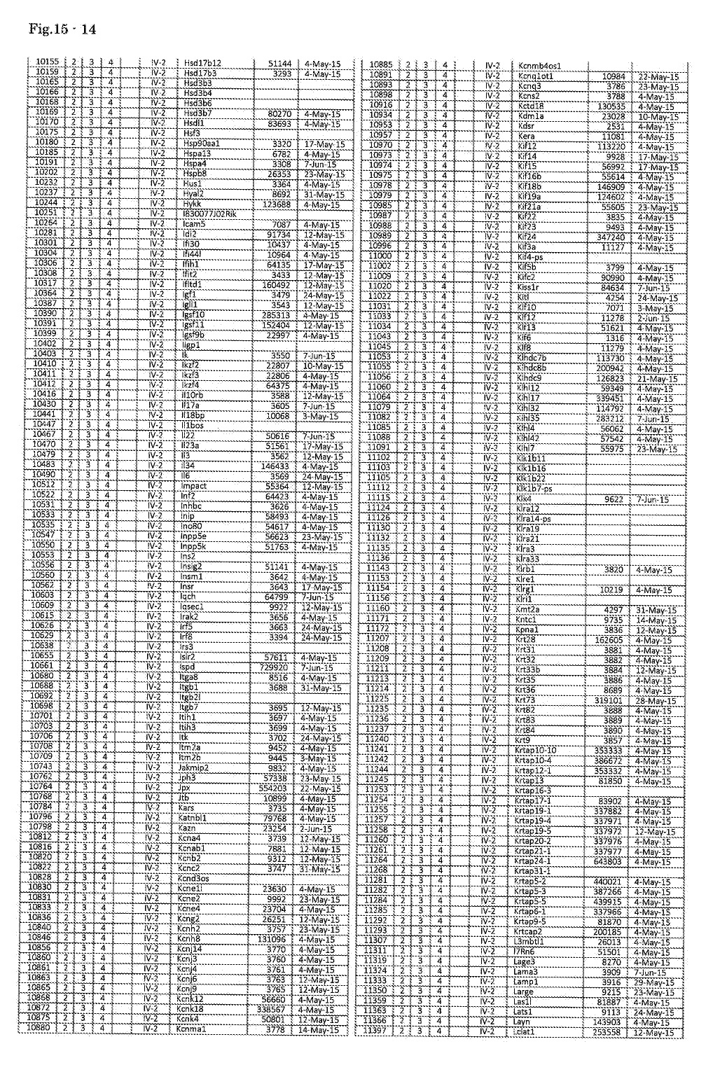

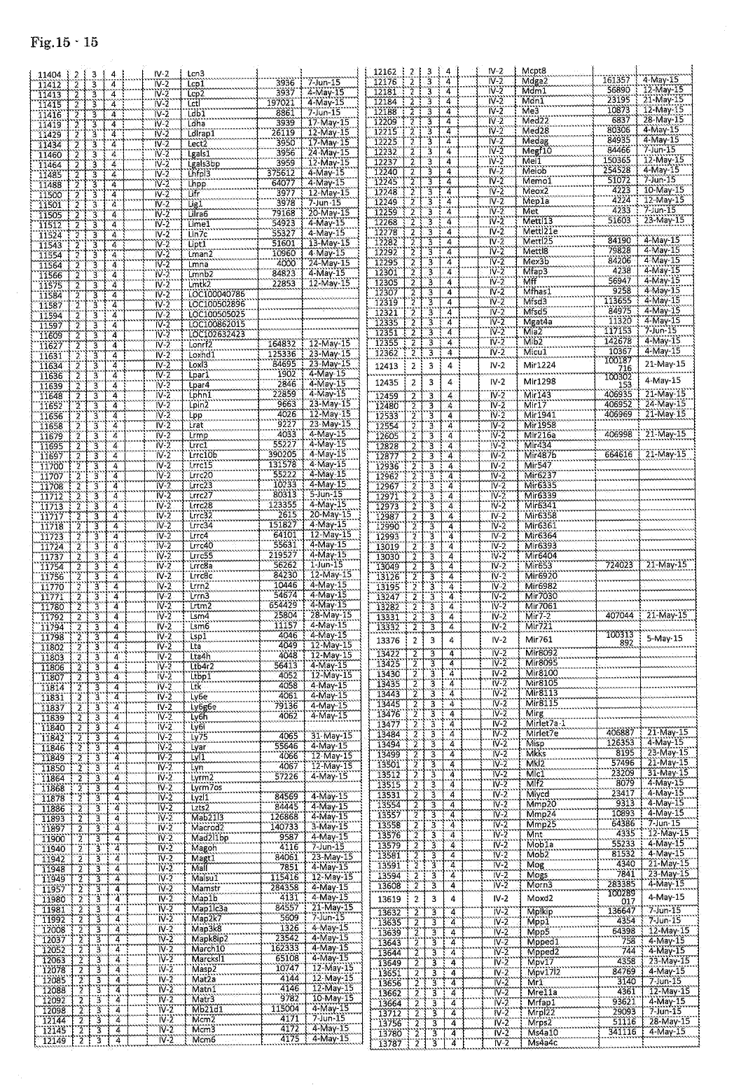

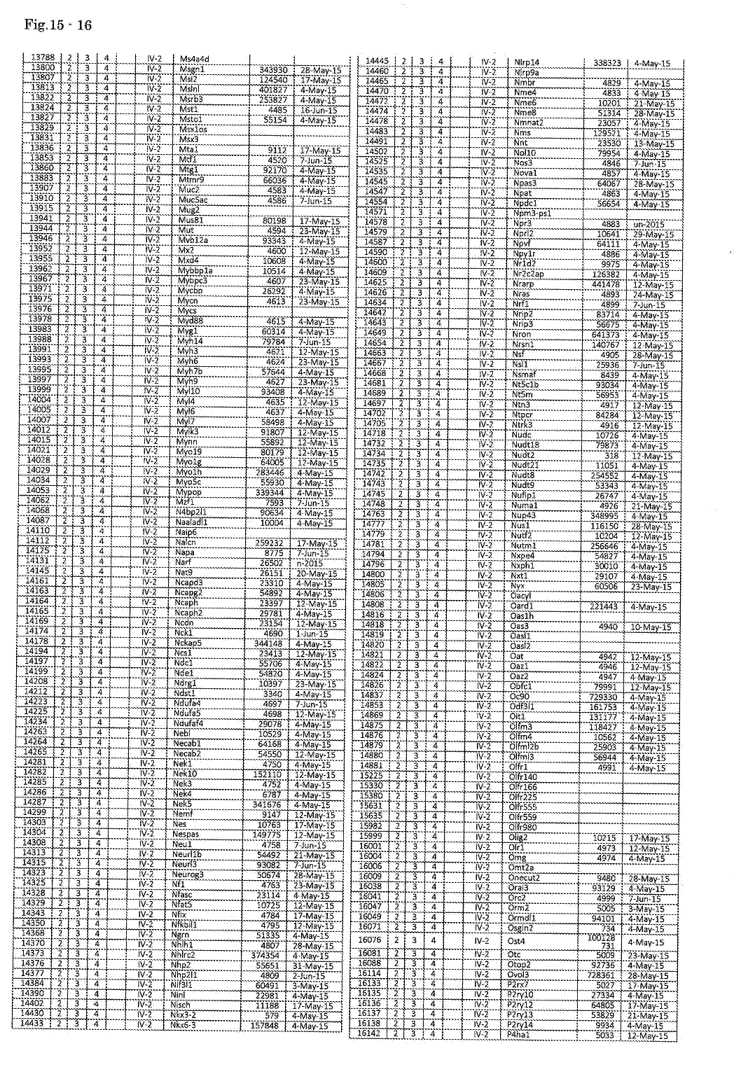

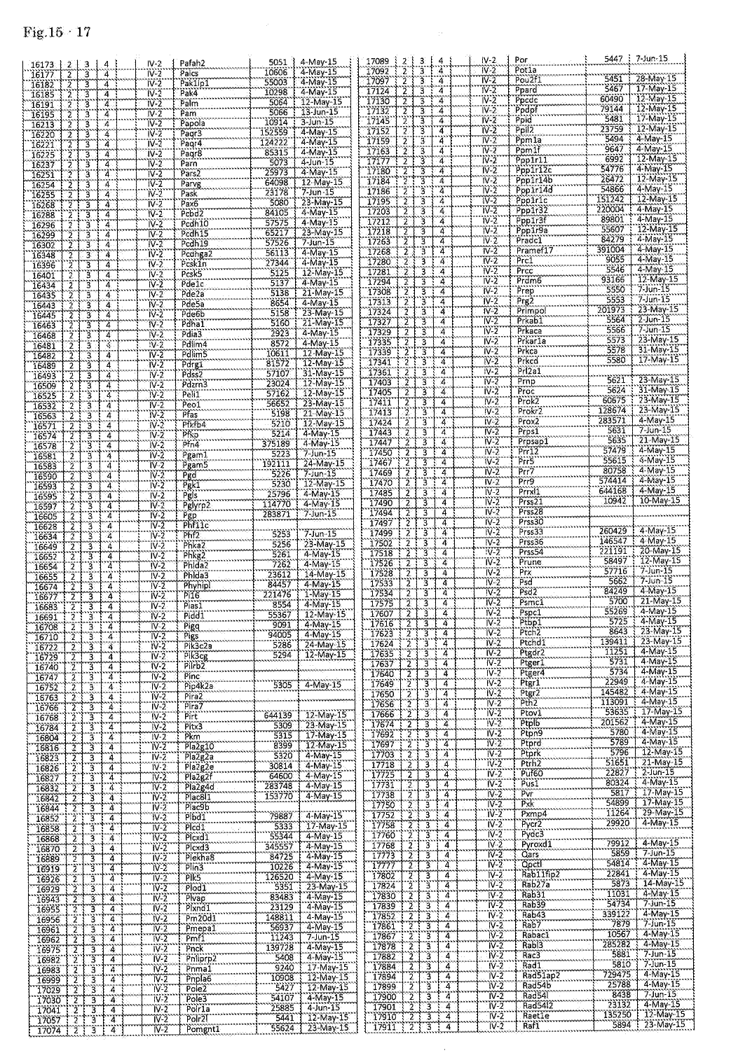

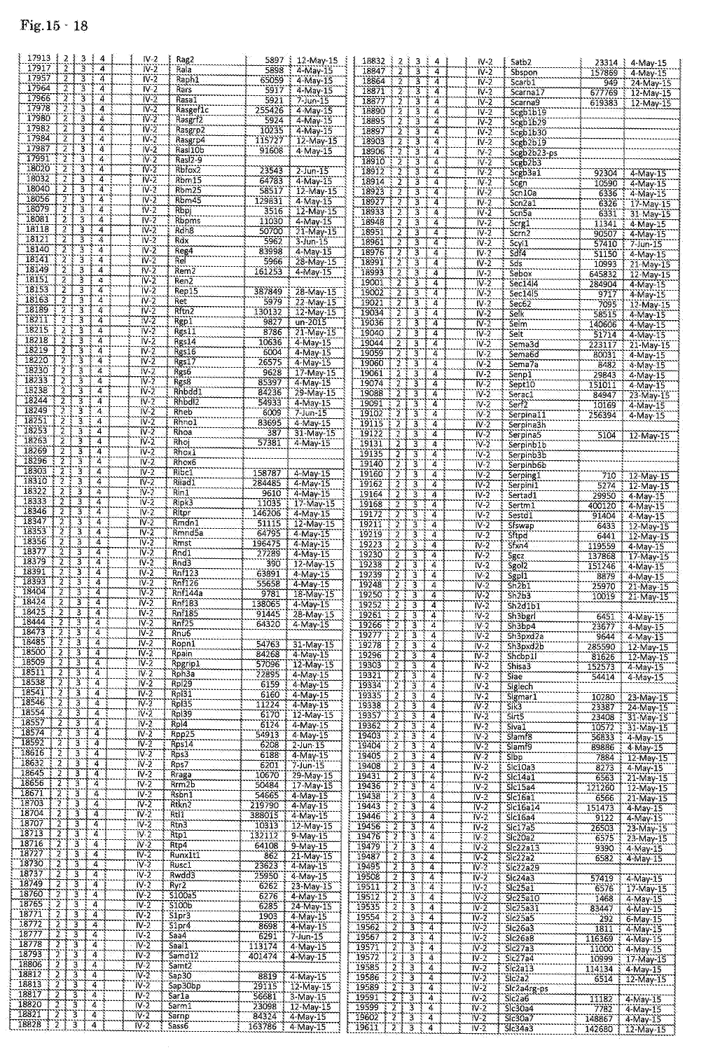

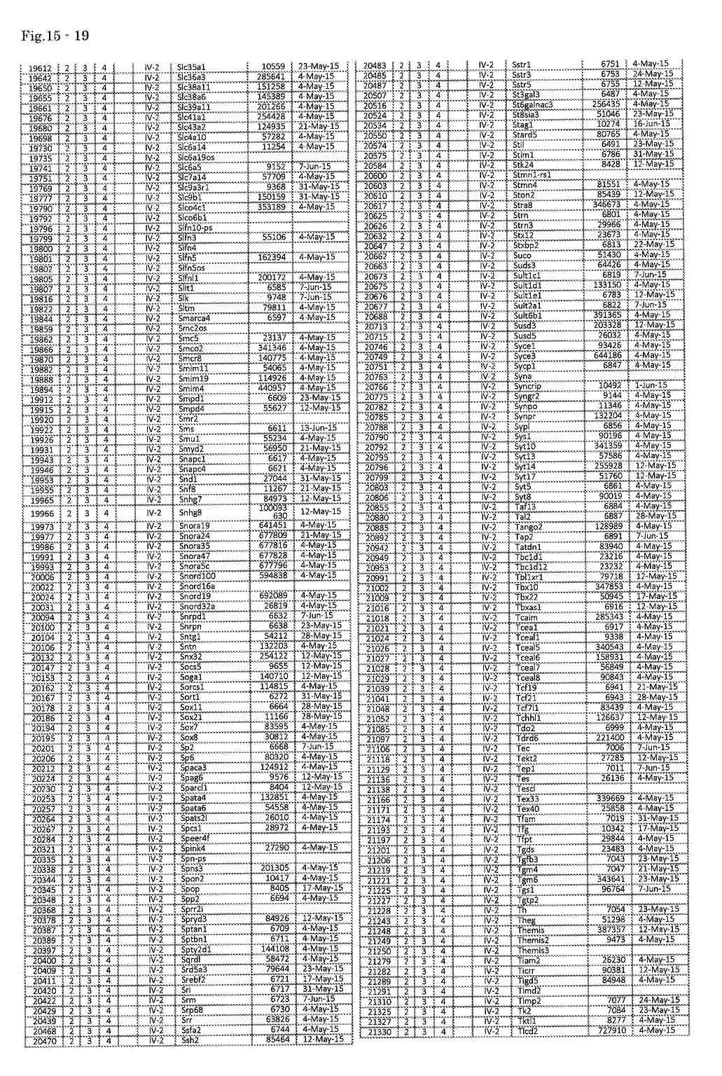

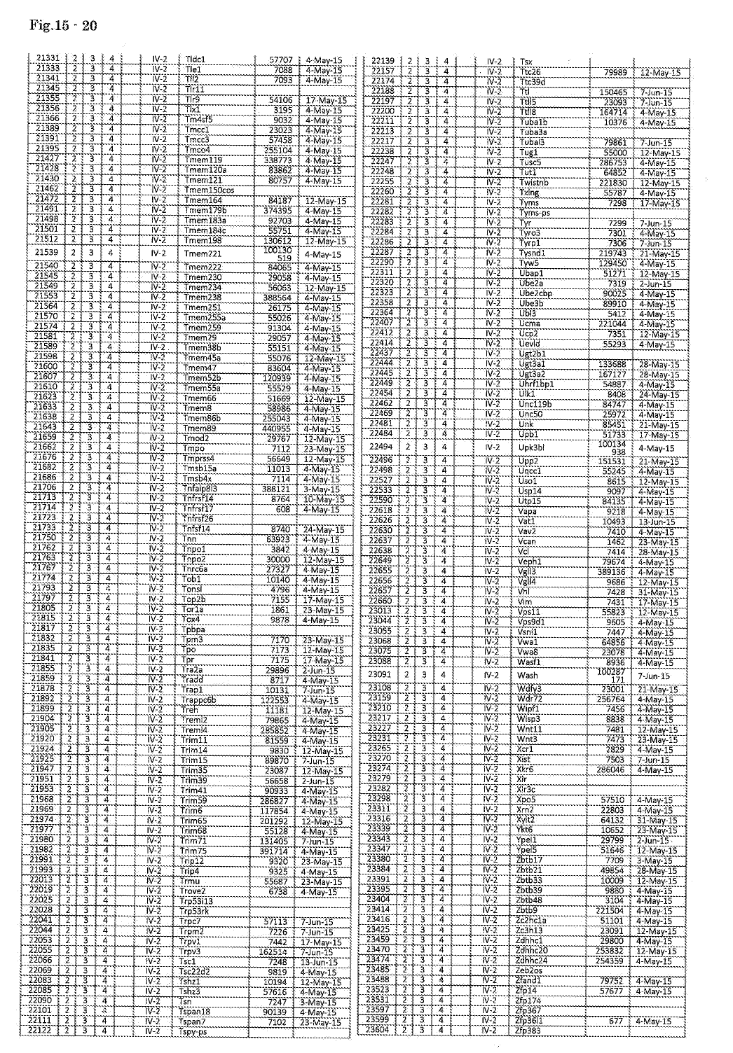

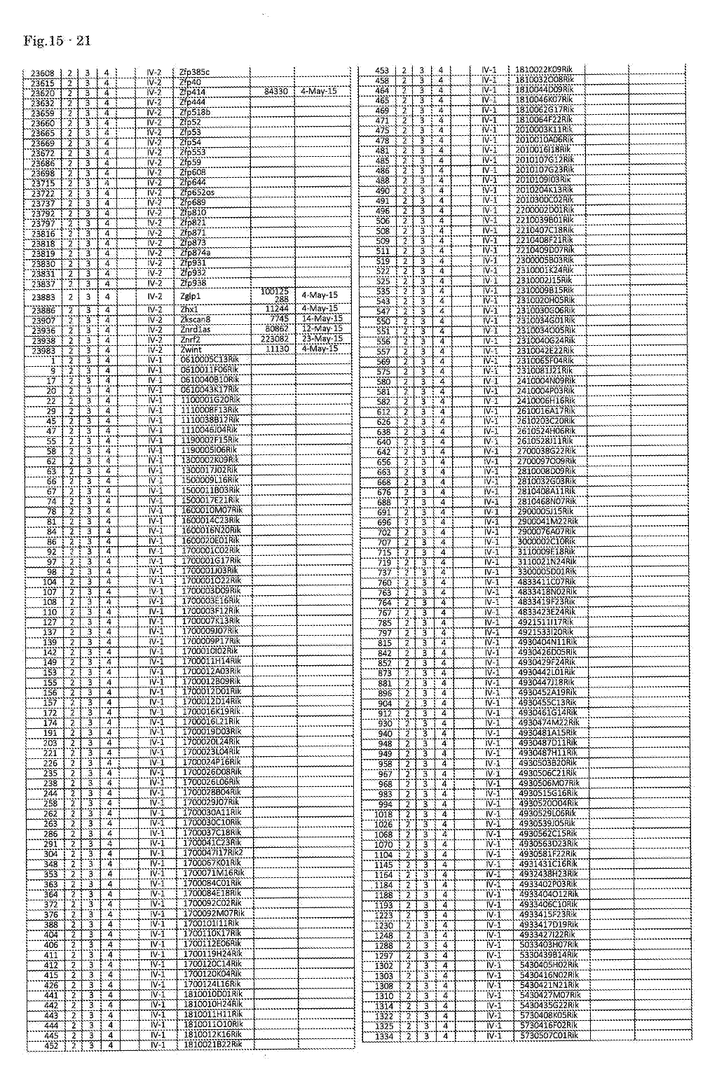

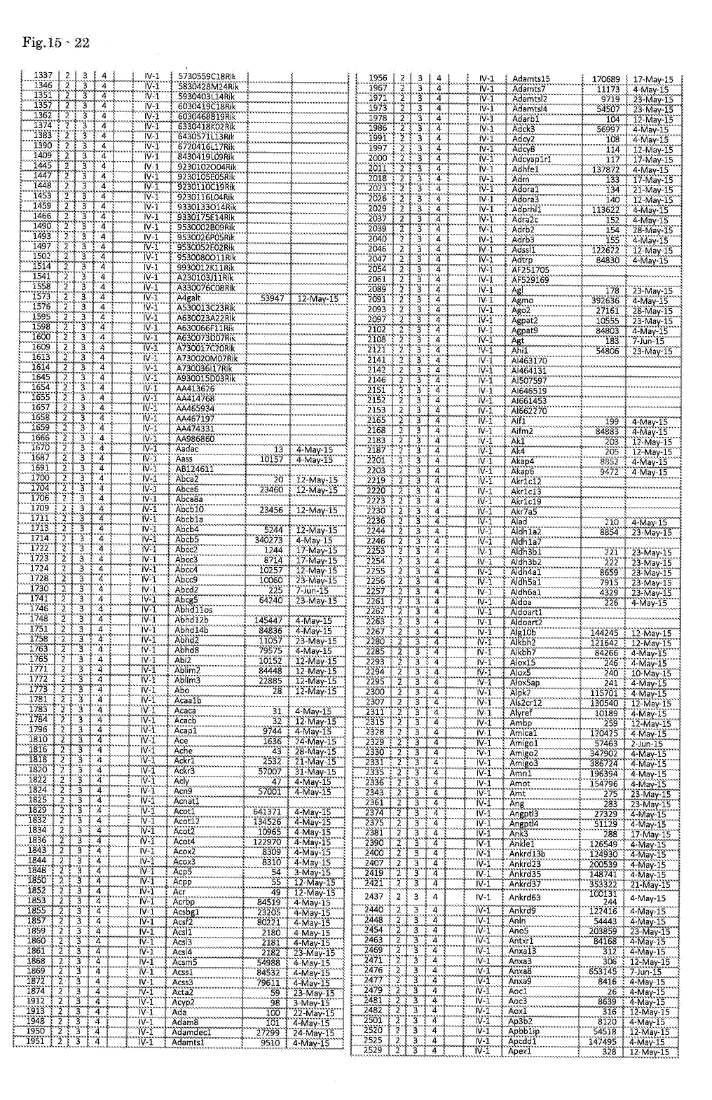

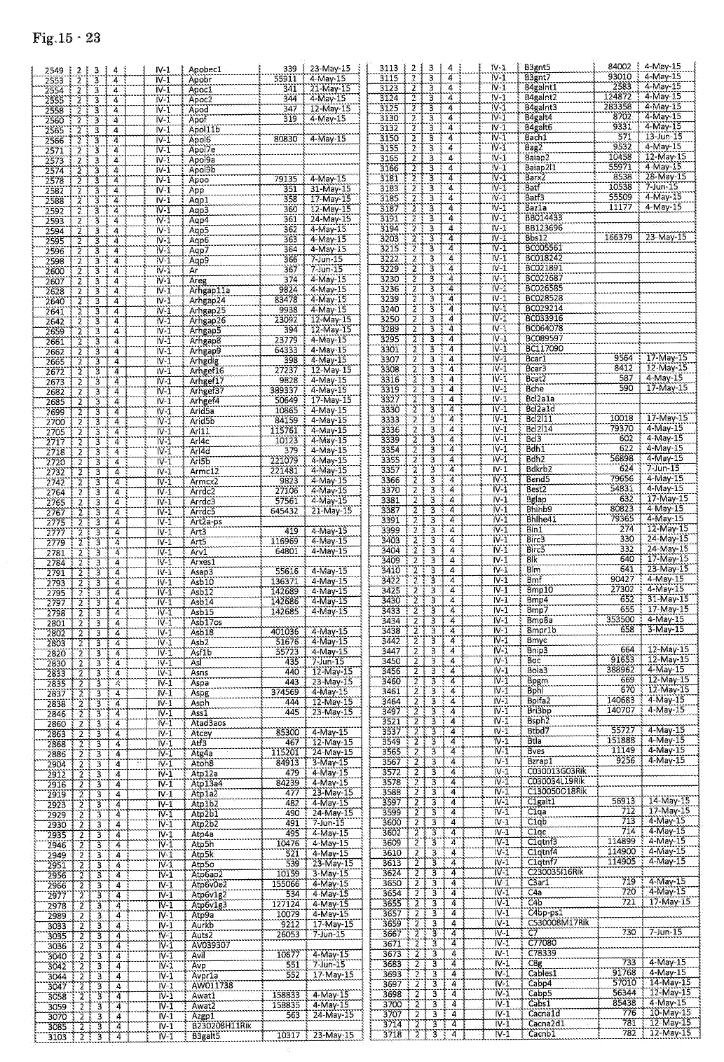

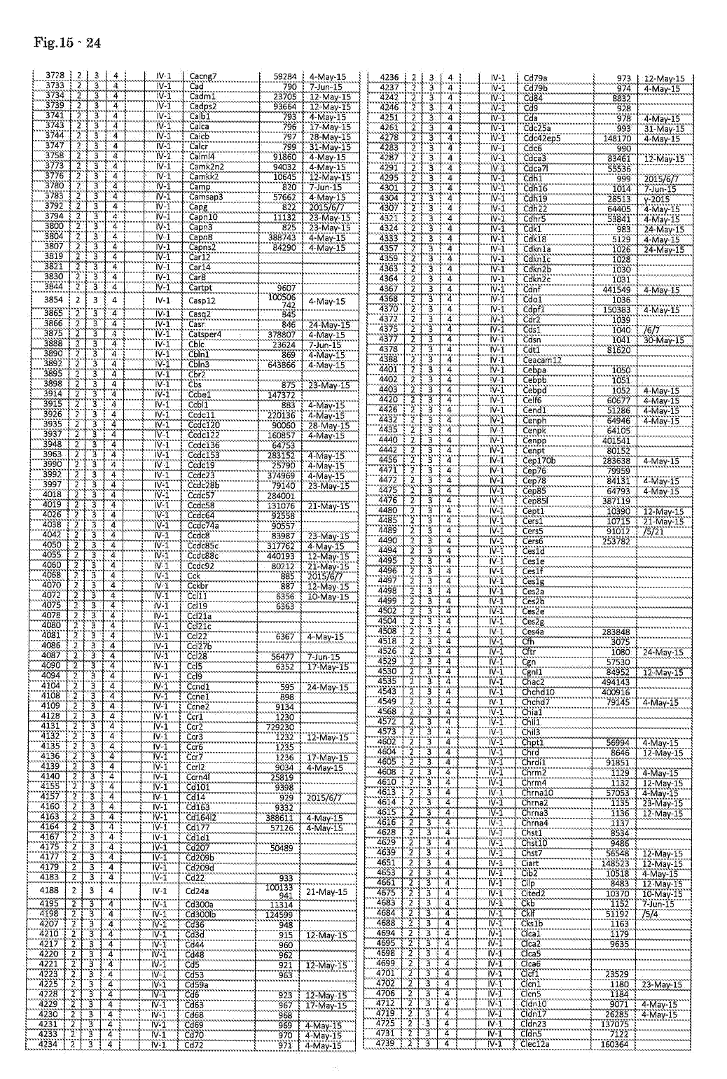

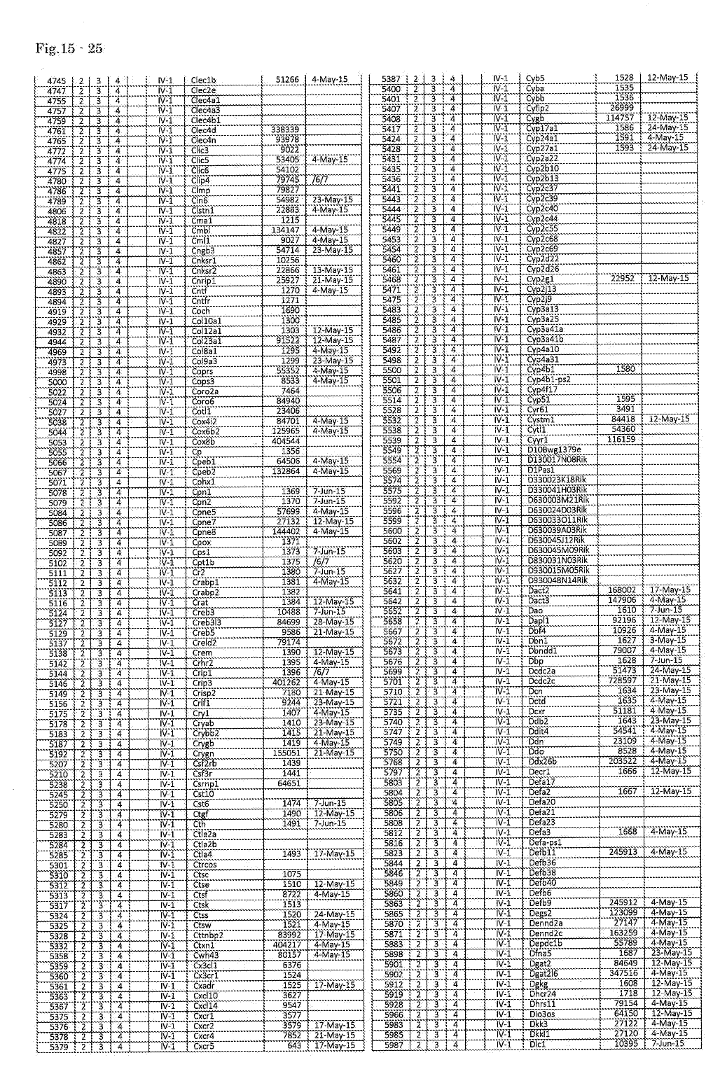

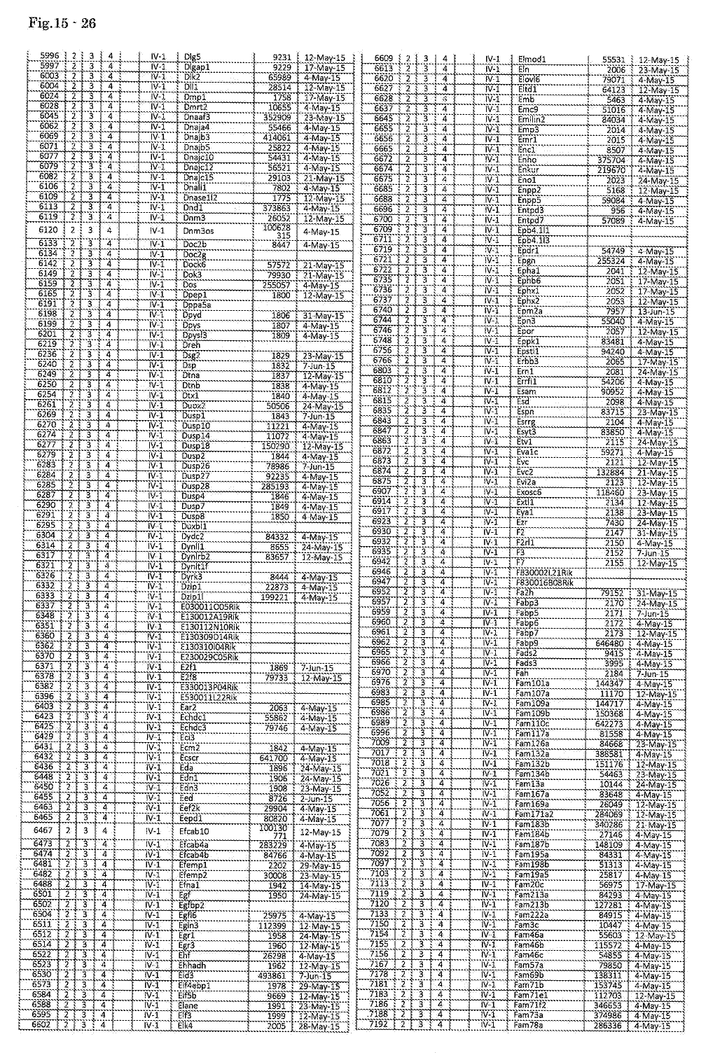

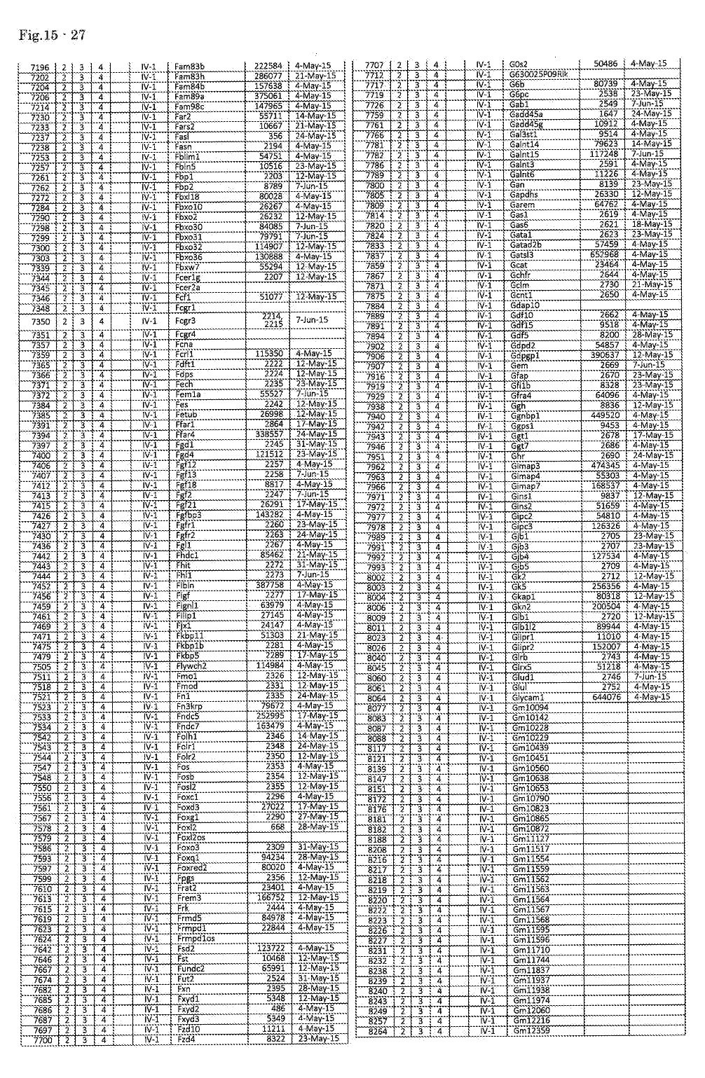

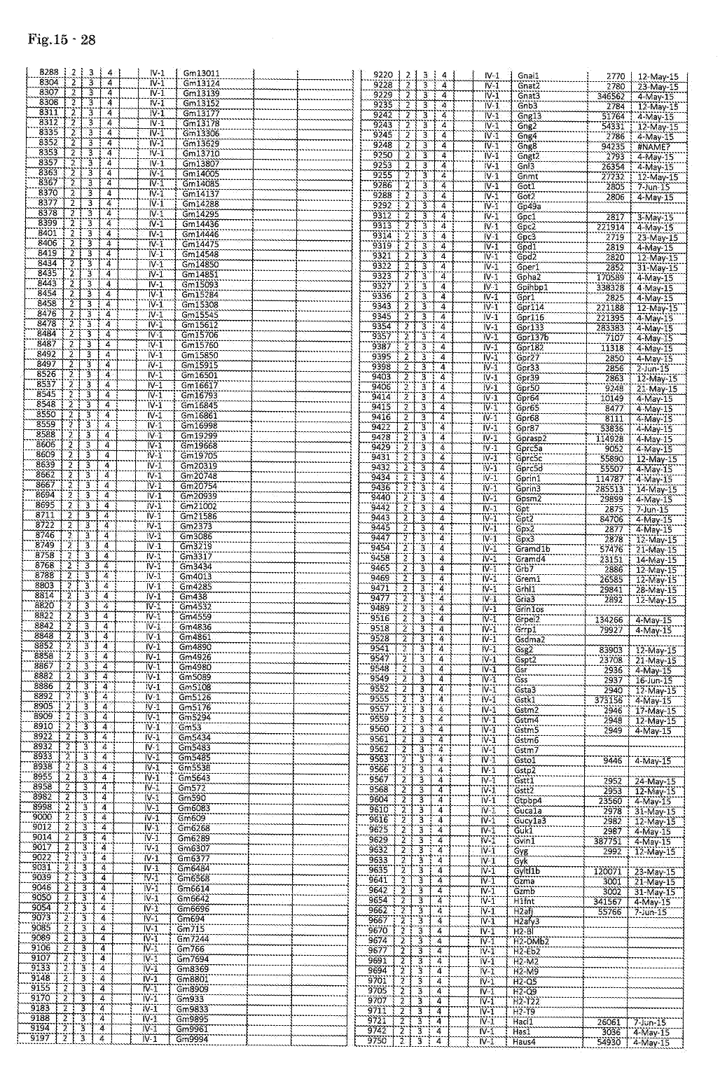

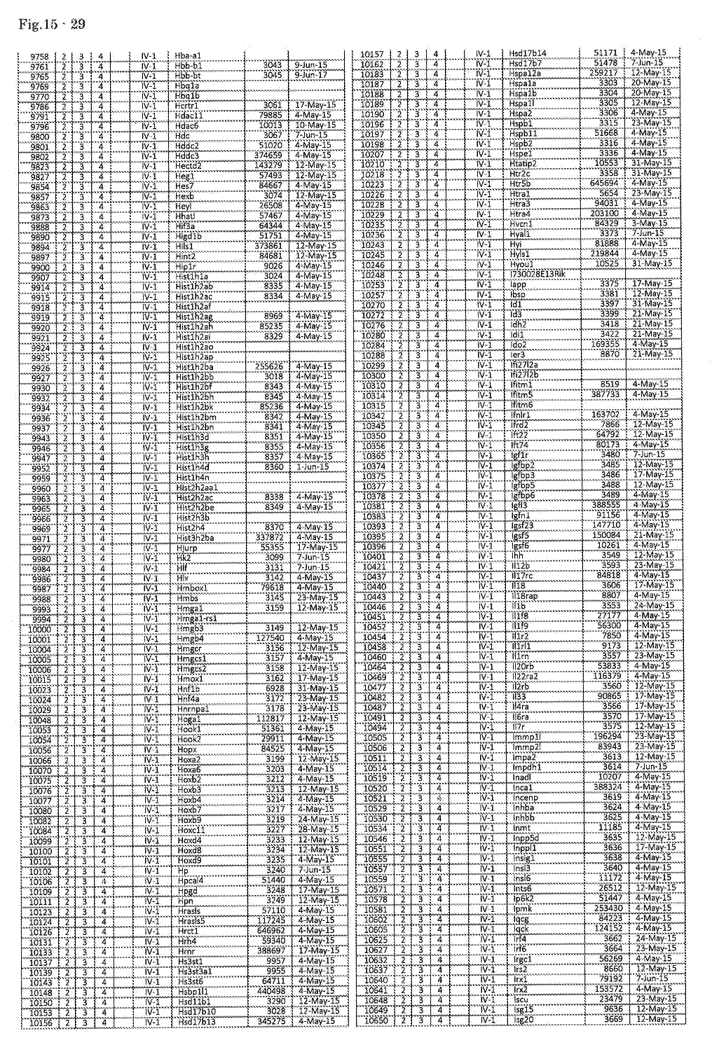

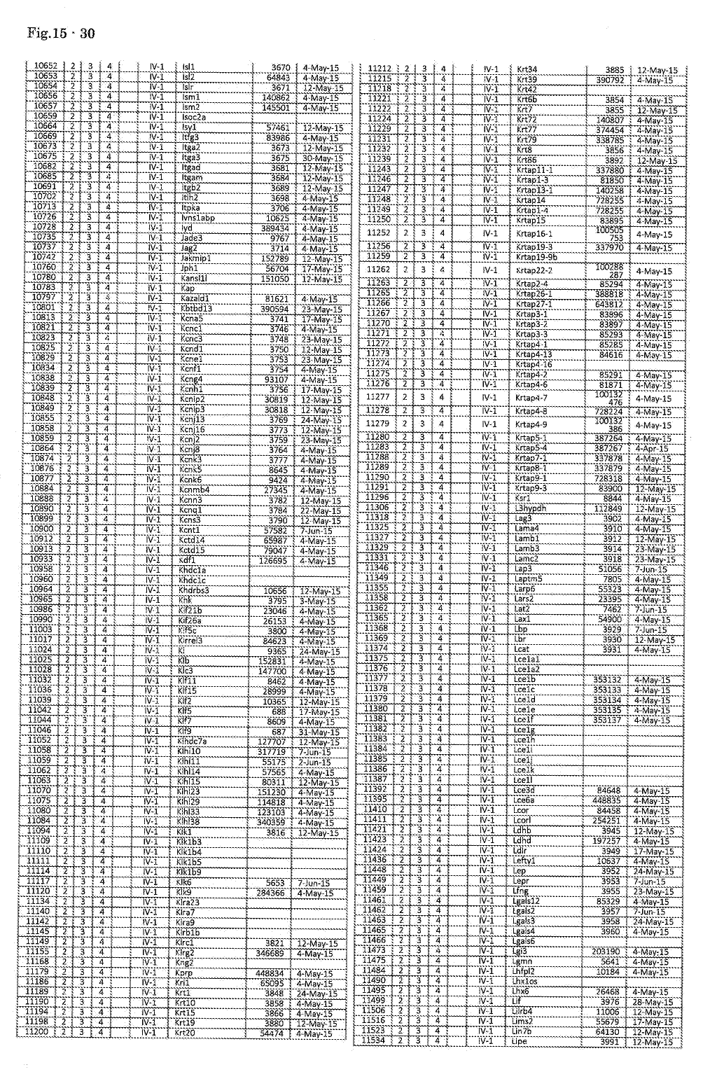

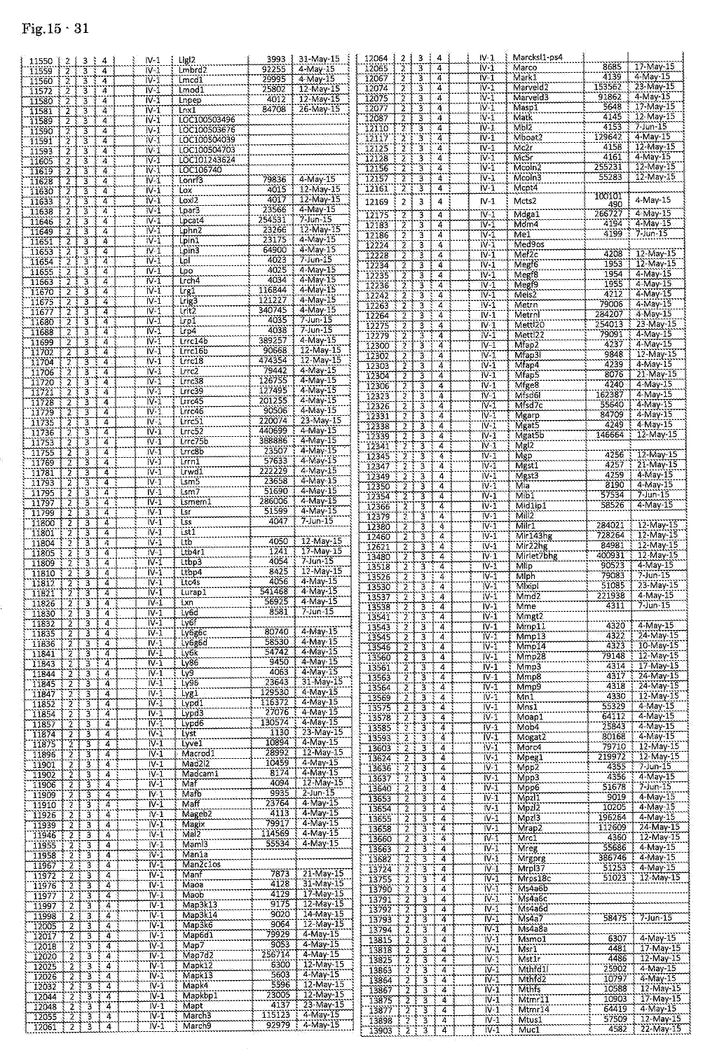

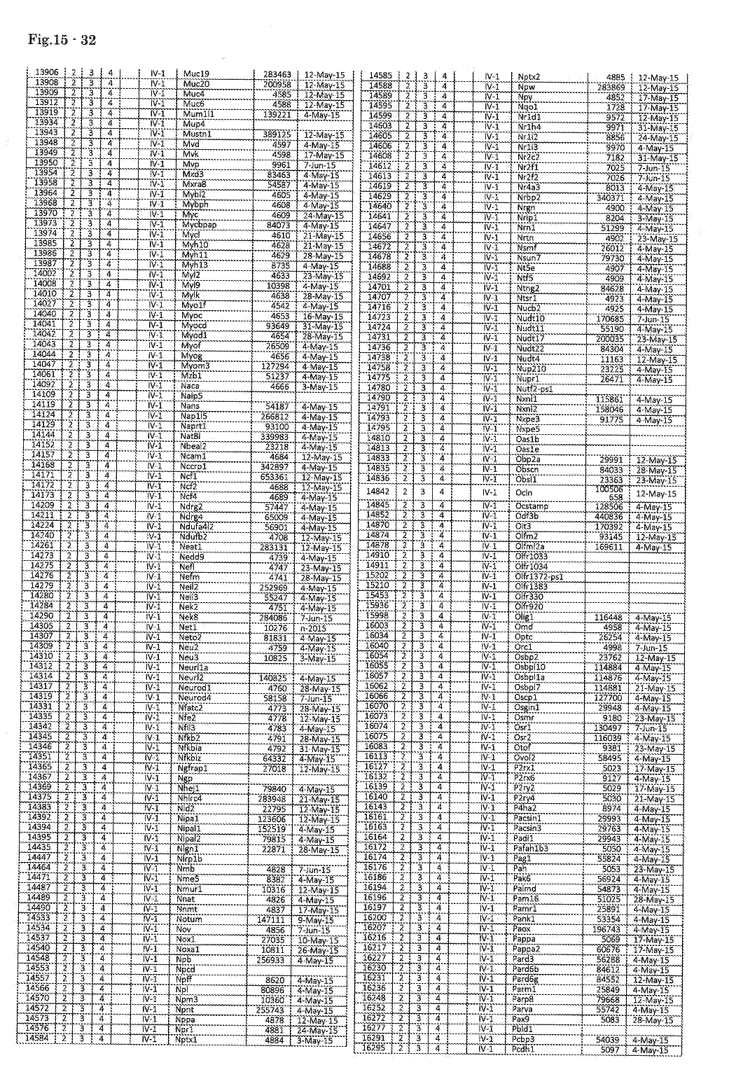

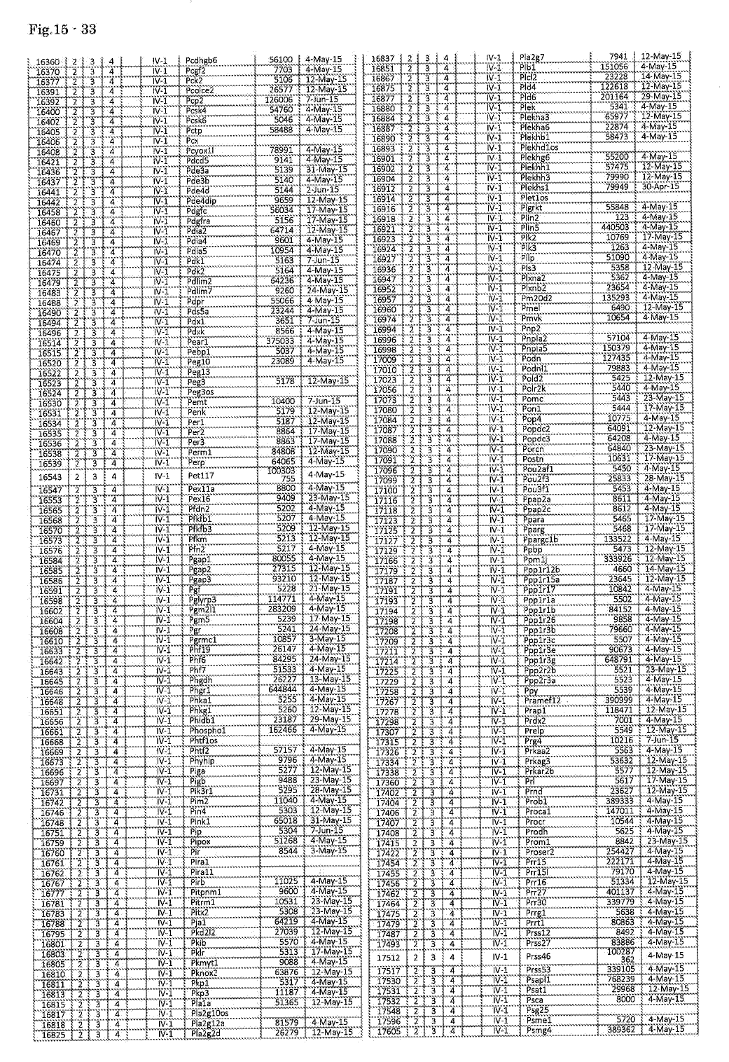

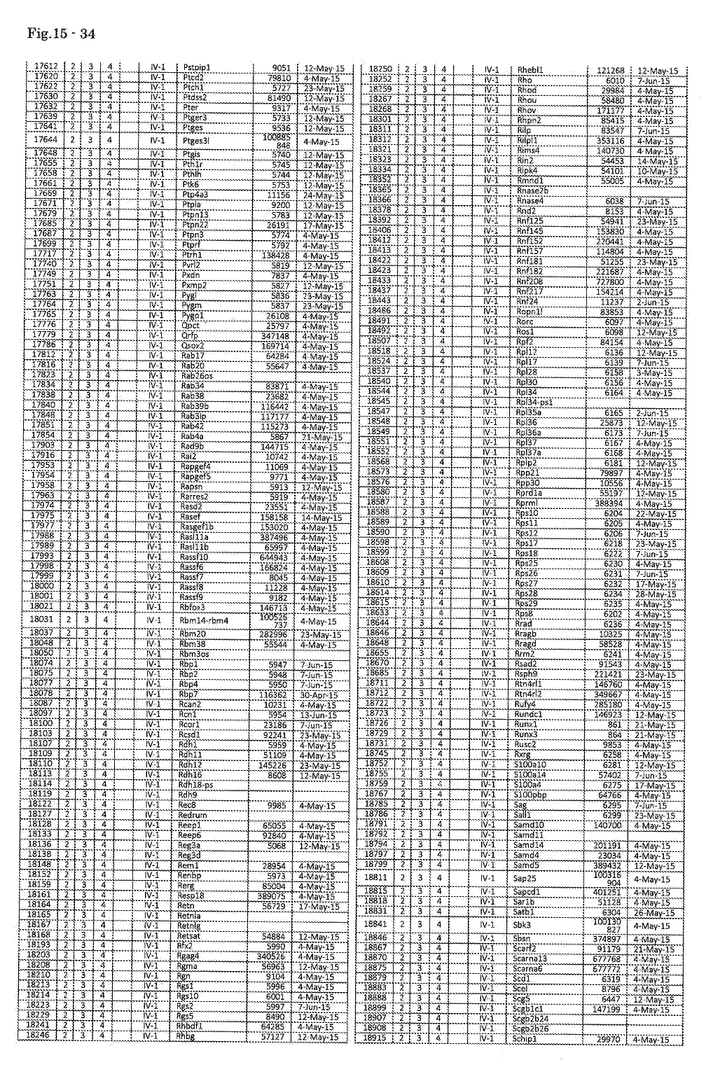

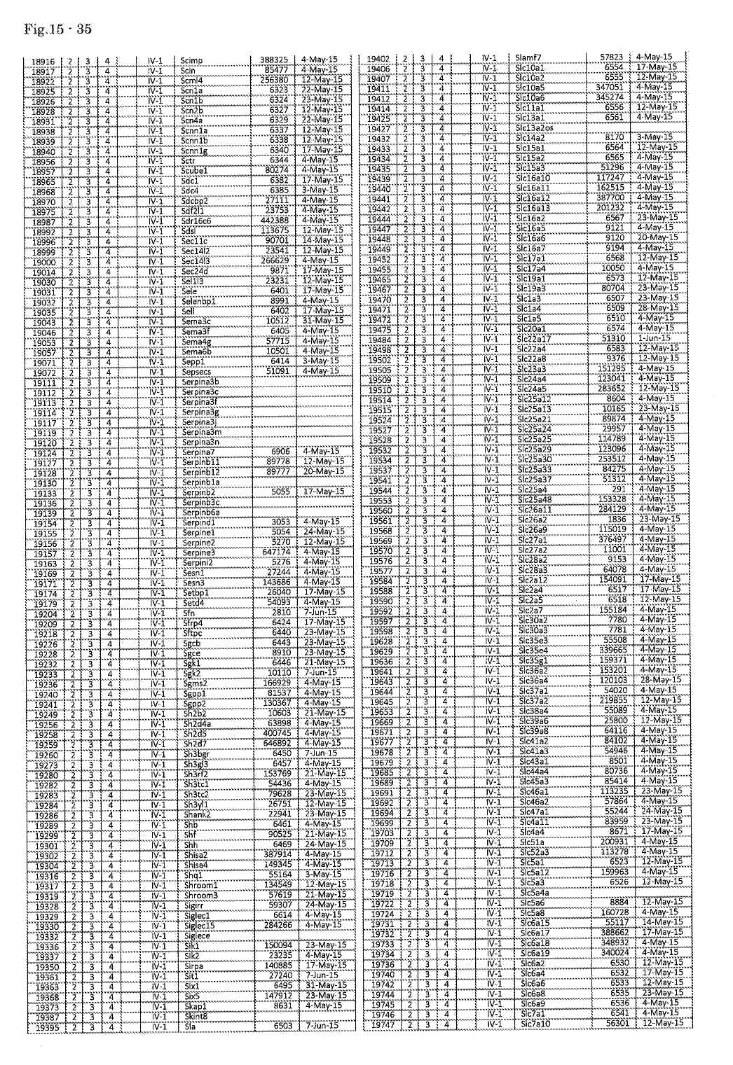

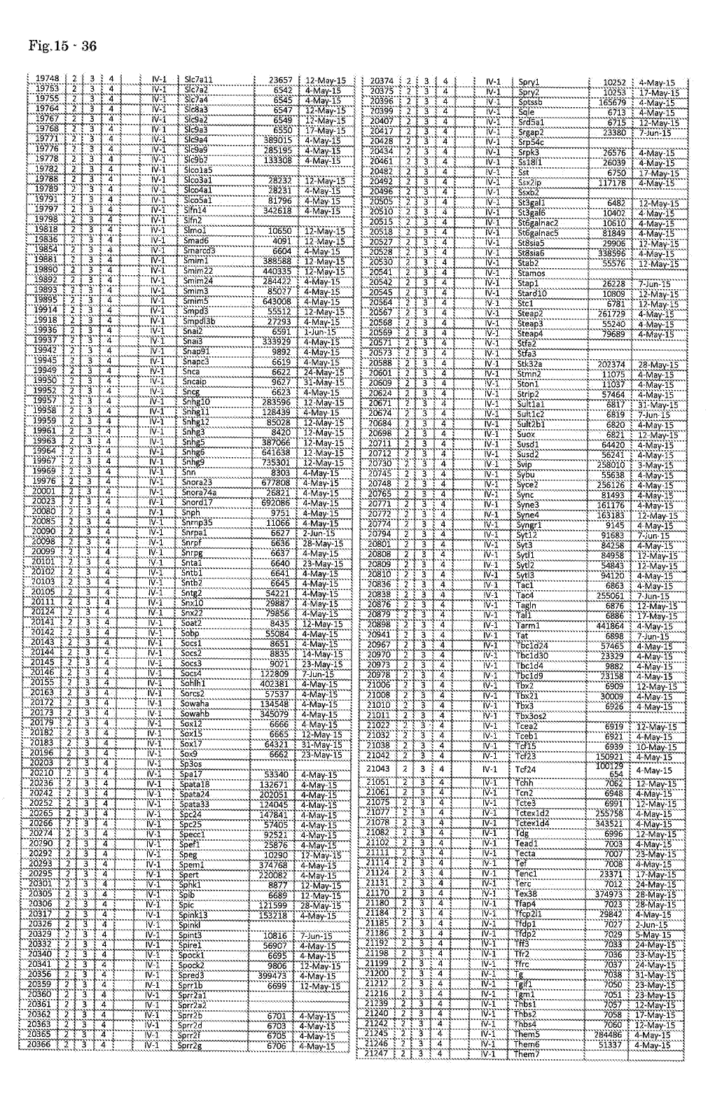

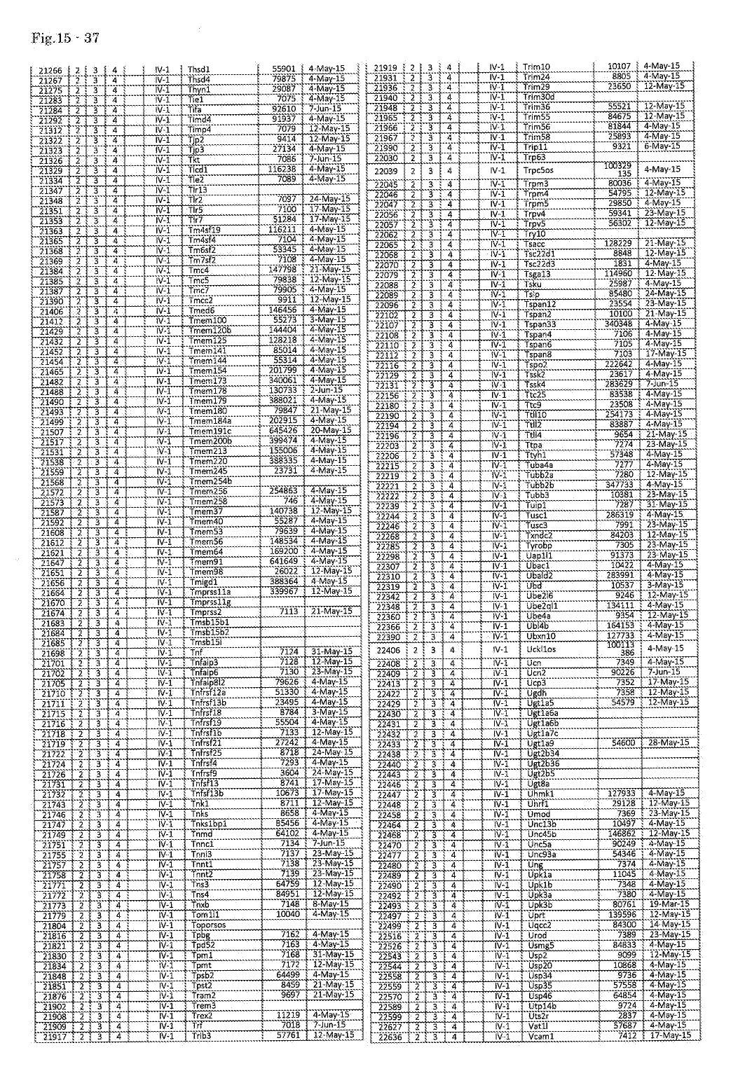

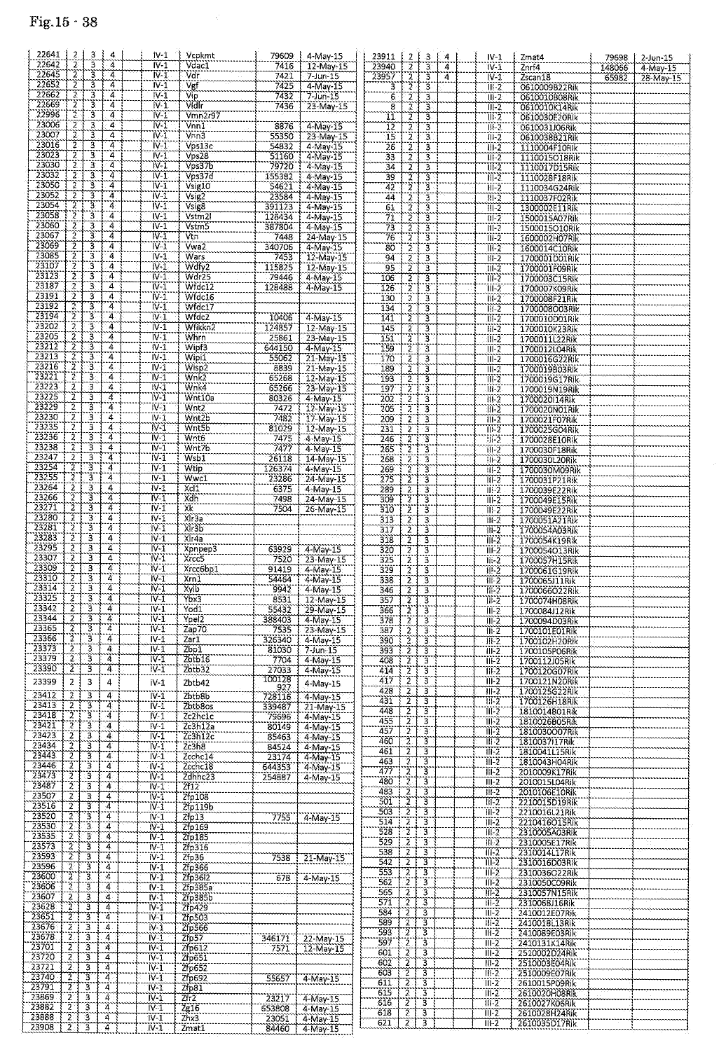

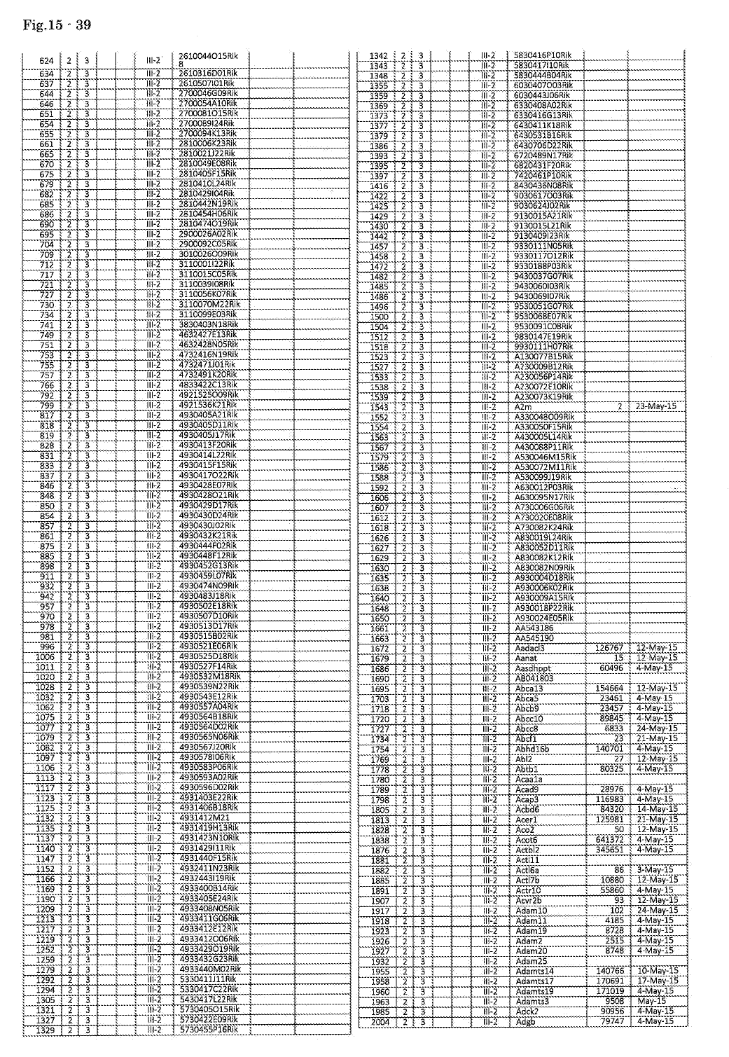

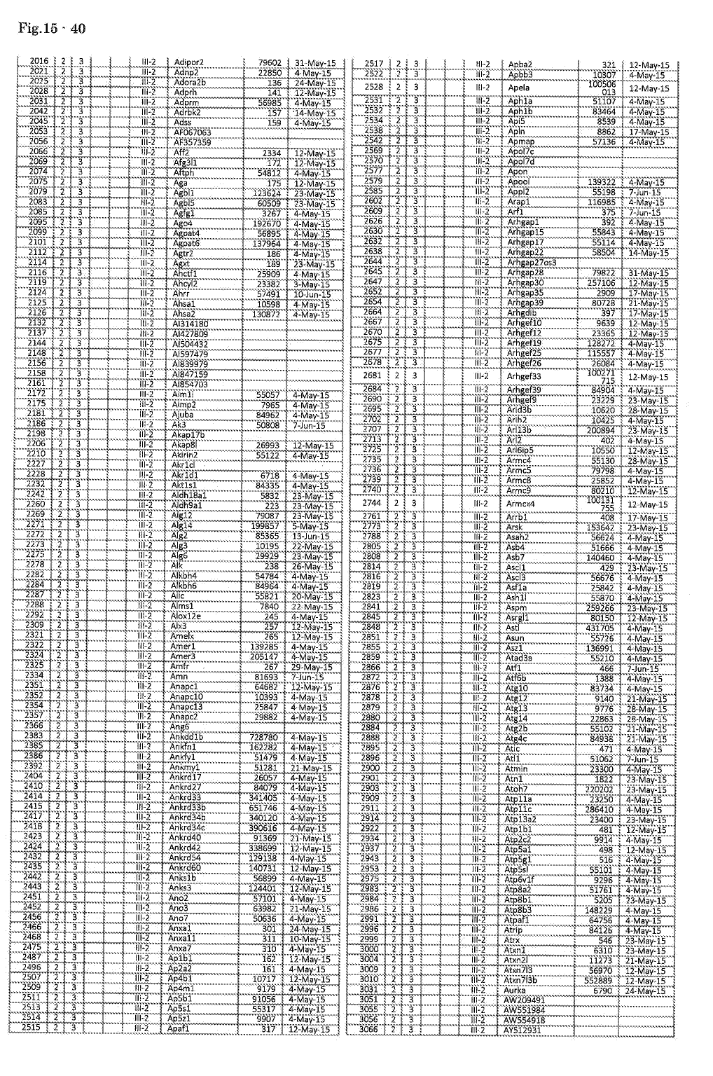

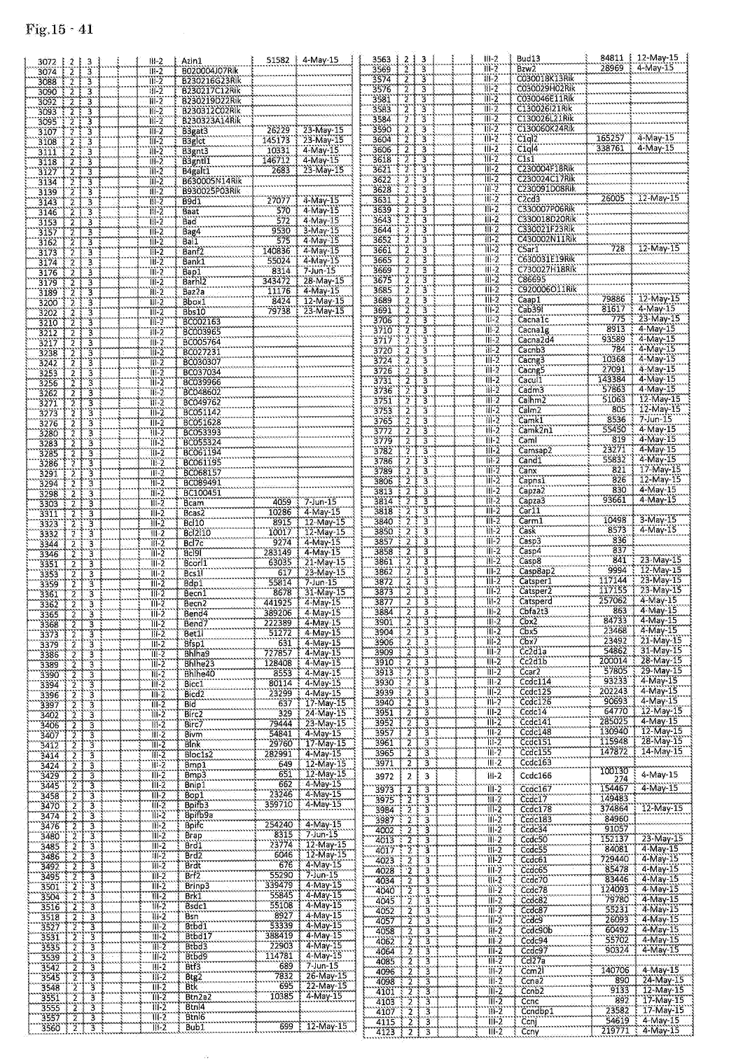

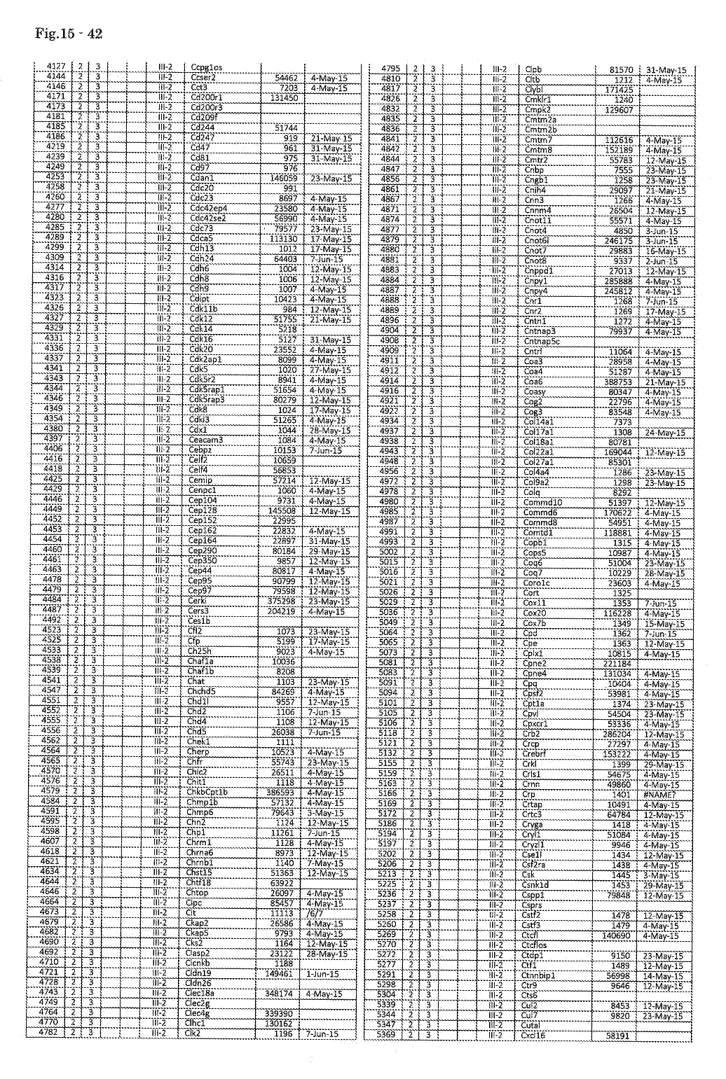

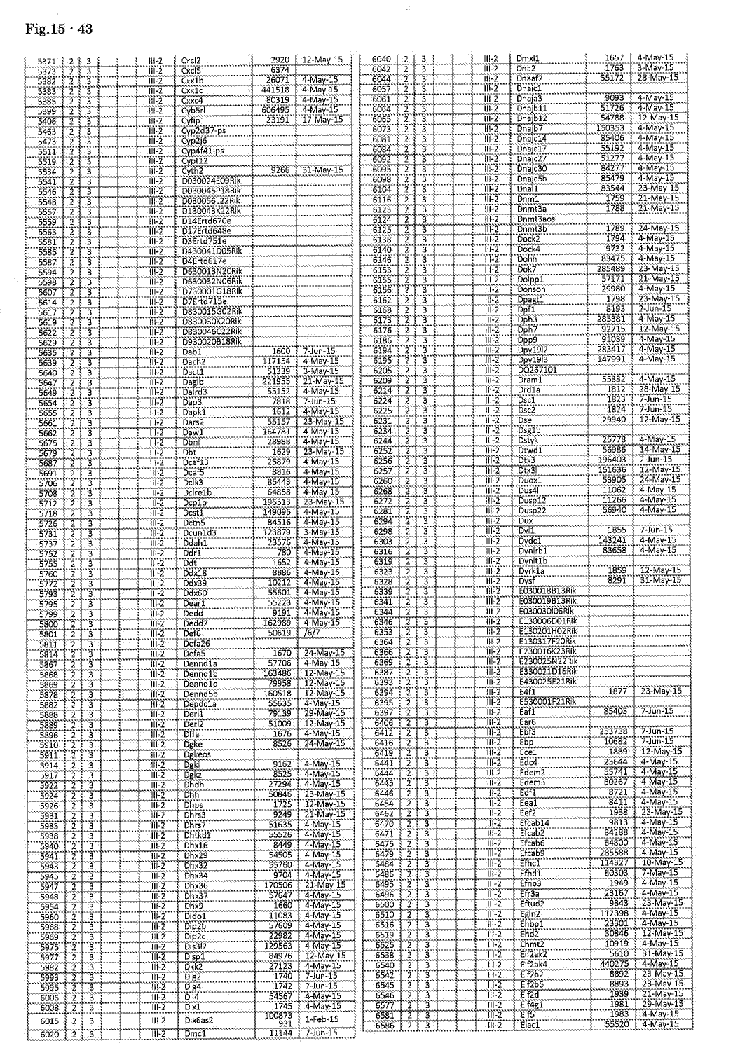

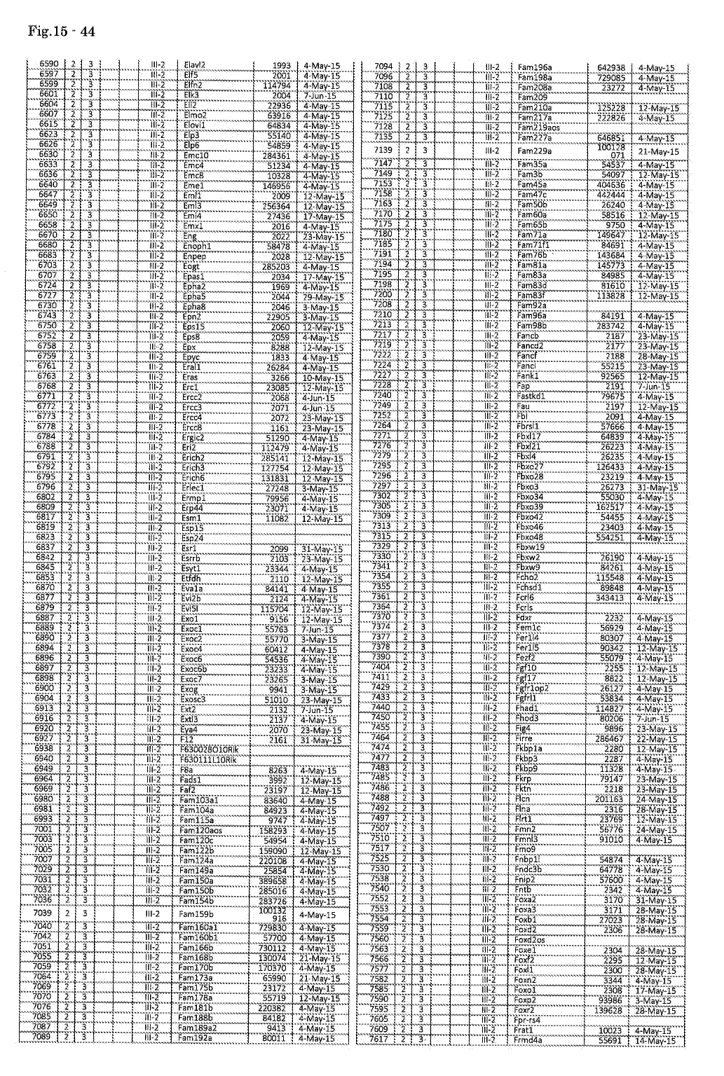

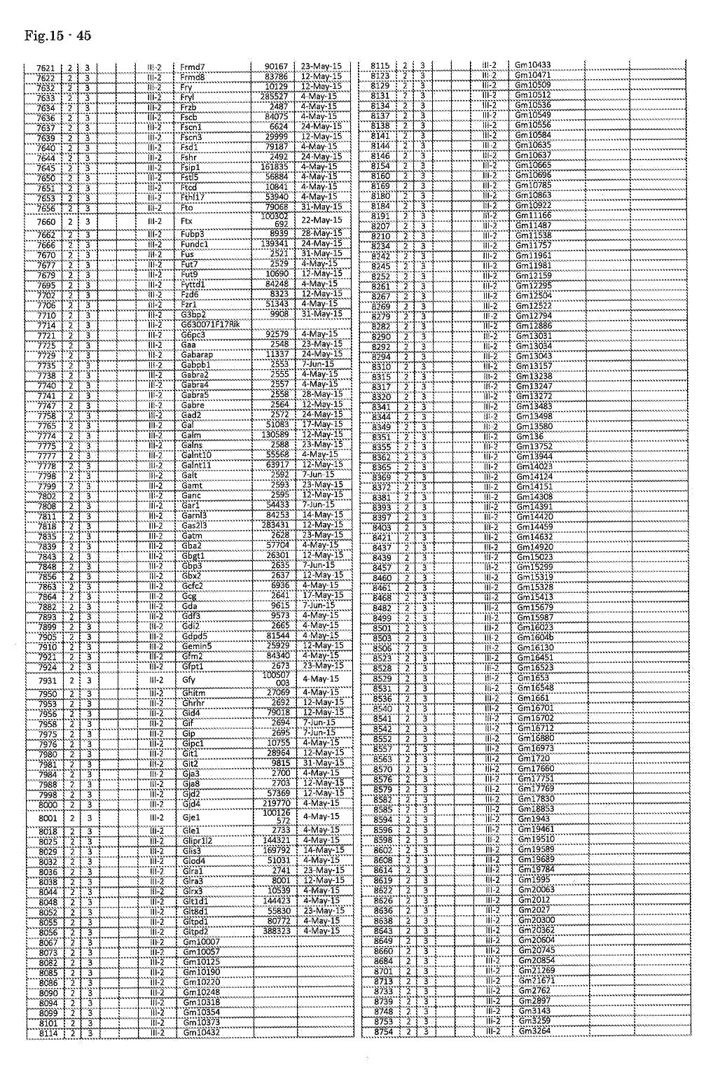

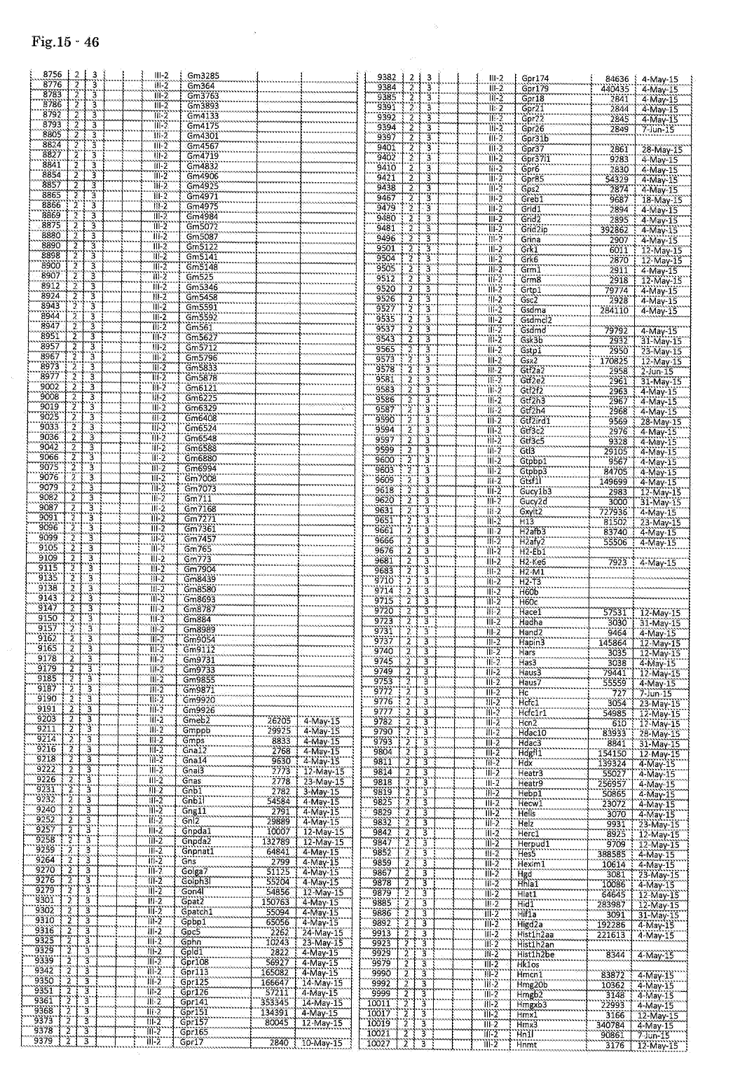

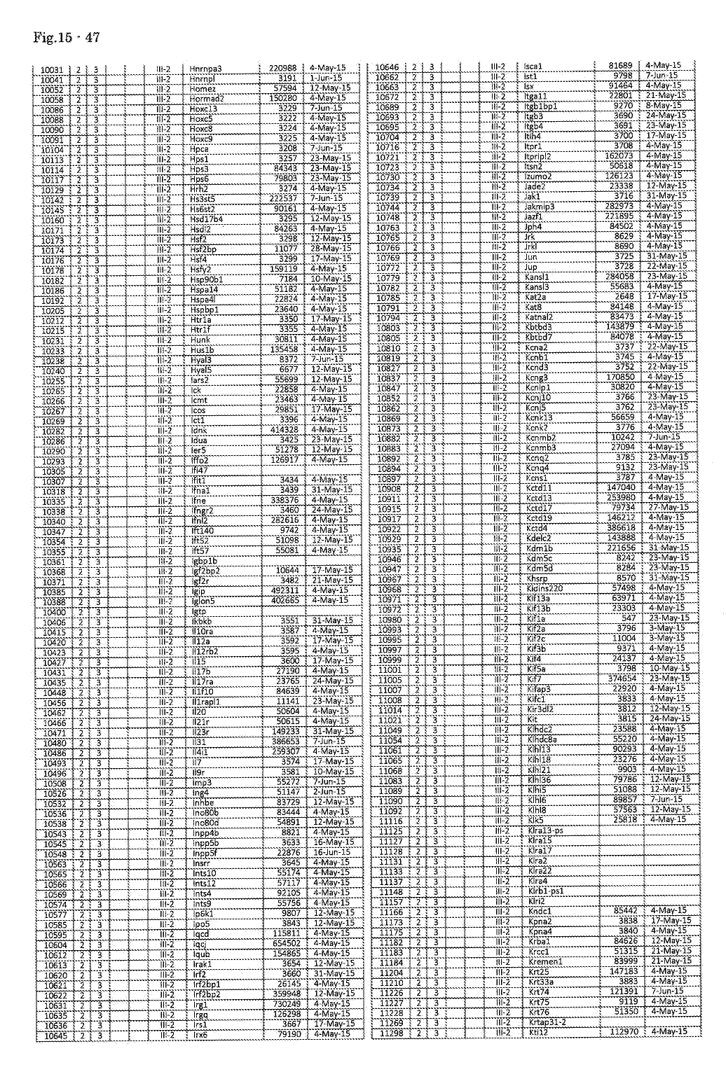

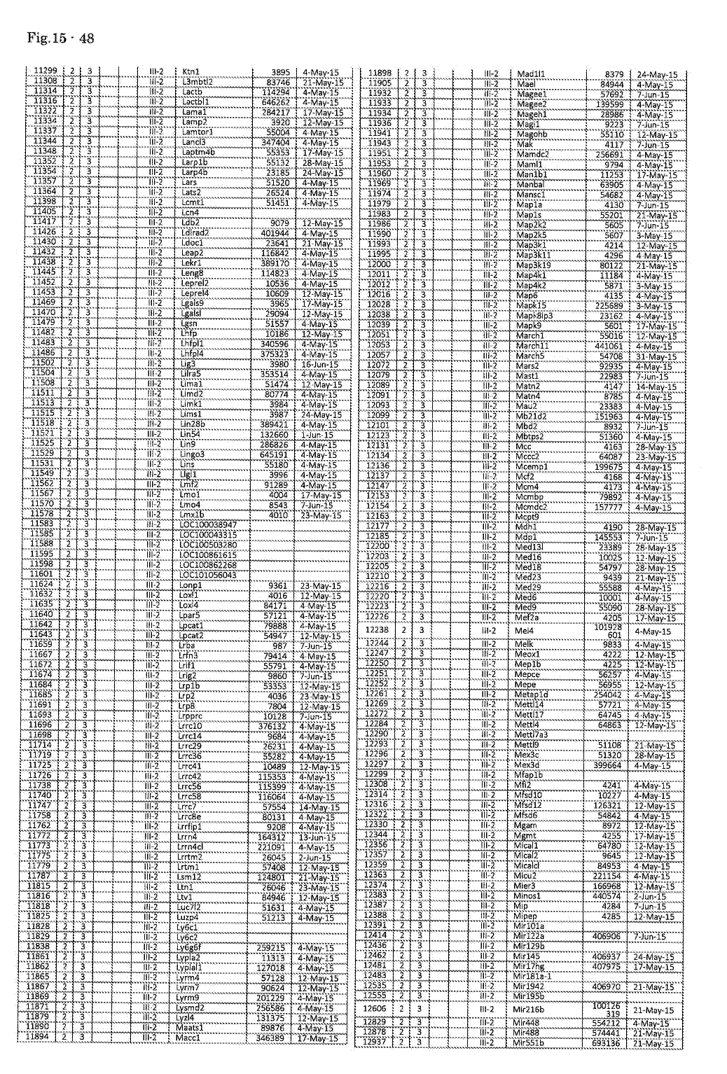

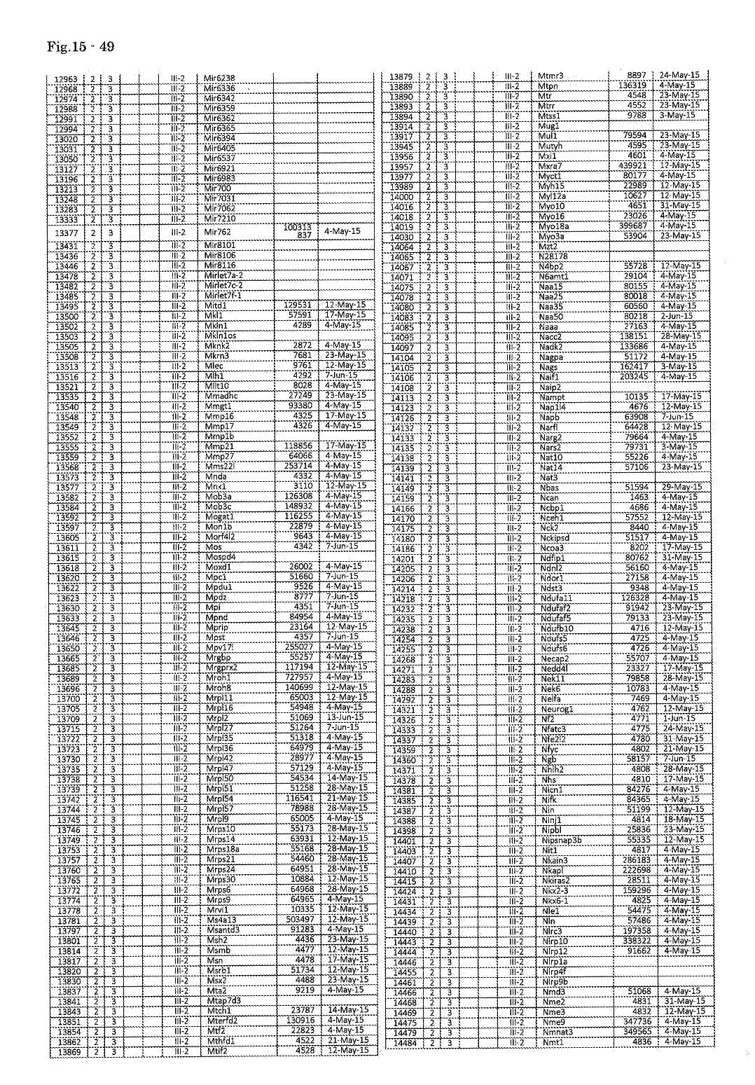

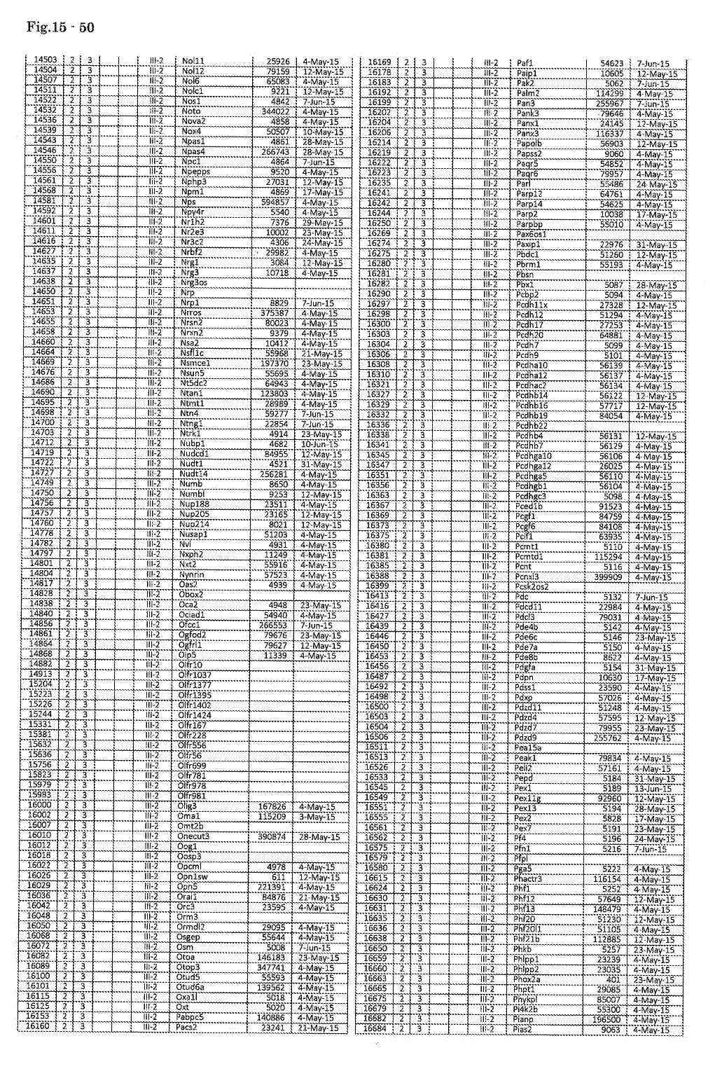

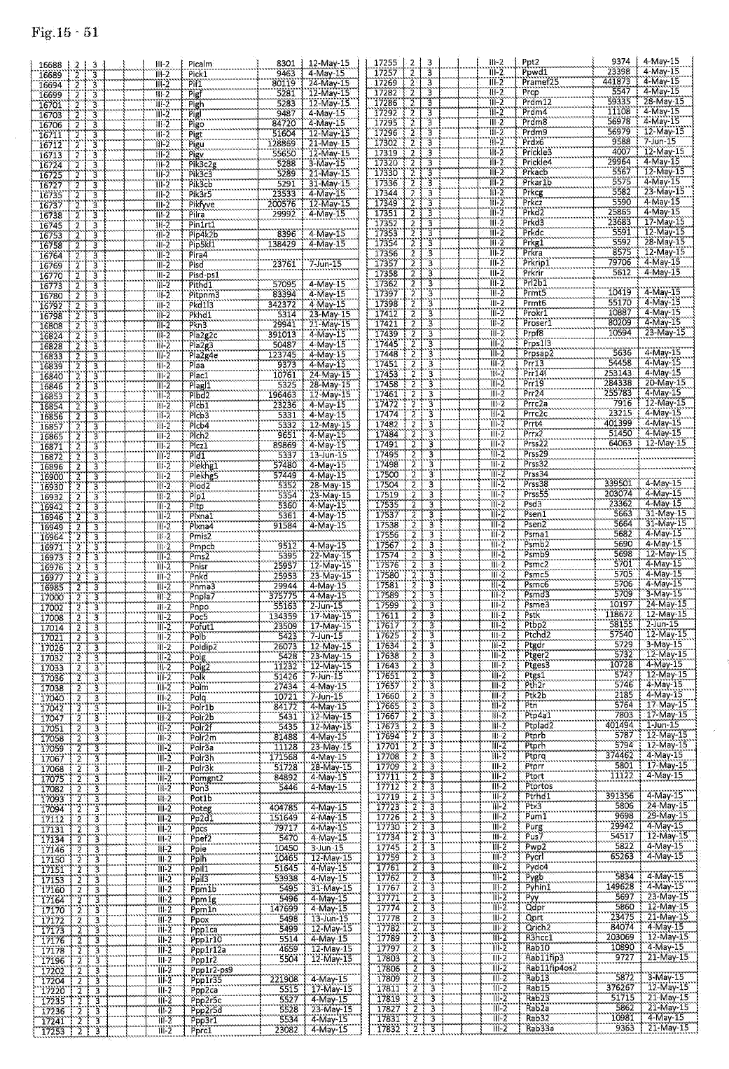

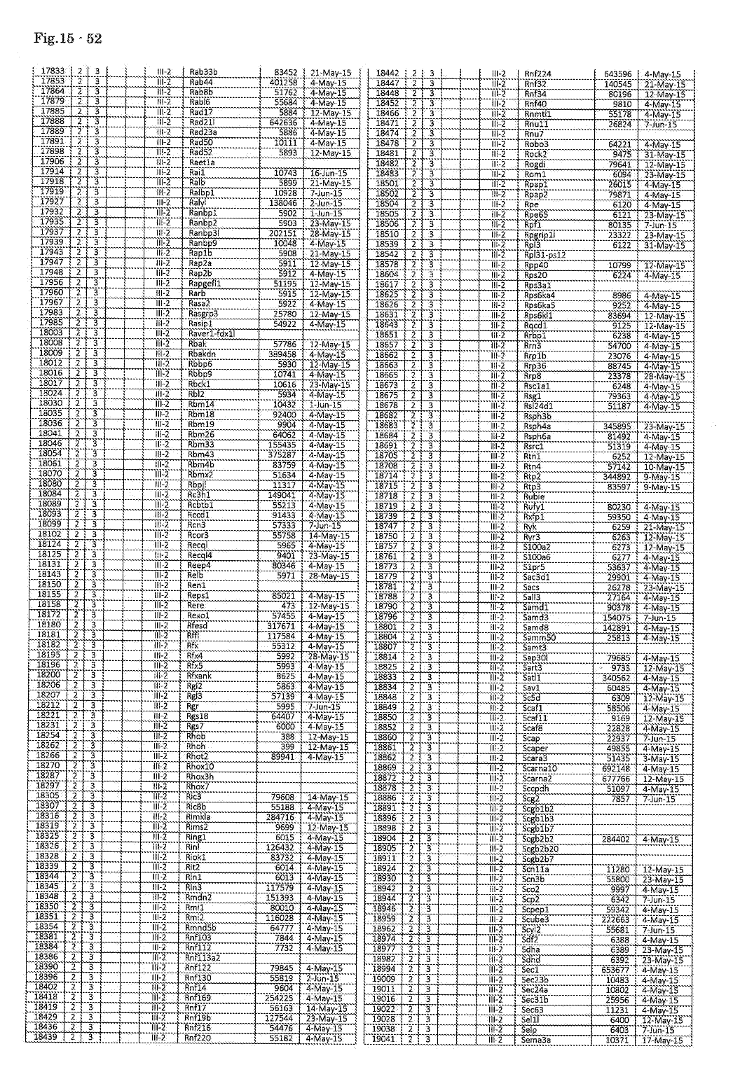

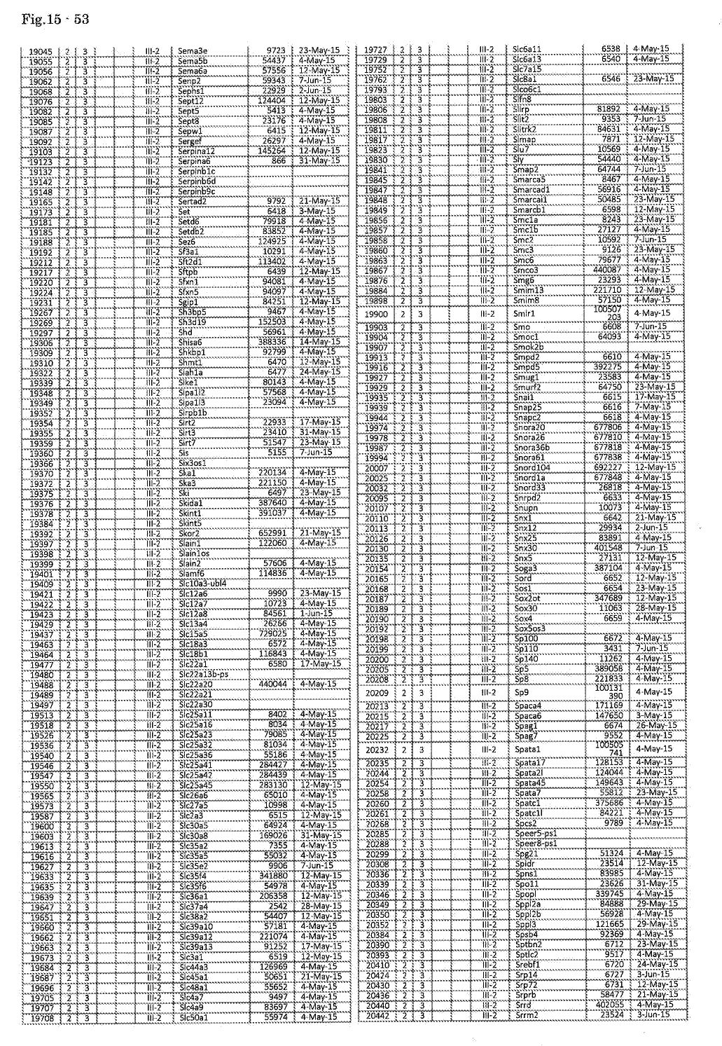

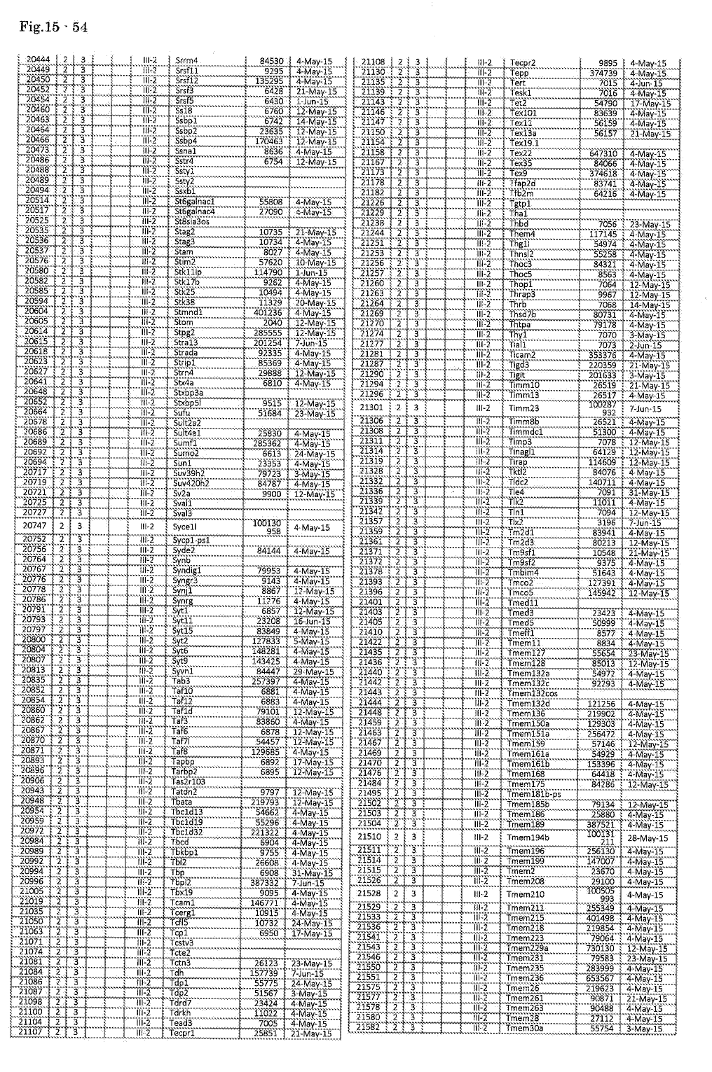

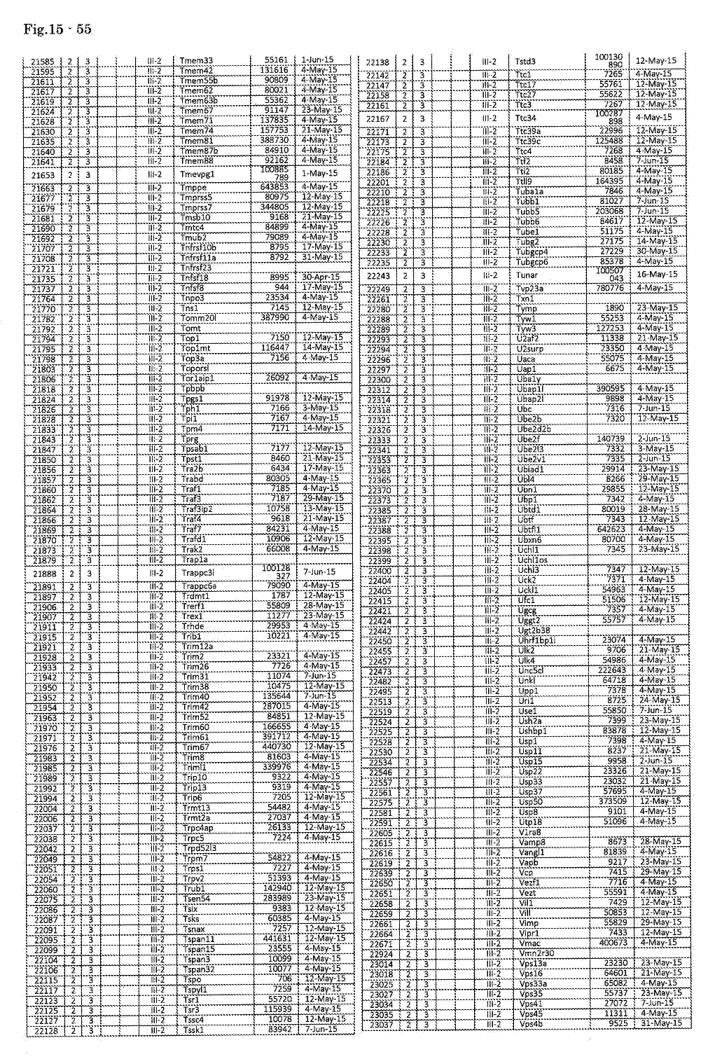

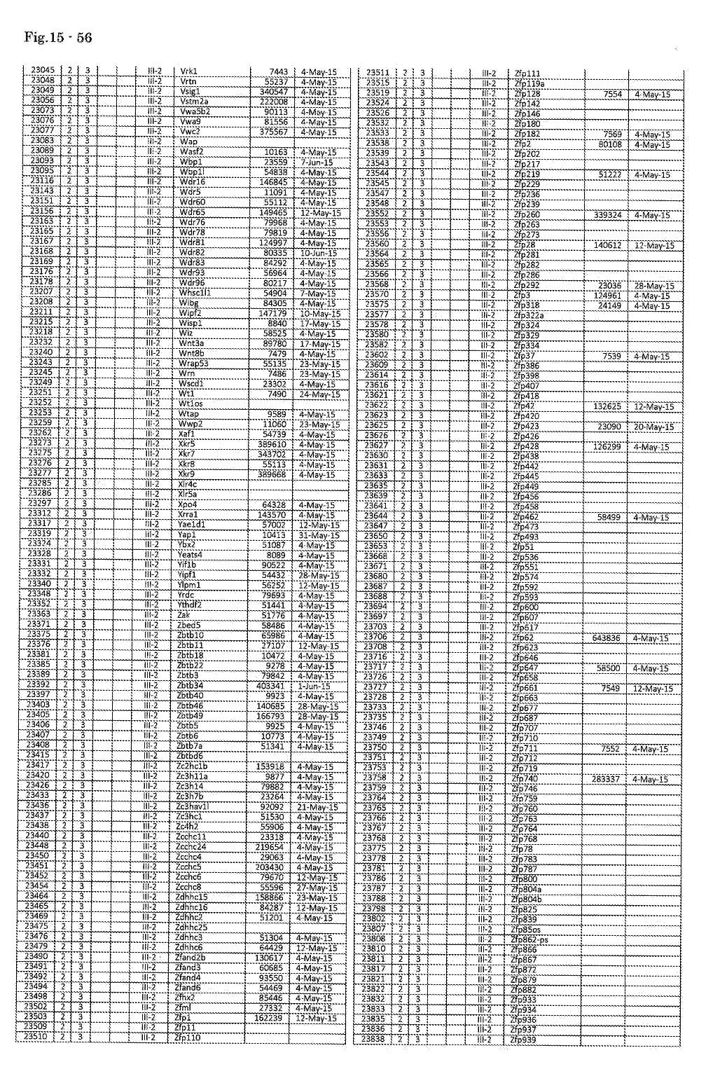

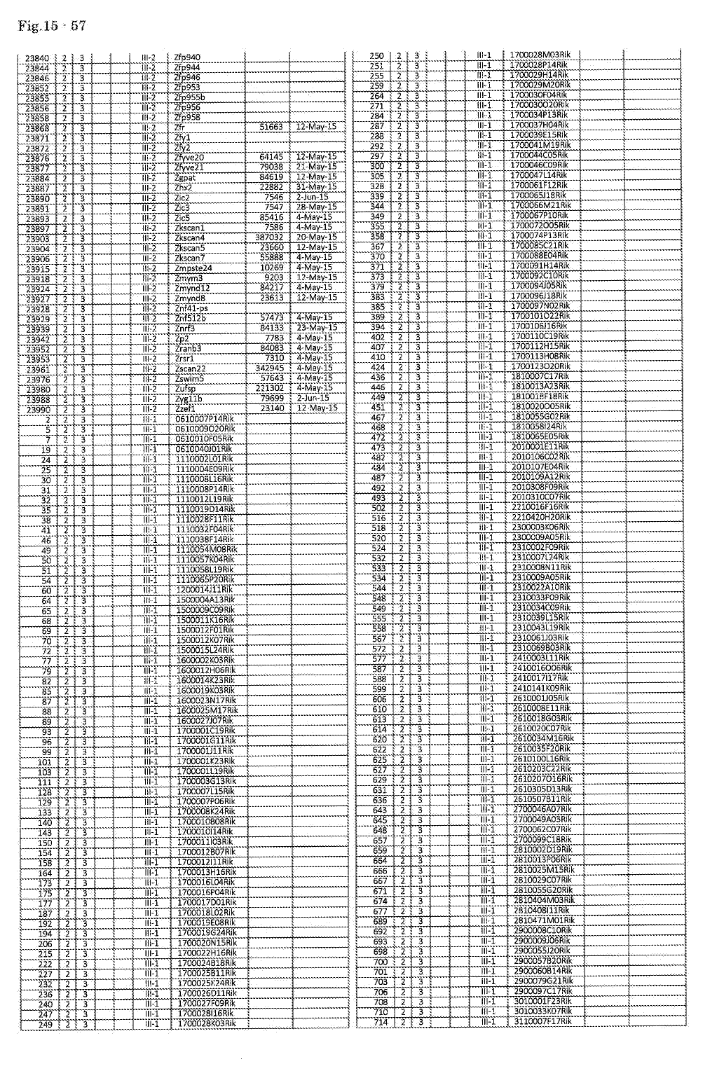

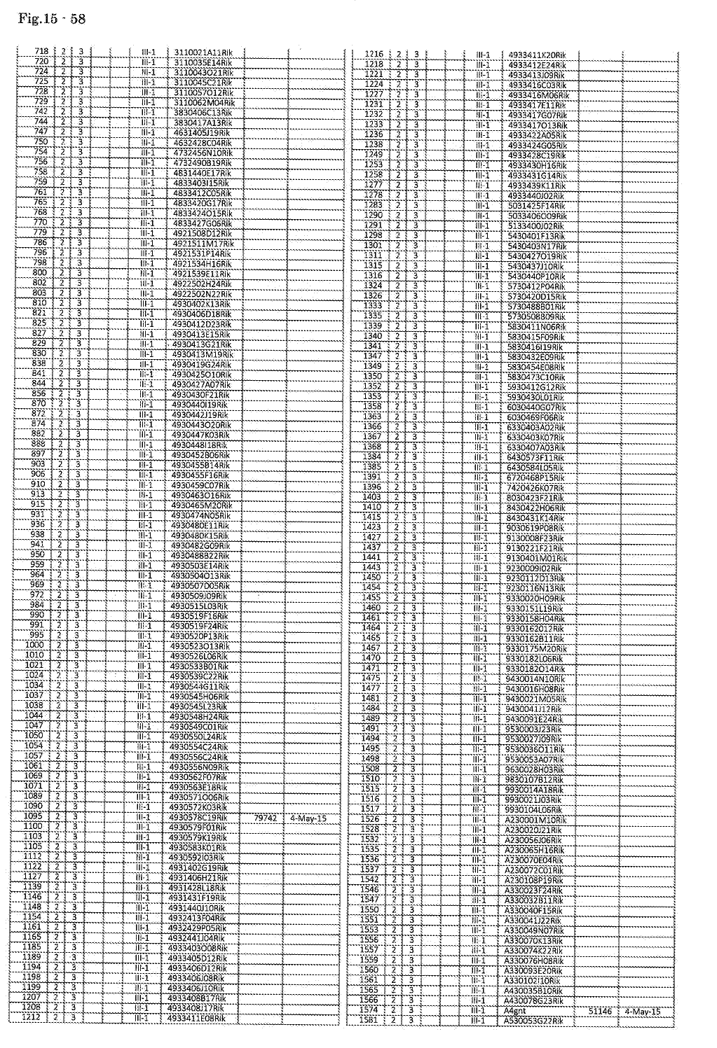

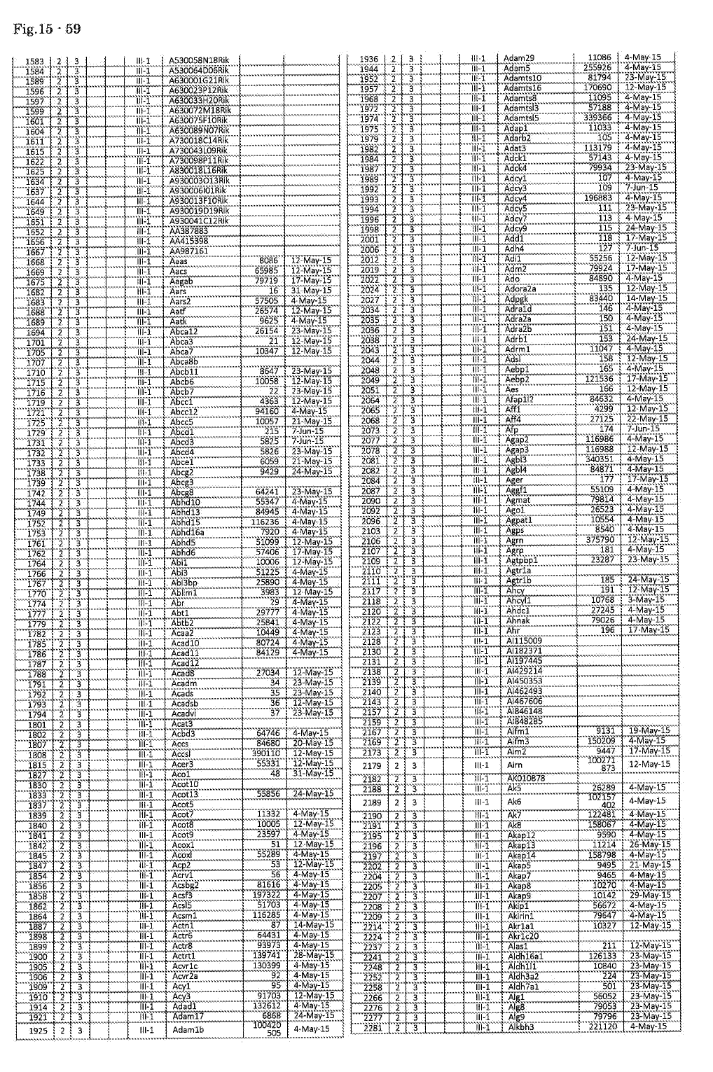

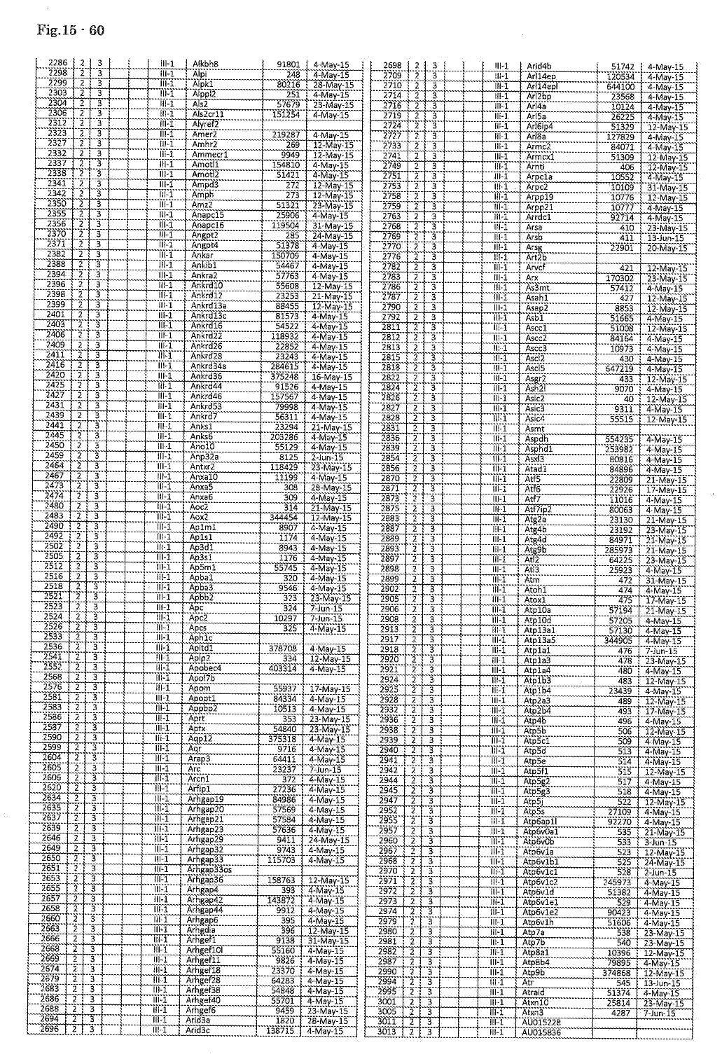

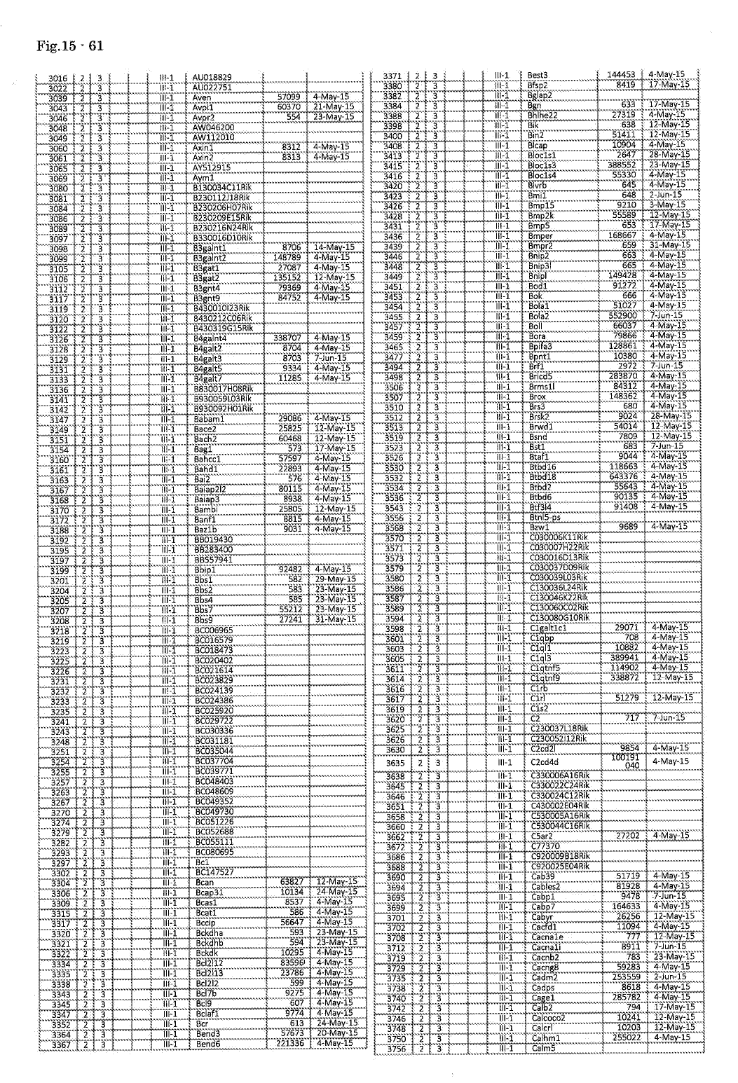

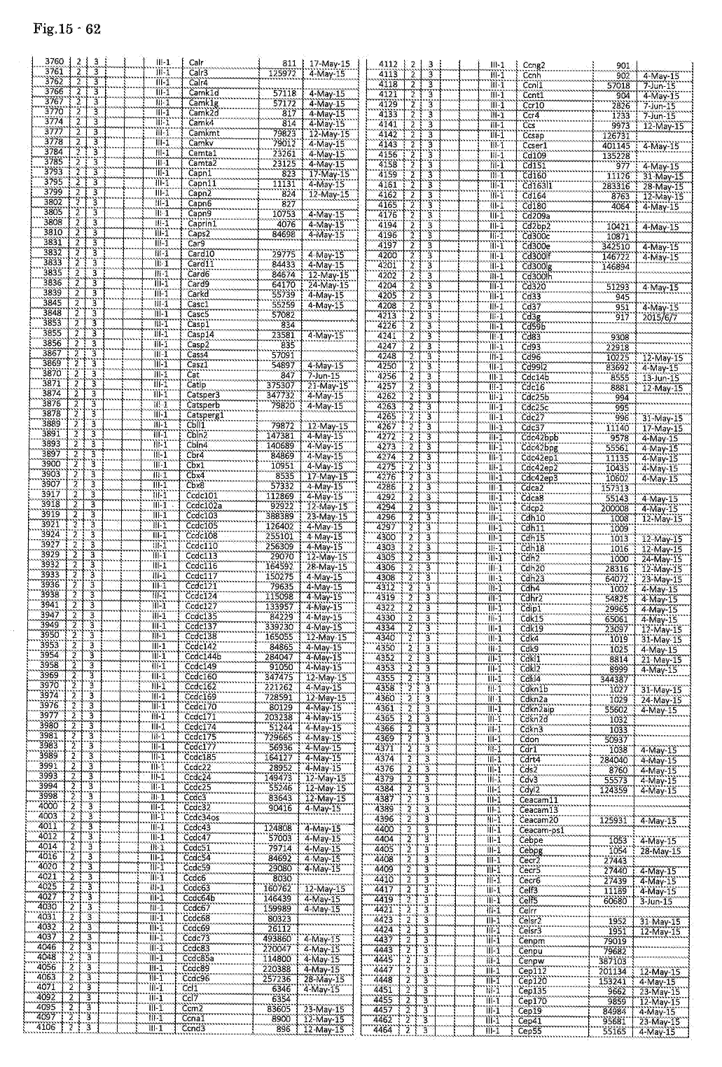

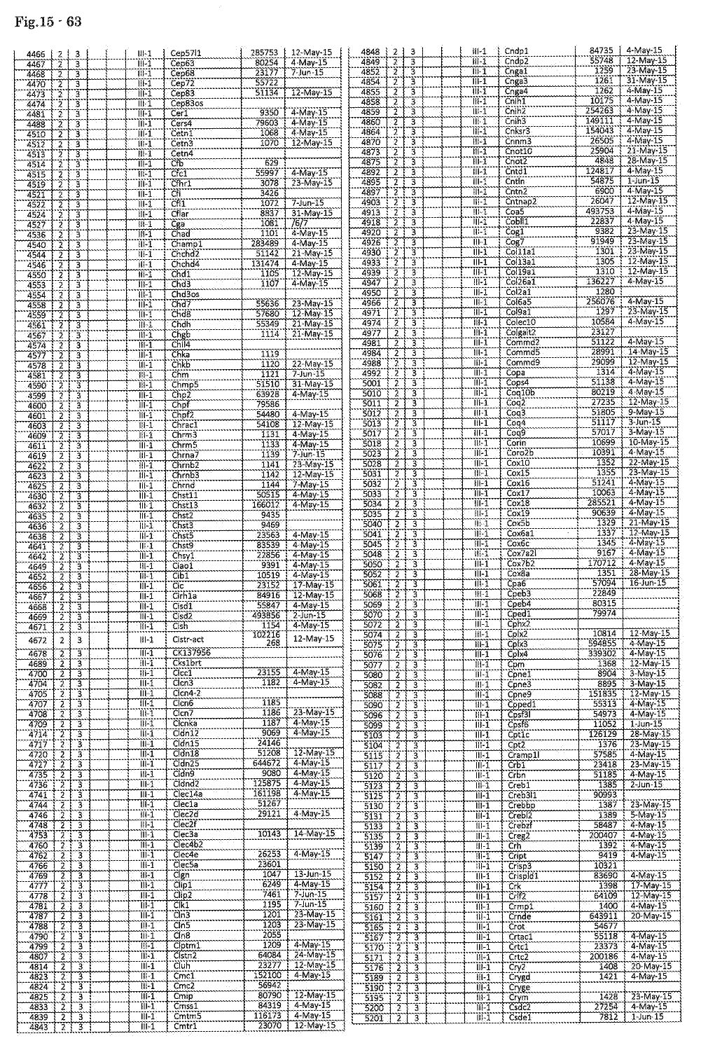

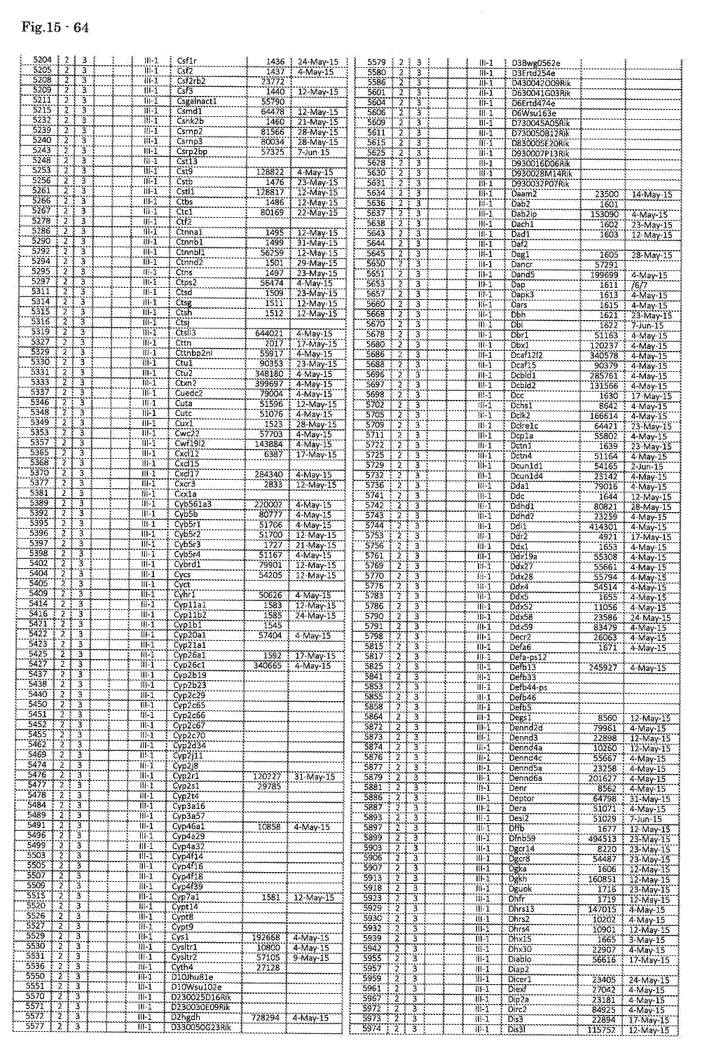

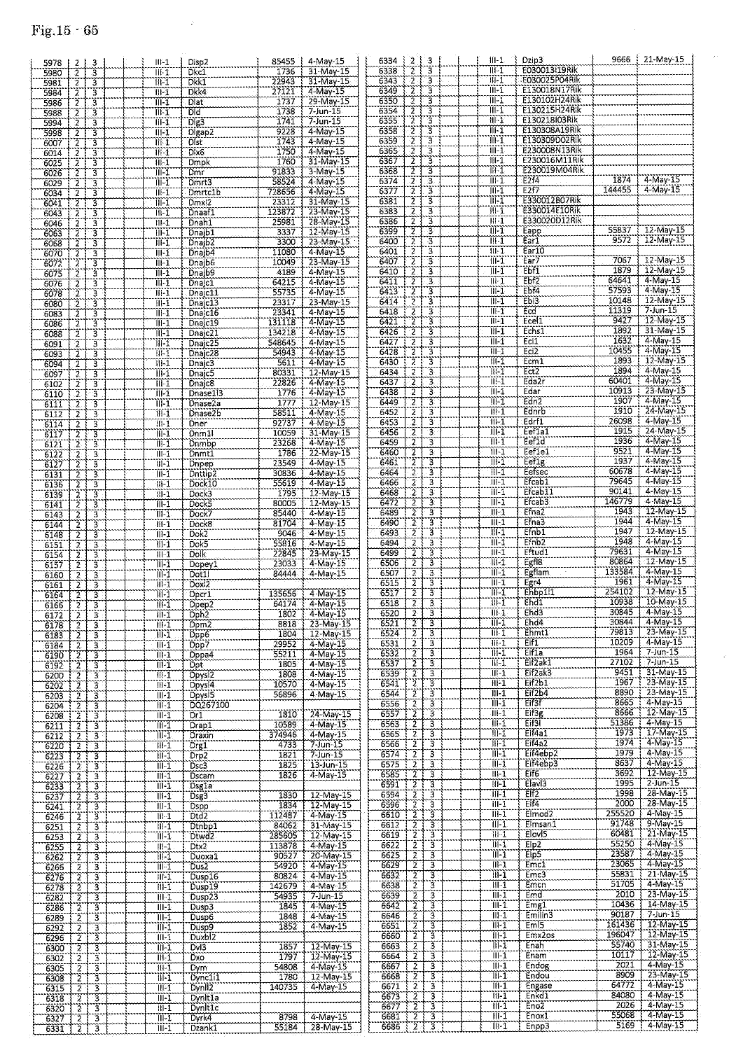

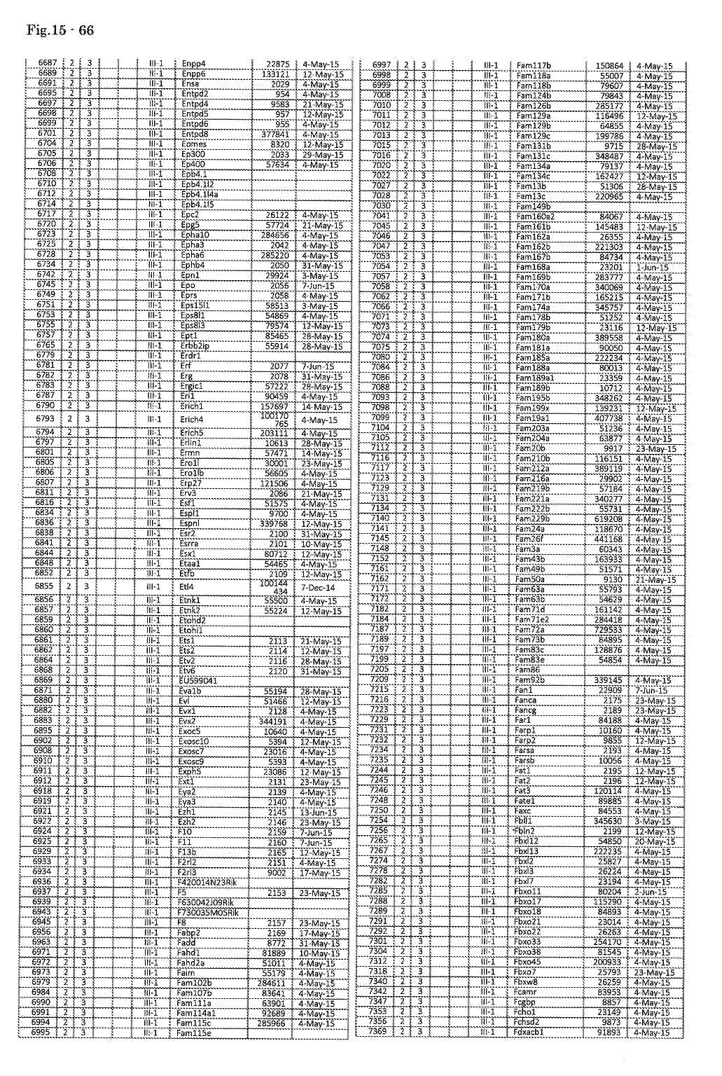

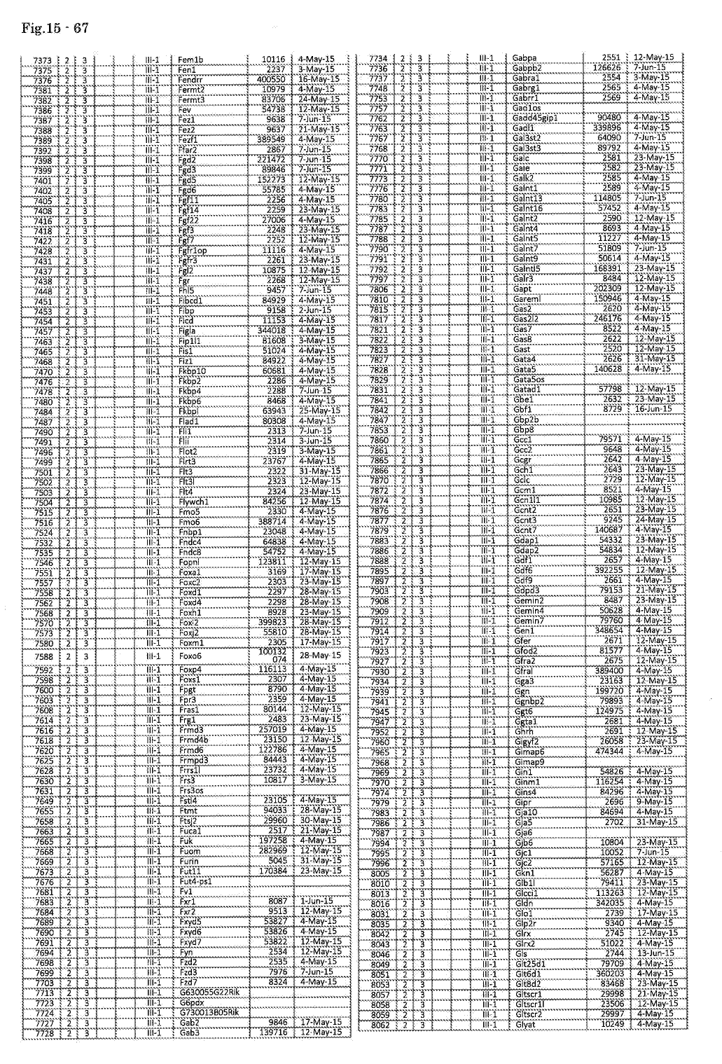

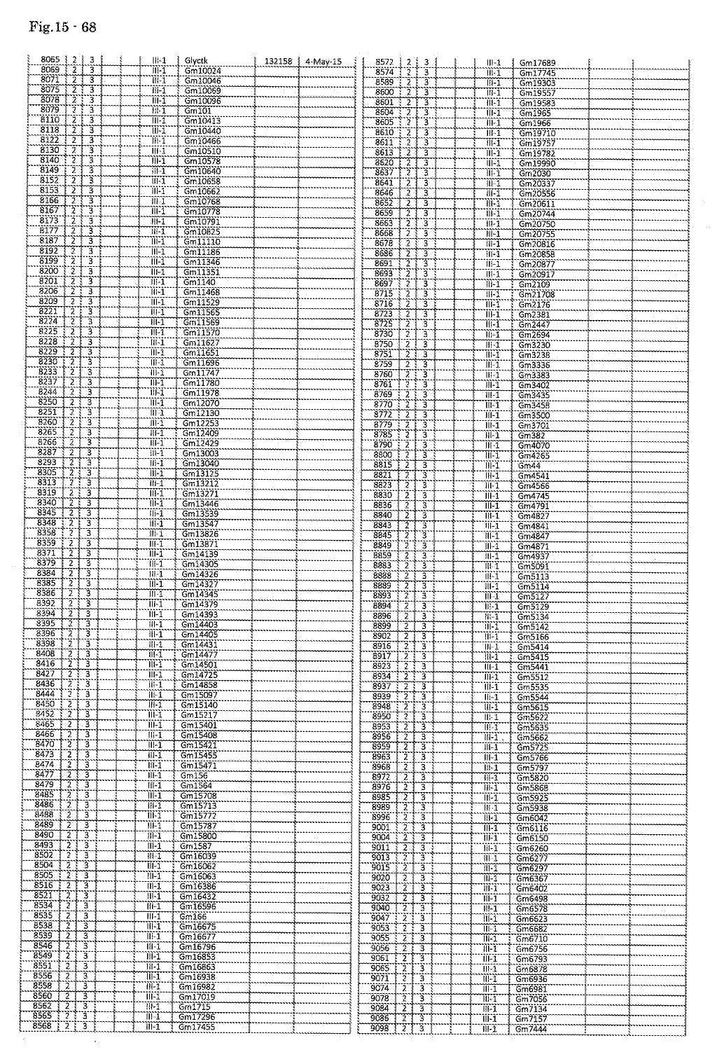

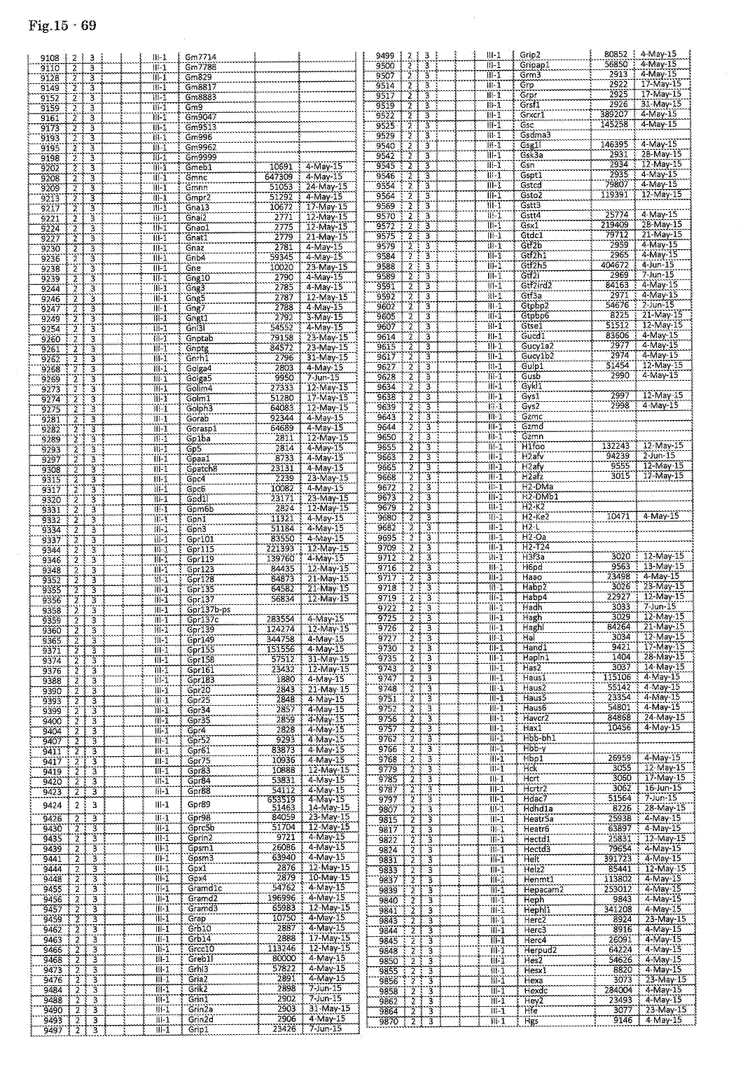

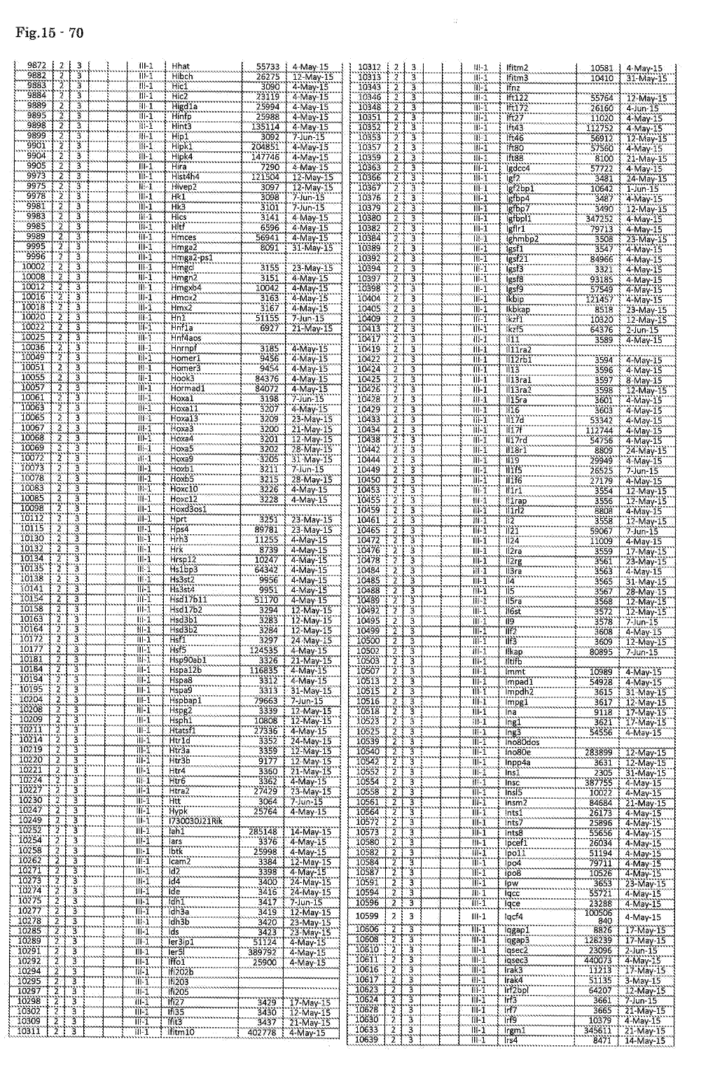

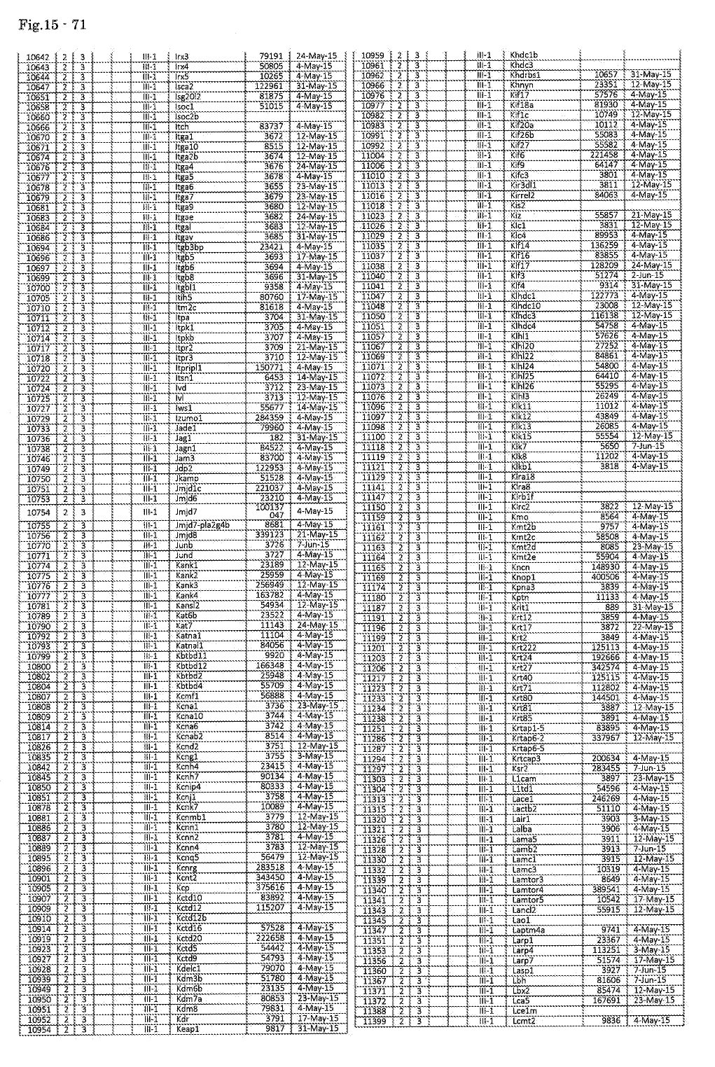

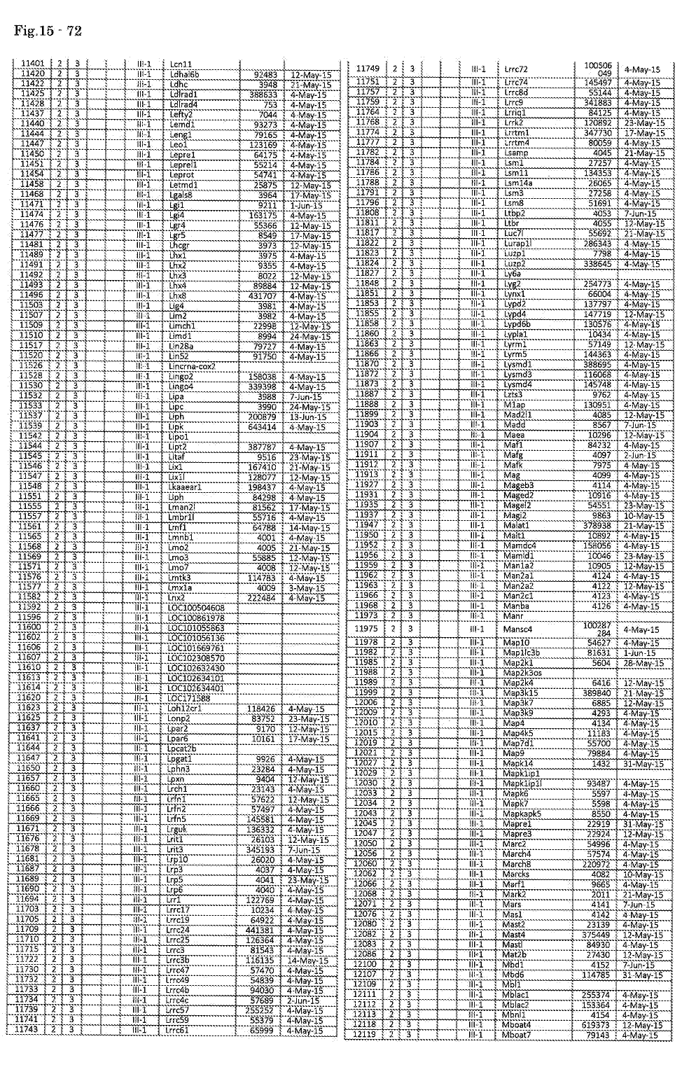

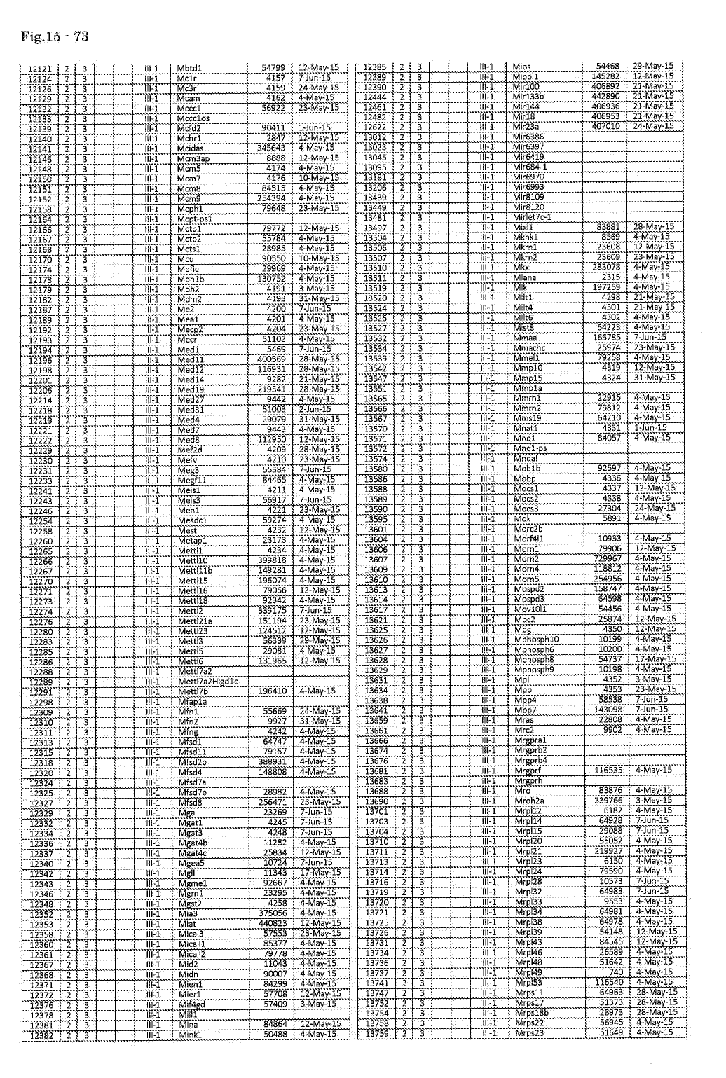

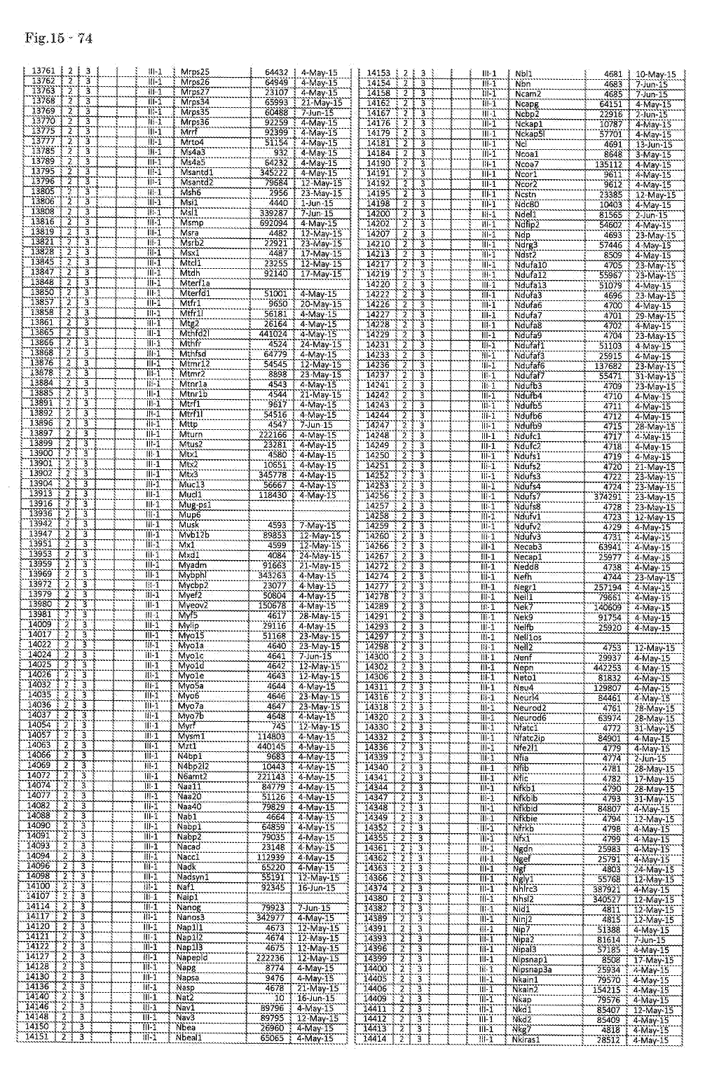

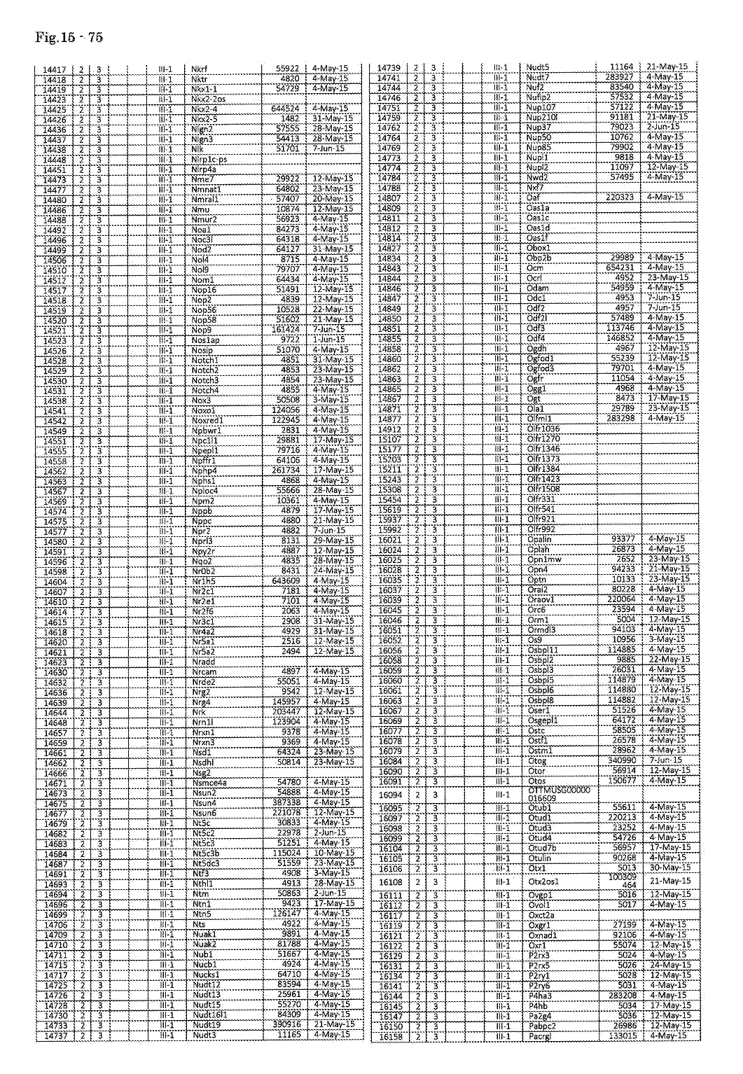

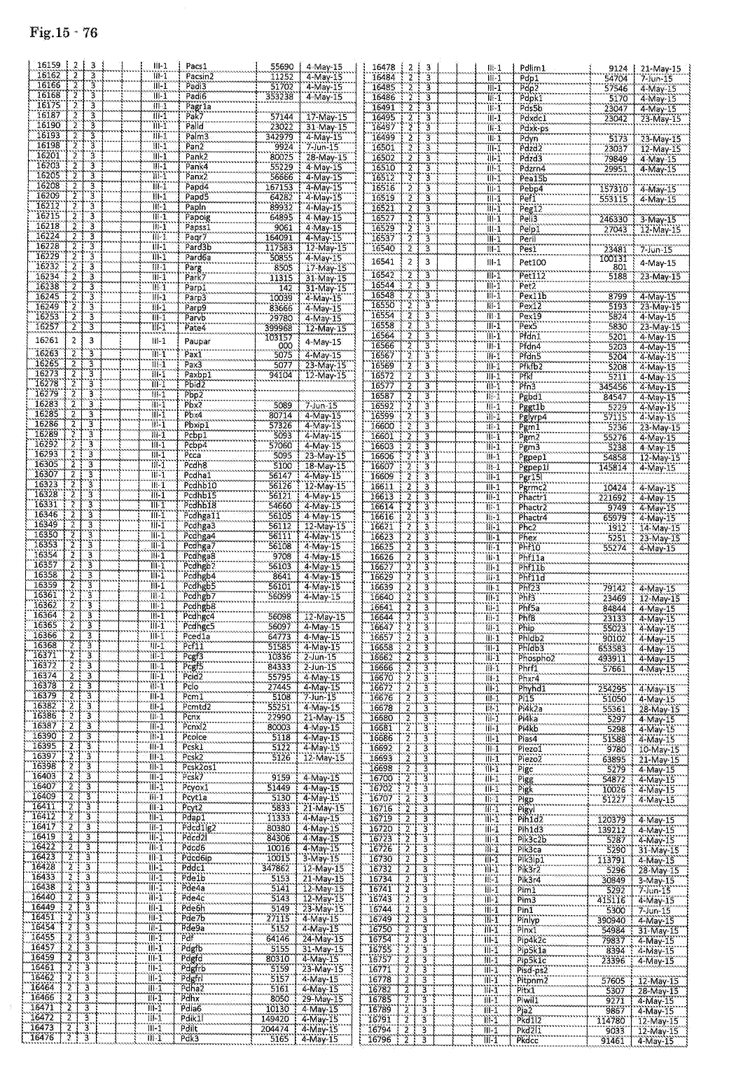

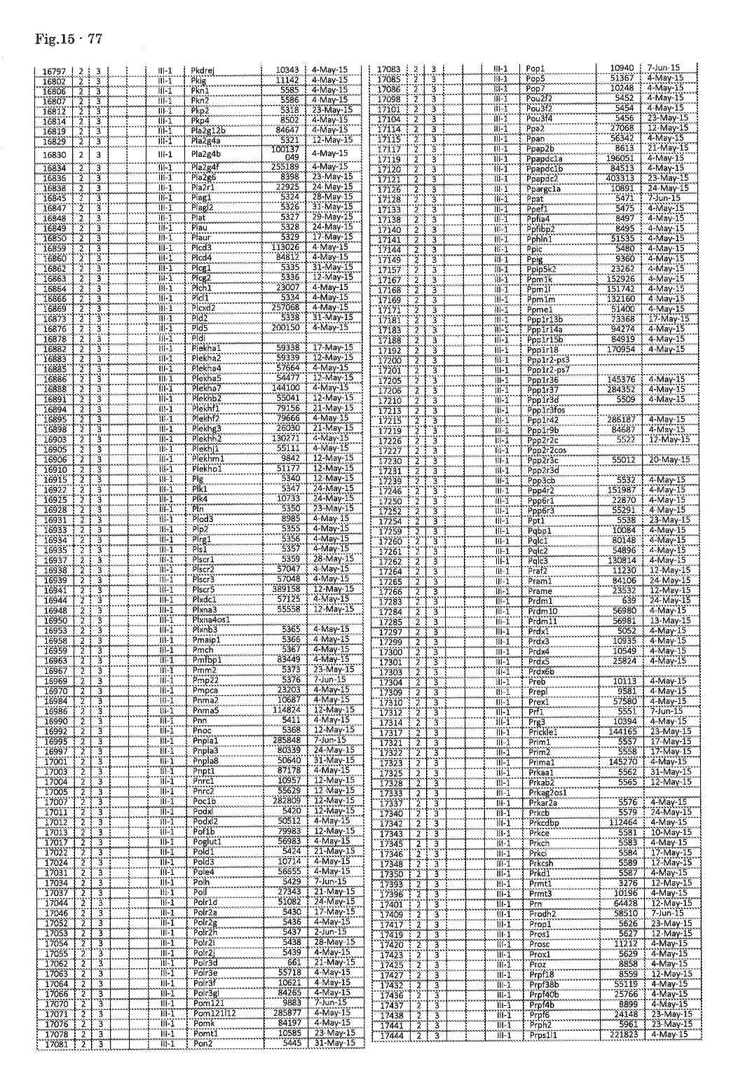

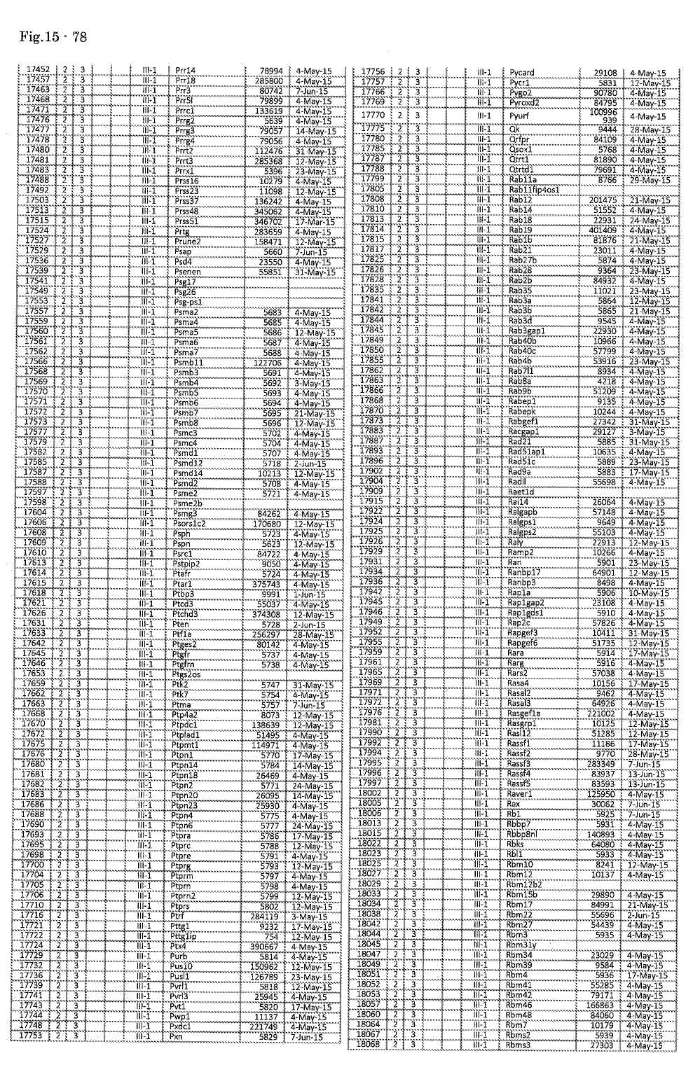

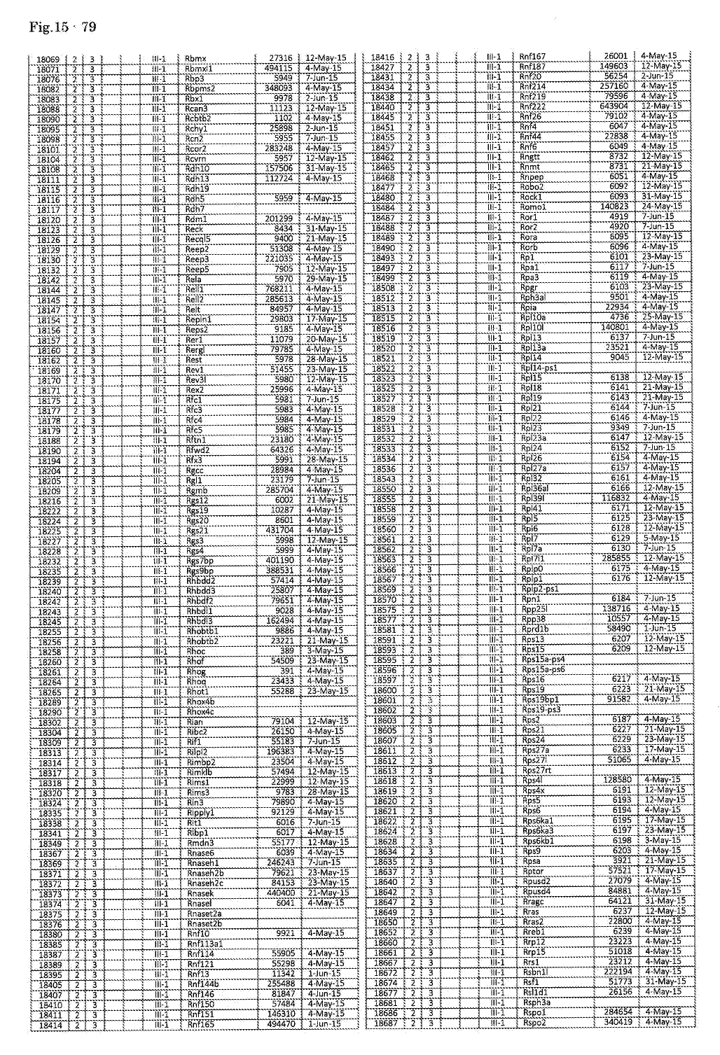

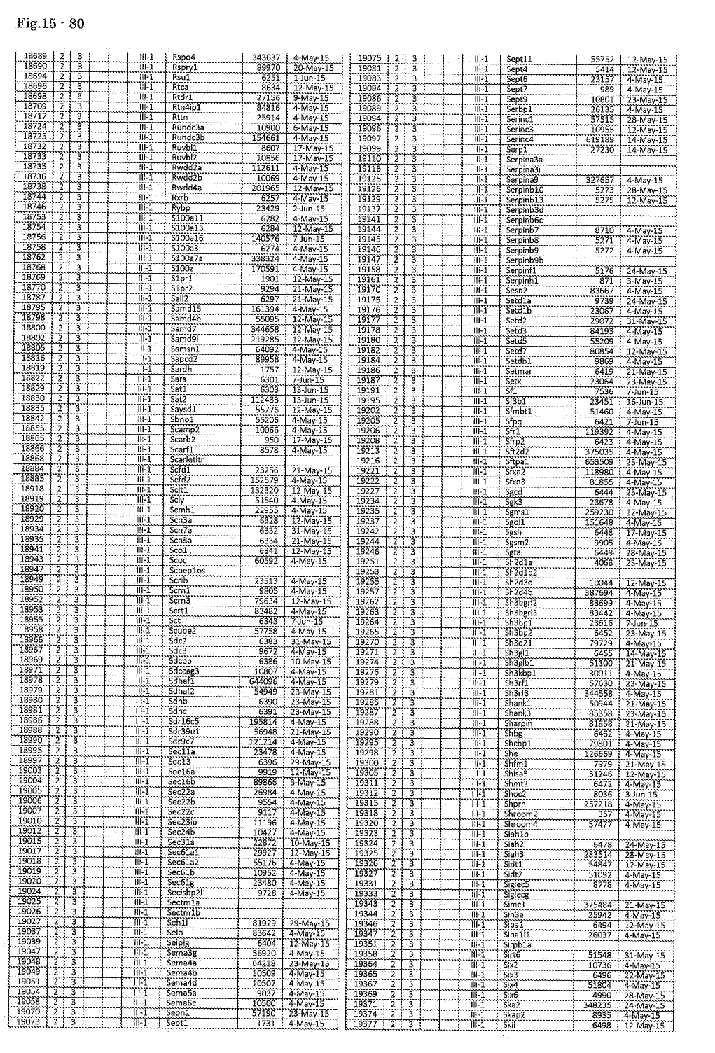

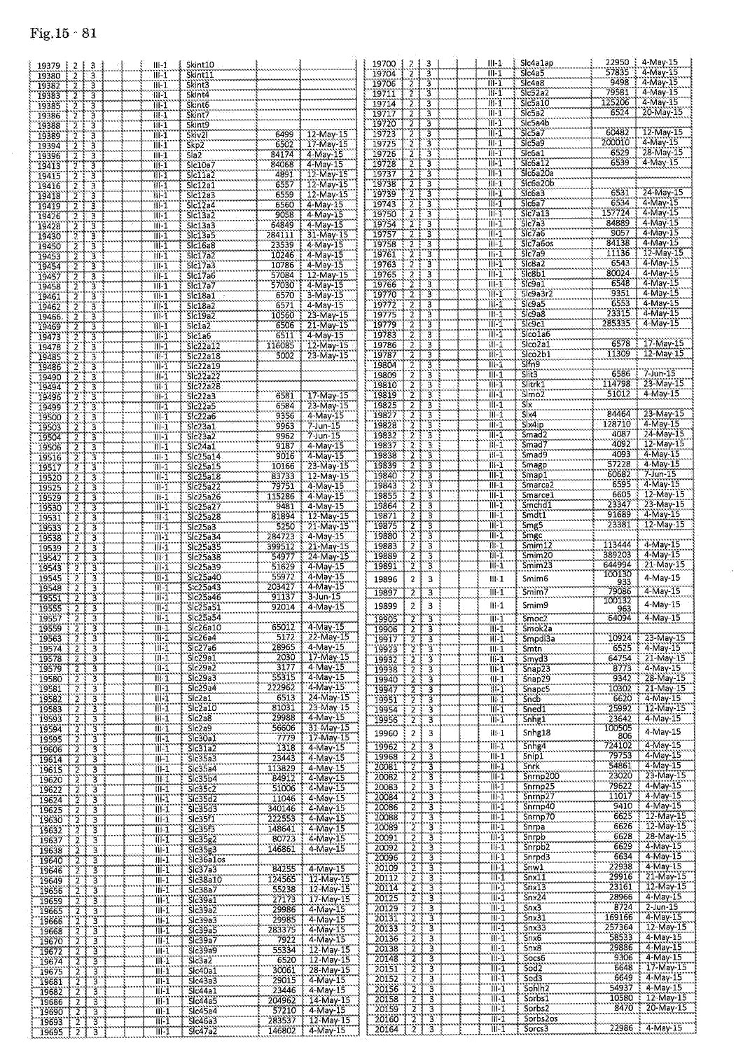

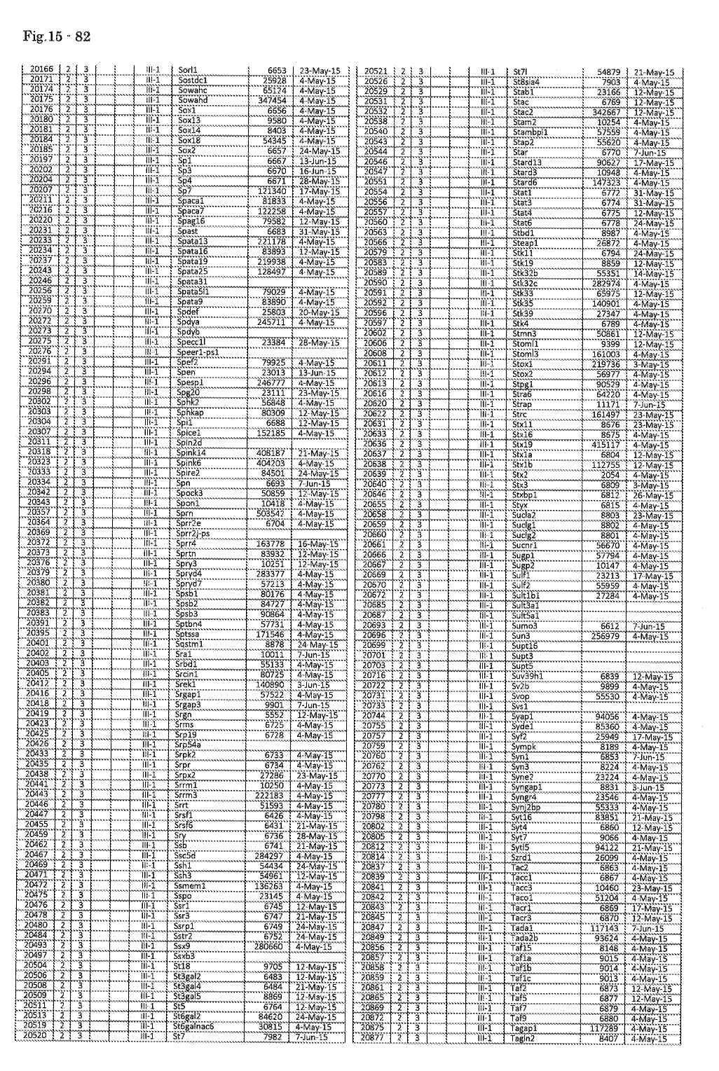

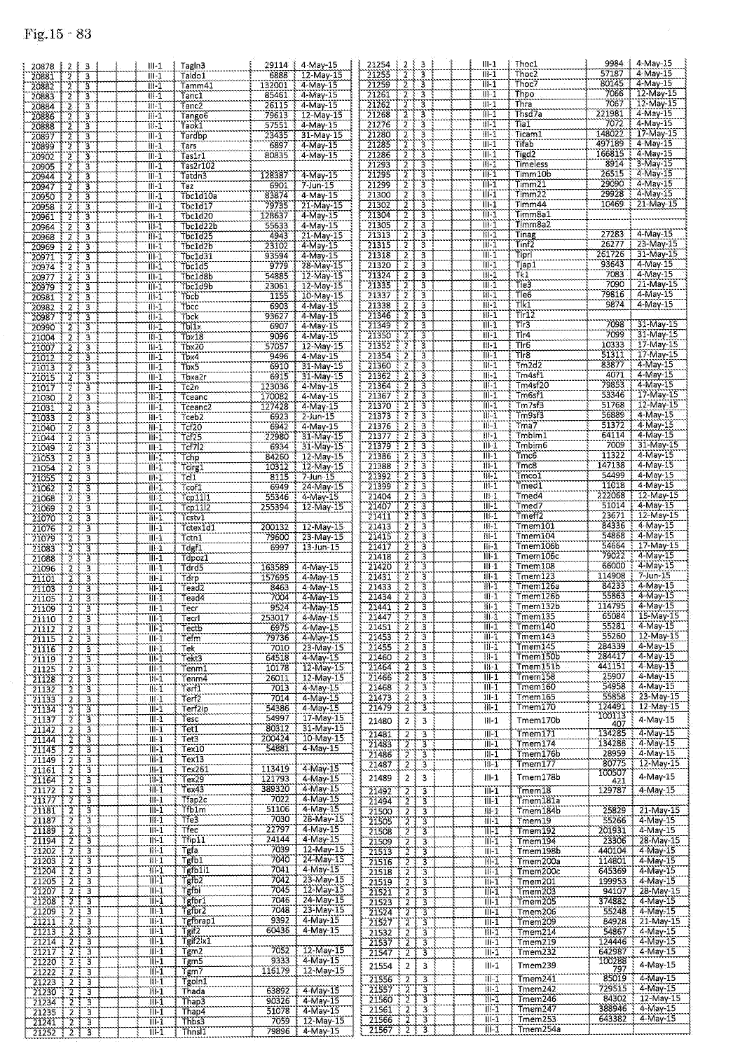

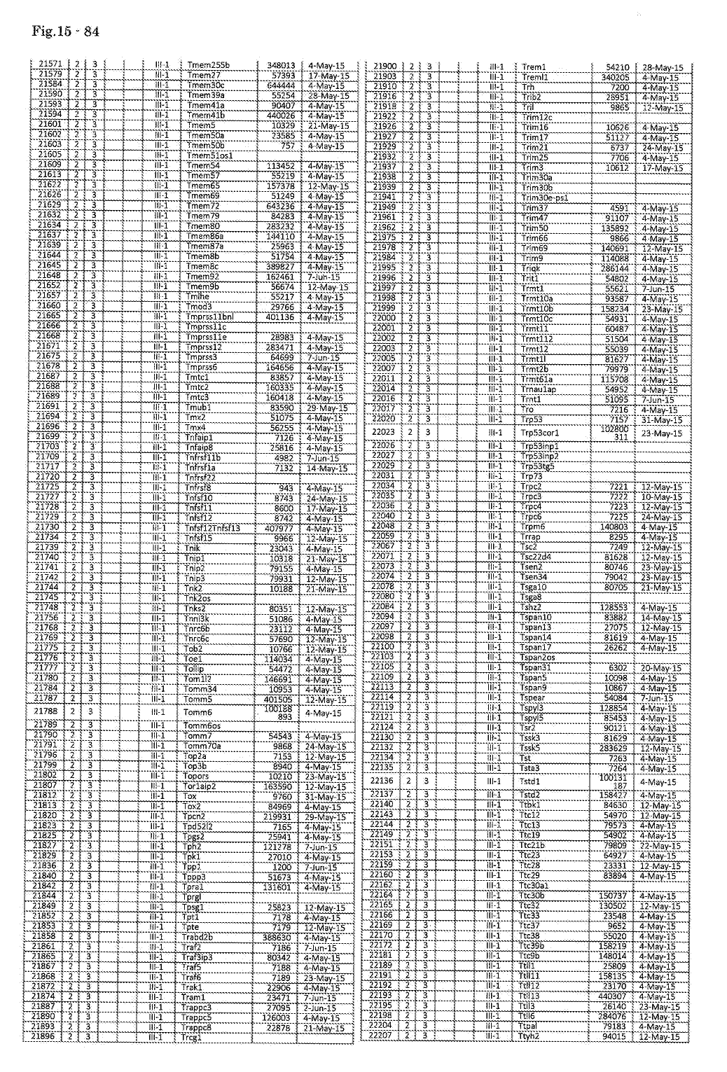

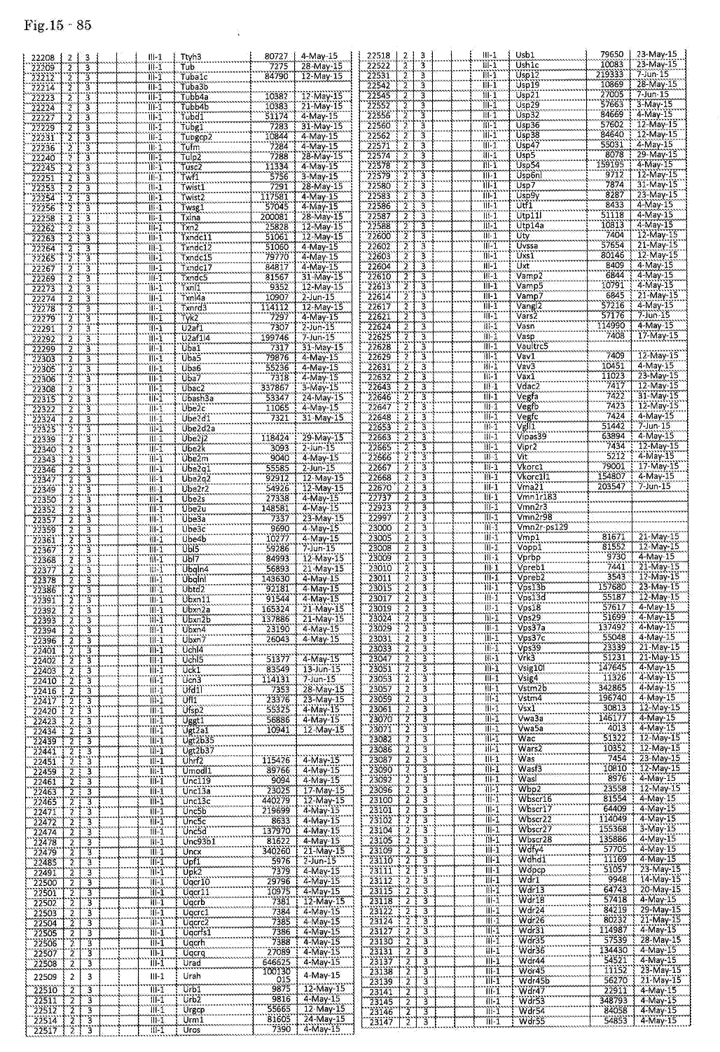

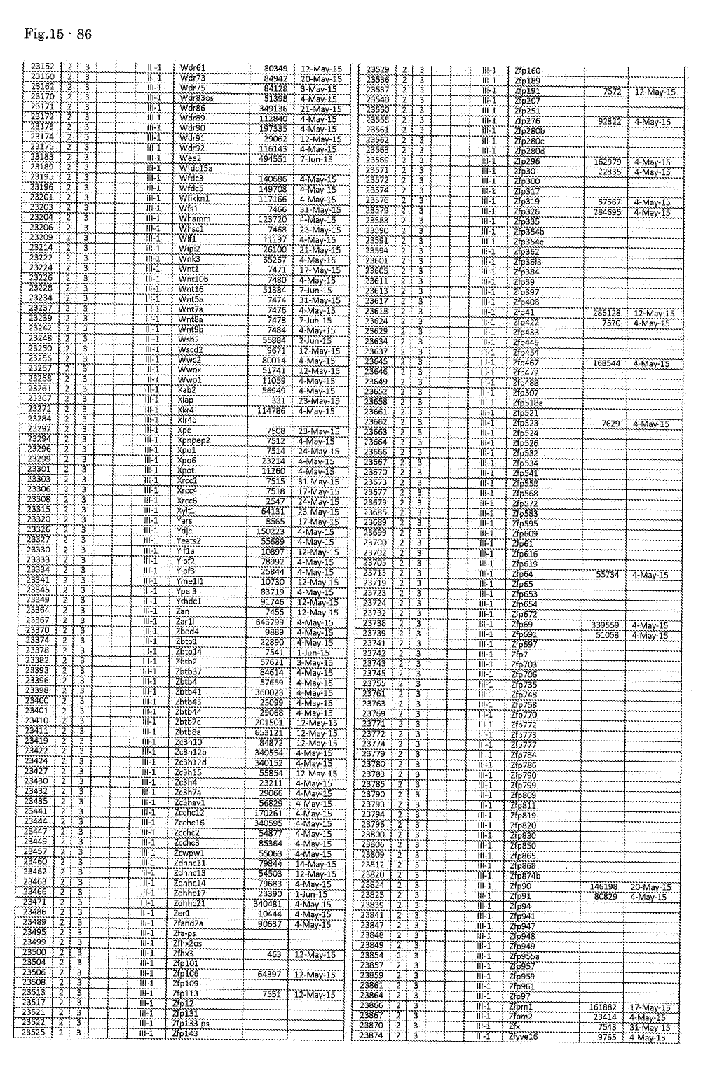

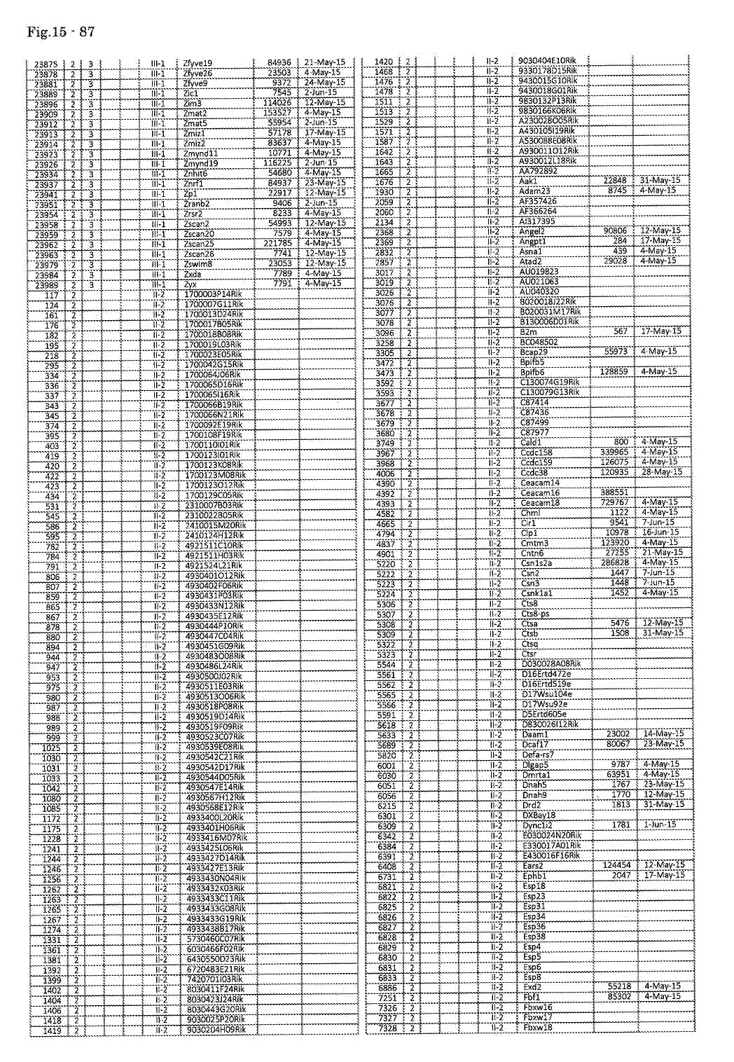

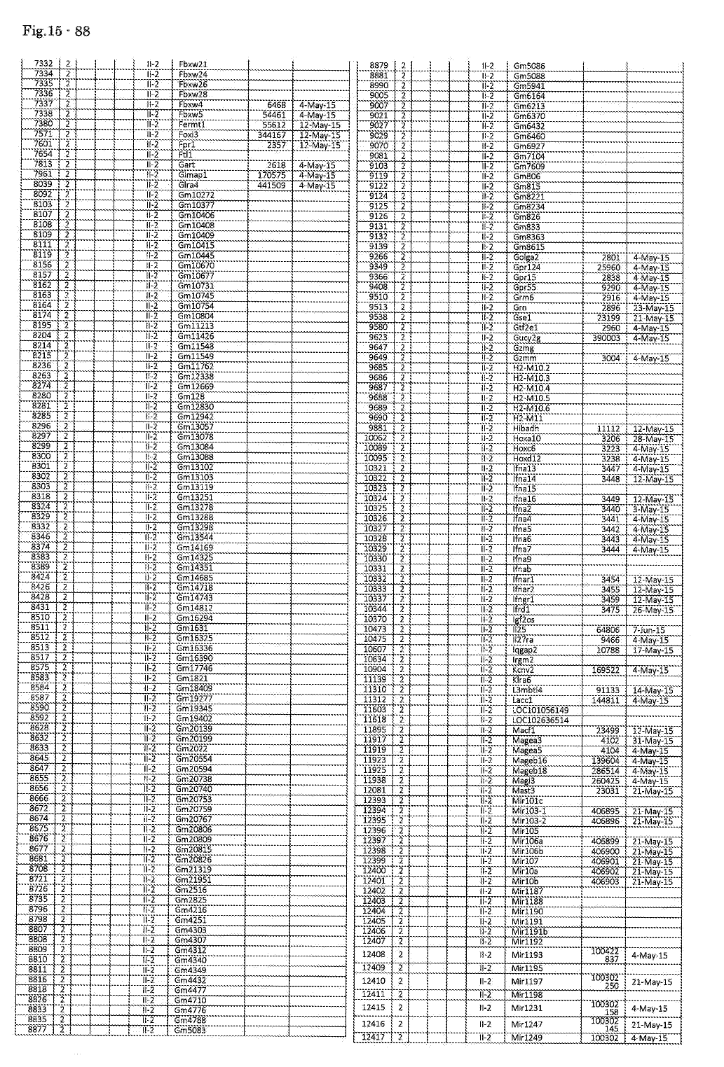

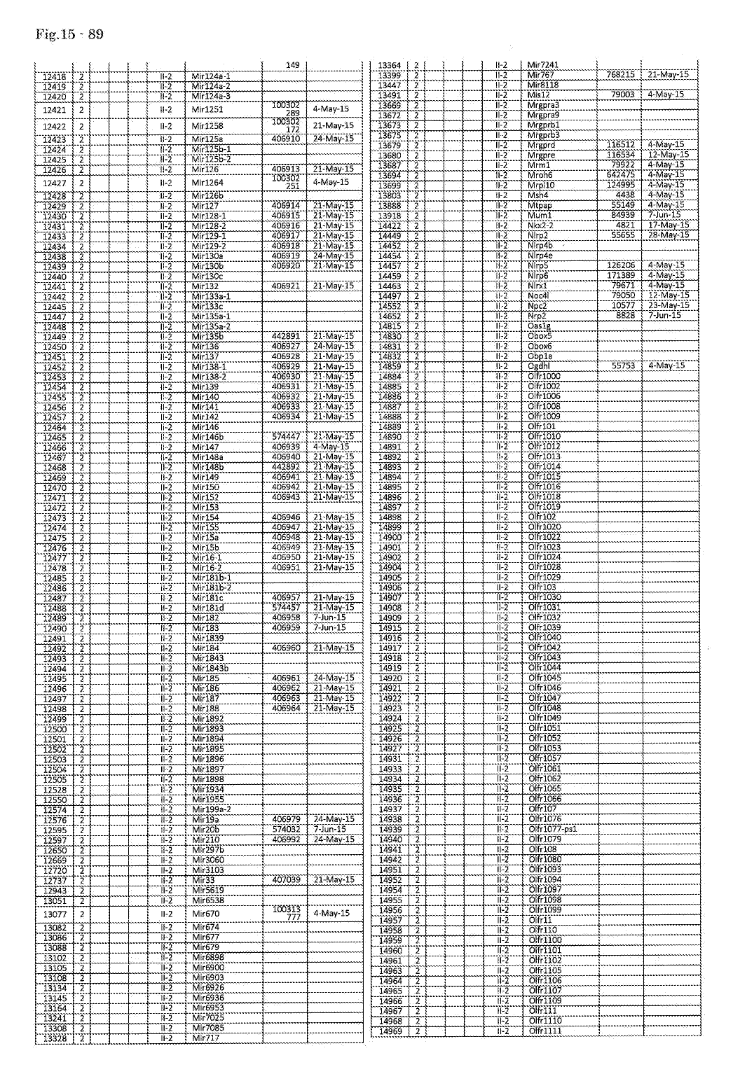

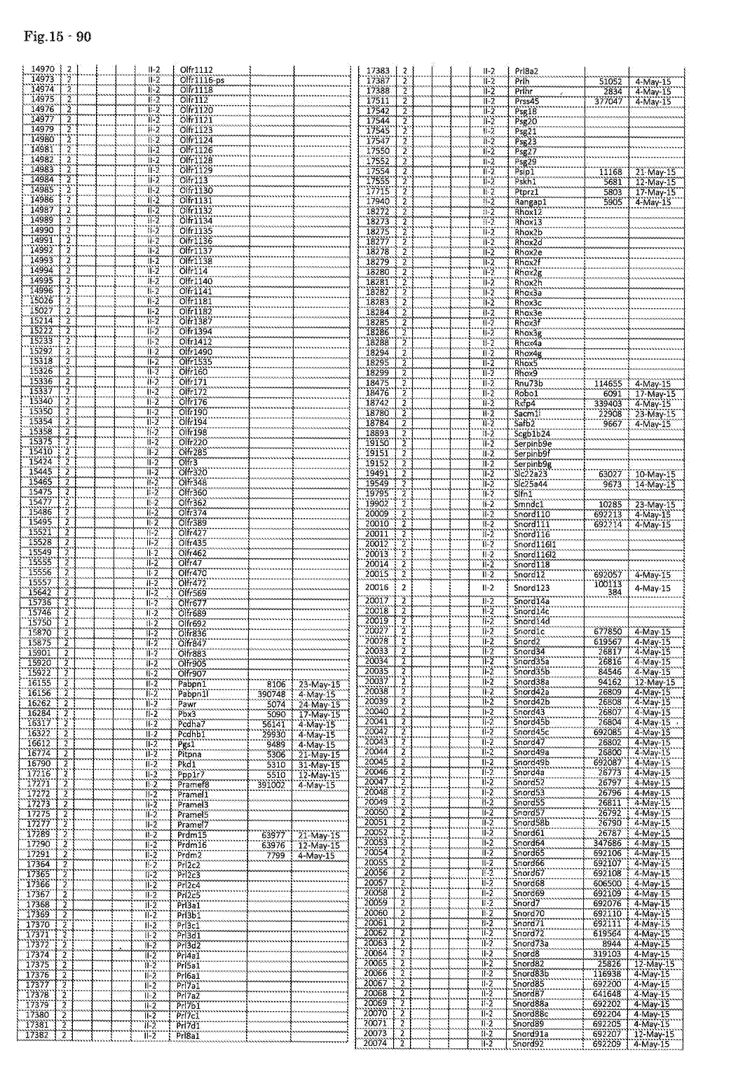

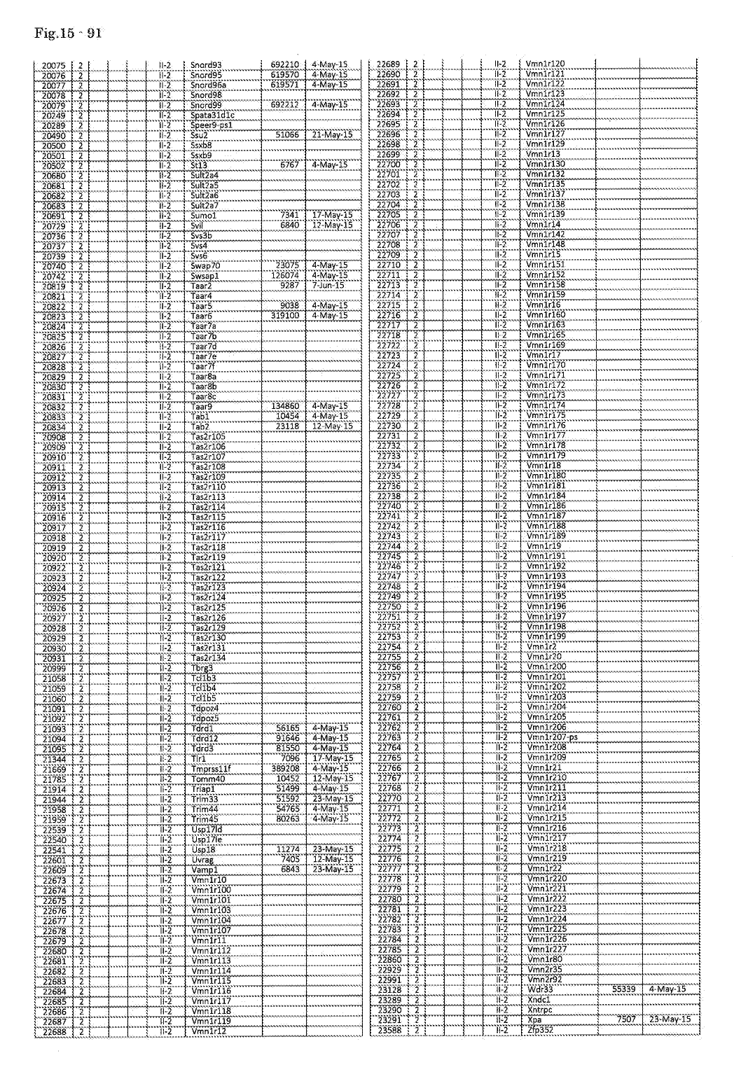

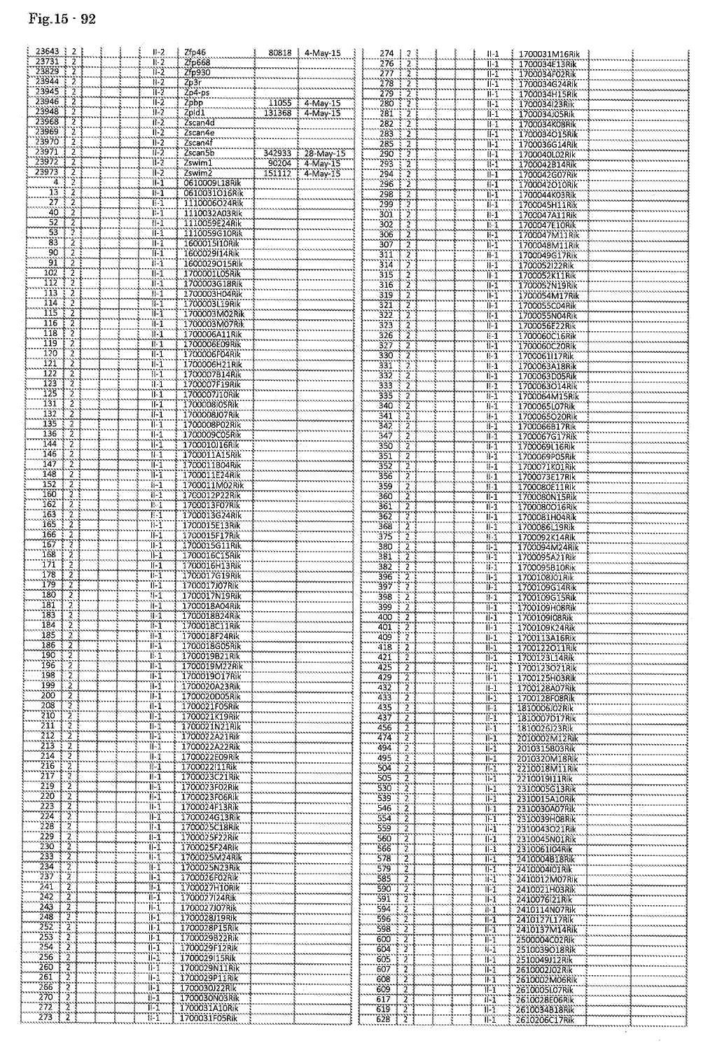

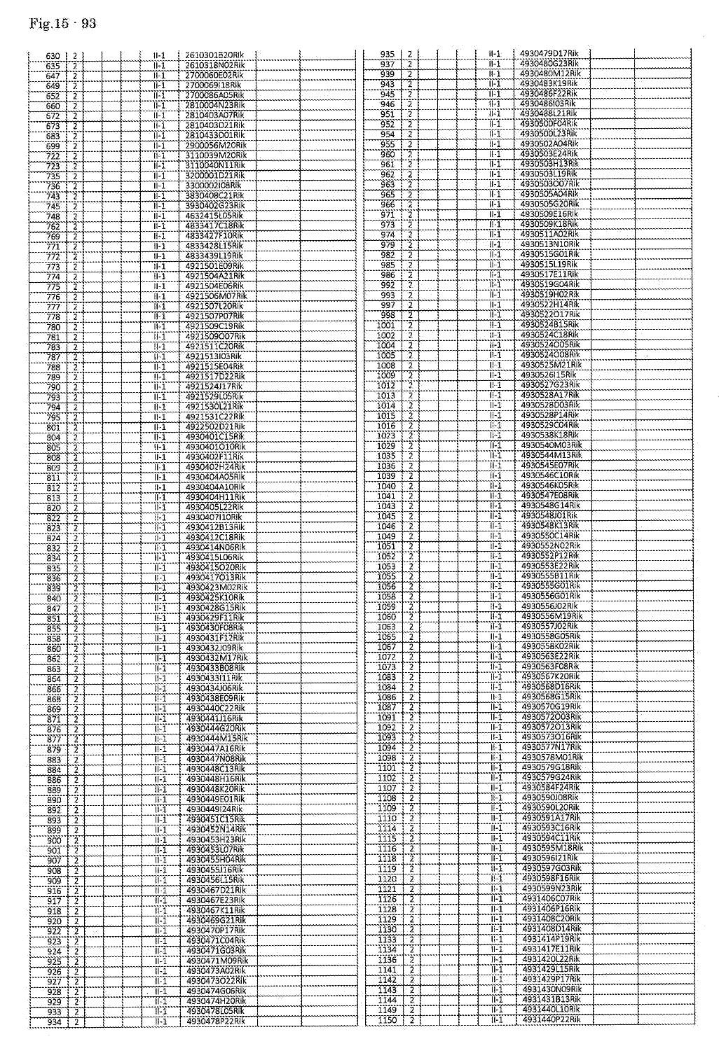

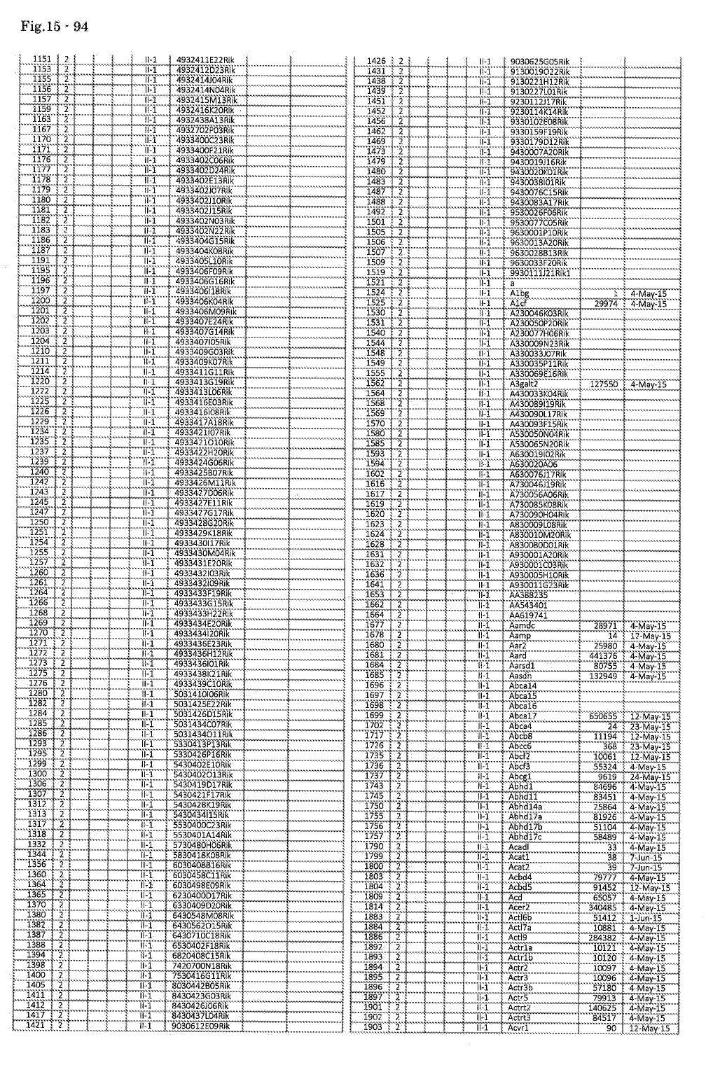

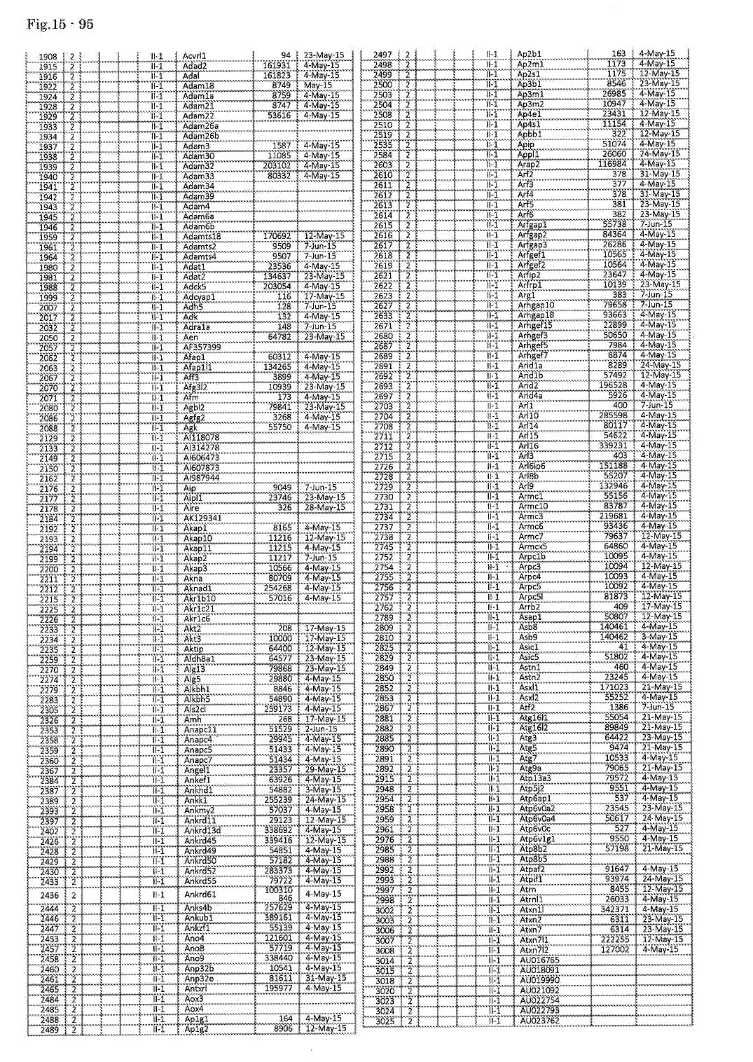

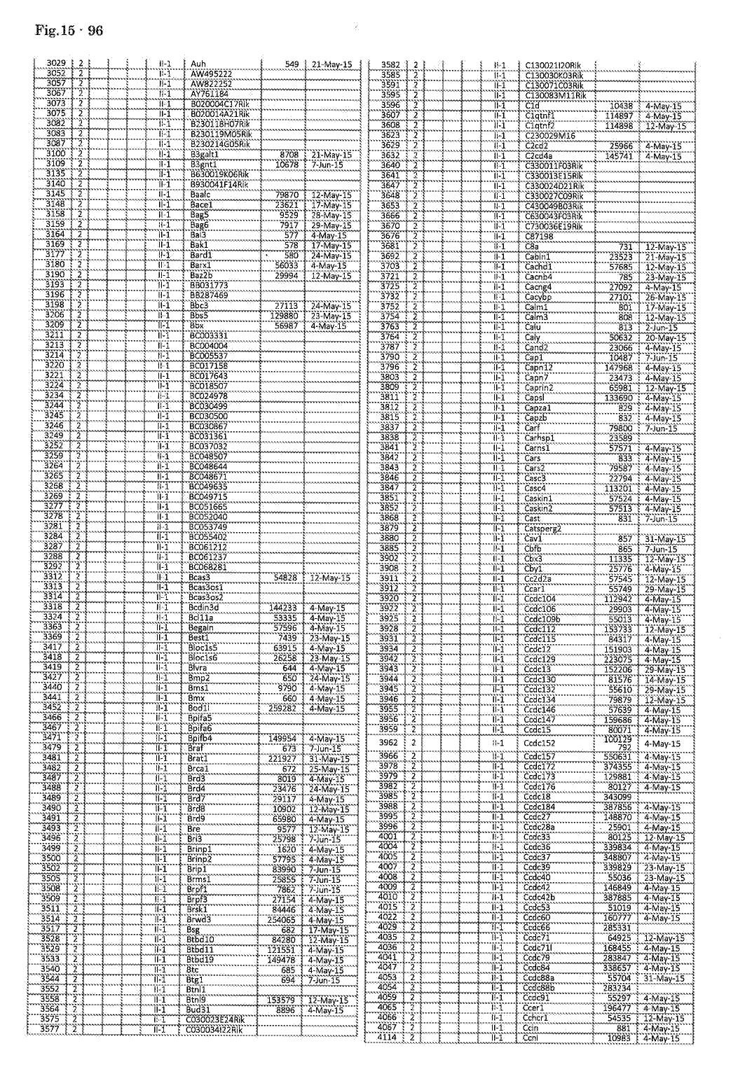

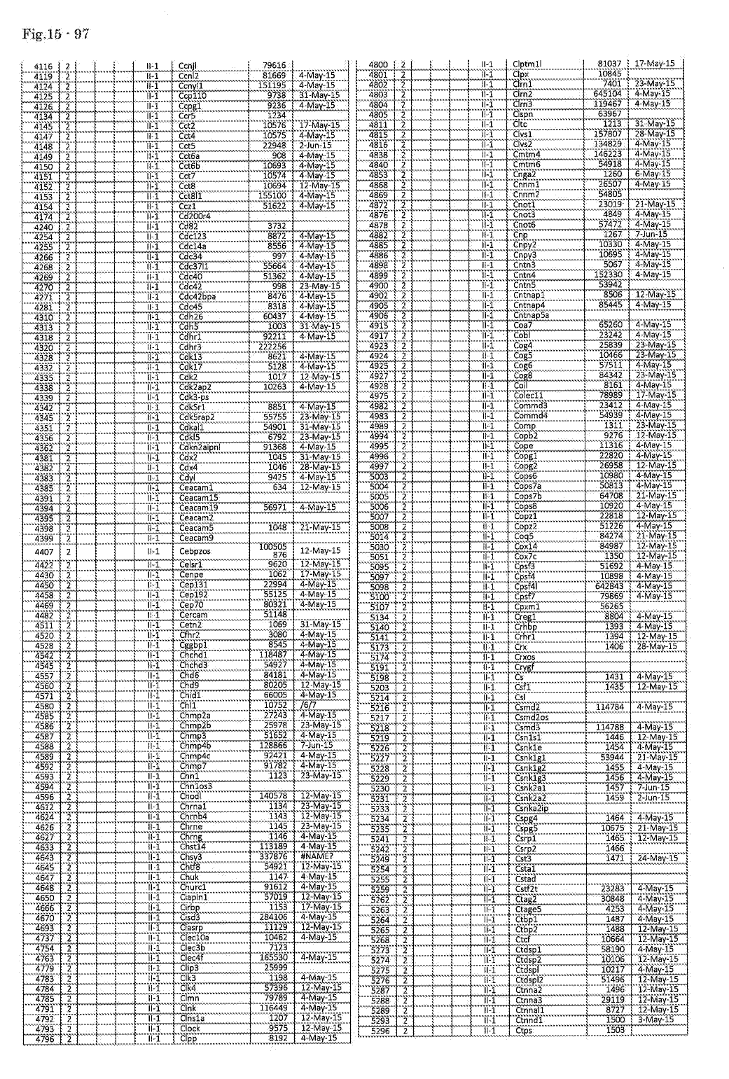

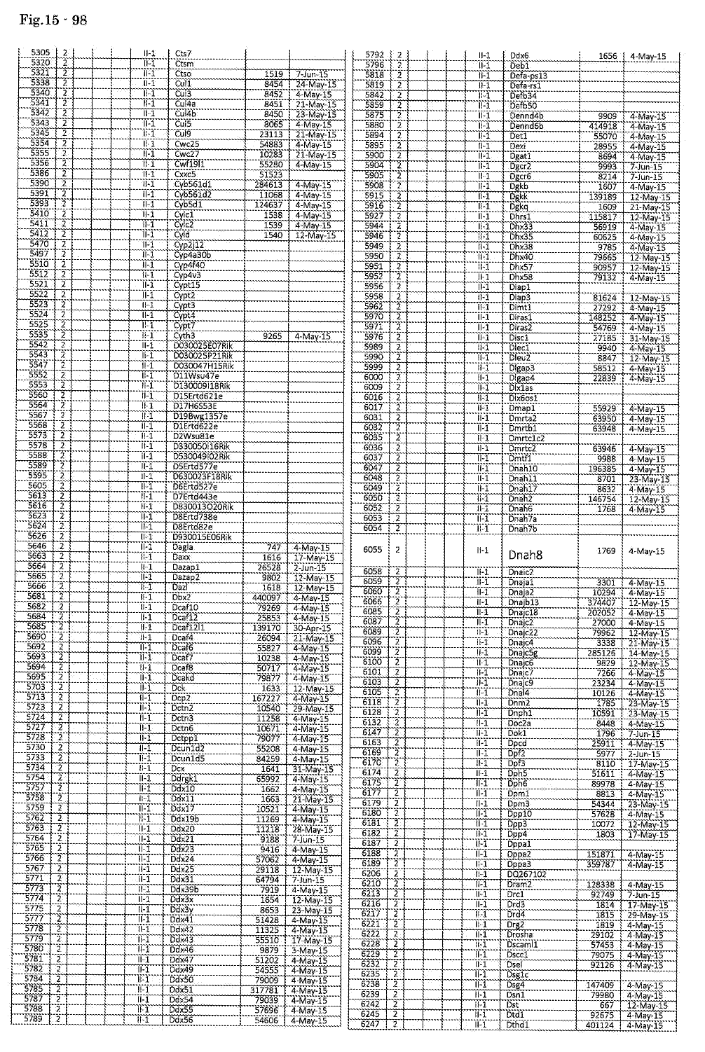

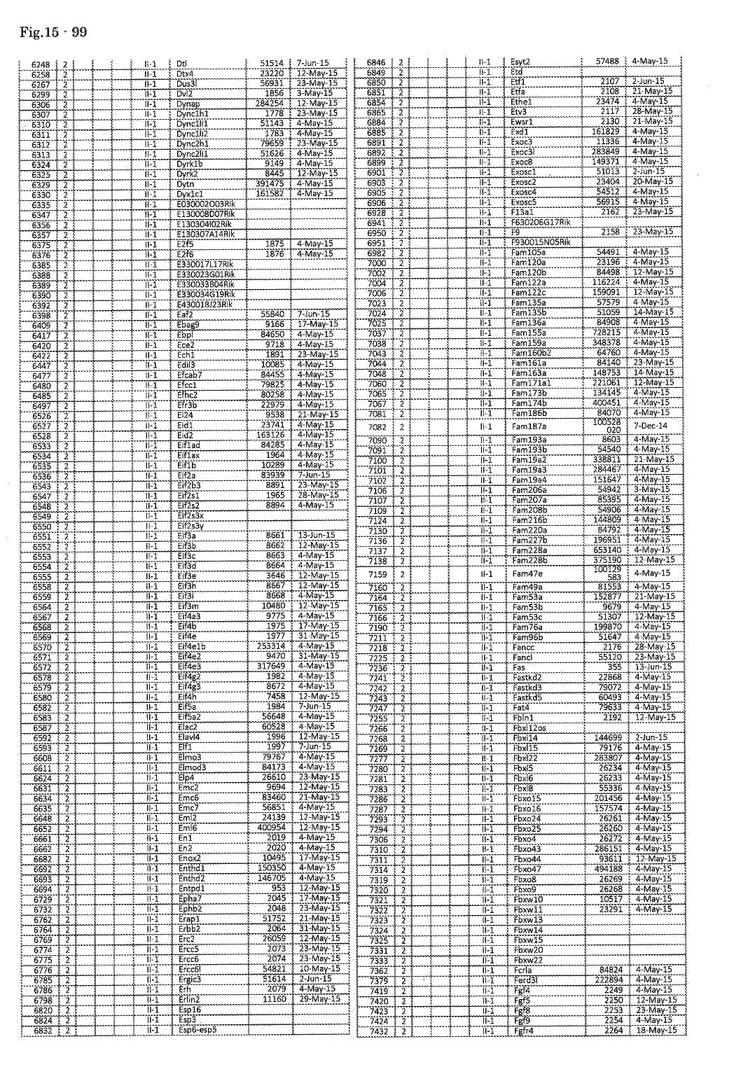

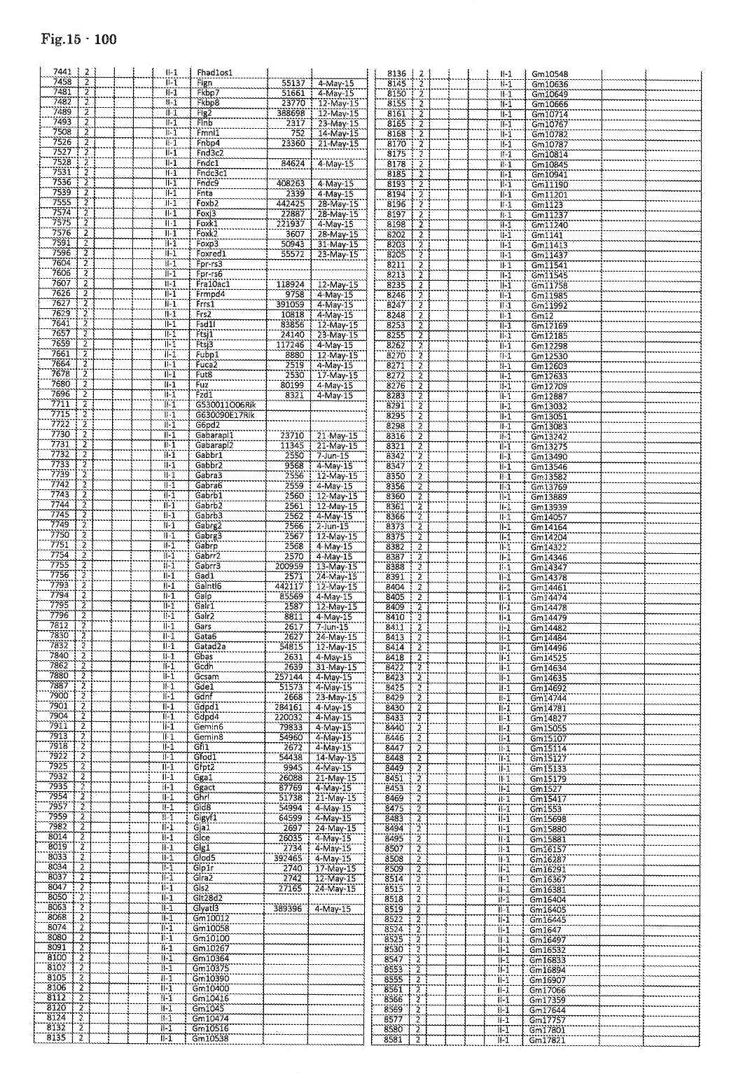

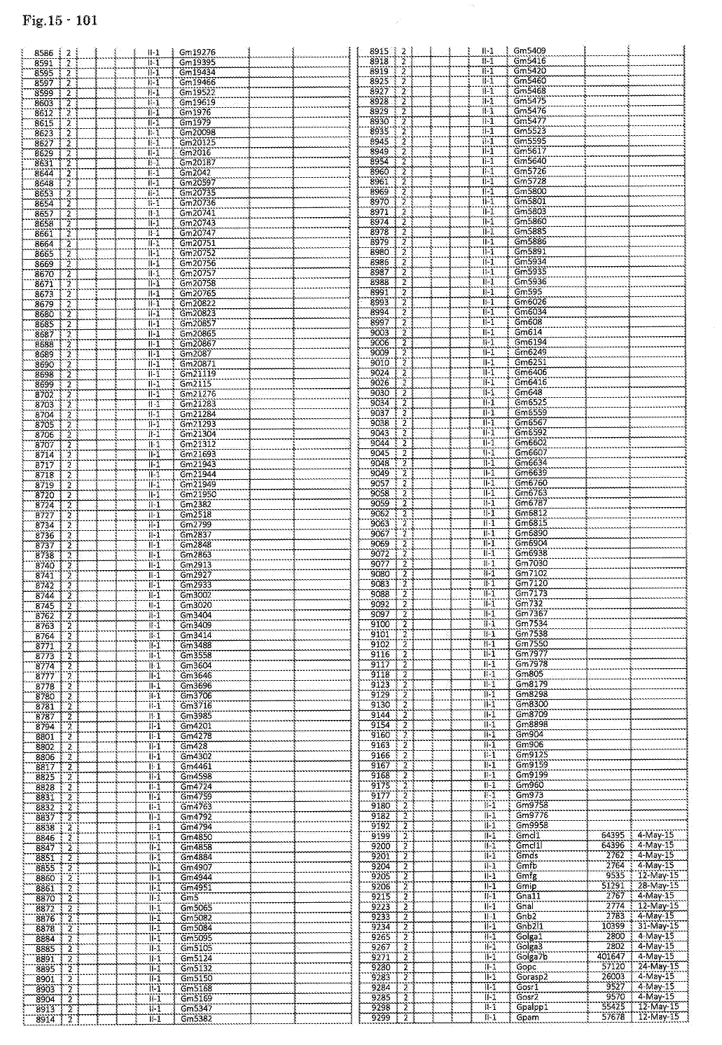

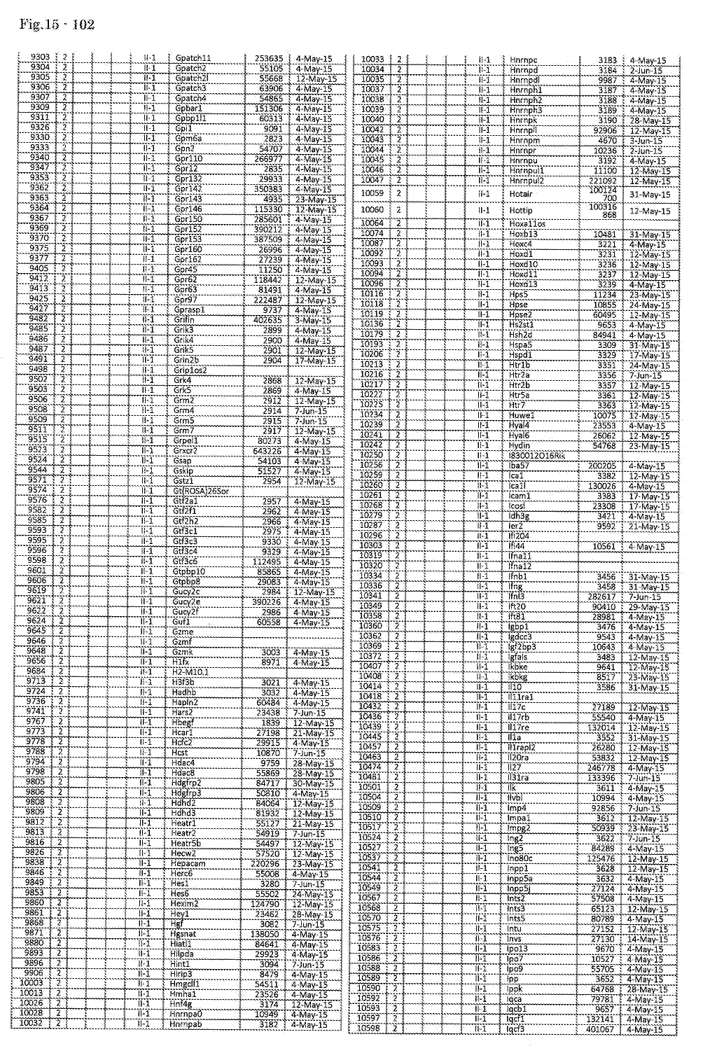

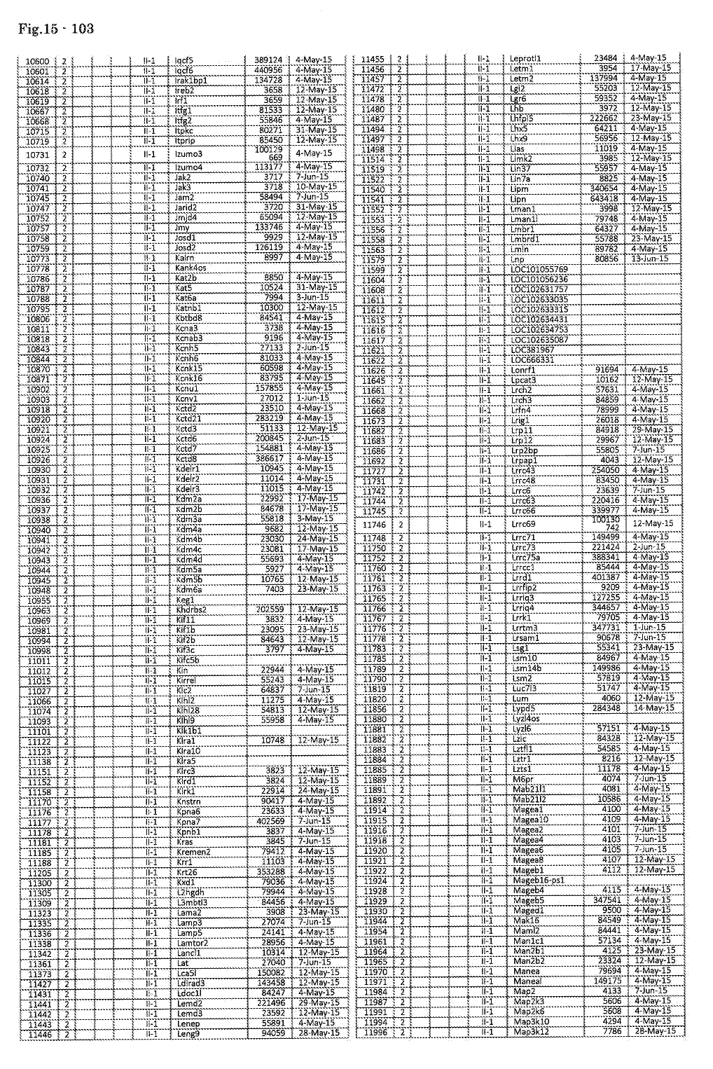

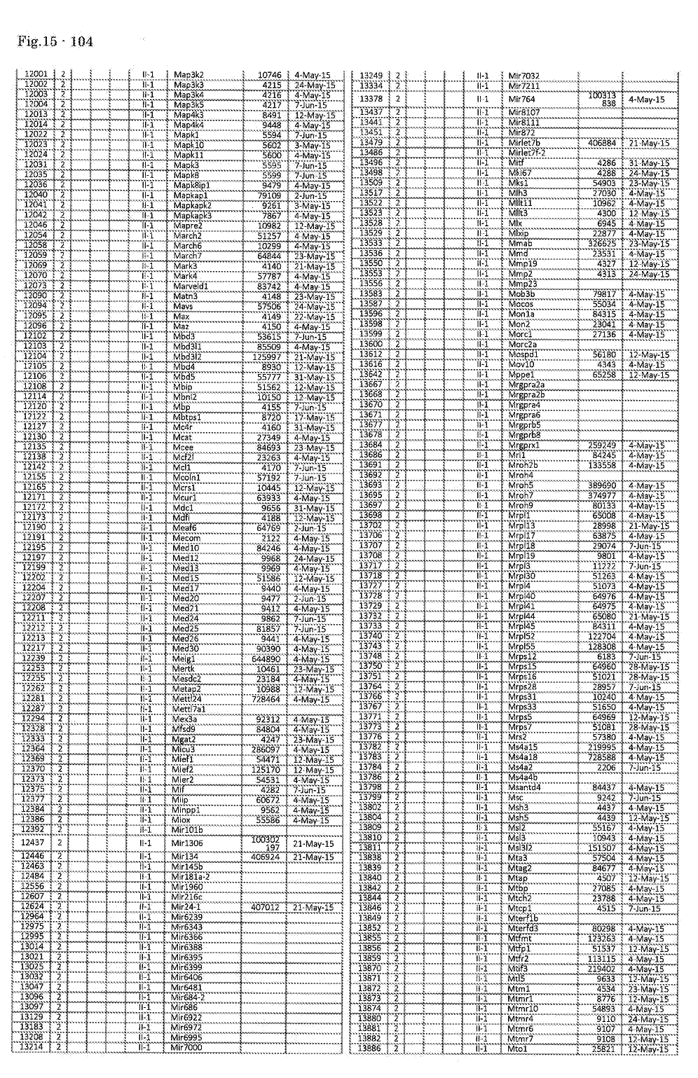

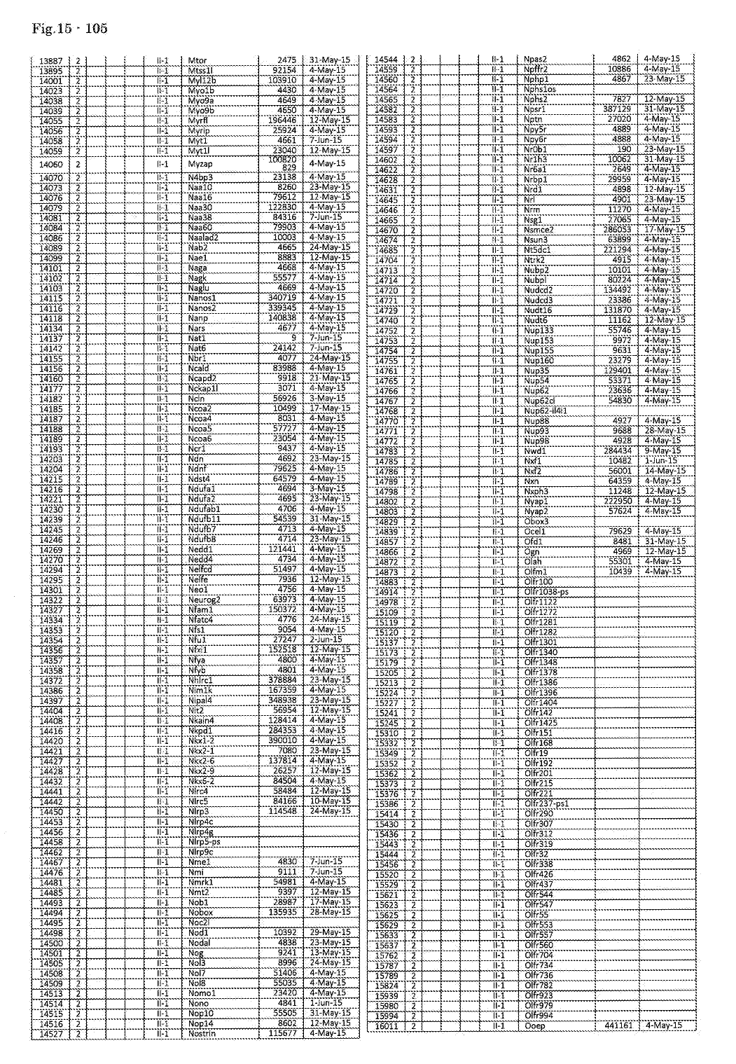

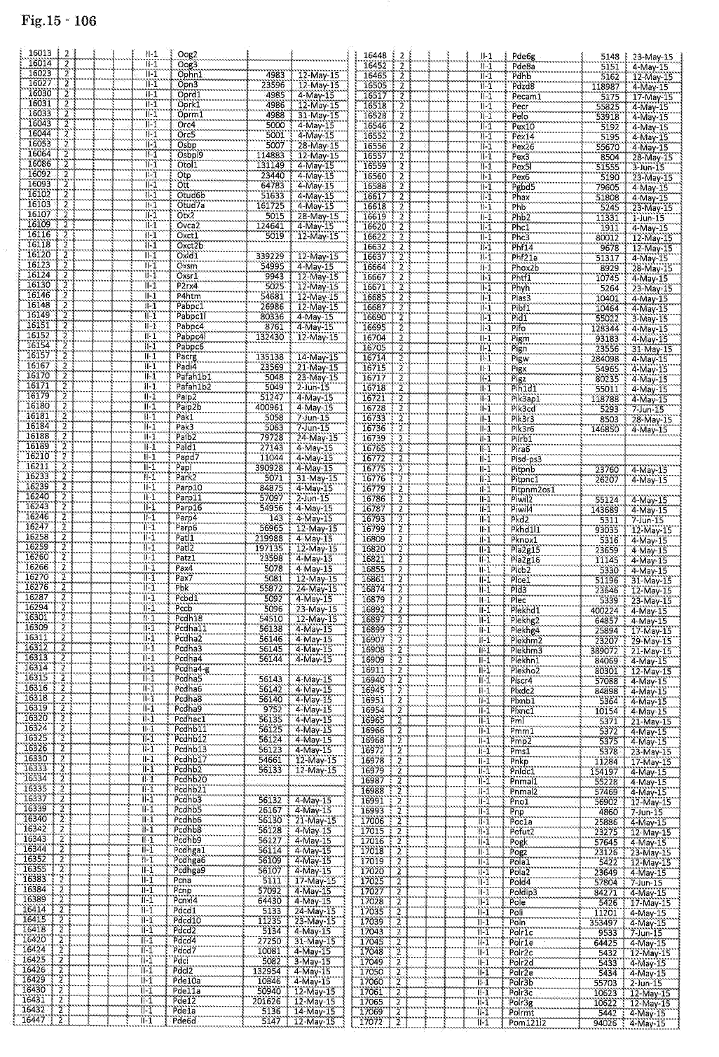

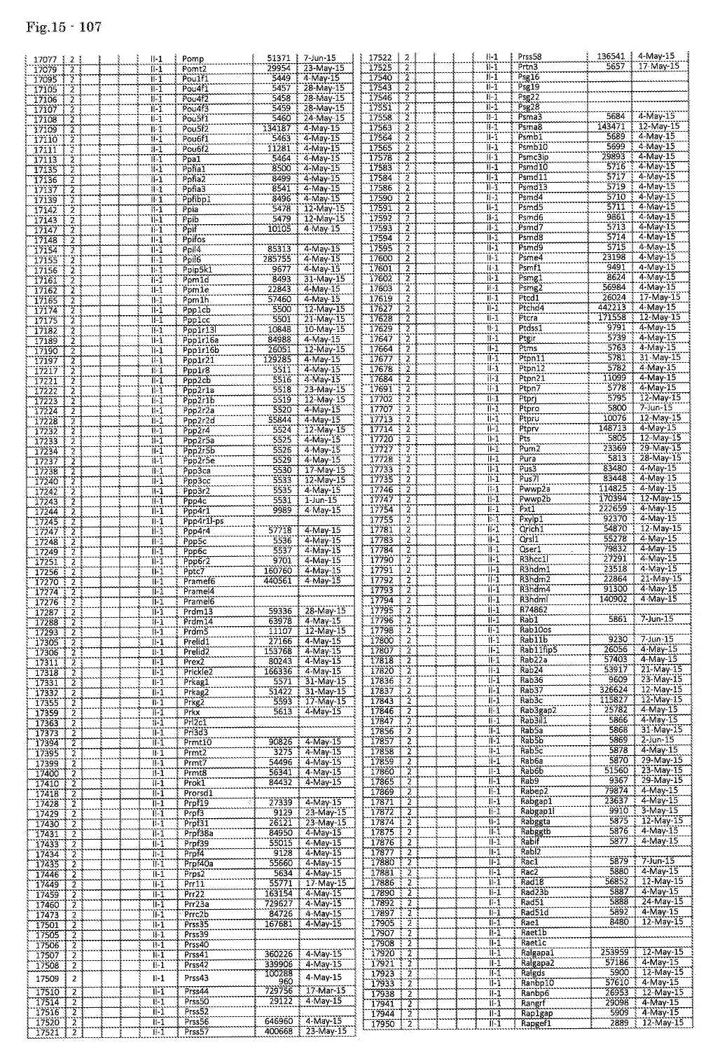

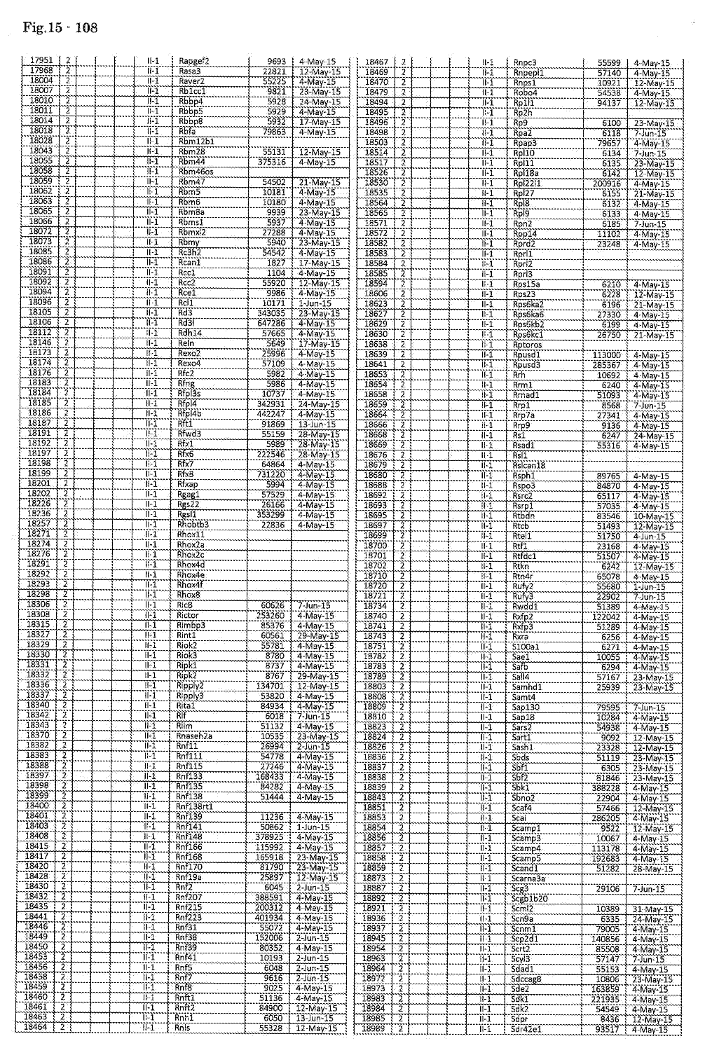

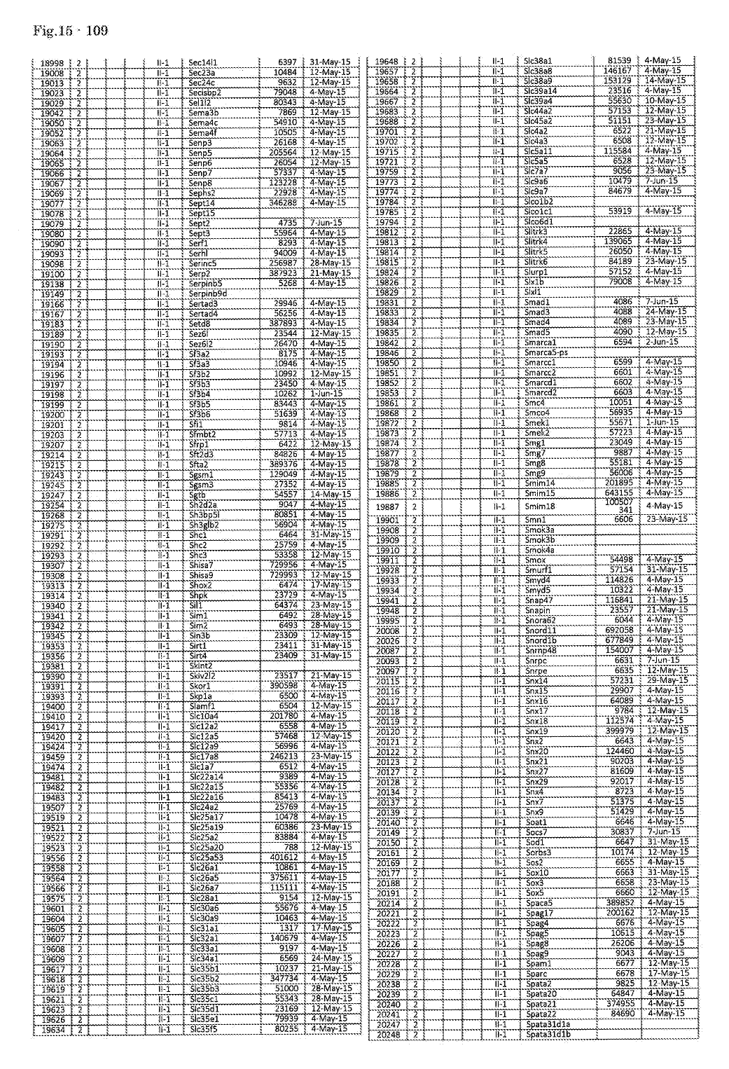

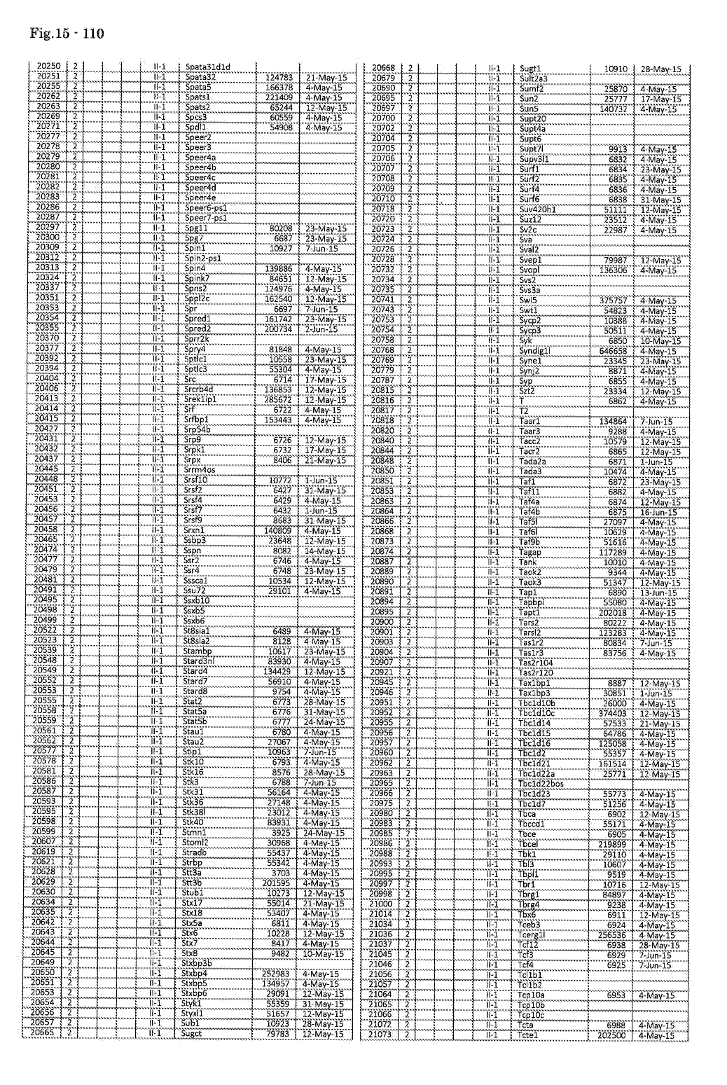

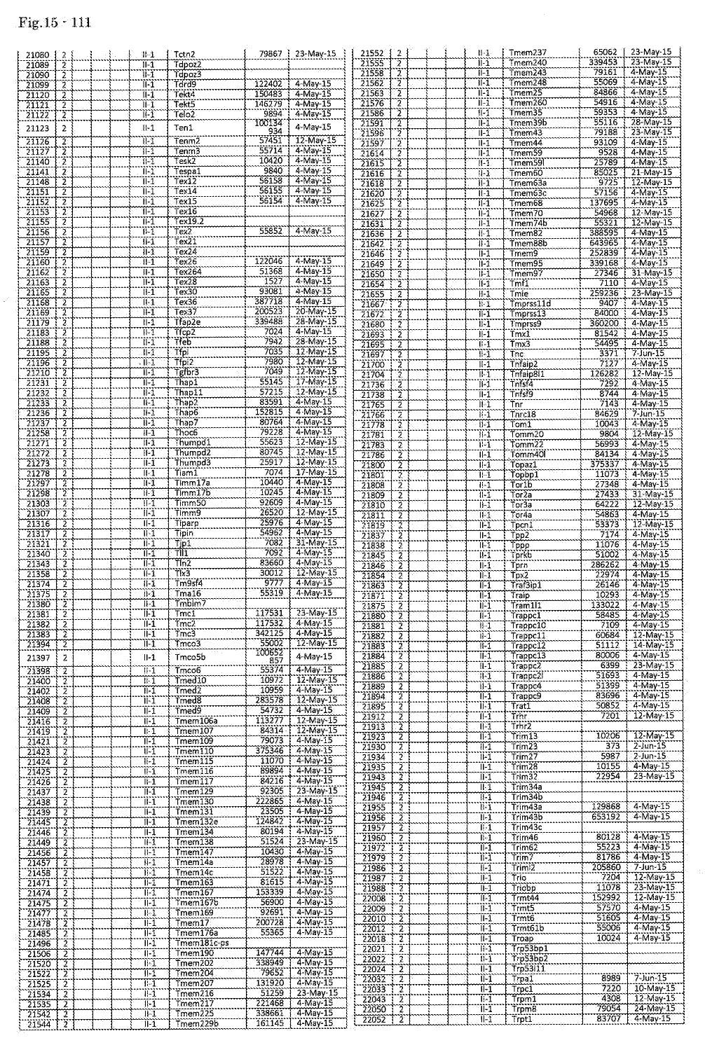

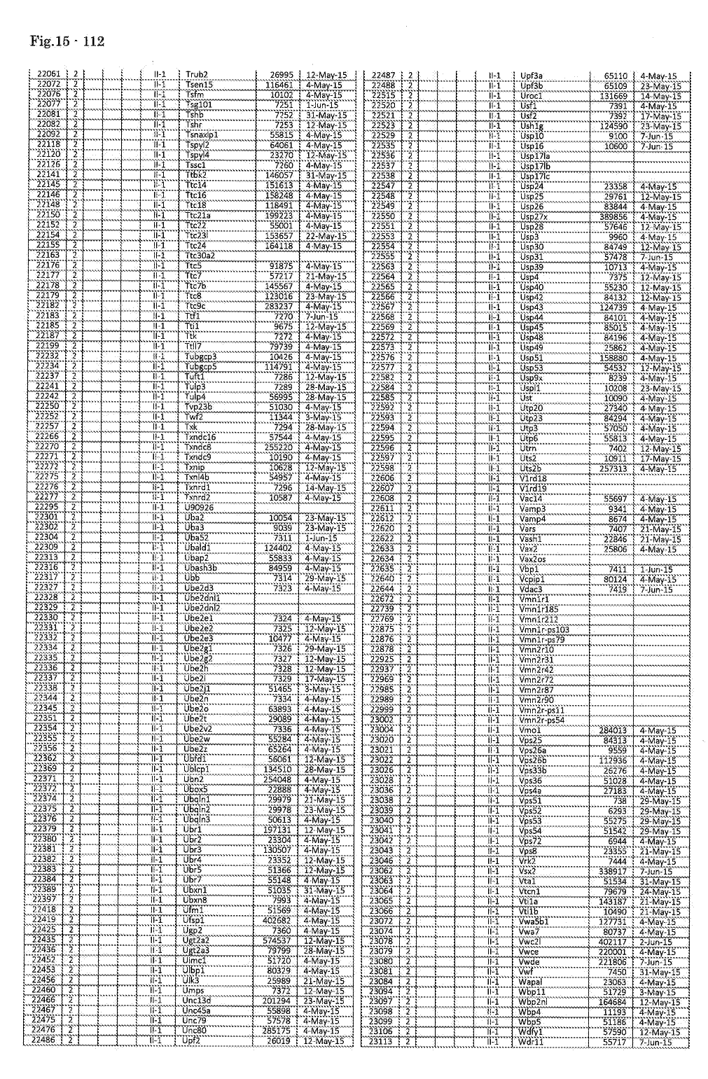

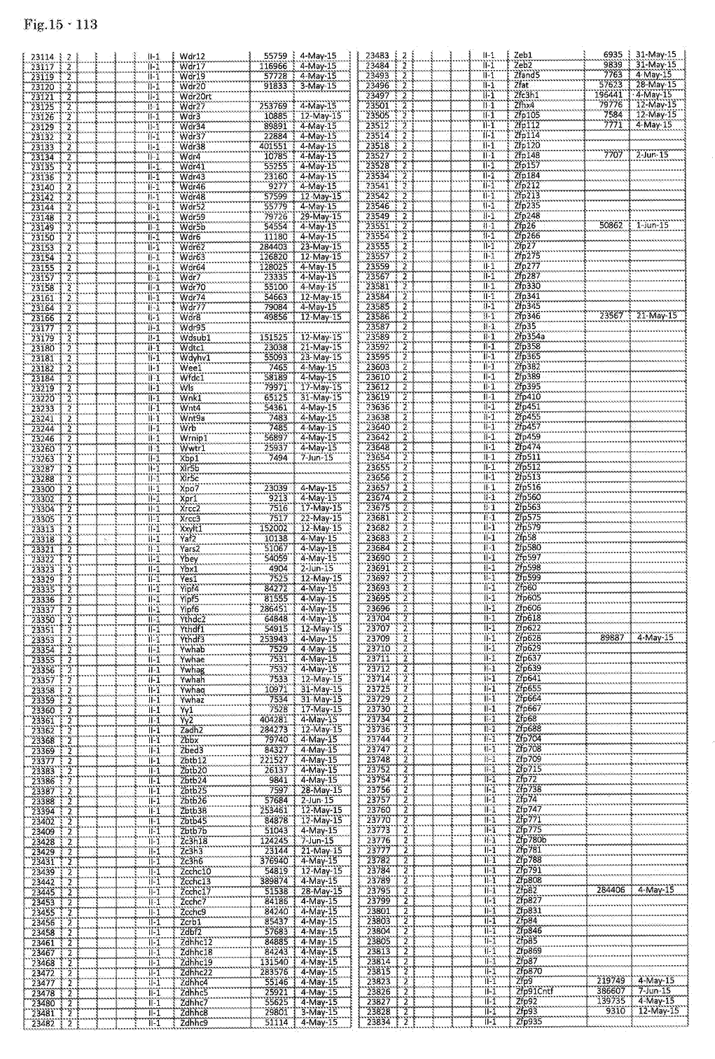

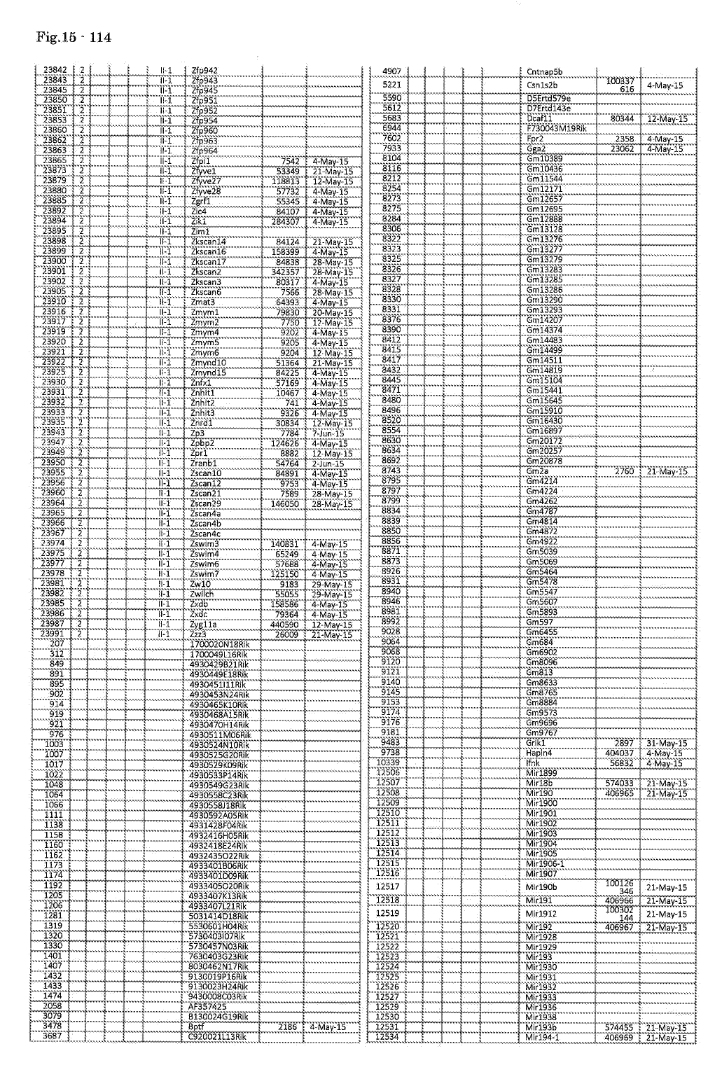

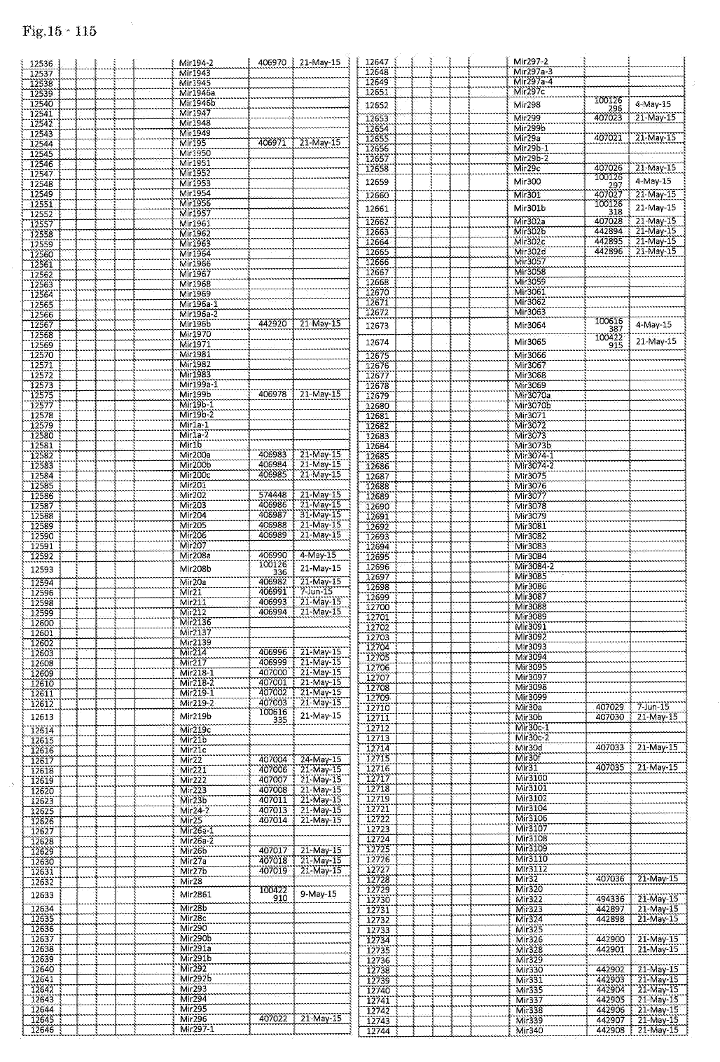

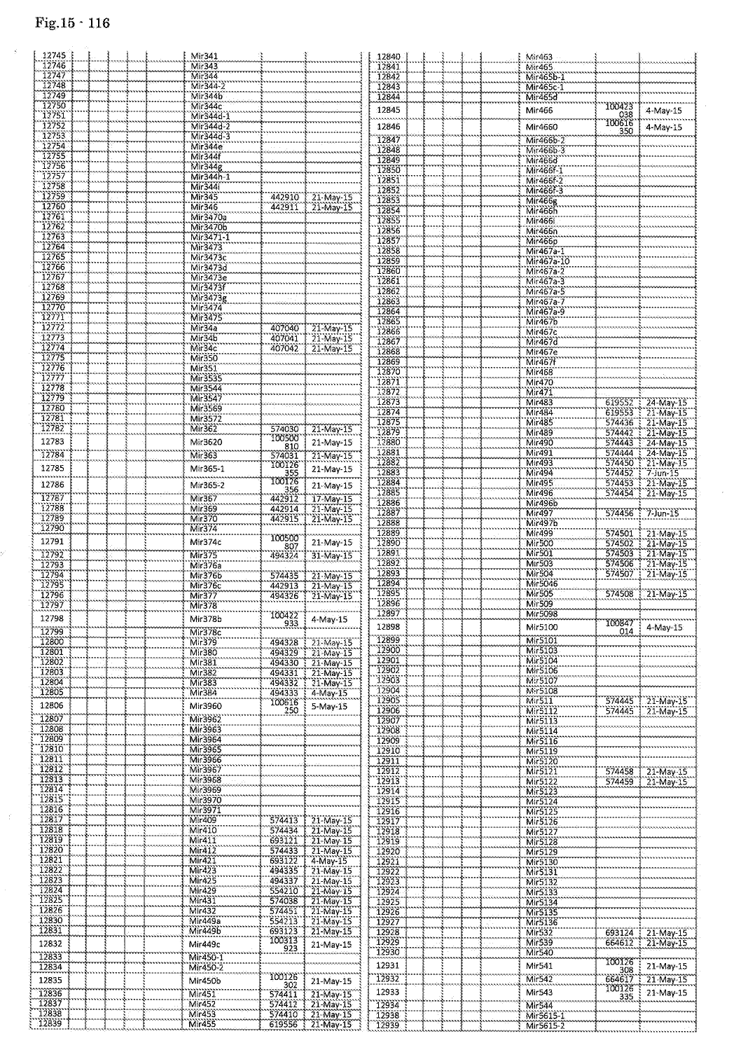

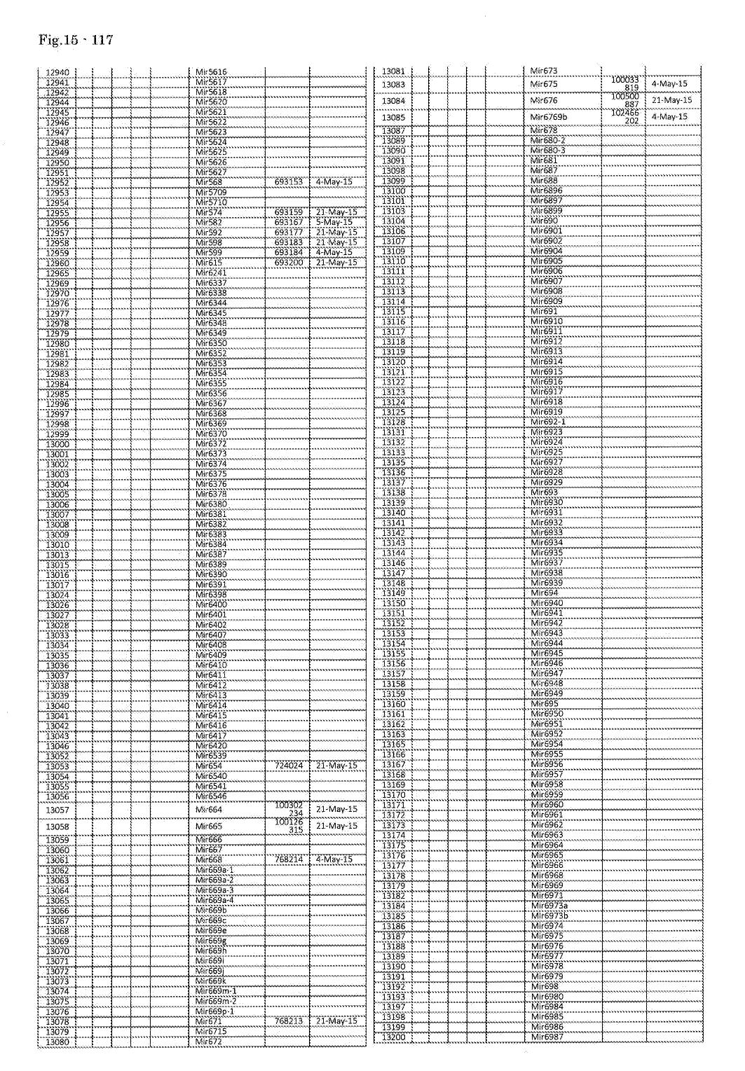

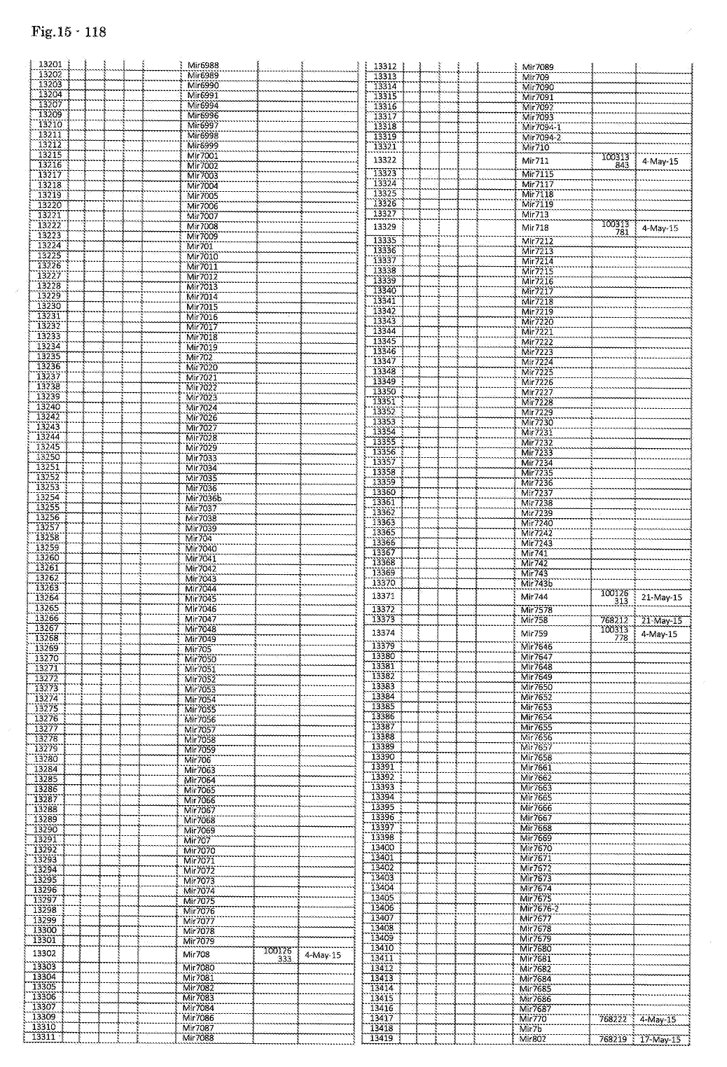

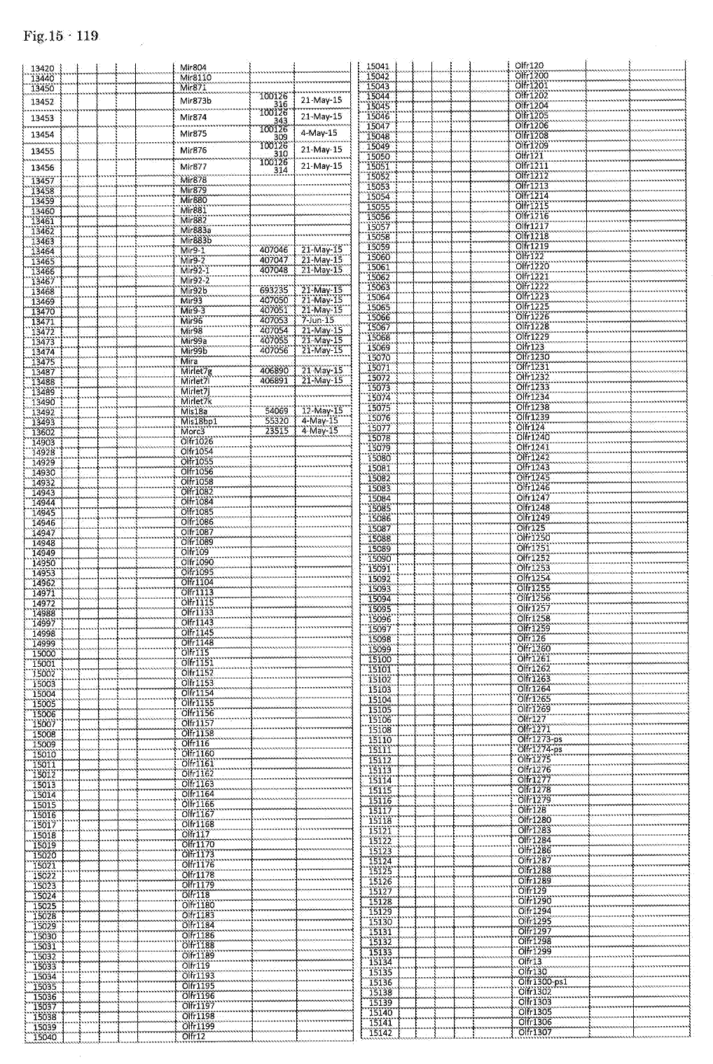

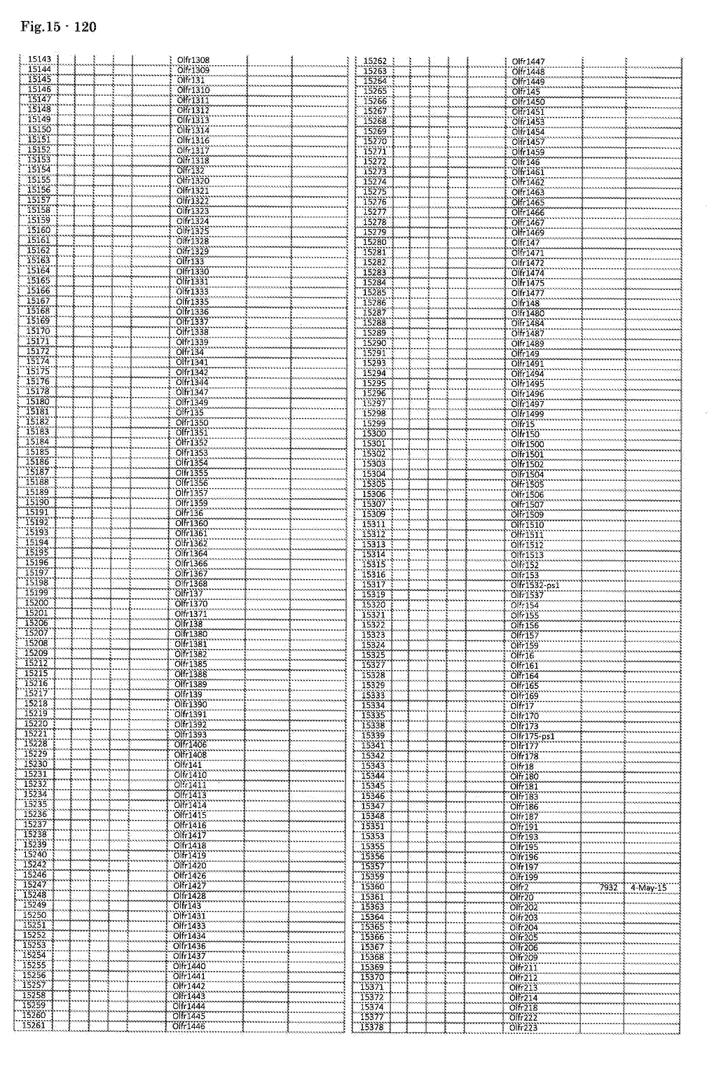

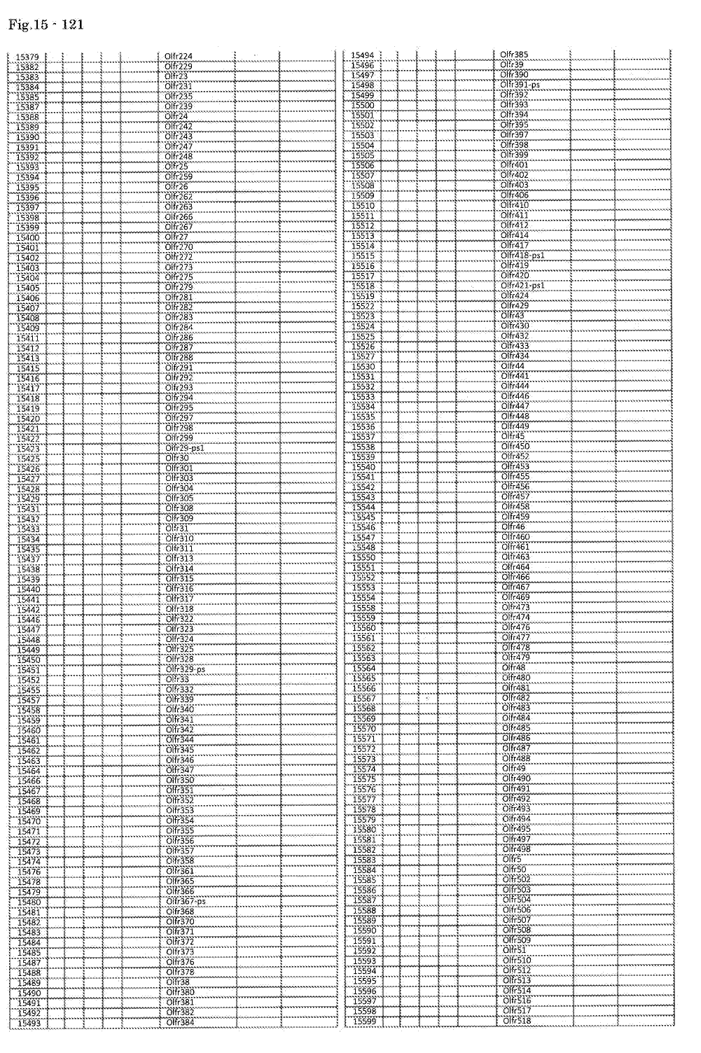

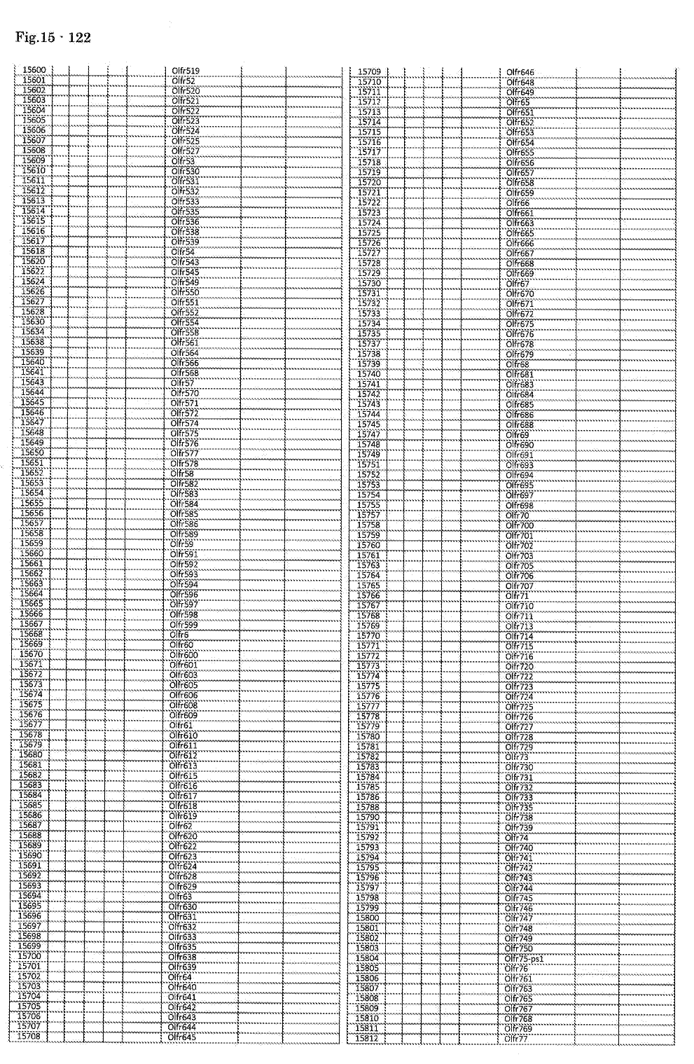

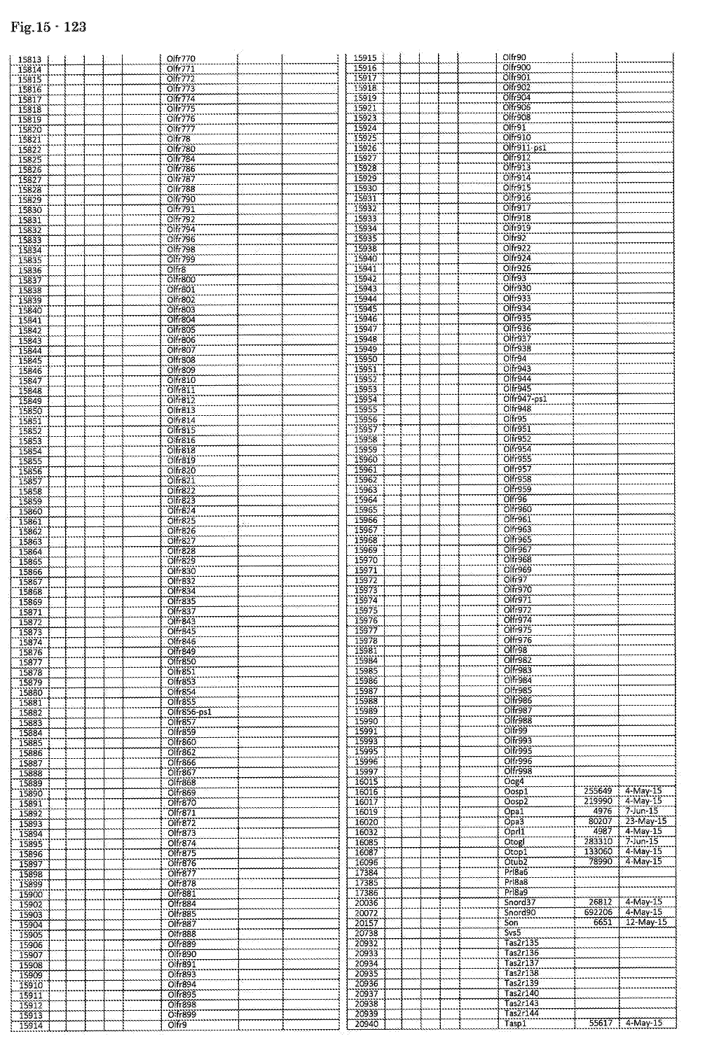

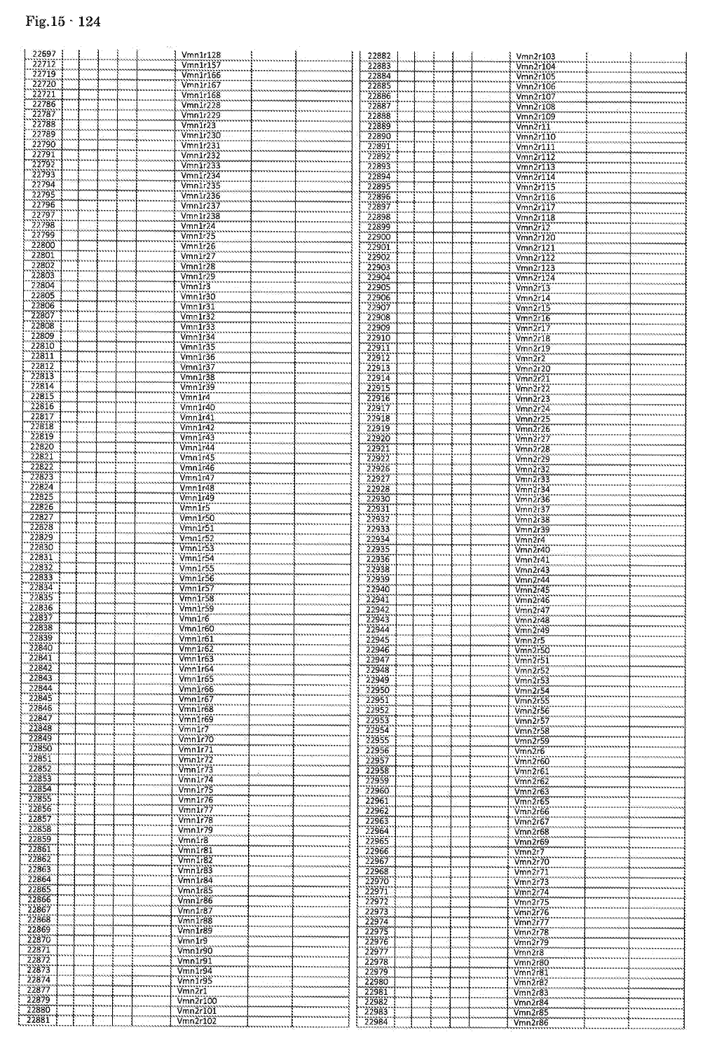

“Kidney function prediction marker” as used herein is, for example, a protein or mRNA that is expressed in the body of a subject. Specifically, “kidney function prediction marker” includes at least one member selected from the group consisting of kidney function prediction markers expressed from the genes shown in FIG. 14 (“group 1”). More specifically, the kidney function prediction marker includes at least one member selected from the group consisting of kidney function prediction markers of group 2 expressed from genes shown in FIG. 15, preferably at least one member selected from the group consisting of the kidney function prediction markers of group 3 expressed from genes shown in FIG. 15, more preferably at least one member selected from the group consisting of the kidney function prediction markers of group 4 expressed from genes shown in FIG. 15, and even more preferably at least one member selected from the group consisting of the kidney function prediction markers of group 5 expressed from genes shown in FIG. 15. Most preferably, the kidney function prediction marker is at least one member selected from the group consisting of the kidney function prediction markers of group 6 expressed from genes shown in FIG. 16, in particular, at least one member selected from the group consisting of proline-rich proteins, defensins (Defa and Defb), Aplnr, Spp1, Dnase1, Slco1a1, Anpep, Slc7a8, Oscar, and Hamp2. Moreover, these groups may also include splicing variants of the individual kidney function prediction markers.

“Proline-rich proteins (PRPs)” as used herein include acidic PRPs (aPRPs) including PRH1 and PRH2; basic PRPs (bPRPs) including PRB1, PRB2, and PRB4; glycosylated PRPs (GPRPs) including PRB3; PRPMP5; PRP2; splicing variants thereof; post-translationally modified variants thereof; and the like.

PRPs expressed from a group of genes that cluster around 132055403 to 132601236 of chromosome 6 (mm10 database: GRnC38/mm10: December, 2011) in the case of mice, PRPs expressed from a group of genes that cluster around 10824960 to 11395565 of chromosome 12 (hg38 database: GRCh38/hg38: December, 2013) in the case of humans, splicing variants thereof, post-translationally modified variants thereof, and the like are preferable. At least one member selected from the group consisting of PRH1, PRP2, PRB1, and PRPMP5, splicing variants thereof, and post-translationally modified variants thereof is more preferable.

The PRH1 protein is preferably NCBI Reference sequence ID: NP_035304.4 in the case of mice, and a protein expressed from the gene shown in NCBI Gene ID: 5554 (updated on Nov. 22, 2015) in the case of humans. The PRH1 protein may also include splicing variants thereof, post-translationally modified variants thereof, and the like.

The PRP2 protein is preferably NCBI Reference sequence ID: NP_113687.2 in the case of mice, and may also include splicing variants thereof, post-translationally modified variants thereof, and the like.

The PRB1 protein is preferably NCBI Reference sequence ID: NP_941071.1 in the case of mice, and a protein expressed from the gene shown in NCBI Gene ID: 5542 (updated on Jan. 3, 2016) in the case of humans. The PRB1 protein may also include splicing variants thereof, post-translationally modified variants thereof, and the like.

The PRPMP5 protein is preferably NCBI Reference sequence ID: NP_001019876.2 in the case of mice, and may also include splicing variants thereof, post-translationally modified variants thereof, and the like.

The PRH1 mRNA is preferably NCBI Reference sequence ID: NM_011174.4 in the case of mice, and mRNA expressed from the gene shown in NCBI Gene ID: 5554 (updated on Nov. 22, 2015) in the case of humans. The PRH1 mRNA may also include splicing variants thereof and the like.

The PRP2 mRNA is preferably NCBI Reference sequence ID: NM_031499.2 in the case of mice, and may also include splicing variants thereof and the like.

The PRB1 mRNA is preferably NCBI Reference sequence ID: NM_198669.1 in the case of mice, and mRNA expressed from the gene shown in NCBI Gene ID: 5542 (updated on Jan. 3, 2016) in the case of humans. The PRB1 mRNA may also include splicing variants thereof and the like.

The PRPMP5 mRNA is preferably NCBI Reference sequence ID: NM_001024705.2 in the case of mice, and may also include splicing variants thereof and the like.

“Measurement value relating to at least one protein selected from the group consisting of kidney function prediction markers” refers to a value reflecting the amount or concentration of at least one protein selected from the group consisting of kidney function prediction markers. The measurement value relating to at least one protein selected from the group consisting of kidney function prediction markers includes not only the amount or concentration of the protein itself, but also a value reflecting the amount or concentration of a peptide or amino acid obtained by decomposition of the protein. When the measurement value is indicated by “amount,” it may be expressed on either a mole basis or a mass basis; however, it is preferable to indicate the amount on a mass basis. When the value is expressed in terms of “concentration,” it may be a molar concentration or a ratio of a mass per constant volume of a specimen (mass/volume), preferably a mass/volume ratio. The value reflecting the amount or concentration may be the above or the intensity of a signal such as fluorescence or luminescence.

“Measurement value of at least one mRNA selected from the group consisting of kidney function prediction markers” may be represented by the number of copies (absolute amount) of the kidney function prediction marker mRNA present in a certain amount of a specimen; or may be a value reflecting the relative expression level to a housekeeping gene, such as β2-microglobulin mRNA, GAPDH mRNA, Maea mRNA, or β-actin mRNA. The measurement value may also be represented by the intensity of a signal such as fluorescence or luminescence.

“Predetermined threshold” of a measurement value relating to a kidney function prediction marker protein refers to a threshold determined based on a measurement value relating to the kidney function prediction marker protein in a specimen of an individual who has decreased kidney function and/or a measurement value relating to the kidney function prediction marker protein in a specimen of a healthy individual.

For example, measurement values relating to a kidney function prediction marker protein measured using specimens of multiple individuals that have decreased kidney function, and measurement values relating to the kidney function prediction marker protein measured using specimens of multiple healthy individuals are obtained. Based on these multiple values, a value that allows the most accurate determination of the presence or absence of decreased kidney function can be determined and used as a “threshold.” The “value that allows the most accurate determination” can be appropriately set based on indices, such as sensitivity, specificity, positive predictive value, and negative predictive value, depending on the purpose of the examination.

For example, in one embodiment, the highest measurement value among measurement values relating to a kidney function prediction marker protein in specimens obtained from multiple healthy individuals may be used as a threshold.

In another embodiment, when the threshold is determined based on a measurement value relating to a kidney function prediction marker protein in a specimen of an individual who has decreased kidney function, the lowest measurement value among measurement values relating to the kidney function prediction marker protein in specimens of multiple individuals who have decreased kidney function can be determined as a threshold.

In another embodiment, the threshold may be a measurement value per se relating to a kidney function prediction marker protein in a specimen of a healthy individual; or a mean, median, or mode of multiple measurement values relating to a kidney function prediction marker protein in healthy individuals.

Further, a measurement value relating to a kidney function prediction marker obtained in the past from the same subject when the subject was healthy (which may be one value; or a mean, median, or mode of multiple values) may be used as a threshold.

“Threshold” of a measurement value of a kidney function prediction marker mRNA can be determined as in the above-described “threshold” of a measurement value relating to a kidney function prediction marker protein, using a measurement value of a kidney function prediction marker mRNA instead of a measurement value relating to a kidney function prediction marker protein.

“Healthy individual” is not particularly limited. Preferably, the healthy individual is a human or non-human mammal that is described in the explanation of the term “individual” and that does not show abnormal data in biochemical tests, blood tests, urine tests, serum tests, physiological tests, etc. The age and sex of the healthy individual are not particularly limited.

“Multiple specimens” refers to two or more, preferably five or more, and more preferably ten or more specimens. These may be specimens collected from different individuals, or may be multiple specimens of the same individual collected at different times.

“Multiple values” refers to two or more, preferably five or more, and more preferably ten or more measurement values relating to a kidney function prediction marker protein or measurement values of a kidney function prediction marker mRNA.

“Multiple individuals” refers to two or more, preferably five or more, and more preferably ten or more individuals.

The species, age, sex, etc., of a subject are not necessarily the same as those of an individual from whom a measurement value relating to a kidney function prediction marker protein and a measurement value of a kidney function prediction marker mRNA are obtained for determining a threshold. It is preferred that the species of the subject is the same as that of the individual. It is also preferred that the individual is of the same age and/or sex as the subject.

“Antibody against kidney function prediction marker” is not limited, as long as the antibody specifically binds to at least one protein selected from the group consisting of the above-described kidney function prediction markers; and any of polyclonal antibodies, monoclonal antibodies, and fragments thereof (for example, Fab, F(ab)₂, etc.) obtained by immunizing a non-human animal with at least one protein selected from the group consisting of the kidney function prediction markers or a part thereof as an antigen can be used. Additionally, immunoglobulin classes and subclasses are not particularly limited. Moreover, the antibody against kidney function prediction marker may be a chimeric antibody. Further, the antibody against kidney function prediction marker may be scFv or the like.

Examples of a kidney function prediction marker protein used as an antigen for preparing an antibody against kidney function prediction marker include the entirety or a part of at least one protein selected from the group consisting of the above-described kidney function prediction markers.

“Nucleic acid for kidney function prediction marker mRNA detection” as used herein is not limited, as long as it contains a sequence that specifically hybridizes to at least one mRNA selected from the group consisting of the above-described kidney function prediction markers, or to a reverse transcription product of the mRNA. The nucleic acid for detection may be DNA or RNA, and the nucleotides contained in the nucleic acid for detection may be naturally occurring nucleotides or artificially synthesized nucleotides.

The length of the nucleic acid for detection is not particularly limited. When the nucleic acid for detection is used as a capture probe in, for example, a microarray, the length of sequence that hybridizes to a target nucleic acid is preferably about 100 mer, more preferably about 60 mer, and even more preferably about 20 to 30 mer. The capture probe can be produced with, for example, a known oligonucleotide synthesizer. The capture probe may contain a sequence that does not hybridize to the target nucleic acid.

When the nucleic acid for detection is a primer used for PCR reactions, the length of sequence that hybridizes to a target nucleic acid is preferably about 50 mer, more preferably about 30 mer, and even more preferably about 15 to 25 mer. The primer can be produced with, for example, a known oligonucleotide synthesizer. The primer may contain a sequence that does not hybridize to the target nucleic acid. The primer may be labeled with a fluorescent dye or the like.

A probe for quantification that is decomposed during a PCR reaction may also be used for real-time quantification of a PCR product in RT-PCR, in addition to primers. The probe for quantification is not limited as long as it hybridizes to a target nucleic acid. The probe for quantification is preferably a nucleic acid with a length of about 5 to 20 mer that contains a sequence that hybridizes to a target nucleic acid. Further, it is preferred that the probe for quantification is labeled at one end with a fluorescent dye, and at the other end with a quencher of the fluorescent dye.

### 2. Method for Obtaining Each Measurement Value

The methods for obtaining a measurement value relating to at least one protein selected from the group consisting of kidney function prediction markers and a measurement value of at least one mRNA selected from the group consisting of kidney function prediction markers in the present invention are not limited, as long as the measurement values can be obtained. For example, they can be obtained according to the methods described below.

### 2-1. Obtaining Measurement Value Relating to Kidney Function Prediction Marker Protein

### 2-1-1. Obtaining Measurement Value of Kidney Function Prediction Marker Protein

When a measurement value relating to at least one protein selected from the group consisting of kidney function prediction markers (hereinafter may be abbreviated as “measurement value of a kidney function prediction marker protein” in the present specification) is obtained, an antibody against kidney function prediction marker described in the section “1. Explanation of terms” can be used in the process for obtaining the measurement value. Alternatively, the “9-1. Test reagent comprising antibody against kidney function prediction marker” described later may be used.

The measurement value of a kidney function prediction marker protein can be obtained by using a method such as a known ELISA method.

In this embodiment, an antibody against kidney function prediction marker for antigen capture can be immobilized on a solid phase such as a microplate in advance, and a complex between the immobilized antibody against kidney function prediction marker and a kidney function prediction marker protein in a specimen can be formed. The amount or concentration of the kidney function prediction marker protein contained in the specimen can be measured by detecting the complex immobilized on the solid phase or the complex formed on the solid phase by a method known in the art.

The method for immobilizing an antibody against kidney function prediction marker for antigen capture on a solid phase is not particularly limited. An antibody against kidney function prediction marker may be directly immobilized or indirectly immobilized with another substance interposed therebetween by using a known method. Examples of direct binding include physical adsorption and the like. Preferably, for example, an immunoplate may be used to directly physically bind an antibody against kidney function prediction marker to the microplate.

The material of the solid phase is not particularly limited. Examples include polystyrene, polypropylene, and the like. The shape of the solid phase is not particularly limited. Examples include microplates, microtubes, test tubes, and the like.

This method may comprise, following the formation of the complex, an operation of washing the solid phase. In washing, for example, PBS containing a surfactant or the like may be used.

In this method, the complex can be detected by using an antibody against kidney function prediction marker for detection labeled with a labeling substance, or using an unlabeled antibody against kidney function prediction marker, an anti-immunoglobulin antibody labeled with a labeling substance and capable of binding to the unlabeled antibody against kidney function prediction marker, etc. It is preferable to use a labeled antibody against kidney function prediction marker for detection. It is also preferable that the epitope in the kidney function prediction marker protein of the antibody against kidney function prediction marker for detection is different from the epitope in the kidney function prediction marker protein of the antibody against kidney function prediction marker for antigen capture.

The labeling substance used for the antibody against kidney function prediction marker for detection or the labeled anti-immunoglobulin antibody is not particularly limited as long as the labeling substance generates a detectable signal. Examples include fluorescent substances, radioactive isotopes, enzymes, and the like. Examples of enzymes include alkaline phosphatase, peroxidase, and the like. Examples of fluorescent substances include fluorescent dyes such as fluorescein isothiocyanate (FITC), rhodamine, and Alexa Fluor (registered trademark), fluorescent proteins such as GFP, and the like. Examples of radioactive isotopes include ¹²⁵I, ¹⁴C, ³²P, and the like. Among them, alkaline phosphatase or peroxidase is preferable as the labeling substance.

The antibody against kidney function prediction marker for detection is obtained by labeling an antibody against kidney function prediction marker with the above-mentioned labeling substance by a labeling method known in the art. Alternatively, such labeling may be performed using a commercially available labeling kit or the like. For the labeled immunoglobulin antibody, the same method as the labeling of the antibody against kidney function prediction marker may be used, or a commercially available product may be used.

In this method, the measurement value of the kidney function prediction marker contained in the specimen can be obtained by detecting a signal generated by the labeling substance of the labeled antibody against kidney function prediction marker contained in the complex. Here, “detecting a signal” includes qualitatively detecting the presence or absence of a signal, quantifying the signal intensity, and semi-quantitatively detecting the signal intensity. Such semi-quantitative detection means to indicate the signal intensity in stages such as “no signal generation,” “weak,” “medium,” and “strong.” In this step, it is preferable to detect the signal intensity quantitatively or semi-quantitatively.

As the method for detecting a signal, a known method may be used. In this method, a measurement method according to the type of signal derived from the above-mentioned labeling substance may be appropriately selected. For example, when the labeling substance is an enzyme, detection of a signal may be performed by measuring a signal such as light or color generated by the reaction of the enzyme with a substrate using a known device such as a luminometer or a spectrophotometer.

The substrate of an enzyme can be appropriately selected from known substrates depending on the type of enzyme. For example, when alkaline phosphatase is used as an enzyme, examples of substrates include chemiluminescent substrates such as CDP-Star (registered trademark) (disodium 4-chloro-3-(methoxyspiro[1,2-dioxetane-3,2′-(5′-chloro)tricyclo[3.3.1.13,7]decan]-4-yl)phenyl phosphate), and chromogenic substrates such as 5-bromo-4-chloro-3-indolyl phosphate (BCIP), disodium 5-bromo-6-chloro-3-indolyl phosphate, and p-nitrophenyl phosphate. When the labeling substance is peroxidase, examples of substrates include tetramethylbenzidine (TMB) and the like.

When the labeling substance is a radioactive isotope, a signal, i.e., radiation, can be measured using a known device such as a scintillation counter. When the labeling substance is a fluorescent substance, a signal, i.e., fluorescence, can be measured using a known device such as a fluorescence microplate reader. The excitation wavelength and the fluorescence wavelength can be appropriately determined according to the type of fluorescent substance used.

The detection results of the signal can be used as the measurement value of the kidney function prediction marker protein. For example, when the signal intensity is quantitatively detected, the measurement value itself of the signal intensity or a value calculated from the measurement value of the signal intensity can be used as the measurement value of the kidney function prediction marker protein.

2-1-1. Obtaining Measurement Value of Peptide or Amino Acid Derived from Kidney Function Prediction Marker Protein

In the present invention, the measurement value of a peptide or an amino acid derived from a kidney function prediction marker protein can also be obtained instead of obtaining the measurement value of the kidney function prediction marker protein itself.

For example, the specimen may be decomposed (pretreated) with an acid, an alkali, a proteolytic enzyme, or the like, and a peptide or an amino acid contained in the decomposition product may be measured. The obtained measurement value may be used as a measurement value relating to at least one protein selected from the group consisting of kidney function prediction markers.

Preferably, since PRPs are proline-rich proteins, the specimen may be decomposed with an acid, an alkali, a proteolytic enzyme, or the like; and proline contained in the decomposition product may be measured. The obtained measurement value may be used as a measurement value relating to at least one protein selected from the group consisting of PRPs.

When the specimen is pretreated with an acid, the specimen may be treated, for example, with hydrochloric acid, preferably 6N hydrochloric acid, at about 100 to 150° C. for about 12 to 24 hours (see J. J. Wren and P. H. Wiggall; Biochem. J. (1965) 94, 216-220).

When the specimen is pretreated with an alkali, the specimen may be treated, for example, with sodium hydroxide, preferably 4N sodium hydroxide, at about 80 to 100° C. for about 4 to 10 hours.

When the specimen is decomposed with a proteolytic enzyme, the proteolytic enzyme is not particularly limited as long as it is an enzyme capable of decomposing a protein into amino acids. Examples include pronase, proteinase K, and the like. Pronase is preferable. The specimen may be decomposed with a proteolytic enzyme under the following conditions: a treatment at about 4 to 80° C., for about 1 to 90 hours.

The specimen may be pretreated unmodified, or may be diluted with, for example, physiological saline, PBS, or buffer for enzymatic reaction. When the specimen is a body fluid, solids such as cells may be removed by centrifugation or filtration before the pretreatment.

The method for obtaining the measurement value of a peptide or an amino acid is not limited as long as it is a method that can measure the target peptide or amino acid. Examples of the method for measuring the measurement value of proline include a measurement method using a mass spectrometer, such as GCMS, LCMS, or CEMS, and a measurement method using a color reaction (including a color reaction using a fluorescent dye), such as a method using a ninhydrin reaction (Bates L, et al. 1973. Rapid determination of free proline for water-stress studies. Plant and Soil, 39, 205-207), isatin paper assay (Abraham et al., Chapter 20: Methods for Determination of Proline in Plants, Method in Molecular Biology, 2010, 639, 317-331), the method described in Ying Zhou and Juyoung Yoon (Chem. Soc. Rev., 2012, 41, 52-67), or a method using a genipin reaction (Fujikawa et al. Brilliant skyblue pigment formation from gardenia fruits. J. Ferment. Technol., 1987, 65, 419).

When proline is measured by mass spectrometry, the amount or concentration of proline can be, for example, calculated from the peak areas of an internal standard of known concentration and the specimen in a mass spectrum. The calculated value can be used as the measurement value of proline.

When proline is measured by using a color reaction, such as a ninhydrin reaction, the intensity of color (absorbance) of the reaction liquid exhibited by proline in the specimen may be measured with an absorption spectrometer or the like, and the absorbance value may be used as the measurement value of proline. Moreover, the amount or concentration of proline may be calculated from the absorbance value by using a calibration curve or the like, and the calculated value may be used as the measurement value of proline.

More specifically, when proline is measured by GCMS, it can be measured by, for example, the following method.

### (1) Pretreatment of Saliva

Saliva is centrifuged, for example, at 800 to 1,200×g for about 10 to 20 minutes at 2 to 10° C. to remove solids, and the resulting saliva is used as a saliva sample in measurement. 10 to 20 μL of the saliva sample is diluted about 10- to 30-fold with, for example, a buffer containing 0.1 M Tris-HCl (pH 7.5) and 0.5% SDS. Pronase is added to 50 to 200 μL of the diluted saliva sample at a final concentration of 1 to 10 μg/mL, followed by incubation at about room temperature to 60° C. for about 1 to 90 hours in the shade. The reaction liquid after the incubation is used as a pronase reaction liquid.

1.5 μL of 2-isopropylmalic acid (internal standard) is added per mL of chromatography grade methanol, and a requisite amount of the resulting solution is prepared. 500 μL of the methanol solution containing 2-isopropylmalic acid is added to the pronase reaction liquid, and the mixture is stirred by vortexing for about 30 seconds for spin-down. After the mixture is allowed to stand at room temperature for about 5 minutes, 200 μL of ultrapure water is added, and the mixture is stirred by vortexing for about 30 seconds and centrifuged at about 4600×g for 5 minutes at 4° C. About 400 μL of the first supernatant is transferred from the mixture after the centrifugation to a fresh tube. About 200 μL of ultrapure water is added to the first supernatant, the mixture is stirred by vortexing for about 30 seconds and centrifuged at about 4600×g for about 5 minutes at 4° C., and about 400 μL of the second supernatant is collected. The second supernatant is transferred to an ultrafiltration unit cup (Hydrophilic PTFE membrane, 0.2 μm; Millipore) and centrifuged at about 9100×g for about 15 minutes at 4° C., thereby obtaining an ultrafiltrate. The ultrafiltrate is dried under reduced pressure at about 65° C. for about 90 minutes, 50 μL of a pyridine solution containing 20 mg/mL methoxyamine hydrochloride is added to the residue, and the mixture is shaken with a shaker at 37° C. for 90 minutes. Thereafter, 50 μL of N-methyl-N-trimethylsilyltrifluoroacetamide (MSTFA) is further added, and the mixture is shaken with a shaker at 37° C. for 30 minutes and trimethylsilylated, thereby obtaining a GC sample.

### (2) GCMS Measurement

For example, GCMS-TQ8030 (Shimadzu Corporation) is used for GCMS; and, for example, DB-5 (30 m×0.25 mm (inner diameter)×1.00 μm (film thickness)) (Agilent Technologies) is used as a capillary column for GC. GC is performed under, for example, the following temperature increase conditions: the temperature is increased at a rate of 4° C./min from 100° C. to 320° C. The injector port temperature is, for example, 280° C. Helium is used as a carrier gas, and made to flow at a rate of about 39.0 cm/sec. The energy of the electron ionization is, for example, 150 eV, the ion source temperature is, for example, 200° C., and proline-2TMS {142.10/73.0} and 2-isopropylmalic acid {216.10/147.10} are measured in MRM mode. 1 μL of the GC sample is injected, the splitless mode is used, and measurement is performed at a detector voltage of, for example, 1.50 kV.

### (3) Analysis of GCMS Data

For analysis of GCMS data, GCMS solution Ver. 4.2 data analysis software and GCMS Metabolites Database (Shimadzu Corporation), for example, can be used. A dilution series of purified proline at, for example, the following six points: 0.02, 0.01, 0.005, 0.0005, 0.00005, and 0.000005 (nmol/μL) is prepared, and a calibration curve is thereby prepared. The concentration of proline can be determined by dividing the peak area of proline in the specimen by the peak area of the internal standard (2-isopropylmalic acid) to obtain a ratio, and applying the ratio to the calibration curve.

2-2. Obtaining Measurement Value of Kidney Function Prediction Marker mRNA

A known method, such as a microarray method, an RNA-Seq analysis method, or a quantitative RT-PCR method, can be used to obtain the measurement value of at least one mRNA selected from the group consisting of kidney function prediction markers (hereinafter may be abbreviated as “measurement value of a kidney function prediction marker mRNA” in the present specification). As probes used for the microarray method, probes of one's choosing, or known probes, may be synthesized and used; or a commercially available microarray chip may be used. Alternatively, the “9-2. Test reagent comprising nucleic acid for kidney function prediction marker mRNA detection” may be used.

In this method, any of total RNA and mRNA extracted from a specimen may be used. It is preferred that the specimen used for total RNA and mRNA extraction is subjected to RNA extraction immediately after being collected from an individual; or is frozen (preferably under an atmosphere at −196° C. or less (rapidly cooled in liquid nitrogen)) immediately after being collected from an individual, and stored at −80° C. or less until RNA extraction.

The method for extracting total RNA and mRNA from a specimen is not particularly limited, and a known extraction method may be used.

Quantification by the microarray method may be performed according to a known method. The expression level of a kidney function prediction marker mRNA may be expressed as the relative expression level to that of a housekeeping gene; or expressed as the measurement value of the signal intensity of, for example, a fluorescent dye.

Quantification by RT-PCR may be performed by conducting a reverse transcription reaction using total RNA or mRNA extracted from a specimen as a template, and performing analysis by a real-time PCR method or the like with the obtained cDNA as a template by using specific primers for a kidney function prediction marker mRNA. In this case, the expression level of the kidney function prediction marker mRNA may be expressed as the relative expression level to that of a housekeeping gene or expressed as the measurement value of the signal intensity of, for example, a fluorescent dye.

In the RNA-Seq analysis method, mRNA extracted from a specimen is fragmented, cDNA is synthesized by reverse transcription reaction using these fragments as a temperate, and libraries are prepared. The nucleotide sequence of each fragment contained in each library is determined by using a next-generation sequencer, the obtained information is mapped to a reference gene sequence, and the expression level of mRNA is represented as RPKM (Reads Per Kilobase per Million). RPKM may be represented as the intensity of a signal in, for example, a heat map.

The detection results of the signal can be used as the expression level of the kidney function prediction marker mRNA. For example, when the signal intensity is quantitatively detected, the measurement value itself of the signal intensity or a value calculated from the measurement value of the signal intensity can be used as the expression level of the kidney function prediction marker mRNA.

Examples of the value calculated from the measurement value of the signal intensity include a value obtained by subtracting, from the measurement value of the signal intensity, the measurement value of the signal intensity of a negative control sample; a value obtained by dividing the measurement value of the signal intensity by the measurement value of the signal intensity of a positive control sample; a combination thereof; and the like. Examples of negative control samples include specimens of healthy subjects and the like. Examples of positive control samples include specimens containing the kidney function prediction marker mRNA at a predetermined expression level.

### 3. System Configuration

In the present invention, the below-described “4. Evaluation of kidney function,” “5. Estimation of phosphorus intake,” “6. Prediction of possibility of developing complication associated with kidney disease,” and “7. Determination of effect of dietary therapy” are performed using a measurement value obtained in the above “2. Method for obtaining each measurement value.” First, the system configuration for performing these processes is described.

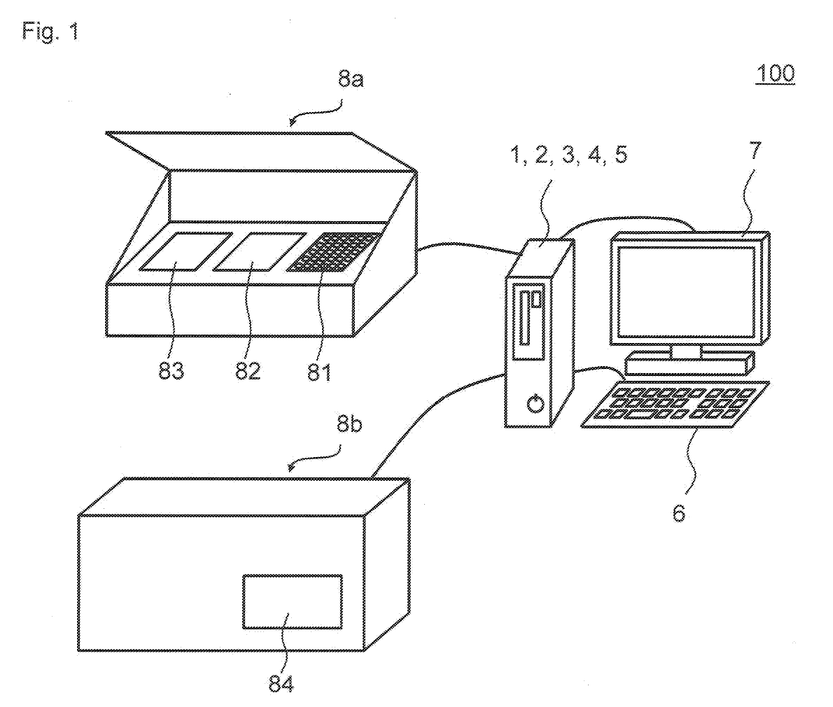

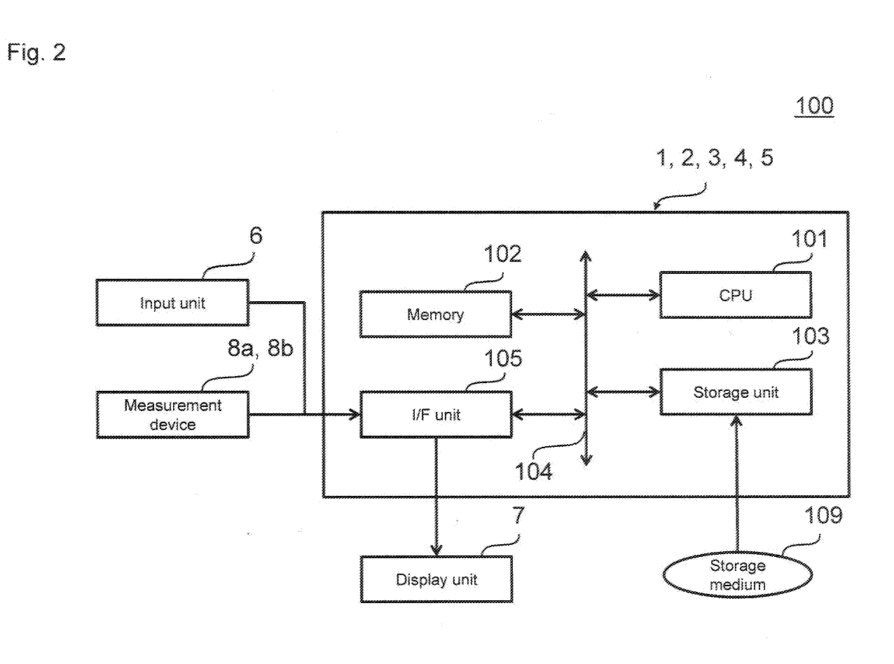

FIG. 1 is an overview of a system 100 according to one embodiment of the present invention, and FIG. 2 is a block diagram illustrating a hardware configuration of the system 100. The system 100 comprises a computing device (since computing devices having the same hardware configuration, but having different functional configurations described later are mentioned below, these computing devices having different functional configurations are collectively referred to as “computing device 1, 2, 3, 4, 5” hereinafter), an input unit 6, a display unit 7, a measurement device 8a, and a measurement device 8b.

The computing device 1, 2, 3, 4, 5 includes, for example, a general-purpose personal computer, and comprises a CPU 101 for performing data processing described later, a memory 102 serving as a work area for data processing, a storage unit 103 for storing processed data, a bus 104 for transmitting data between the units, and an interface unit 105 (hereinafter referred to as “I/F unit”) for performing data input and output between the computing device and external devices. The input unit 6 and the display unit 7 are connected to the computing device 1, 2, 3, 4, 5. The input unit 6 includes, for example, a keyboard; and the display unit 7 includes, for example, a liquid crystal display. The input unit 6 and the display unit 7 may be integrated and implemented as a display with a touch panel. The computing device 1, 2, 3, 4, 5 need not be a single device, and the CPU 101, the memory 102, the storage unit 103, and the like may be located in separate places and connected via a network. The computing device may also be a device that omits the input unit 6 and the display unit 7, and that does not require an operator.

The computing device 1, 2, 3, 4, 5, the measurement device 8a, and the measurement device 8b are also not necessarily located in one place, and may be configured such that the devices located in separate places are communicatively connected to each other via a network.

In the explanation below, a process performed by the computing device 1, 2, 3, 4, 5 means a process performed by the CPU 101 of the computing device 1, 2, 3, 4, 5 based on a program stored in the storage unit 103 or the memory 102 unless otherwise specified. The CPU 101 temporarily stores necessary data (such as intermediate data being processed) in the memory 102 that serves as a work area, and suitably stores data that are stored for a long period of time, such as computation results, in the storage unit 103.

The measurement device 8a is a device for obtaining a measurement value relating to at least one protein selected from the group consisting of kidney function prediction markers, and comprises a sample placement area 81, a reaction unit 82, and a detection unit 83. A specimen, collected from a subject, set in the sample placement area 81 is dispensed into and incubated in a microplate that is placed in the reaction unit 82 and on which an antibody against kidney function prediction marker for antigen capture is immobilized. The unreacted antigen is removed, if necessary. Thereafter, a detection antibody is dispensed into the microplate, followed by incubation. The unreacted antigen is removed if necessary, and a substrate for detecting the detection antibody is dispensed into the microplate. The microplate is transferred to the detection unit 83, and a signal generated by reaction with the substrate is measured. Another embodiment of the measurement device 8a is a device for obtaining the measurement value of at least one mRNA selected from the group consisting of kidney function prediction markers by microarray analysis. A reverse transcription reaction product set in the sample placement area 81 is dispensed into a microarray chip set in the reaction unit 82, followed by hybridization. After the microarray chip is washed, it is transferred to the detection unit 83, and fluorescent signal is measured.

Further, another embodiment of the measurement device 8a is a device for obtaining the measurement value of at least one mRNA selected from the group consisting of kidney function prediction markers by RT-PCR. A reverse transcription reaction product set in the sample placement area 81 is dispensed into a microtube set in the reaction unit 82, and a reagent for quantitative PCR is subsequently dispensed into the microtube. PCR reaction is performed in the reaction unit 82, and fluorescent signal in the tube is detected by the detection unit 83 during the PCR reaction.

Furthermore, another embodiment of the measurement device 8a is a device for obtaining the measurement value of proline as a measurement value relating to at least one protein selected from the group consisting of kidney function prediction markers by color reaction. A pretreated specimen set in the sample placement area 81 is dispensed into a microtube set in the reaction unit 82, and a reagent for color reaction is subsequently dispensed into the microtube. Incubation is performed in the reaction unit 82, and absorbance in the tube is detected in the detection unit 83.

The measurement device 8b is a device for measuring at least one mRNA selected from the group consisting of kidney function prediction markers, and comprises an analysis unit 84. A sample subjected to a reaction for RNA-Seq is set in the analysis unit 84, and analysis of nucleotide sequences is performed in the analysis unit 84.

Another embodiment of the measurement device 8b is a device for obtaining a measurement value relating to at least one protein selected from the group consisting of kidney function prediction markers by measuring proline by mass analysis, and comprises an analysis unit 84. A sample subjected to a treatment for mass analysis is set in the analysis unit 84, and mass analysis is performed in the analysis unit 84.

The measurement devices 8a and 8b are connected to the computing device 1, 2, 3, 4, 5 by a wired or wireless connection. The measurement device 8a A/D converts a measurement value relating to at least one protein selected from the group consisting of kidney function prediction markers, or the measurement value of least one mRNA selected from the group consisting of kidney function prediction markers, and transmits it as digital data to the computing device 1, 2, 3, 4, 5. Similarly, the measurement device 8b A/D converts the measurement value of at least one mRNA selected from the group consisting of kidney function prediction markers and transmits it as digital data to the computing device 1, 2, 3, 4, 5. Therefore, the computing device 1, 2, 3, 4, 5 can obtain, as digital data that can be computed, the measurement value relating to at least one protein selected from the group consisting of kidney function prediction markers, and the measurement value of at least one mRNA selected from the group consisting of kidney function prediction markers. The measurement value relating to at least one protein selected from the group consisting of kidney function prediction markers and the measurement value of at least one mRNA selected from the group consisting of kidney function prediction markers are, for example, transmitted as digital data from a medical institution (not shown) via the internet. Thus, the computing device 1, 2, 3, 4, 5 can obtain, as digital data, the measurement value relating to at least one protein selected from the group consisting of kidney function prediction markers and the measurement value of at least one mRNA selected from the group consisting of kidney function prediction markers.

The measurement value of a kidney disease marker is, for example, transmitted as digital data from a medical institution (not shown) to the computing device 1, 2, 3, 4, and 5 via the internet; therefore, the computing device 1, 2, 3, 4, 5 can obtain the measurement value of a kidney disease marker as digital data.

### 4. Evaluation of Kidney Function

### 4-1. Outline

In this embodiment, the kidney function of a subject is evaluated using a measurement value obtained by performing a method described in the section “2. Method for obtaining each measurement value” above.

Specifically, this embodiment is a method for evaluating the kidney function of a subject, comprising the following steps; or a method for supporting the evaluation of the kidney function of a subject, comprising the following steps:

a step of obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

a step of evaluating the kidney function based on the measurement value(s) obtained in the obtaining step.

More specifically, a measurement value obtained by the “2. Method for obtaining each measurement value” is compared with a predetermined threshold; and when the measurement value is equal to or higher than the threshold, or higher than the threshold, it can be determined that the subject has decreased kidney function.

Further, when the specimen collected from the subject is a blood sample or a body fluid, the measurement value of a kidney function prediction marker protein and the measurement value of a kidney function prediction marker mRNA can be obtained in the obtaining step. When the specimen collected from the subject is tissue, the measurement value of a kidney function prediction marker mRNA can be obtained in the obtaining step.

Moreover, in this embodiment, a decrease in kidney function can be detected before the value of at least one kidney disease marker described in the above section “1. Explanation of terms” falls outside of the range of the threshold. Thus, even if the measurement value of the kidney disease marker obtained from a subject is within the range of the threshold of the corresponding kidney disease marker when compared with each other, it can be determined that the kidney function of the subject has already decreased when the measurement value of the kidney function prediction marker protein and/or kidney function prediction marker mRNA is higher than the threshold of the protein and/or mRNA.

The expression of Defb8, Slco1a1, and Aplnr decreases with a decrease in kidney function; therefore, regarding each of these kidney function prediction markers, it can be determined that the kidney function of the subject has already decreased when the measurement value of the kidney function prediction marker protein and/or kidney function prediction marker mRNA is lower than the threshold of the protein and/or mRNA.

### 4-2. Device and Program for Evaluating Kidney Function

The present invention includes, as a first embodiment, a device for evaluating the kidney function of a subject, the device executing the following computation functions by the CPU 101:

a function for obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

a function for evaluating the kidney function based on the measurement value(s) obtained by the obtaining function.

Preferably, the device further comprises a function for obtaining a measurement value of at least one kidney disease marker in the subject, and a function for comparing the measurement value of the kidney disease marker obtained by the kidney disease marker value obtaining function with a threshold of a corresponding kidney disease marker and identifying a subject in which the measurement value of the kidney disease marker is within a range of the threshold of the kidney disease marker.

In this embodiment, the kidney function can be evaluated by a system 100 (FIGS. 1 and 2) comprising the computing device 1 as the device described above.

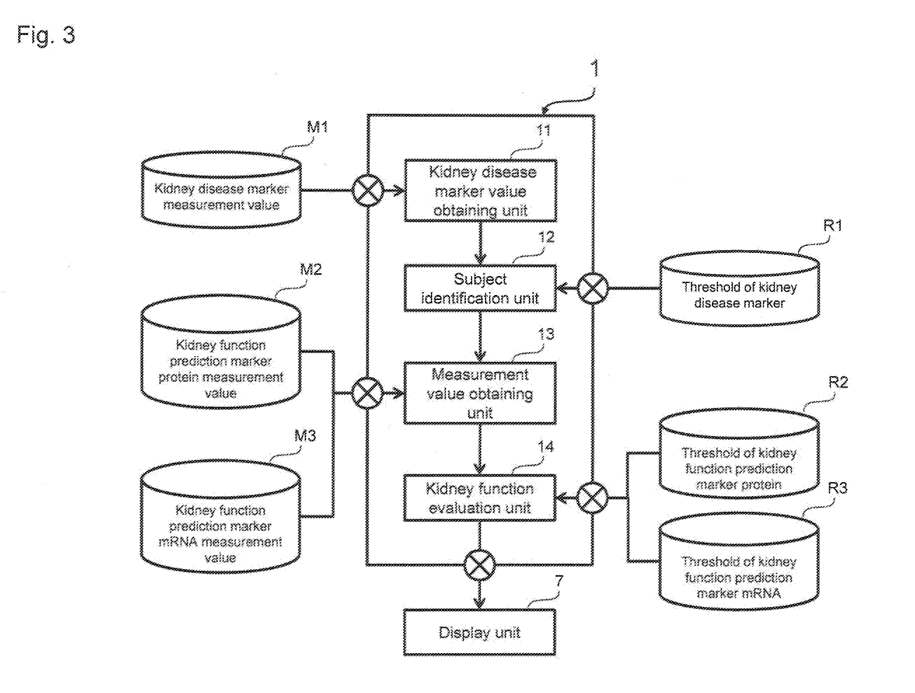

FIG. 3 is a block diagram to illustrate functions of the computing device 1 according to the first embodiment of the present invention. The computing device 1 comprises a kidney disease marker value obtaining unit 11, a subject identification unit 12, a measurement value obtaining unit 13, and a kidney function evaluation unit 14. The kidney disease marker value obtaining unit 11 and the subject identification unit 12 may be optional. These functional blocks are implemented by installing the program according to the present invention in the storage unit 103 or the memory 102 of the computing device 1, and causing the CPU 101 to execute the program. The kidney disease marker value obtaining means, identification means, obtaining means, and evaluation means recited in the claims correspond to the kidney disease marker value obtaining unit 11, subject identification unit 12, measurement value obtaining unit 13, and kidney function evaluation unit 14 shown in FIG. 3, respectively.

In this embodiment, a measurement value M2 of a kidney function prediction marker protein in a subject is put into the computing device 1 from the measurement device 8a or the measurement device 8b, and a measurement value M3 of a kidney function prediction marker mRNA is put into the computing device 1 from the measurement device 8a or the measurement device 8b. A measurement value M1 of a kidney disease marker in the subject, a threshold R1 of the kidney disease marker, a threshold R2 of the kidney function prediction marker protein, and a threshold R3 of the kidney function prediction marker mRNA are stored outside the computing device 1 and put into the computing device 1 via, for example, the internet.

The measurement value M2 of the kidney function prediction marker protein and the measurement value M3 of the kidney function prediction marker mRNA in the subject may be put into the device from a medical institution (not shown) via a network. The threshold R1 of the kidney disease marker, the threshold R2 of the kidney function prediction marker protein, and the threshold R3 of the kidney function prediction marker mRNA may be stored in the storage unit 103 or the memory 102 of the computing device 1 beforehand.

Moreover, the functional blocks, i.e., the kidney disease marker value obtaining unit 11, the subject identification unit 12, the measurement value obtaining unit 13, and the kidney function evaluation unit 14, are not necessarily executed by a single CPU, and may be processed by multiple CPUs in a distributed manner. For example, these functional blocks may be configured such that the functions of the kidney disease marker value obtaining unit 11 and the subject identification unit 12 are executed by a CPU of a first computer, and such that the functions of the measurement value obtaining unit 13 and the kidney function evaluation unit 14 are executed by a CPU of a second computer, i.e., another computer.

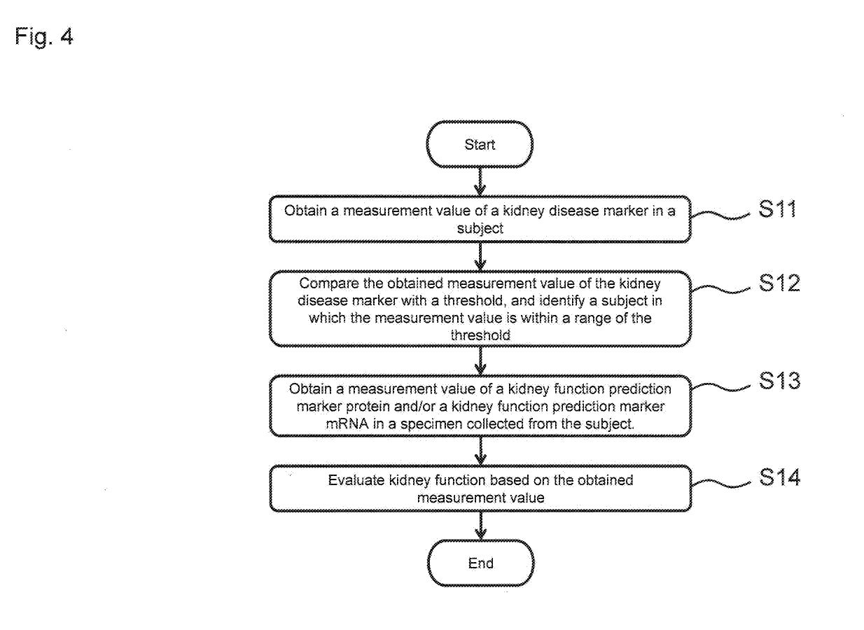

Further, in order to carry out steps S11 to S14 in FIG. 4 described below, the computing device 1 stores the program according to the present invention in the storage unit 103 beforehand, for example, in an executable format (for example, a form in which the program can be produced by conversion from a programming language using a compiler). The computing device 1 carries out processing using the program stored in the storage unit 103. The above program may be installed in the computing device 1 from a computer-readable non-transitory tangible storage medium 109, such as a CD-ROM; alternatively, the computing device 1 may be connected to the internet (not shown) to download the program code of the program via the internet.

### 4-3. Method for Evaluating Kidney Function

The computing device 1 according to the first embodiment of the present invention carries out the following kidney function evaluation method of the present invention. The kidney function evaluation method of the present invention includes a method for evaluating kidney function of a subject, comprising the following steps of:

obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

evaluating the kidney function based on the measurement value(s) obtained in the obtaining step.

Preferably, the above method further comprises, before the obtaining step, the steps of:

obtaining a measurement value of at least one kidney disease marker in the subject; and

comparing the measurement value of the kidney disease marker obtained in the kidney disease marker value obtaining processing with a threshold of a corresponding kidney disease marker, and identifying a subject in which the measurement value of the kidney disease marker is within a range of the threshold of the kidney disease marker.

FIG. 4 is a flow chart illustrating a flow of data processing performed by the computing device 1 according to the first embodiment of the present invention to carry out the method above. The processing for steps S11, S12, S13, and S14 shown in FIG. 4 is performed by the kidney disease marker value obtaining unit 11, subject identification unit 12, measurement value obtaining unit 13, and kidney function evaluation unit 14 shown in FIG. 3, respectively. Steps S11 and S12 are optional steps. Steps S11 and S12 may be performed after S14.

In step S11, the kidney disease marker value obtaining unit 11 obtains a measurement value M1 of a kidney disease marker in a subject. The measurement value M1 of the kidney disease marker is a measurement value obtained by a known method and transmitted from an external medical institution (not shown) to the computing device 1 via the internet.

In step S12, the subject identification unit 12 compares the obtained measurement value M1 of the kidney disease marker with a threshold R1 of the corresponding kidney disease marker, and identifies a subject in which the measurement value M1 of the kidney disease marker is within the range of the threshold R1.

In step S13, the measurement value obtaining unit 13 obtains a measurement value M2 of a kidney function prediction marker protein and/or a measurement value M3 of a kidney function prediction marker mRNA in the subject.

Specifically, when the specimen collected from the subject is a blood sample or a body fluid, the measurement value obtaining unit 13 obtains the measurement value M2 of a kidney function prediction marker protein and the measurement value M3 of a kidney function prediction marker mRNA. When the specimen collected from the subject is tissue, the measurement value obtaining unit 13 obtains the measurement value M3 of a kidney function prediction marker mRNA. The measurement value M2 of a kidney function prediction marker protein and the measurement value M3 of a kidney function prediction marker mRNA are measurement values obtained by the “2. Method for obtaining each measurement value.”

In step S14, the kidney function evaluation unit 14 evaluates the kidney function of the subject based on the obtained measurement value M2 of the kidney function prediction marker protein and/or the obtained measurement value M3 of the kidney function prediction marker mRNA.

Specifically, when the obtained measurement value M2 of the kidney function prediction marker protein is compared with a threshold R2 of the kidney function prediction marker protein, which is a predetermined threshold, and the measurement value M2 is greater than the threshold R2, or when the obtained measurement value M3 of the kidney function prediction marker mRNA is compared with a threshold R3 of the kidney function prediction marker mRNA, which is a predetermined threshold, and the measurement value M3 is greater than the threshold R3, it can be determined as an evaluation result that the subject has decreased kidney function.

The obtained evaluation result is displayed on the display unit 7 of the computing device 1, or stored in the storage unit 103 in the computing device 1. Alternatively, the evaluation result may be displayed on a display unit of a computer terminal that is connected to the computing device 1 via the internet and that is external to the computing device 1, for example, a display unit of a computer terminal in a medical institution.

### 5. Estimation of Phosphorus Intake

### 5-1. Outline

In this embodiment, phosphorus intake in a subject is estimated using a measurement value obtained by performing a method described in the section “2. Method for obtaining each measurement value” above.

Specifically, this embodiment is a method for estimating phosphorus intake in a subject, comprising the following steps, or a method for supporting the estimation of phosphorus intake in a subject, comprising the following steps:

a step of obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

a step of estimating the phosphorus intake in the subject based on the measurement value(s) obtained in the obtaining step.

More specifically, a measurement value obtained by the “2. Method for obtaining each measurement value” is compared with a predetermined threshold, and when the measurement value is equal to or higher than the threshold, or higher than the threshold, it can be determined that the phosphorus intake in the subject is high.

High phosphorus intake is known to be likely to cause a decrease in kidney function in the future. Thus, when the phosphorus intake in the subject is determined to be high in this embodiment, it can be determined that the subject is at risk of a future decrease in kidney function.

Further, when the specimen collected from the subject is a blood sample or a body fluid, the measurement values of a kidney function prediction marker protein and a kidney function prediction marker mRNA can be obtained in the obtaining step. When the specimen collected from the subject is tissue, the measurement value of a kidney function prediction marker mRNA can be obtained in the obtaining step.

Furthermore, the present invention is a method for predicting the risk of a future decrease in kidney function. Thus, also in the case of a subject in which the measurement value of at least one kidney disease marker described in the above section “1. Explanation of terms” is within the range of the threshold of the corresponding kidney disease marker when compared with each other, it can be determined that the subject is at risk of a future decrease in kidney function when the measurement value of the kidney function prediction marker protein and/or kidney function prediction marker mRNA is higher than the threshold of the corresponding protein and/or mRNA.

The expression of Defb8, Slco1a1, and Aplnr decreases with an increase in phosphorus intake; therefore, regarding each of these kidney function prediction markers, it can be determined that the phosphorus intake in the subject is high when the measurement value of the kidney function prediction marker protein and/or kidney function prediction marker mRNA is lower than the threshold of the corresponding protein and/or mRNA.

Here, “future” means 10 years or more, preferably 5 years or more, more preferably 1 year or more, and even more preferably 6 months or more after the date of collection of a specimen used for the measurement.

This embodiment can also be applied to a subject in which the measurement value of at least one kidney disease marker described in the section “1. Explanation of terms” is within the threshold.

As another embodiment, this embodiment can be applied to a subject diagnosed with kidney disease. This embodiment can also be applied to a subject diagnosed with GFR category G1 or G2 shown in Table 3 above.

### 5-2. Device and Program for Estimating-Phosphorus Intake

The present invention includes, as a second embodiment, a device for estimating phosphorus intake in a subject, the device executing the following computation functions by the CPU 101:

a function for obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

a function for estimating the phosphorus intake in the subject based on the measurement value(s) obtained by the obtaining function.

Preferably, the device further comprises a prediction function for determining that the subject is at risk of a future decrease in kidney function when the phosphorus intake in the subject is determined to be high.

Preferably, the device further comprises, before the obtaining function obtains the measurement value(s), the following functions for:

obtaining a measurement value of at least one kidney disease marker in the subject; and

comparing the measurement value of the kidney disease marker obtained by the kidney disease marker value obtaining function with a threshold of a corresponding kidney disease marker, and identifying a subject in which the measurement value of the kidney disease marker is within a range of the threshold of the kidney disease marker.

In this embodiment, the phosphorus intake can be estimated by a system 100 (FIGS. 1 and 2) comprising the computing device 2 as the device described above.

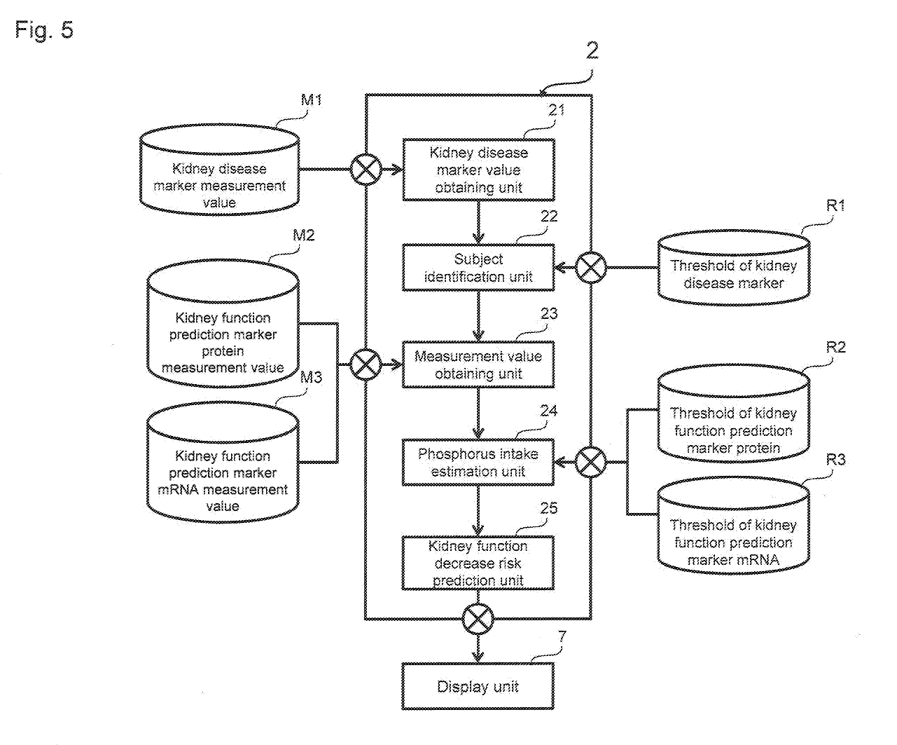

FIG. 5 is a block diagram to illustrate functions of the computing device 2 according to the second embodiment of the present invention. The computing device 2 comprises a kidney disease marker value obtaining unit 21, a subject identification unit 22, a measurement value obtaining unit 23, a phosphorus intake estimation unit 24, and a kidney function decrease risk prediction unit 25. The kidney disease marker value obtaining unit 21 and the subject identification unit 22 may be optional. These functional blocks are implemented by installing the program according to the present invention in the storage unit 103 or the memory 102 of the computing device 2, and causing the CPU 101 to execute the program. The kidney disease marker value obtaining means, identification means, obtaining means, estimation means, and prediction means recited in the claims respectively correspond to the kidney disease marker value obtaining unit 21, subject identification unit 22, measurement value obtaining unit 23, phosphorus intake estimation unit 24, and kidney function decrease risk prediction unit 25 shown in FIG. 5.

In this embodiment, a measurement value M2 of a kidney function prediction marker protein in a subject is put into the computing device 2 from the measurement device 8a or the measurement device 8b, and a measurement value M3 of a kidney function prediction marker mRNA is put into the computing device 2 from the measurement device 8a or the measurement device 8b. A measurement value M1 of a kidney disease marker in the subject, a threshold R1 of the kidney disease marker, a threshold R2 of the kidney function prediction marker protein, and a threshold R3 of the kidney function prediction marker mRNA are stored outside the computing device 2, and put into the computing device 2 via, for example, the internet.

The measurement value M2 of the kidney function prediction marker protein and the measurement value M3 of the kidney function prediction marker mRNA in the subject may be put into the device from a medical institution (not shown) via a network. The threshold R1 of the kidney disease marker, the threshold R2 of the kidney function prediction marker protein, and the threshold R3 of the mRNA may be stored in the storage unit 103 or the memory 102 of the computing device 2 beforehand.

Moreover, the functional blocks, i.e., the kidney disease marker value obtaining unit 21, the subject identification unit 22, the measurement value obtaining unit 23, the phosphorus intake estimation unit 24, and the kidney function decrease risk prediction unit 25, are not necessarily executed by a single CPU, and may be processed by multiple CPUs in a distributed manner. For example, these functional blocks may be configured such that the functions of the kidney disease marker value obtaining unit 21 and the subject identification unit 22 are executed by a CPU of a first computer, and such that the functions of the measurement value obtaining unit 23, the phosphorus intake estimation unit 24, and the kidney function decrease risk prediction unit 25 are executed by a CPU of a second computer, i.e., another computer.

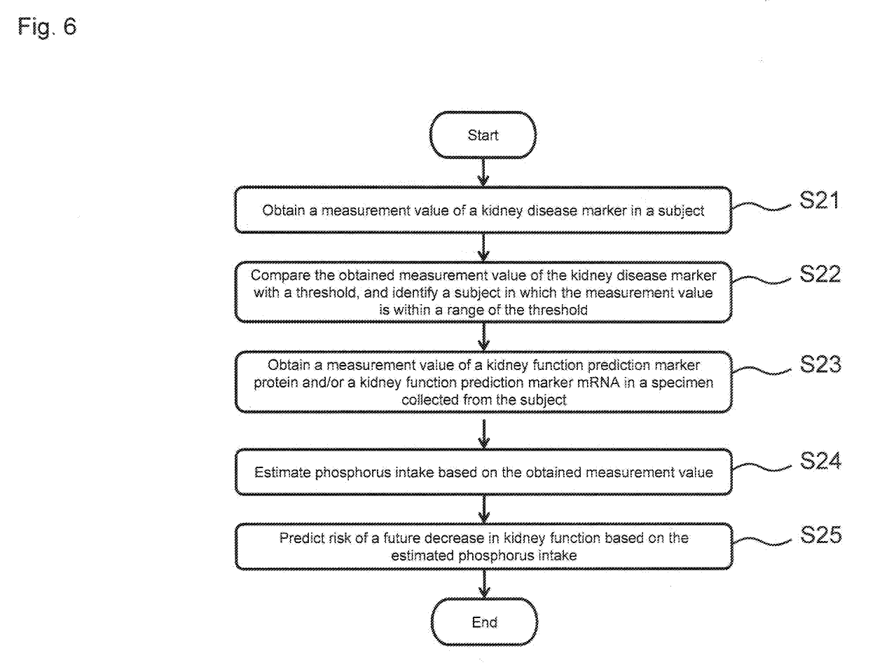

Further, in order to carry out steps S21 to S25 in FIG. 6 described below, the computing device 2 stores the program according to the present invention in the storage unit 103 beforehand, for example, in an executable format (for example, a form in which the program can be produced by conversion from a programming language using a compiler). The computing device 2 carries out processing using the program stored in the storage unit 103. The above program may be installed in the computing device 2 from a computer-readable non-transitory tangible storage medium 109, such as a CD-RCM; alternatively, the computing device 2 may be connected to the internet (not shown) to download the program code of the program via the internet.

### 5-3. Method for Estimating Phosphorus Intake

The computing device 2 according to the second embodiment of the present invention carries out the following phosphorus intake estimation method of the present invention. The phosphorus intake estimation method of the present invention includes a method for estimating phosphorus intake in a subject, comprising the steps of:

obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

estimating the phosphorus intake in the subject based on the measurement value(s) obtained in the obtaining step.

Preferably, the above method further comprises a prediction step of determining that the subject is at risk of a future decrease in kidney function when the phosphorus intake in the subject is determined to be high.

Preferably, the above method further comprises, before the obtaining step, the steps of:

obtaining a measurement value of at least one kidney disease marker in the subject; and

comparing the measurement value of the kidney disease marker obtained in the kidney disease marker value obtaining step with a threshold of a corresponding kidney disease marker, and identifying a subject in which the measurement value of the kidney disease marker is within a range of the threshold of the kidney disease marker.

FIG. 6 is a flow chart illustrating a flow of data processing performed by the computing device 2 according to the second embodiment of the present invention to carry out the method above. The processing for steps S21, S22, S23, S24, and S25 shown in FIG. 6 is respectively performed by the kidney disease marker value obtaining unit 21, subject identification unit 22, measurement value obtaining unit 23, phosphorus intake estimation unit 24, and kidney function decrease risk prediction unit 25 shown in FIG. 5. Steps S21 and S22 are optional steps. Steps S21 and S22 may be performed after S24 or S25.

The processing for steps S21 to S23 by the computing device 2 is the same as the processing for steps S11 to S13 by the computing device 1.

In step S24, the phosphorus intake estimation unit 24 estimates phosphorus intake in the subject based on the obtained measurement value M2 of the kidney function prediction marker protein and/or the measurement value M3 of the kidney function prediction marker mRNA.

Specifically, when the obtained measurement value M2 of the kidney function prediction marker protein is compared with a threshold R2 of the kidney function prediction marker protein, which is a predetermined threshold, and the measurement value M2 is greater than the threshold R2, or when the obtained measurement value M3 of the kidney function prediction marker mRNA is compared with a threshold R3 of the kidney function prediction marker mRNA, which is a predetermined threshold, and the measurement value M3 is greater than the threshold R3, it can be determined that the phosphorus intake in the subject is high.

In step S25, when the phosphorus intake in the subject is determined to be high in step S24, the kidney function decrease risk prediction unit 25 can determine as a prediction result that the subject is at risk of a future decrease in kidney function.

The obtained prediction result is displayed on the display unit 7 of the computing device 2, or stored in the storage unit 103 in the computing device 2. Alternatively, the prediction result may be displayed on a display unit of a computer terminal that is connected to the computing device 2 via the internet and that is external to the computing device 2, for example, a display unit of a computer terminal in a medical institution.

### 6. Prediction of Possibility of Developing Complication Associated with Kidney Disease

### 6-1. Outline

In this embodiment, whether a subject has the possibility of developing a complication(s) associated with kidney disease in the future is predicted using a measurement value obtained by performing a method described in the section “2. Method for obtaining each measurement value” above.

Specifically, a third embodiment is a method for predicting a possibility of developing a complication(s) associated with kidney disease in a subject in the future, comprising the following steps; or a method for supporting the prediction of a possibility of developing a complication(s) associated with kidney disease in a subject in the future, comprising the following steps:

a step of obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

a step of predicting the possibility of developing a complication(s) associated with kidney disease in the future based on the measurement value(s) obtained in the obtaining step.

More specifically, in the third embodiment of the present invention, a measurement value obtained by the “2. Method for obtaining each measurement value” is compared with a predetermined threshold, and when the measurement value is equal to or higher than the threshold, or higher than the threshold, it can be determined that the subject has a possibility of developing a complication(s) associated with kidney disease in the future.

In a fourth embodiment of the present invention, whether a subject has the possibility of developing a complication(s) associated with kidney disease in the future is predicted by using first and second measurement values by performing a method described in the section “2. Method for obtaining each measurement value” above at least twice using the same kind of specimen obtained from the same subject at different times.

Specifically, the fourth embodiment is a method for predicting a possibility of developing a complication(s) associated with kidney disease in a subject in the future, comprising the following steps:

a first obtaining step of obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

a second obtaining step of obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

a step of predicting the possibility of developing a complication(s) associated with kidney disease in the future based on the first measurement value(s) obtained in the first obtaining step and the second measurement value(s) obtained in the second obtaining step.

More specifically, first and second measurement values obtained by using a method described in the section “2. Method for obtaining each measurement value” above are compared with each other, and when the second measurement value is higher than the first measurement value, it can be determined that the subject has the possibility of developing a complication(s) associated with kidney disease in the future. In this case, the collection time is not particularly limited, as long as the specimen for obtaining the first measurement value is collected 1 day or more before collection of the specimen for obtaining the second measurement value. The specimen for obtaining the first measurement value is collected preferably 3 to 7 days or more before, more preferably 7 to 15 days or more before, and even more preferably 15 to 50 days or more before.

In the third and fourth embodiments, the possibility of developing a complication(s) associated with kidney disease in the future can be predicted before the value of at least one kidney disease marker described in the above section “1. Explanation of terms” falls outside of the range of the threshold. Thus, even if the measurement value of the kidney disease marker obtained from a subject is within the range of the threshold of the corresponding kidney disease marker when compared with each other, it can be determined that the subject has the possibility of developing a complication(s) associated with kidney disease in the future when the measurement value of the kidney function prediction marker protein and/or kidney function prediction marker mRNA is higher than the threshold of the protein and/or mRNA.

The expression of Defb8, Slco1a1, and Aplnr decreases with a decrease in kidney function; therefore, regarding each of these kidney function prediction markers, it can be determined that the subject has the possibility of developing a complication(s) associated with kidney disease in the future when the measurement value of the kidney function prediction marker protein and/or kidney function prediction marker mRNA is lower than the threshold of the protein and/or mRNA. Moreover, when the first measurement value is compared with the second measurement value, and the second measurement value is lower than the first measurement value, it can also be determined that the subject has the possibility of developing a complication(s) associated with kidney disease in the future.

Further, when the specimen collected from the subject is a blood sample or a body fluid, the first measurement values of a kidney function prediction marker protein and a kidney function prediction marker mRNA can be obtained in the first obtaining step, and the second measurement values of the kidney function prediction marker protein and the kidney function prediction marker mRNA can be obtained in the second obtaining step. When the specimen collected from the subject is tissue, the first measurement value of a kidney function prediction marker mRNA can be obtained in the first obtaining step, and the second measurement value of the kidney function prediction marker mRNA can be obtained in the second obtaining step.

As other embodiments, the third and fourth embodiments can be applied to a subject diagnosed with kidney disease. Further, the third and fourth embodiments can also be applied to a subject diagnosed with GFR category G1 or G2 shown in Table 3.

### 6-2. Device and Program for Predicting Possibility of Developing Complication

(1) Embodiment in which Comparison with Predetermined Threshold is Performed

The present invention includes, as the third embodiment, a device for predicting a possibility of developing a complication(s) associated with kidney disease in a subject in the future, the device executing the following computation functions by the CPU 101:

a function for obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

a function for predicting the possibility of developing a complication(s) associated with kidney disease in the future based on the measurement value(s) obtained by the obtaining function.

Preferably, the device further comprises, before the obtaining function obtains the measurement value(s), the following functions for:

obtaining a measurement value of at least one kidney disease marker in the subject; and

comparing the measurement value of the kidney disease marker obtained by the kidney disease marker value obtaining function with a threshold of a corresponding kidney disease marker, and identifying a subject in which the measurement value of the kidney disease marker is within a range of the threshold of the kidney disease marker.

In this embodiment, the possibility of developing a complication(s) can be predicted by a system 100 (FIGS. 1 and 2) comprising the computing device 3 as the device described above.

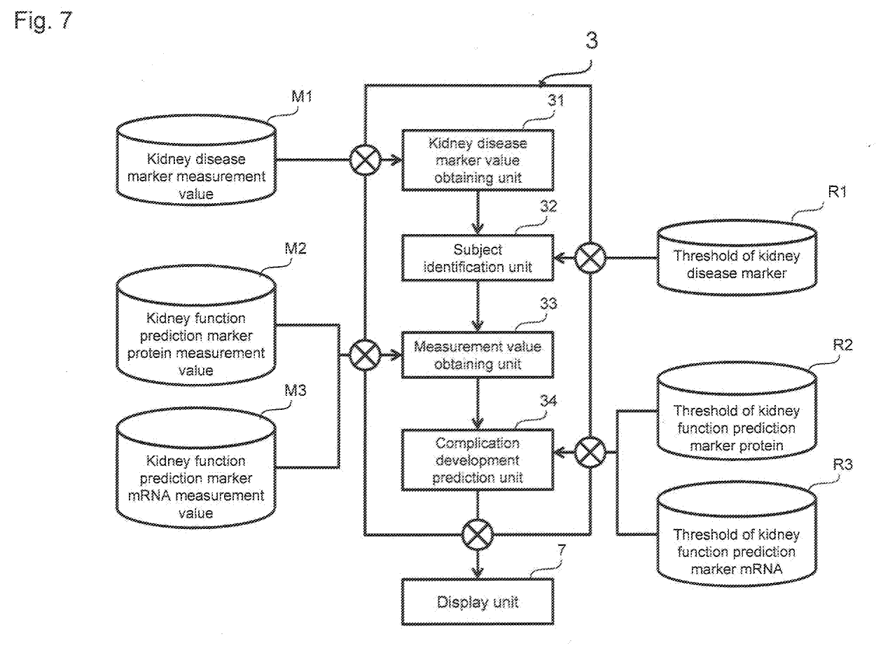

FIG. 7 is a block diagram to illustrate functions of the computing device 3 according to the third embodiment of the present invention. The computing device 3 comprises a kidney disease marker value obtaining unit 31, a subject identification unit 32, a measurement value obtaining unit 33, and a complication(s) development prediction unit 34. The kidney disease marker value obtaining unit 31 and the subject identification unit 32 may be optional. These functional blocks are implemented by installing the program according to the present invention in the storage unit 103 or the memory 102 of the computing device 3, and causing the CPU 101 to execute the program. The kidney disease marker value obtaining means, identification means, obtaining means, and prediction means recited in the claims correspond to the kidney disease marker value obtaining unit 31, subject identification unit 32, measurement value obtaining unit 33, and complication development prediction unit 34 shown in FIG. 7, respectively.

In this embodiment, a measurement value M2 of a kidney function prediction marker protein in a subject is put into the computing device 3 from the measurement device 8a or the measurement device 8b, and a measurement value M3 of a kidney function prediction marker mRNA is put into the computing device 3 from the measurement device 8a or the measurement device 8b. A measurement value M1 of a kidney disease marker in the subject, a threshold R1 of the kidney disease marker, a threshold R2 of the kidney function prediction marker protein, and a threshold R3 of the kidney function prediction marker mRNA are stored outside the computing device 3 and put into the computing device 3 via, for example, the internet.

The measurement value M2 of the kidney function prediction marker protein and the measurement value M3 of the kidney function prediction marker mRNA in the subject may be put into the device from a medical institution (not shown) via a network. The threshold R1 of the kidney disease marker, the threshold R2 of the kidney function prediction marker protein, and the threshold R3 of the mRNA may be stored in the storage unit 103 or the memory 102 of the computing device 3 beforehand.

Moreover, the functional blocks, i.e., the kidney disease marker value obtaining unit 31, the subject identification unit 32, the measurement value obtaining unit 33, and the complication development prediction unit 34, are not necessarily executed by a single CPU, and may be processed by multiple CPUs in a distributed manner. For example, these functional blocks may be configured such that the functions of the kidney disease marker value obtaining unit 31 and the subject identification unit 32 are executed by a CPU of a first computer, and such that the functions of the measurement value obtaining unit 33 and the complication development prediction unit 34 are executed by a CPU of a second computer, i.e., another computer.

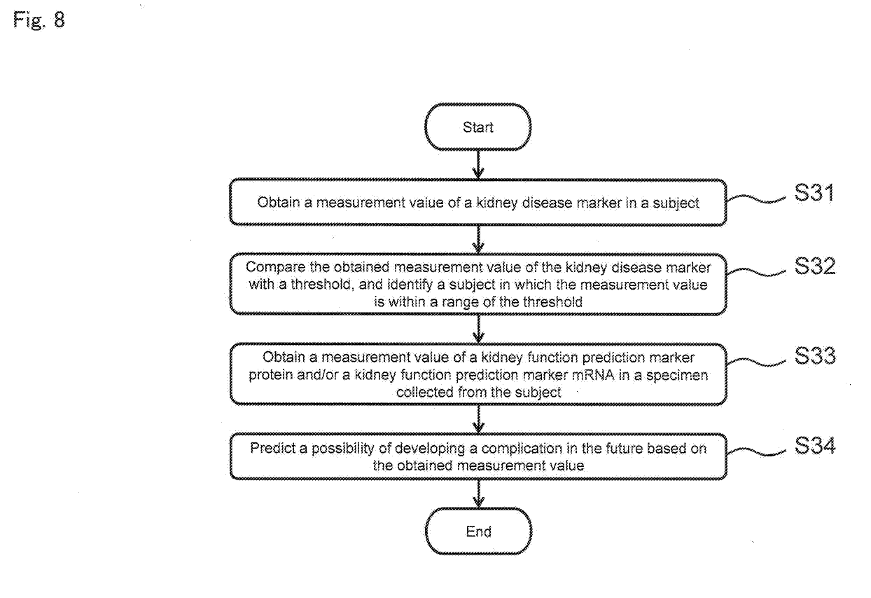

Further, in order to carry out the processing for steps S31 to S34 in FIG. 8 described below, the computing device 3 stores the program according to the present invention in the storage unit 103 beforehand, for example, in an executable format (for example, a form in which the program can be produced by conversion from a programming language using a compiler). The computing device 3 carries out the processing using the program stored in the storage unit 103. The above program may be installed in the computing device 3 from a computer-readable non-transitory tangible storage medium 109, such as a CD-ROM; alternatively, the computing device 3 may be connected to the internet (not shown) to download the program code of the program via the internet.

(2) Embodiment in which Measurement Values Obtained at Different Times are Compared with Each Other

The present invention includes, as the fourth embodiment, a device for predicting a possibility of developing a complication(s) associated with kidney disease in a subject in the future, the device executing the following computation functions by the CPU 101:

a first obtaining function for obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

a second obtaining function for obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

a function for predicting the possibility of developing a complication(s) associated with kidney disease in the future based on the first measurement value(s) obtained by the first obtaining function and the second measurement value(s) obtained by the second obtaining function.

Preferably, the device further comprises, before the first and second obtaining functions respectively obtain the first and second measurement values, the following functions for:

obtaining a measurement value of at least one kidney disease marker in the subject; and

comparing the measurement value of the kidney disease marker obtained by the kidney disease marker value obtaining function with a threshold of a corresponding kidney disease marker, and identifying a subject in which the measurement value of the kidney disease marker is within a range of the threshold of the kidney disease marker.

In this embodiment, the possibility of developing a complication(s) can be predicted by a system 100 (FIGS. 1 and 2) comprising the computing device 4 as the device described above.

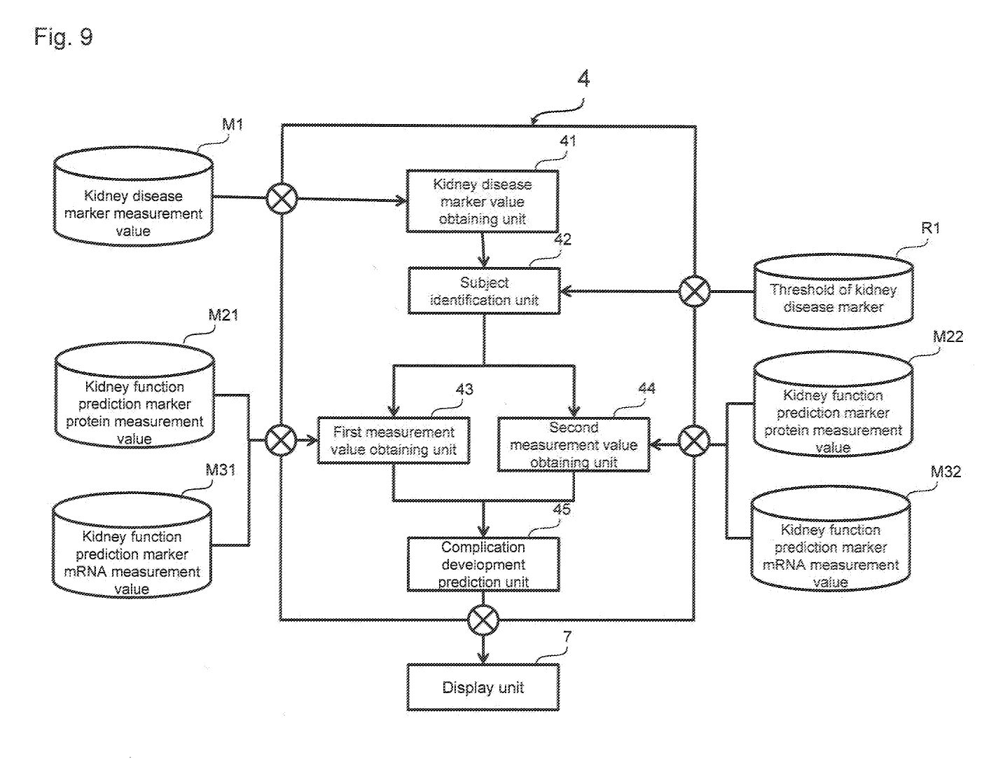

FIG. 9 is a block diagram to illustrate functions of the computing device 4 according to the fourth embodiment of the present invention. The computing device 4 comprises a kidney disease marker value obtaining unit 41, a subject identification unit 42, a first measurement value obtaining unit 43, a second measurement value obtaining unit 44, and a complication(s) development prediction unit 45. The kidney disease marker value obtaining unit 41 and the subject identification unit 42 may be optional. These functional blocks are implemented by installing the program according to the present invention in the storage unit 103 or the memory 102 of the computing device 4, and causing the CPU 101 to execute the program. The kidney disease marker value obtaining means, identification means, first obtaining means, second obtaining means, and prediction means recited in the claims correspond to the kidney disease marker value obtaining unit 41, subject identification unit 42, first measurement value obtaining unit 43, second measurement value obtaining unit 44, and complication development prediction unit 45 shown in FIG. 9, respectively.

In this embodiment, measurement values M21 and M22 of a kidney function prediction marker protein in a subject are put into the computing device 4 from the measurement device 8a or the measurement device 8b, and measurement values M31 and M32 of a kidney function prediction marker mRNA are put into the computing device 4 from the measurement device 8a or the measurement device 8b. A measurement value M1 of a kidney disease marker in the subject and a threshold R1 of the kidney disease marker are stored outside the computing device 4 and put into the computing device 4 via, for example, the internet.

The measurement values M21 and M22 of the kidney function prediction marker protein and the measurement values M31 and M32 of the kidney function prediction marker mRNA in the subject may be put into the device from a medical institution (not shown) via a network. The threshold R1 of the kidney disease marker may be stored in the storage unit 103 or the memory 102 of the computing device 4 beforehand.

Moreover, the functional blocks, i.e., the kidney disease marker value obtaining unit 41, the subject identification unit 42, the first measurement value obtaining unit 43, the second measurement value obtaining unit 44, and the complication development prediction unit 45, are not necessarily executed by a single CPU, and may be processed by multiple CPUs in a distributed manner. For example, these functional blocks may be configured such that the functions of the kidney disease marker value obtaining unit 41 and the subject identification unit 42 are executed by a CPU of a first computer, and such that the functions of the first measurement value obtaining unit 43, the second measurement value obtaining unit 44, and the complication development prediction unit 45 are executed by a CPU of a second computer, i.e., another computer.

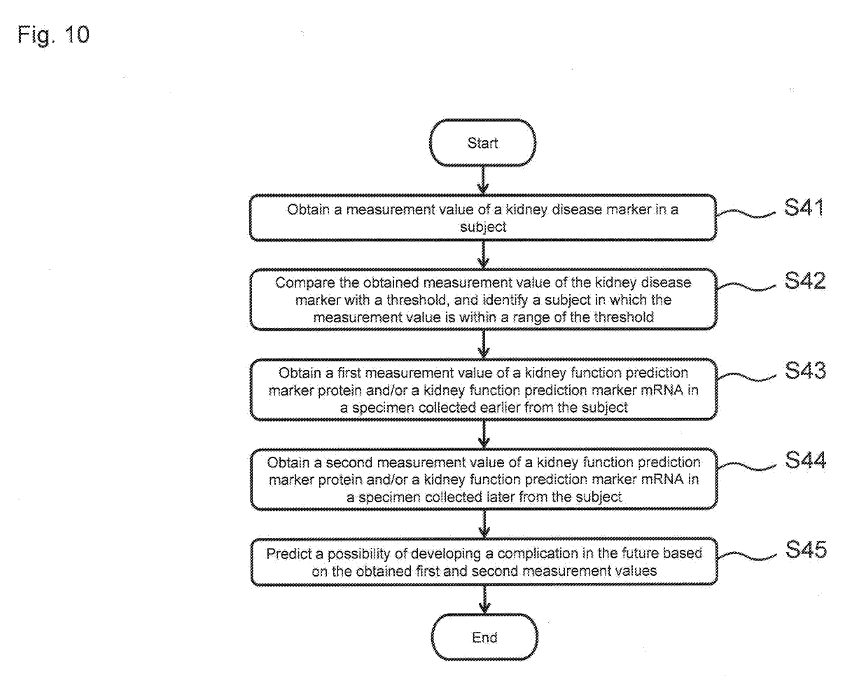

Further, in order to carry out the processing for steps S41 to S45 in FIG. 10 described below, the computing device 4 stores the program according to the present invention in the storage unit 103 beforehand, for example, in an executable format (for example, a form in which the program can be produced by conversion from a programming language using a compiler). The computing device 4 carries out the processing using the program stored in the storage unit 103. The above program may be installed in the computing device 4 from a computer-readable non-transitory tangible storage medium 109, such as a CD-ROM; alternatively, the computing device 4 may be connected to the internet (not shown) to download the program code of the program via the internet.

### 6-3. Method for Predicting Possibility of Developing Complication

(1) Embodiment in which Comparison with Predetermined Threshold is Performed

The computing device 3 according to the third embodiment of the present invention carries out the following method for predicting the possibility of developing a complication(s) according to the third embodiment of the present invention. The method for predicting the possibility of developing a complication(s) according to the third embodiment of the present invention includes a method for predicting a possibility of developing a complication(s) associated with kidney disease in a subject in the future, comprising the steps of:

obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and

predicting the possibility of developing a complication(s) associated with kidney disease in the future based on the measurement value(s) obtained in the obtaining step.

Preferably, the above method further comprises, before the obtaining step, the steps of:

obtaining a measurement value of at least one kidney disease marker in the subject; and

comparing the measurement value of the kidney disease marker obtained in the kidney disease marker value obtaining step with a threshold of a corresponding kidney disease marker, and identifying a subject in which the measurement value of the kidney disease marker is within a range of the threshold of the kidney disease marker.

FIG. 8 is a flow chart illustrating a flow of data processing performed by the computing device 3 according to the third embodiment of the present invention to carry out the method above. The processing for steps S31, S32, S33, and S34 shown in FIG. 8 is respectively performed by the kidney disease marker value obtaining unit 31, subject identification unit 32, measurement value obtaining unit 33, and complication development prediction unit 34 shown in FIG. 7. Steps S31 and S32 are optional steps. Steps S31 and S32 may be performed after S34.

The processing for steps S31 to S33 by the computing device 3 is the same as the processing for steps S11 to S13 by the computing device 1.

In step S34, the complication development prediction unit 34 predicts the possibility of developing a complication(s) in the subject based on the obtained measurement value M2 of the kidney function prediction marker protein and/or the measurement value M3 of the kidney function prediction marker mRNA.

Specifically, when the obtained measurement value M2 of the kidney function prediction marker protein is compared with a threshold R2 of the kidney function prediction marker protein, which is a predetermined threshold, and the measurement value M2 is greater than the threshold R2, or when the obtained measurement value M3 of the kidney function prediction marker mRNA is compared with a threshold R3 of the mRNA, which is a predetermined threshold, and the measurement value M3 is greater than the threshold R3, it can be determined as a prediction result that the subject has the possibility of developing a complication(s) associated with kidney disease in the future.

The obtained prediction result is displayed on the display unit 7 of the computing device 3, or stored in the storage unit 103 in the computing device 3. Alternatively, the prediction result may be displayed on a display unit of a computer terminal that is connected to the computing device 3 via the internet and that is external to the computing device 3, for example, a display unit of a computer terminal in a medical institution.

(2) Embodiment in which Measurement Values Obtained at Different Times are Compared with Each Other

The computing device 4 according to the fourth embodiment of the present invention carries out the following method for predicting the possibility of developing a complication(s) according to the fourth embodiment of the present invention. The method for predicting the possibility of developing a complication(s) according to the fourth embodiment of the present invention includes a method for predicting a possibility of developing a complication(s) associated with kidney disease in a subject in the future, comprising the following steps:

a first obtaining step of obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

a second obtaining step of obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

a step of predicting the possibility of developing a complication(s) associated with kidney disease in the future based on the first measurement value(s) obtained in the first obtaining step and the second measurement value(s) obtained in the second obtaining step.

Preferably, the above method further comprises, before the first and second obtaining steps, the steps of:

obtaining a measurement value of at least one kidney disease marker in the subject; and

comparing the measurement value of the kidney disease marker obtained in the kidney disease marker value obtaining step with a threshold of the corresponding kidney disease marker, and identifying a subject in which the measurement value of the kidney disease marker is within a range of the threshold of the kidney disease marker.

FIG. 10 is a flow chart illustrating a flow of data processing performed by the computing device 4 according to the fourth embodiment of the present invention to carry out the method above. The processing for steps S41, S42, S43, S44, and S45 shown in FIG. 10 is respectively performed by the kidney disease marker value obtaining unit 41, subject identification unit 42, first measurement value obtaining unit 43, second measurement value obtaining unit 44, and complication development prediction unit 45 shown in FIG. 9. Steps S41 and S42 are optional steps. Steps S41 and S42 may be performed after S45.

The processing for steps S41 and S42 by the computing device 4 is the same as the processing for steps S11 and S12 by the computing device 1.

In step S43, the first measurement value obtaining unit 43 obtains a measurement value M21 of a kidney function prediction marker protein and/or a measurement value M31 of a kidney function prediction marker mRNA contained in an earlier collected specimen of an identified subject, as a first measurement value obtained earlier.

Specifically, when the specimen collected earlier from the subject is a blood sample or a body fluid, the first measurement value obtaining unit 43 obtains the measurement value M21 of a kidney function prediction marker protein and the measurement value M31 of a kidney function prediction marker mRNA as first measurement values. When the specimen collected earlier from the subject is tissue, the first measurement value obtaining unit 43 obtains the measurement value M31 of an mRNA as a first measurement value.

In step S44, the second measurement value obtaining unit 44 obtains a measurement value M22 of the kidney function prediction marker protein and/or a measurement value M32 of the kidney function prediction marker mRNA contained in a later obtained specimen of the identified subject, as a second measurement value obtained later.

Specifically, when the specimen collected later from the subject is a blood sample or a body fluid, the second measurement value obtaining unit 44 obtains the measurement value M22 of the kidney function prediction marker protein and the measurement value M32 of the kidney function prediction marker mRNA as second measurement values. When the specimen collected later from the subject is tissue, the second measurement value obtaining unit 44 obtains the measurement value M32 of the kidney function prediction marker mRNA as a second measurement value.

In step S45, the complication development prediction unit 45 compares the first measurement value earlier obtained with the second measurement value later obtained, and predicts the possibility of developing a complication(s) in a subject.

Specifically, in the case where the specimen collected from the subject is a blood sample or a body fluid, when the measurement value M22 of the kidney function prediction marker protein, which is a second measurement value, is higher than the measurement value M21 of the kidney function prediction marker protein, which is a first measurement value, or when the measurement value M32 of the kidney function prediction marker mRNA, which is a second measurement value, is higher than the measurement value M31 of the kidney function prediction marker mRNA, which is a first measurement value, it can be determined as a prediction result that the subject has the possibility of developing a complication(s) associated with kidney disease in the future. In the case where the specimen collected from the subject is tissue, when the measurement value M32 of the kidney function prediction marker mRNA, which is a second measurement value, is higher than the measurement value M31 of the kidney function prediction marker mRNA, which is a first measurement value, it can be determined as a prediction result that the subject has the possibility of developing a complication(s) associated with kidney disease in the future.

The obtained prediction result is displayed on the display unit 7 of the computing device 4, or stored in the storage unit 103 in the computing device 4. Alternatively, the prediction result may be displayed on a display unit of a computer terminal that is connected to the computing device 4 via the internet and that is external to the computing device 4, for example, a display unit of a computer terminal in a medical institution.

### 7. Determination of Effect of Dietary Therapy

### 7-1. Outline

In this embodiment, the effect of dietary therapy in a subject is determined using measurement values obtained by performing a method described in the section “2. Method for obtaining each measurement value” above.

Specifically, this embodiment is a method for determining an effect of dietary therapy in a subject, comprising the following steps; or a method for supporting the determination of an effect of dietary therapy in a subject, comprising the following steps:

a first obtaining step of obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

a second obtaining step of obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

a step of determining the effect of dietary therapy based on the first measurement value(s) obtained in the first obtaining step and the second measurement value(s) obtained in the second obtaining step.

More specifically, the effect of dietary therapy in a subject is determined by using first and second measurement values obtained from the same subject at different times by performing a method described in the section “2. Method for obtaining each measurement value” above at least twice. More specifically, first and second measurement values obtained by using a method described in the section “2. Method for obtaining each measurement value” above are compared with each other, and when the second measurement value is lower than the first measurement value, it can be determined that the dietary therapy is effective in the subject. In this case, the collection time is not particularly limited, as long as the specimen for obtaining the first measurement value is collected 1 week or more before collection of the specimen for obtaining the second measurement value. The specimen for obtaining the first measurement value is collected preferably 2 to 7 days before, more preferably 1 to 4 weeks or more before, and even more preferably 1 to 3 months or more before.

Further, when the specimen collected from the subject is a blood sample or a body fluid, the first measurement values of a kidney function prediction marker protein and a kidney function prediction marker mRNA can be obtained in the first obtaining step, and the second measurement values of the kidney function prediction marker protein and the kidney function prediction marker mRNA can be obtained in the second obtaining step. When the specimen collected from the subject is tissue, the first measurement value of a kidney function prediction marker mRNA can be obtained in the first obtaining step, and the second measurement value of the kidney function prediction marker mRNA can be obtained in the second obtaining step.

The expression of Defb8, Slco1a1, and Aplnr decreases with an increase in phosphorus intake; therefore, regarding each of these kidney function prediction markers, when the first measurement value is compared with the second measurement value, and the second measurement value is higher than the first measurement value, it can be determined that the dietary therapy is effective in the subject.

This embodiment can also be applied to a subject in which the measurement value of the at least one kidney disease marker described in the section “1. Explanation of terms” is within the threshold.

As another embodiment, this embodiment can be applied to a subject diagnosed with kidney disease. Further, this embodiment can also be applied to a subject diagnosed with GFR category G1 or G2 shown in Table 3.

### 7-2. Device and Program for Determining Effect of Dietary Therapy

The present invention includes, as a fifth embodiment, a device for determining an effect of dietary therapy in a subject, the device executing the following computation functions by the CPU 101:

a first obtaining function for obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

a second obtaining function for obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

a function for determining the effect of dietary therapy based on the first measurement value(s) obtained by the first obtaining function and the second measurement value(s) obtained by the second obtaining function.

Preferably, the above device further comprises, before the first and second obtaining functions respectively obtain the first and second measurement values, the following functions for:

obtaining a measurement value of at least one kidney disease marker in the subject; and

comparing the measurement value of the kidney disease marker obtained by the kidney disease marker value obtaining function with a threshold of a corresponding kidney disease marker, and identifying a subject in which the measurement value of the kidney disease marker is within a range of the threshold of the kidney disease marker.

In this embodiment, the effect of dietary therapy can be determined by a system 100 (FIGS. 1 and 2) comprising the computing device 5 as the device described above.

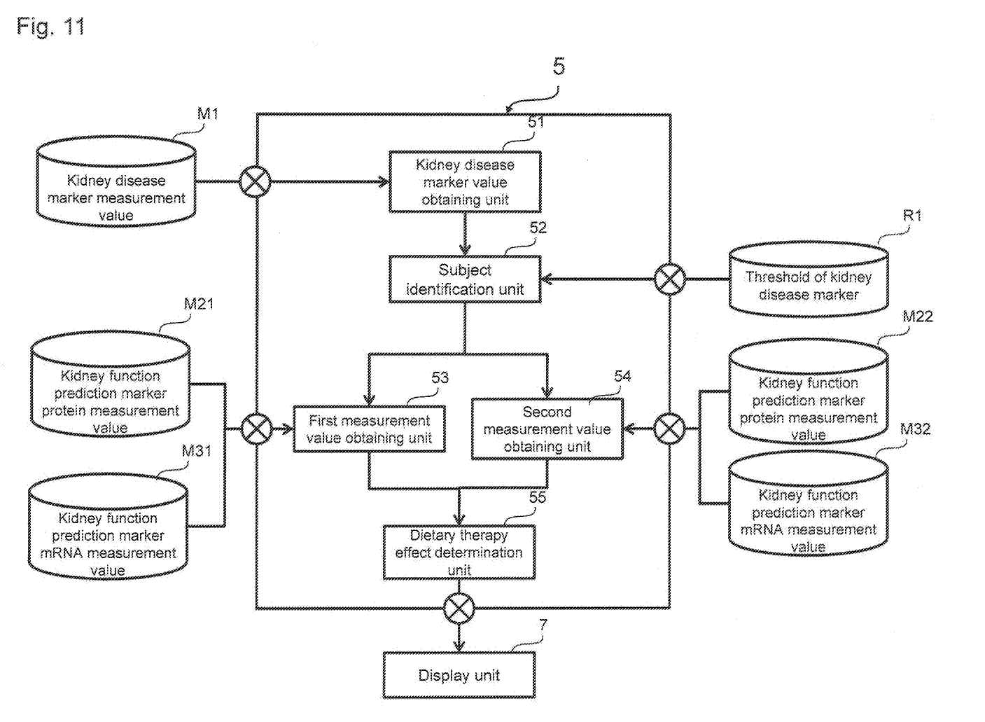

FIG. 11 is a block diagram to illustrate functions of the computing device 5 according to the fifth embodiment of the present invention. The computing device 5 comprises a kidney disease marker value obtaining unit 51, a subject identification unit 52, a first measurement value obtaining unit 53, a second measurement value obtaining unit 54, and a dietary therapy effect determination unit 55. The kidney disease marker value obtaining unit 51 and the subject identification unit 52 may be optional. These functional blocks are implemented by installing the program according to the present invention in the storage unit 103 or the memory 102 of the computing device 5, and causing the CPU 101 to execute the program. The kidney disease marker value obtaining means, identification means, first obtaining means, second obtaining means, and determination means recited in the claims respectively correspond to the kidney disease marker value obtaining unit 51, subject identification unit 52, first measurement value obtaining unit 53, second measurement value obtaining unit 54, and dietary therapy effect determination unit 55 shown in FIG. 11.

In this embodiment, measurement values M21 and M22 of a kidney function prediction marker protein in a subject are put into the computing device 5 from the measurement device 8a or the measurement device 8b, and measurement values M31 and M32 of a kidney function prediction marker mRNA are put into the computing device 5 from the measurement device 8a or the measurement device 8b. A measurement value M1 of a kidney disease marker in the subject and a threshold R1 of the kidney disease marker are stored outside the computing device 5 and put into the computing device 5 via, for example, the internet.

The measurement values M21 and M22 of the kidney function prediction marker protein and the measurement values M31 and M32 of the kidney function prediction marker mRNA in the subject may be put into the device from a medical institution (not shown) via a network. The threshold R1 of the kidney disease marker may be stored in the storage unit 103 or the memory 102 of the computing device 5 beforehand.

Moreover, the functional blocks, i.e., the kidney disease marker value obtaining unit 51, the subject identification unit 52, the first measurement value obtaining unit 53, the second measurement value obtaining unit 54, and the dietary therapy effect determination unit 55, are not necessarily executed by a single CPU, and may be processed by multiple CPUs in a distributed manner. For example, these functional blocks may be configured such that the functions of the kidney disease marker value obtaining unit 51 and the subject identification unit 52 are executed by a CPU of a first computer, and such that the functions of the first measurement value obtaining unit 53, the second measurement value obtaining unit 54, and the dietary therapy effect determination unit 55 are executed by a CPU of a second computer, i.e., another computer.

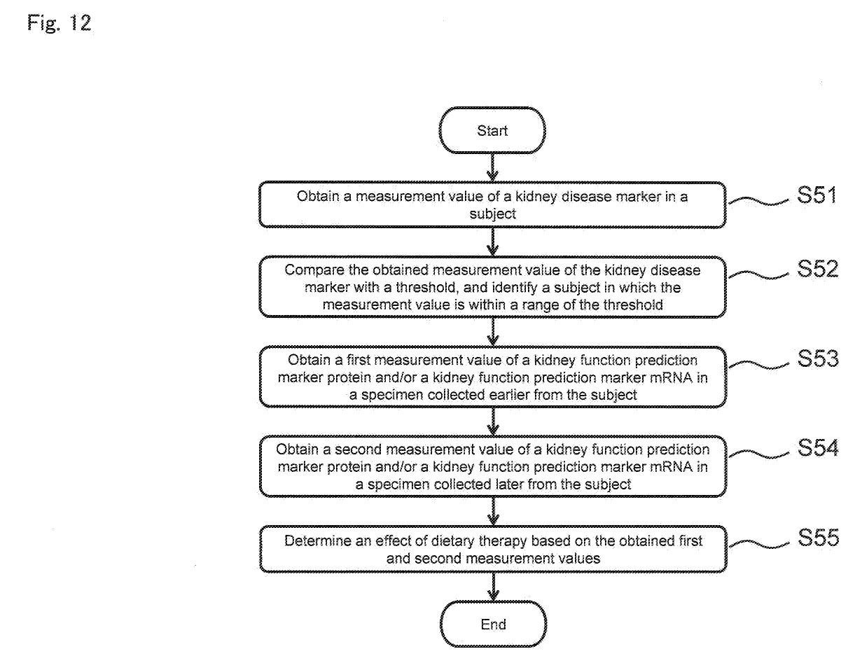

Further, in order to carry out the processing for steps S51 to S55 in FIG. 12 described below, the computing device 5 stores the program according to the present invention in the storage unit 103 beforehand, for example, in an executable format (for example, a form in which the program can be produced by conversion from a programming language using a compiler). The computing device 5 carries out the processing using the program stored in the storage unit 103. The above program may be installed in the computing device 5 from a computer-readable non-transitory tangible storage medium 109, such as a CD-ROM; alternatively, the computing device 5 may be connected to the internet (not shown) to download the program code of the program via the internet.

### 7-3. Method for Determining Effect of Dietary Therapy

The computing device 5 according to the fifth embodiment of the present invention carries out the following method for determining an effect of dietary therapy according to the present invention. The method for determining an effect of dietary therapy according to the present invention includes a method for determining an effect of dietary therapy in a subject, comprising the following steps:

a first obtaining step of obtaining a first measurement value relating to at least one protein selected from the group consisting of kidney function prediction markers contained in a specimen collected earlier from the subject, and/or a first measurement value of at least one mRNA selected from the group consisting of kidney function prediction markers contained in a specimen collected earlier from the subject;

a second obtaining step of obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and

a step of determining the effect of dietary therapy based on the first measurement value(s) obtained in the first obtaining step and the second measurement value(s) obtained in the second obtaining step.

Preferably, the above method further comprises, before the first and second obtaining steps, the steps of:

obtaining a measurement value of at least one kidney disease marker in the subject; and

comparing the measurement value of the kidney disease marker obtained in the kidney disease marker value obtaining step with a threshold of a corresponding kidney disease marker, and identifying a subject in which the measurement value of the kidney disease marker is within a range of the threshold of the kidney disease marker.

FIG. 12 is a flow chart illustrating a flow of data processing performed by the computing device 5 according to the fifth embodiment of the present invention to carry out the method above. The processing for steps S51, S52, S53, S54, and S55 shown in FIG. 12 is respectively performed by the kidney disease marker value obtaining unit 51, subject identification unit 52, first measurement value obtaining unit 53, second measurement value obtaining unit 54, and dietary therapy effect determination unit 55 shown in FIG. 11. Steps S51 and S52 are optional steps. Steps S51 and S52 may be performed after S55.

The processing for steps S51 to S54 by the computing device 5 is the same as the processing for steps S41 to S44 by the computing device 4.

In step S55, the dietary therapy effect determination unit 55 compares the first measurement value obtained earlier with the second measurement value obtained later, and determines the effect of dietary therapy.

Specifically, in the case where the specimen collected from the subject is a blood sample or a body fluid, when the measurement value M22 of the kidney function prediction marker protein, which is a second measurement value, is lower than the measurement value M21 of the kidney function prediction marker protein, which is a first measurement value, or when the measurement value M32 of the kidney function prediction marker mRNA, which is a second measurement value, is lower than the measurement value M31 of the kidney function prediction marker mRNA, which is a first measurement value, it can be determined as a prediction result that the dietary therapy is effective. In the case where the specimen collected from the subject is tissue, when the measurement value M32 of the kidney function prediction marker mRNA, which is a second measurement value, is lower than the measurement value M31 of the kidney function prediction marker mRNA, which is a first measurement value, it can be determined as a prediction result that the dietary therapy is effective.

The obtained determination result is displayed on the display unit 7 of the computing device 5, or stored in the storage unit 103 in the computing device 5. Alternatively, the determination result may be displayed on a display unit of a computer terminal that is connected to the computing device 5 via the internet and that is external to the computing device 5, for example, a display unit of a computer terminal in a medical institution.

### 8. Treatment or Dietary Improvement Therapy for Subject

A necessary treatment or therapy can be performed for a subject determined to have decreased kidney function in the method for evaluating kidney function of a subject or the method for supporting the evaluation of kidney function of a subject described in section 4 above; a subject determined to have high phosphorus intake or determined to be at risk of a future decrease in kidney function in the method for estimating phosphorus intake in a subject or the method for supporting the estimation of phosphorus intake in a subject described in section 5 above; a subject determined to have the possibility of developing a complication(s) associated with kidney disease in the future in the method for predicting a possibility of developing a complication(s) associated with kidney disease in a subject in the future or the method for supporting the prediction of a possibility of developing a complication(s) associated with kidney disease in a subject in the future described in section 6 above; and a subject in which no effect of dietary therapy was observed in the method for determining an effect of dietary therapy in a subject or the method for supporting the determination of an effect of dietary therapy in a subject described in section 7 above.

Dietary therapy, rest treatment, drug therapy, or the like to prevent worsening kidney function can be performed for a subject determined to have decreased kidney function, and a subject determined to have the possibility of developing a complication(s) associated with kidney disease in the future. The drug therapy is not limited, as long as it does not worsen decreased kidney function. For example, the drug therapy can be performed by using an antihypertensive drug, such as an angiotensin-converting enzyme inhibitor (e.g., captopril, enalapril maleate, alacepril, delapril hydrochloride, cilazapril hydrate, lisinopril hydrate, benazepril, imidapril hydrochloride, temocapril hydrochloride, quinapril hydrochloride, trandolapril, perindopril erbumine) and an angiotensin receptor blocker (e.g., losartan potassium, candesartan cilexetil, valsartan, telmisartan, olmesartan medoxomil, irsarbesartan azilsartan); a diuretic (e.g., Lasix, piretanide, azosemide, torasemide, canrenoic acid, potassium), a therapeutic agent for uremia (e.g., Kremezin), an active vitamin D formulation (e.g., Alfarol, Rocaltrol), a phosphate binding agent (e.g., Caltan), a potassium binding agent (e.g., Kayexalate, Kalimate, Argamate Jelly), an alkalizing agent (e.g., sodium bicarbonate), or the like. The dose of the drug and the administration method can be determined depending on the condition of a subject according to a known method.

For a subject in which no effect of dietary therapy was observed, the above-mentioned drug therapy can be performed in addition to further dietary therapy.

Furthermore, for a subject determined to have high phosphorus intake or determined to be at risk of a future decrease in kidney function, dietary therapy can be performed. In addition, the above-mentioned drug therapy may also be performed, if necessary.

### 9. Test Reagent

### 9-1. Test Reagent Comprising Antibody Against Kidney Function Prediction Marker

The present invention provides a test reagent comprising an antibody against kidney function prediction marker used in the above “2. Method for obtaining each measurement value” for the above “4. Evaluation of kidney function,” “5. Estimation of phosphorus intake,” “6. Prediction of possibility of developing complication associated with kidney disease,” and “7. Determination of effect of dietary therapy.”

As the antibody against kidney function prediction marker, those described in the section “1. Explanation of terms” can be used.

The test reagent of this embodiment comprises at least one antibody against kidney function prediction marker. When the antibody against kidney function prediction marker is a polyclonal antibody, it may be a polyclonal antibody obtained by immunization with one kind of antigen, or may be a polyclonal antibody obtained by immunizing the same individual in parallel with two or more kinds of antigens. Further, polyclonal antibodies obtained by inoculating two or more kinds of antigens individually into different animals may be mixed. When the antibody against kidney function prediction marker is a monoclonal antibody, it may be a monoclonal antibody produced from one kind of hybridoma, or may be two or more kinds of multiple monoclonal antibodies each recognizing the same or different epitopes that are produced from two or more kinds of hybridomas may be contained. Alternatively, at least one kind of polyclonal antibody and at least one kind of monoclonal antibody may be contained as a mixture.

The form of the antibody against the kidney function prediction marker contained in the test reagent is not particularly limited, and may be a dried or liquid state of, for example, antiserum or ascitic fluid containing the antibody against kidney function prediction marker. Alternatively, the form of the antibody against kidney function prediction marker may be a dried state or aqueous solution of the purified antibody against kidney function prediction marker, an immunoglobulin fraction containing the antibody against kidney function prediction marker, or an IgG fraction containing the antibody against kidney function prediction marker.

When the form of the antibody against kidney function prediction marker is a dried or liquid state of antiserum or ascitic fluid containing the antibody against kidney function prediction marker, at least one of stabilizers such as β-mercaptoethanol and DTT; protective agents such as albumin; surfactants such as polyoxyethylene (20) sorbitan monolaurate and polyoxyethylene (10) octylphenyl ether; preservatives such as sodium azide; and the like may be further contained. When the form of the antibody against kidney function prediction marker is a dried state or aqueous solution of the purified antibody against kidney function prediction marker, an immunoglobulin fraction containing the antibody against kidney function prediction marker, or an IgG fraction containing the antibody against kidney function prediction marker, at least one of buffer components such as phosphate buffers; stabilizers such as β-mercaptoethanol and DTT; protective agents such as albumin; salts such as sodium chloride; surfactants such as polyoxyethylene (20) sorbitan monolaurate and polyoxyethylene(10) octylphenyl ether; and preservatives such as sodium azide may be further contained.

In the present invention, the antibody against kidney function prediction marker may be unlabeled, or may be labeled with the above-mentioned labeling substance. The antibody against kidney function prediction marker is preferably labeled with the above-mentioned labeling substance. As the labeling substance, those exemplified in the section “2. Method for obtaining each measurement value” can be used. Moreover, in the present invention, the antibody against kidney function prediction marker for antigen capture may be provided in a state of being immobilized on a solid phase surface or the like. The solid phase and the immobilization are as exemplified in the section “2. Method for obtaining each measurement value.” The solid phase is preferably a microplate.

Further, the test reagent may be provided as a kit.

More specifically, the kit is a test kit for the above “4. Evaluation of kidney function,” “5. Estimation of phosphorus intake,” “6. Prediction of possibility of developing complication associated with kidney disease,” and “7. Determination of effect of dietary therapy,” comprising an antibody against kidney function prediction marker used in the “2. Method for obtaining each measurement value,” including a test reagent containing an antibody against kidney function prediction marker for antigen capture and a test reagent containing an antibody against kidney function prediction marker for detection labeled with a labeling substance.

When the antibody against kidney function prediction marker for antigen capture is bound to a solid phase such as a microplate in advance, the test kit of this embodiment may comprise the solid phase on which the antibody against kidney function prediction marker for antigen capture is immobilized, and the antibody against kidney function prediction marker for detection. Further, when the labeling substance is an enzyme, the kit may comprise a liquid of a substrate for the enzyme.

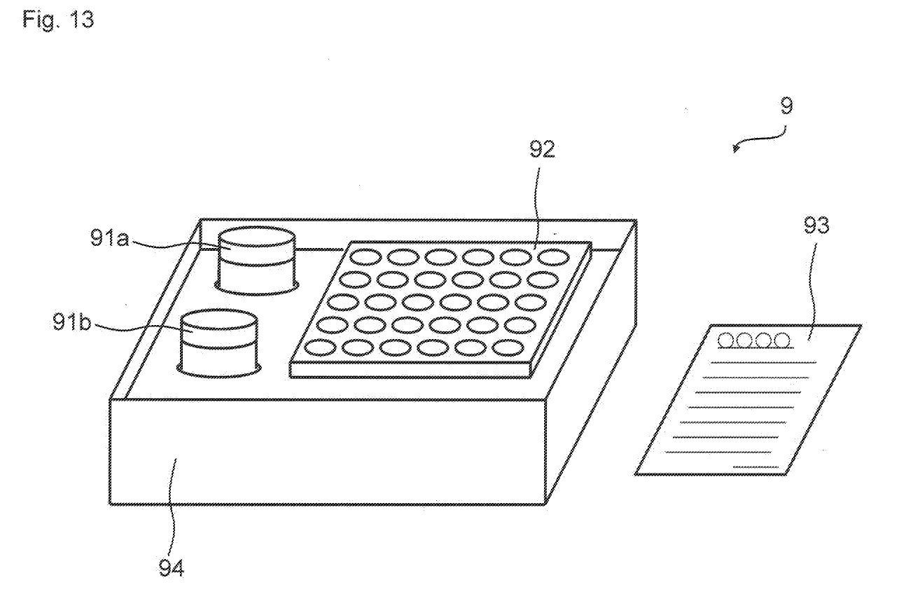

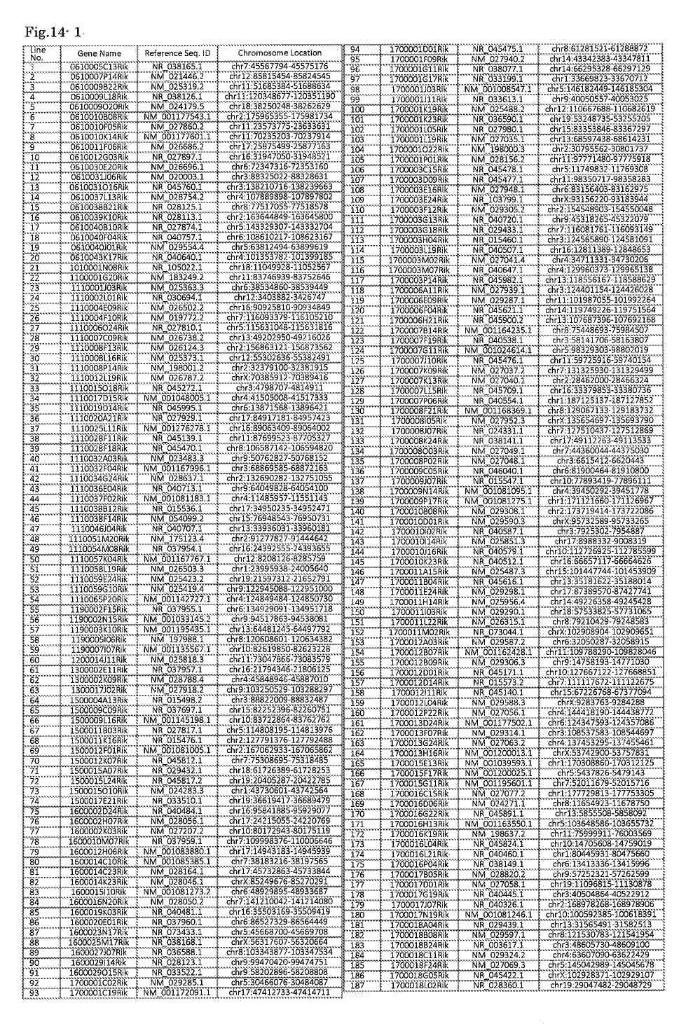

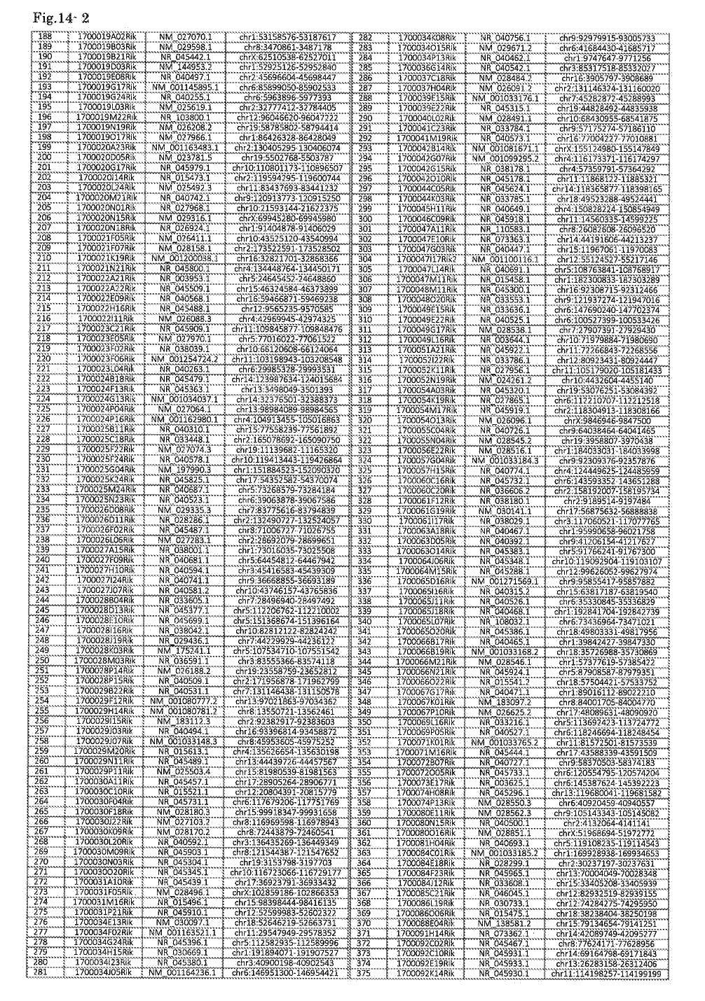

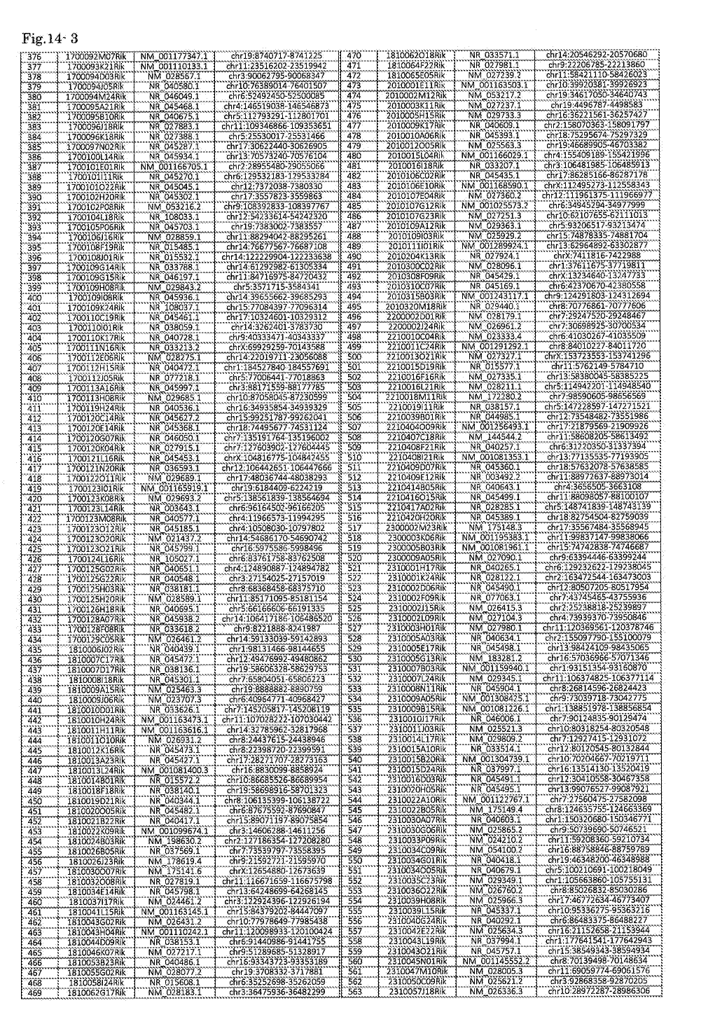

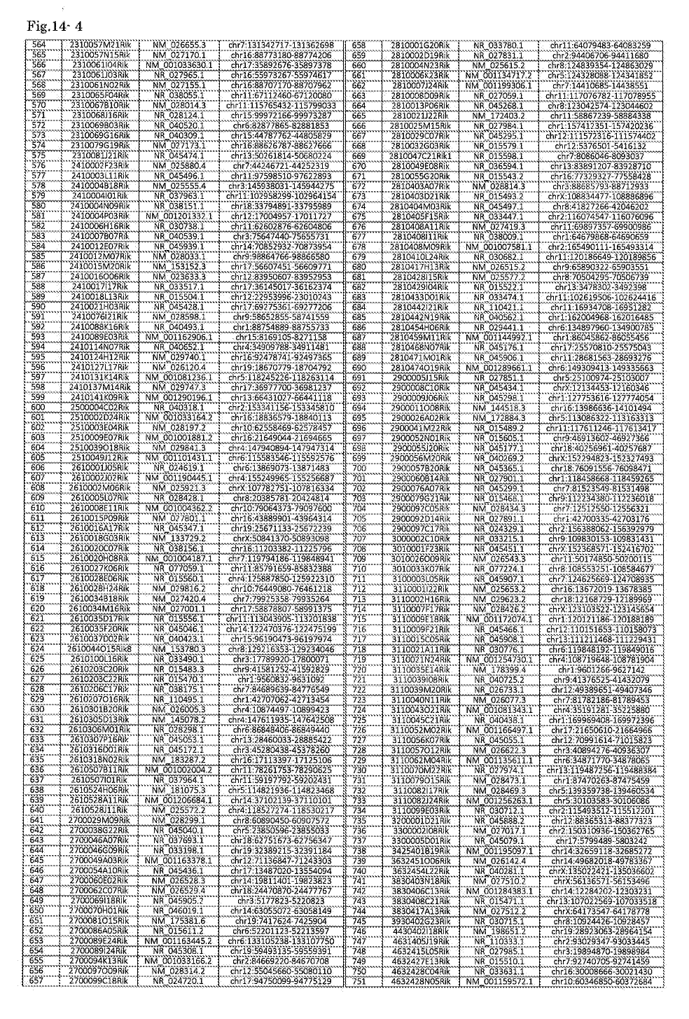

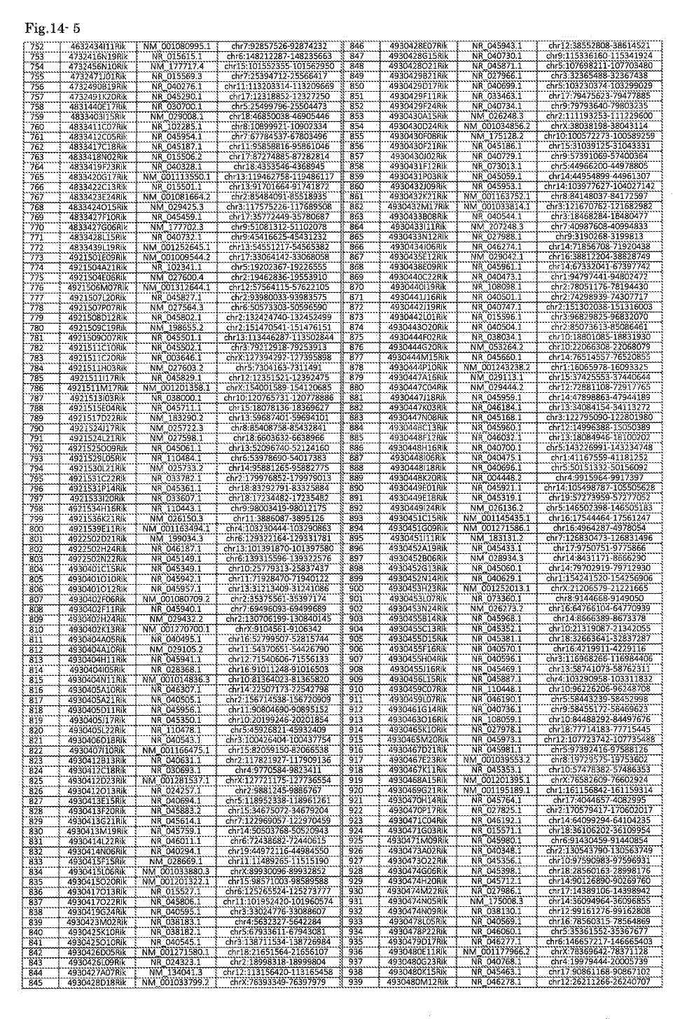

The above-mentioned test kit is, for example, a kit as shown in FIG. 13. A test kit 9 comprises an exterior box 94, a microplate 92 on which an antibody for antigen capture is immobilized, a first container 91a containing an antibody against kidney function prediction marker for detection labeled with a labeling substance, a second container 91b containing a liquid of a substrate that is reactive with the enzyme, and a attachment 93 for the test kit. The attachment 93 can describe the handling method for the test kit, storage conditions, etc. A container containing an aqueous medium for washing or the like may be packed in the exterior box 94.

9-2. Test Reagent Comprising Nucleic Acid for Kidney Function Prediction Marker mRNA Detection

The present invention provides a test reagent for the above “4. Evaluation of kidney function,” “5. Estimation of phosphorus intake,” “6. Prediction of possibility of developing complication associated with kidney disease,” and “6. Determination of effect of dietary therapy,” comprising a nucleic acid for kidney function prediction marker mRNA detection used in the “2. Method for obtaining each measurement value.”

As the nucleic acid for kidney function prediction marker mRNA detection, those described in the section “1. Explanation of terms” can be used.

A test reagent comprising a nucleic acid for kidney function prediction marker mRNA detection used for a microarray may be in a freeze-dried state, or in a state of being dissolved in a solution containing Tris-HCl or like buffer, EDTA, salt, etc. When there are multiple target kidney function prediction marker mRNAs, nucleic acids for detection are preferably placed in different containers. The nucleic acid for kidney function prediction marker mRNA detection may be provided as a microarray chip by immobilizing it on a basal material. The basal material of the microarray is not particularly limited as long as nucleic acids for detection can be immobilized thereon. Examples include glass, polymers such as polypropylene, nylon membranes, and the like. Such a nucleic acid for detection may be immobilized on the basal material according to a known method. For example, a spacer containing a reactive group or a cross-linker for immobilizing the nucleic acid for detection can be used.

Further, the test reagent comprising a nucleic acid for kidney function prediction marker mRNA detection may be provided as a kit comprising not only the reagent, but also a medium, such as paper or a compact disc, on which information about the nucleic acid or information for accessing such information is stored.

A test reagent comprising a nucleic acid for kidney function prediction marker mRNA detection used for RT-PCR may be in a freeze-dried state, or in a state of being dissolved in a solution containing Tris-HCl or like buffer, EDTA, salt, etc. Moreover, regarding primers, forward and reverse primers may be provided in separate containers, or may be provided in a mixed state. Further, when a quantification probe is included, the quantification probe may be provided in a separate container from each primer, or the primers and the quantification probe may be provided in a mixed state.

Further, the test reagent comprising a nucleic acid for kidney function prediction marker mRNA detection may be provided in the form of a kit comprising forward and reverse primers; or forward and reverse primers, a quantification probe, and, if necessary, a attachment. Further, the kit may come with a reagent for quantitative PCR.

### EXAMPLES

The present invention is described in more detail below with reference to examples. The present invention, however, should not be construed as limited to the examples.

Animal experiments in the examples were conducted with the approval of the Animal Care and Use Committee of Advanced Telecommunications Research Institute International. Obtainment of data for humans was approved by the Ethics Committee for Human Tissue Study of Advanced Telecommunications Research Institute International, and conducted after obtaining fully informed consent from the subjects.

### Experimental Example 1: Establishment of Model Mice of Chronic Kidney Disease

Model mice of chronic kidney disease were obtained by feeding them a high-phosphorus diet after unilateral nephrectomy. As a control, mice were obtained by feeding them a low-phosphorus diet after a sham operation.

### 1. Unilateral Nephrectomy

After mice (C57BL/6J, 8 weeks old, male) were anesthetized by intraperitoneal administration of Avertin (250 mg/kg), the skin was incised from the back. The right renal artery and vein, and ureter were ligated. After cutting on the distal side of the ligation, the right kidney was removed, and the incision was closed. The control mice were subjected to a sham operation. In the sham operation, the right renal artery and vein, and ureter were exposed, and the incision was closed without ligation. In order to wait for the mice to completely recover from operative stress, the mice were fed a 0.54% inorganic phosphorus-containing normal diet (CE-2, CLEA Japan, Inc.) for 4 weeks.

### 2. Phosphorus Overload

From 4 weeks after the completion of the operation (12 weeks old), the unilaterally nephrectomized mice were given a 2% inorganic phosphorus-containing high-phosphorus diet (TD.10662, OrientalBioService, Inc.) (hereinafter may be referred to as the “kidney disease group”). The sham-operated mice were given a 0.35% inorganic phosphorus-containing low-phosphorus diet (TD.10662 modified type, OrientalBioService, Inc.) (hereinafter may be referred to as the “Sham group”).

The model mice of chronic kidney disease were obtained by a modification of the method described in Hu M. C. et al. (J Am Soc Nephrol 22, 124-136, 2011). In Hu M. C. et al., the remaining kidney (left kidney) is subjected to ischemia-reperfusion injury at the time of unilateral nephrectomy in Item 1 above. However, in this modification, ischemia-reperfusion was not performed.

### Experimental Example 2: CKD Evaluation

1. 4 weeks after start of high-phosphorus diet (16 weeks old) In histological observation of the kidney 4 weeks after the start of the high-phosphorus diet, a decrease in the stainability of proximal tubule cells to an acidophilic dye (Eosin) was observed in Hematoxylin-Eosin (HE) staining, suggesting that some change occurred in mitochondria. There was no finding of evident fibrosis in the renal interstitium; however, a mild degree of inflammatory cell infiltration was observed.

In physiological observation, an increase in urinary proteins was observed; however, an increase in blood creatinine or phosphorus, or a decrease in creatinine clearance, was not observed.

In gene expression observation by the RNA-Seq analysis described later, it was observed that expression of fibrosis markers (Collagen-1, TGF-1) and inflammatory markers (IL-1, MCP-1) was enhanced.

2. 8 Weeks after Start of High-Phosphorus Diet (20 Weeks Old)

In histological observation of the kidney 8 weeks after the start of the high-phosphorus diet, fibrosis and cell infiltration in the renal interstitium, i.e., kidney fibrosis, was observed.

In physiological observation, an increase in urinary proteins and an increase in blood creatinine, phosphorus, and FGF23 were observed; however, creatinine clearance rather increased, indicating the state of so-called hyperfiltration.

3. 12 Weeks after Start of High-Phosphorus Diet (24 Weeks Old)

12 weeks after the start of the high-phosphorus diet, the above-mentioned findings observed 8 weeks after the start of the high-phosphorus diet further progressed. In some cases, calcification was observed at the cortico-medullary junction of the kidney and around thereof. However, none of the mice died.

### Example 1: Observation of Multiple Organs

### 1. Collection of Each Organ

Organs or tissue (pancreas, skull, brain, hypophysis, kidney, adrenal gland (AG), liver, spleen, thymus, heart, lung, salivary gland (SG), thyroid gland (TG), aorta, skeletal muscle (SM), skin, testis, fat, eye, stomach, jejunum, ileum, and colon) was collected 1 week (early stage) and 4 weeks (middle stage) after the start of the high-phosphorus diet (the low-phosphorus diet in the Sham group).

The animals from which the organs or tissues were to be collected were euthanized by cervical dislocation after blood was collected from the orbit under anesthesia, and the organs and tissue were collected. After the wet weights of the collected organs and tissue were measured, the organs and tissue were rapidly frozen in liquid nitrogen and stored at −80° C.

### 2. Analysis of RNA

(1) Extraction of RNA from Each Tissue

Each cryopreserved tissue was individually homogenized in TRIzol Reagent (Life Technologies) with Cell Destroyer PS1000 (Bio Medical Science Inc.) or PT 10-35 GT Polytron homogenizer (KINEATICA). After incubation at room temperature for 5 minutes to separate proteins, 0.2 mL of chloroform was added per mL of TRIzol, and the tube was capped. Subsequently, the mixture was vortexed vigorously for 15 seconds. After the vortexing, the mixture was incubated at room temperature for 3 minutes and centrifuged at 12,000 g for 15 minutes at 4° C., and the RNA-containing aqueous layer was collected in a fresh tube. An equal amount of 70% ethanol was added to the collected aqueous layer, and mixed. Then, 700 μL of the mixture was applied to each RNeasy Mini column (Qiagen), and purified RNAs were collected according to the RNeasy Mini kit (Qiagen) standard protocol. The quality and concentration of each of the collected RNAs was evaluated by 1% agarose electrophoresis and NanoDrop.

### (2) Obtaining RNA-Seq Data

RNA-Seq data was obtained using the measurement samples described above by the following procedure in Macrogen Japan Corp.

i. Quality Check

Quality testing of the samples was performed based on the following item.
Concentration measurement and quality check using an Agilent 2200 TapeStation System
   ii. Preparation of Sample

A library for sequencing was prepared using 500 to 1000 ng of each total RNA sample that passed the quality testing as a template with Illumina's TruSeq RNA Sample Prep Kit according to the standard protocol in the following manner.

(a) Purification of poly(A)+RNA using Oligo-dT beads
(b) Poly(A)+RNA fragmentation
(c) Reverse transcription 2nd strand cDNA synthesis
(d) Terminus repair and 3′A addition
(e) Adapter ligation
Note: The adapters contain index tags for identification of specimens.
(f) PCR amplification
(g) Purification and removal of low-molecular-weight substances (<200 bp) using AMPure XP beads
iii. Obtaining Data Using Next-Generation Sequencer

Nucleotide sequence data were obtained using an Illumina HiSeq 4000 next-generation sequencer by reading 100 bases according to the paired-end method.

### (3) Analysis of RNA-Seq Data

### (3-1) Analysis of Output Data Obtained Using Next-Generation Sequencer

The following information processing was carried out for the output data.

i. Base calling: text data of nucleotide sequences were obtained from the output raw data of analysis (image data).
ii. Filtering: selection of read data by predetermined filtering was performed.
iii. Sorting based on index sequences: sample data were sorted based on index information.

### (3-2) Secondary Analysis of Output Data

The data file (Fastq format) obtained using Illumina HiSeq was uploaded on Galaxy (https://usegalaxy.org/) downloaded to a local server. Thereafter, analysis was carried out using Bowtie2 (http://bowtie-bio.sourceforge.net/bowtie2/index.shtml) to map each sequence to mouse genome map information mm10. The BAM file obtained using Bowtie2 was analyzed using Cufflinks (http://cole-trapnell-lab.github.io/cufflinks/) to calculate FPKM (RPKM) for each gene.

### (3-3) Results

Values were calculated by dividing the RNA expression level of each gene (FPKM value) by the expression level of the corresponding RNA (FPKM value) in the mice of the sham group (hereinafter also referred to as “CKD/sham”). RNAs in which CKD/sham is more than 1 or less than 1 were classified as group 2, RNAs in which CKD/sham is more than 1.5 or less than 0.67 were classified as group 3, RNAs in which CKD/sham is more than 2 or less than 0.5 were classified as group 4, and RNAs in which CKD/sham is more than 5 or less than 0.2 were classified as group 5 (FIG. 15). All FPKM values of less than 1 were treated as “1.” FIG. 16 lists genes in which Sham>1 and CKD/Sham>5, genes in which Sham<1 and CKD/Sham>10, and genes in which Sham>10 and CKD/Sham <0.3. In particular, the genes shown in FIG. 16 were considered to best reflect the state of kidney function and phosphorus intake. Especially regarding Defb8, CKD/sham was decreased to 0.2 in the skin in the high-phosphorus diet. Regarding Defa24, CKD/sham was 2.5 in the stomach at the early stage of the high-phosphorus diet, but 1.0 at the middle stage. Moreover, regarding Defa24, CKD/sham was 5.3 in the skeletal muscle and 4.5 in the testis, at the middle stage of the high-phosphorus diet. The expression of Defb1, Defb10, Defb12, Defb14, Defb1S, Defb18, Defb19, Defb2, Defb20, Defb21, Defb22, Defb23, Defb25, Defb26, Defb28, Defb29, Defb30, Defb35, Defb37, Defb39, Defb41, Defb42, Defb43, Defb45, Defb47, and Defb48 significantly increased in the adipose tissue at the early stage of the high-phosphorus diet. Meanwhile, regarding Defb1, Defb10, Defb12, Defb14, Defb15, Defb18, Defb19, Defb2, Defb20, Defb21, Defb22, Defb23, Defb25, Defb26, Defb28, Defb29, Defb30, Defb35, Defb37, Defb39, Defb41, Defb47, and Defb48, CKD/sham was 1.0 in the adipose tissue at the middle stage of the high-phosphorus diet. Oscar was observed to show increased expression in the skull at both the early and middle stages of the high-phosphorus diet. Proline-rich proteins (Prb1, Prh1, Prp2, Prpmp5) were observed to show increased expression in the salivary gland at both the early and middle stages of the high-phosphorus diet. Spp1 was observed to show increased expression in the lung, skull, kidney, and heart at both the early and middle stages of the high-phosphorus diet. Dnase1 was observed to show increased expression in the salivary gland and decreased expression in the kidney, at both the early and middle stages of the high-phosphorus diet. Slc7a8 was observed to show increased expression in the aorta at both the early and middle stages of the high-phosphorus diet. Regarding Anpep, significantly increased expression was observed (CKD/sham was 28.3) in the thyroid gland at the early stage of the high-phosphorus diet, but CKD/sham was 4.0 at the middle stage. Regarding Hamp2, significantly increased expression was observed (CKD/sham was 46.1) in the skin at the early stage of the high-phosphorus diet, but CKD/sham was 3.9 at the middle stage. Slco1a1 was observed to show decreased expression in the kidney and liver at the early stage of the high-phosphorus diet. Aplnr was observed to show decreased expression in the adrenal gland, aorta, lung, hypophysis, skin, skull, skeletal muscle, spleen, thyroid gland, kidney, heart, and adipose tissue at both the early and middle stages of the high-phosphorus diet. Thus, the expression of these genes was considered to best reflect, in particular, the state of kidney function and phosphorus intake.

### Example 2: Expression of PRPs in Mouse Model of Excessive Phosphorus Load

### 1. Production of Mouse Model of Phosphorus Load

Mice (C57BL/6, 7 weeks old, male) that had not been subjected to unilateral nephrectomy were fed a 0.54% inorganic phosphorus-containing normal diet (CE-2, CLEA Japan, Inc.) for 1 week. Thereafter, the mice were given a 2% inorganic phosphorus-containing high-phosphorus diet (TD.10662, OrientalBioService, Inc.) as a high-phosphorus diet or 0.35% inorganic phosphorus-containing low-phosphorus diet (TD.10662 modified type, OrientalBioService, Inc.) as a low-phosphorus diet. In each group, n-10.

### 2. Analysis of Proline-Rich Protein (PRP) Gene Expression in Salivary Glands

### (1) Collection of Tissue

The salivary glands and skull were collected 1 week (9 weeks old) and 4 weeks (12 weeks old) after the start of the high-phosphorus diet or the low-phosphorus diet in the mice. Regarding the salivary glands, the submandibular glands, sublingual glands, parotid glands, and surrounding connective tissue (including lymph nodes) were separately collected individually. The animals from which the tissue was to be collected were euthanized by cervical dislocation after being anesthetized by intraperitoneal administration of Avertin (250 mg/kg), and the tissue was collected. After the weight of the collected tissue was measured, the tissue was rapidly frozen in liquid nitrogen, and stored at −80° C.

### (2) Analysis of RNA

i. Extraction of RNA from Each Tissue

Each cryopreserved tissue was individually homogenized in TRIzol Reagent (Life Technologies) with Cell Destroyer PS1000 (Bio Medical Science Inc.). After incubation at room temperature for 5 minutes to separate proteins, 0.2 mL of chloroform was added per mL of TRIzol, and the tube was capped. Subsequently, the mixture was vortexed vigorously for 15 seconds. After the vortexing, the mixture was incubated at room temperature for 3 minutes and centrifuged at 12,000 g for 15 minutes at 4° C., and the RNA-containing aqueous layer was collected in a fresh tube. An equal amount of 70% ethanol was added to the collected aqueous layer, and mixed. Then, the mixture was applied to an RNeasy Mini column (Qiagen), and purified RNAs were collected according to the RNeasy Mini kit (Qiagen) standard protocol. The quality and concentration of each of the collected RNAs was evaluated by 1% agarose electrophoresis and NanoDrop.

ii. cDNA Synthesis and Quantifying Relative Expression Level by Real-Time PCR

1 μg of total RNA obtained from each tissue was used as a template for cDNA synthesis, and cDNA was synthesized using Oligo dT20 primer according to the standard protocol of Superscript III First-Strand Synthesis SuperMix (Life Technologies). After the synthesized cDNA was diluted 20-fold with TE buffer (10 mM Tris-HCl, pH 8.0, 0.1 mM EDTA), real-time PCR was performed with a LightCycler 480 II (Roche) according to the standard protocol of LightCycler 480 SYBR Green I Master (Roche), and Cp values were measured. The relative expression level (2^{-ΔCp}) of each gene to a reference gene was quantified by comparing the Cp value obtained for each gene with the Cp value for β2-microglobulin (B2m) or Maea as the reference gene. The expression of PRP genes (PRPs: Prb1, Prh1, Prp2, Prpmp5) was examined in each salivary gland tissue, and the expression of FGF23 was examined in the skull. The primer pairs used in the real-time PCR are as shown in Table 4.

TABLE 4

| | Gene | Forward (SEQ ID NO) | Reverse (SEQ ID NO) | | |
|---|---|---|---|---|---|
| 1 | B2m | GCTCGGTGACCCTGGTCTTT | (1) | AATGTGAGGCGGGTGGAACT | (2) |
| 2 | Maea | AAGACCTTGAGTAGTTGCCCA | (3) | TGCTCGATCCTACGTTTGCAG | (4) |
| 3 | Prb1 | ACCCCAGCATGGAAACAAAG | (5) | AAGAATGGTATTGAAGTCATCTGTC | (6) |
| 4 | Prh1 | ACCCCGTGAAGAAAATCAGAA | (7) | TAACAGGCGGTCTTGGTTGG | (8) |
| 5 | Prp2 | TGGTGGTCCTGTTTACAGTGG | (9) | TTCTGAAGTTCTTCACGGGGT | (10) |
| 6 | Prpmp5 | CCTACGAAGACTCAAATTCTCAGC | (11) | GAGGACCATGGTGGTGTCC | (12) |

### (3) Results

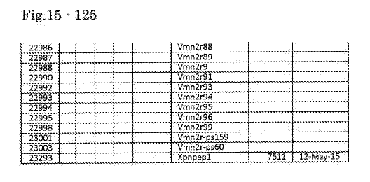

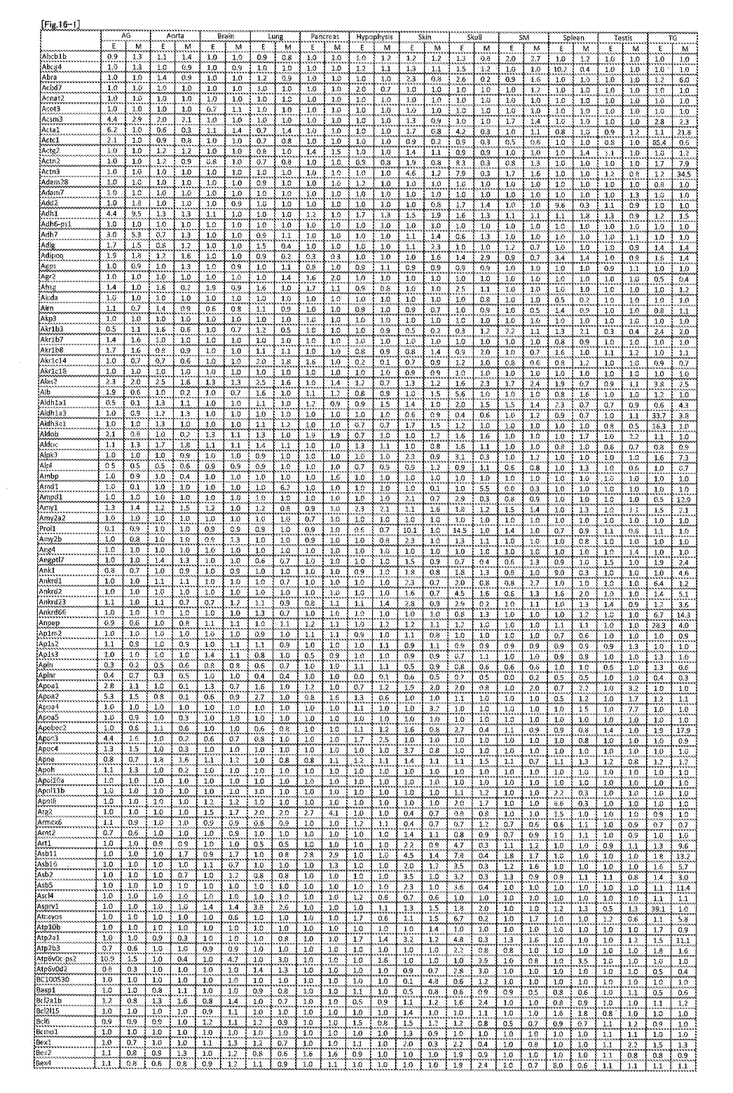

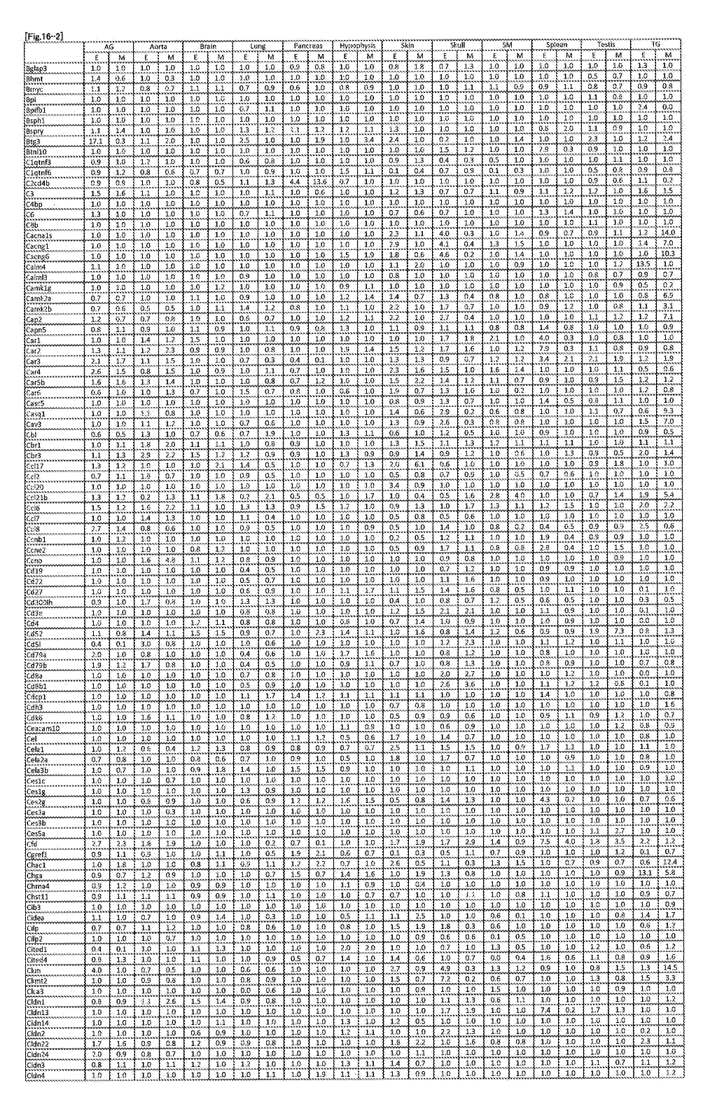

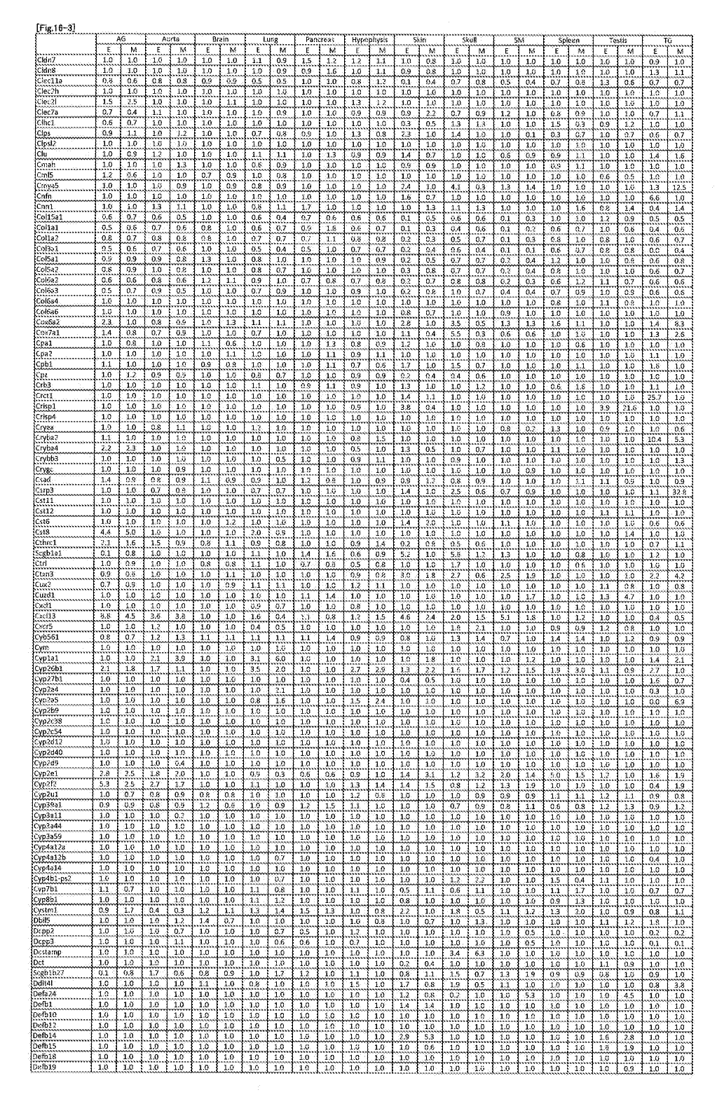

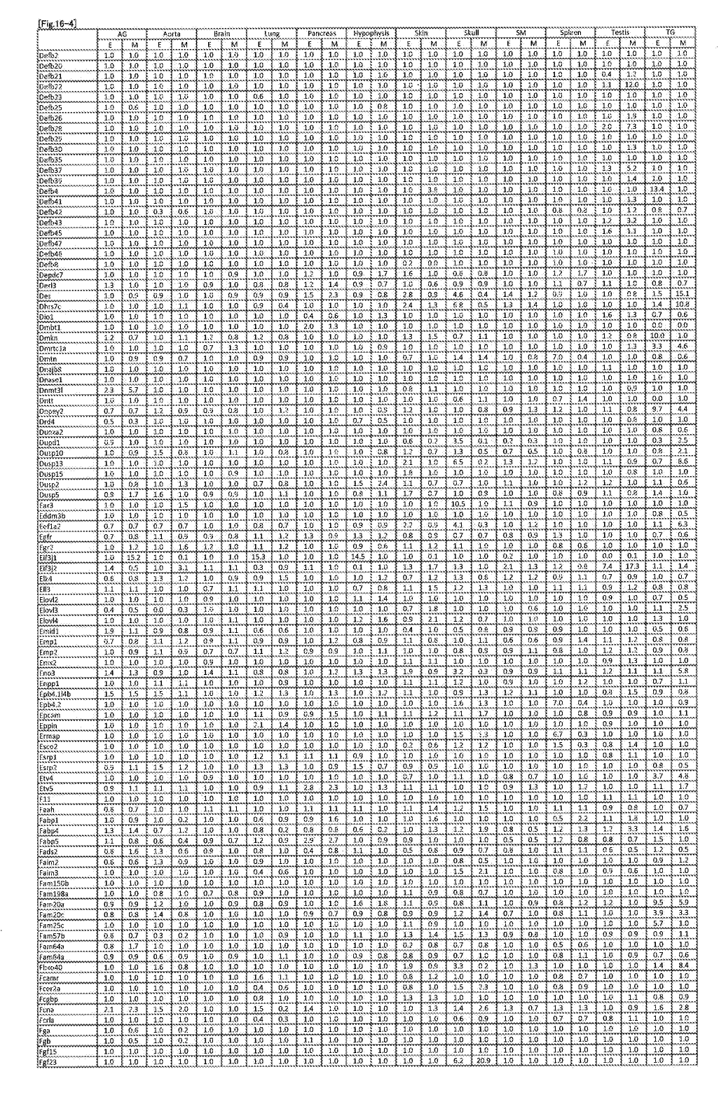

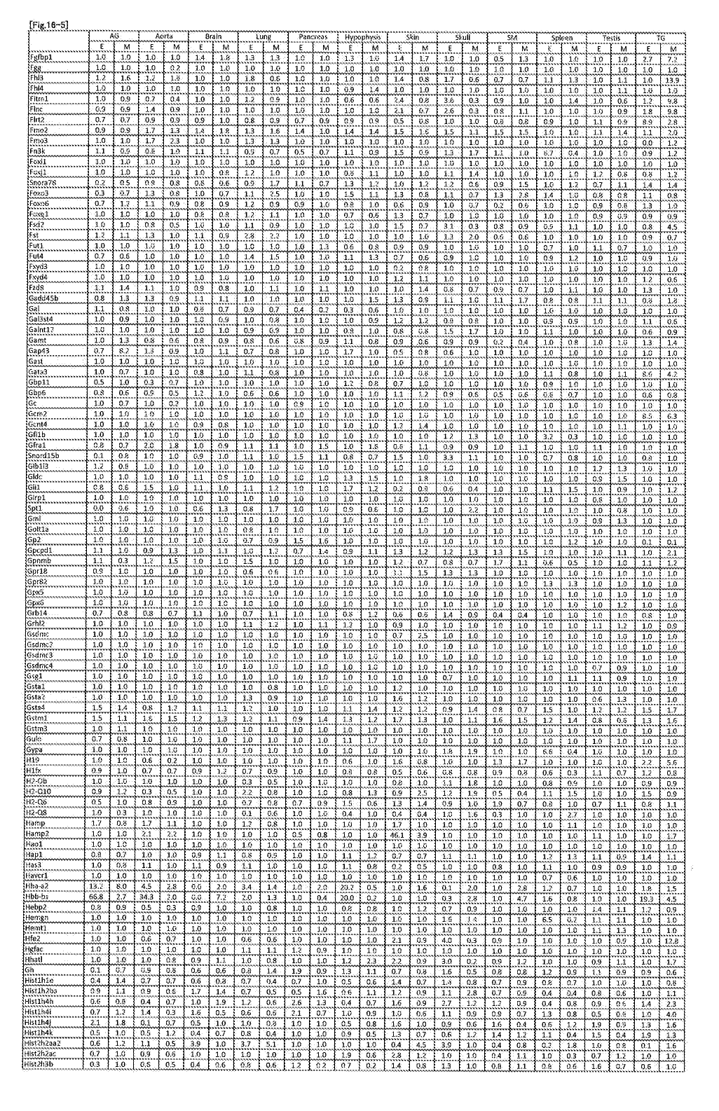

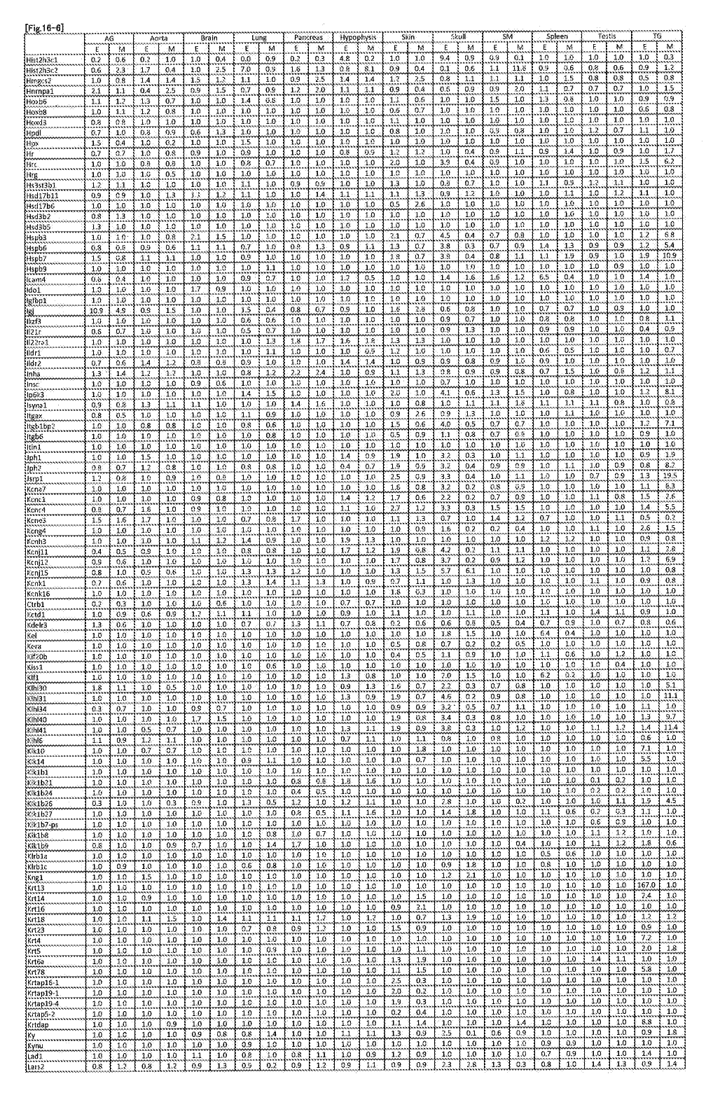

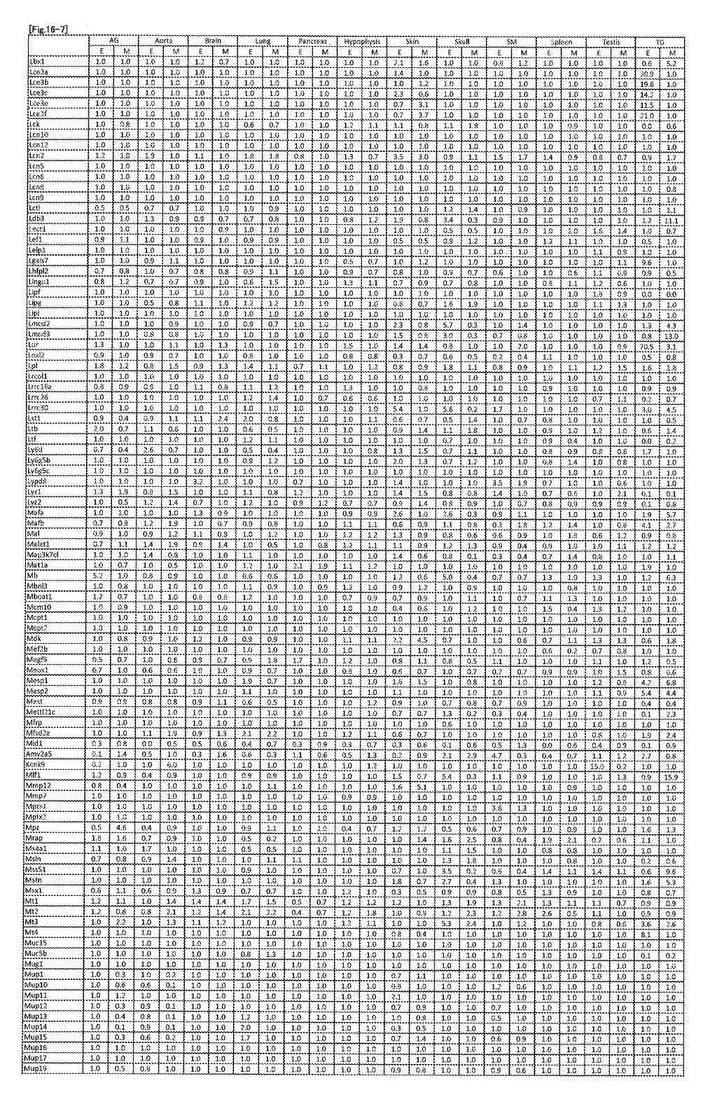

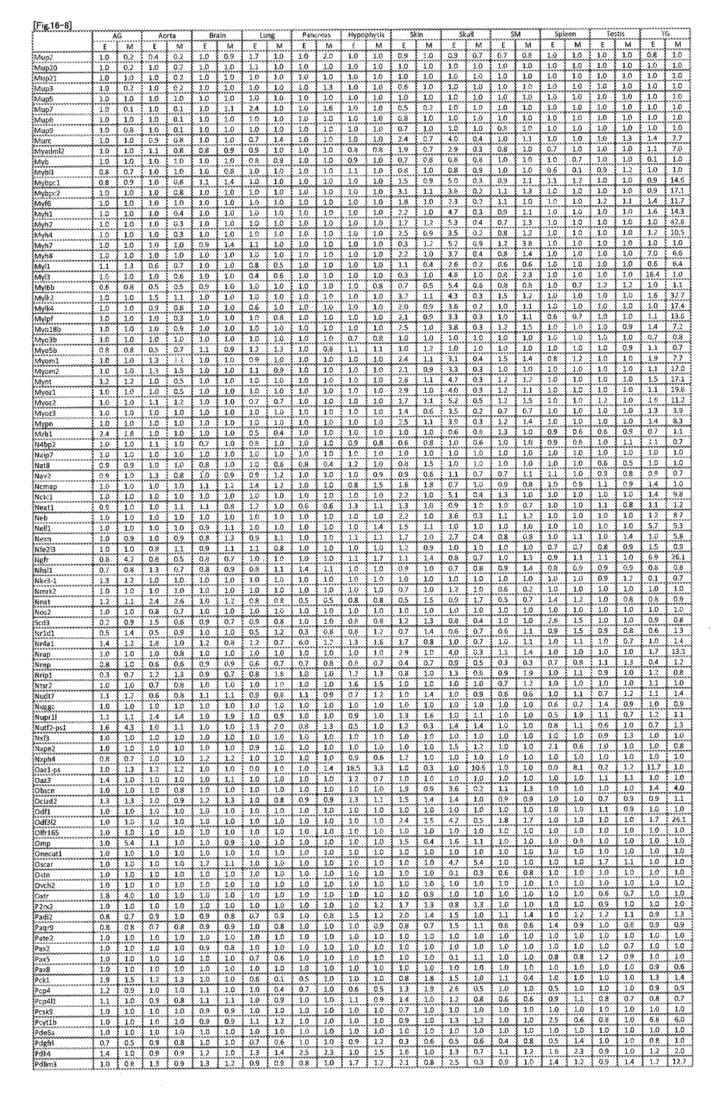

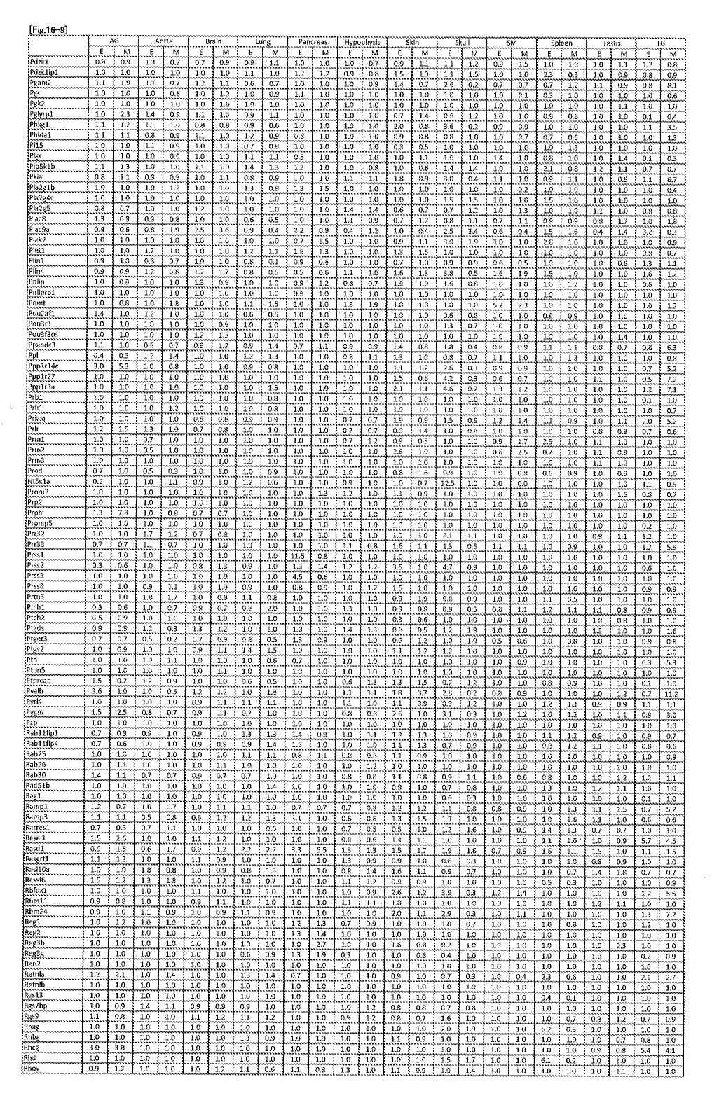

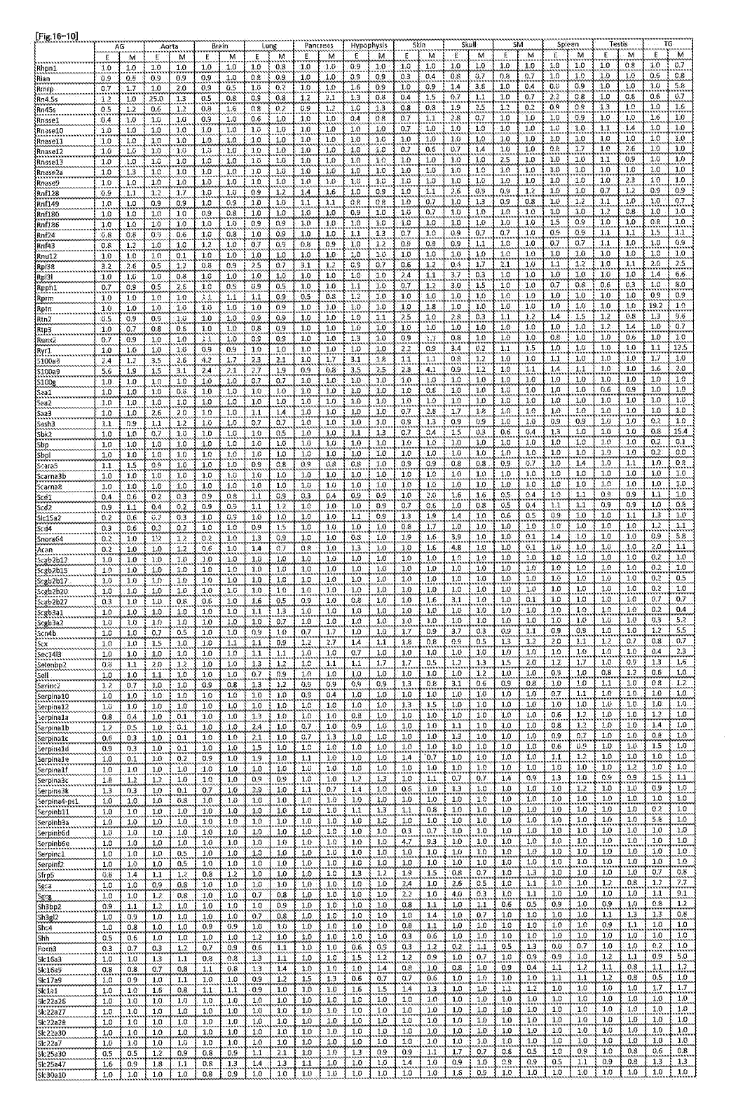

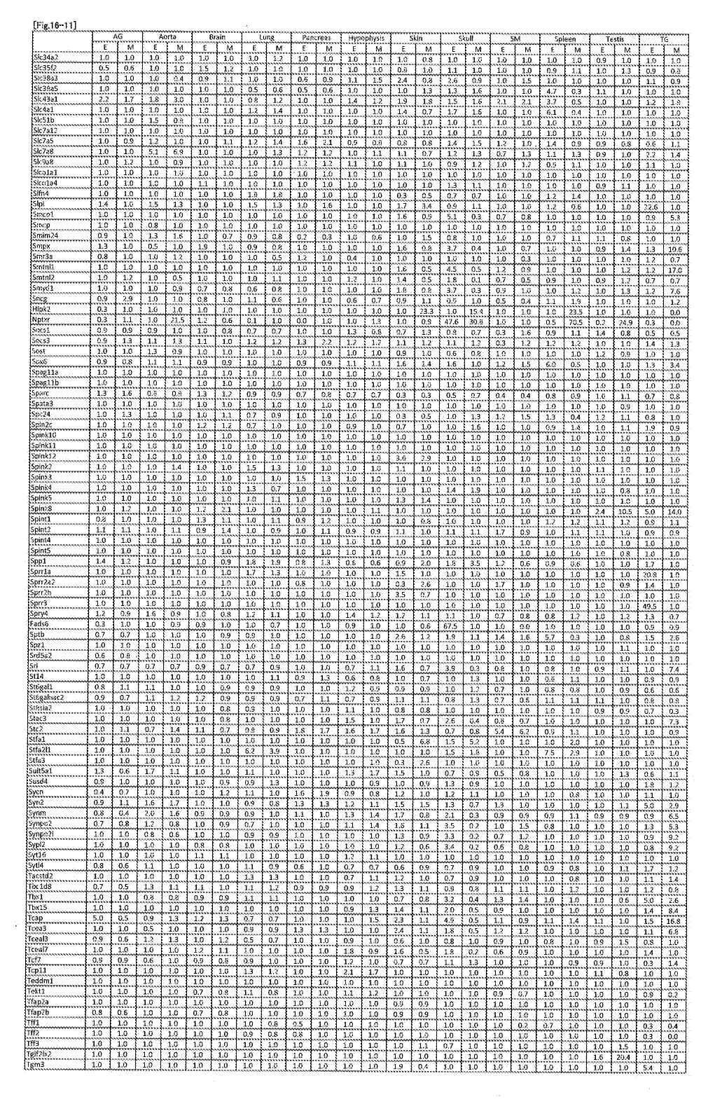

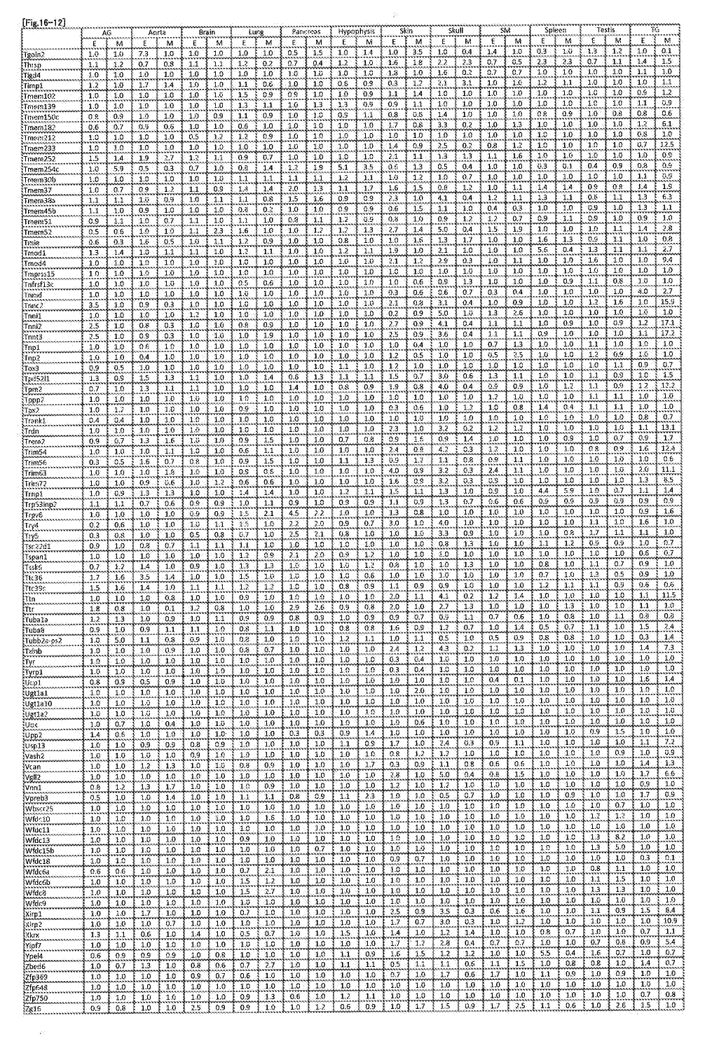

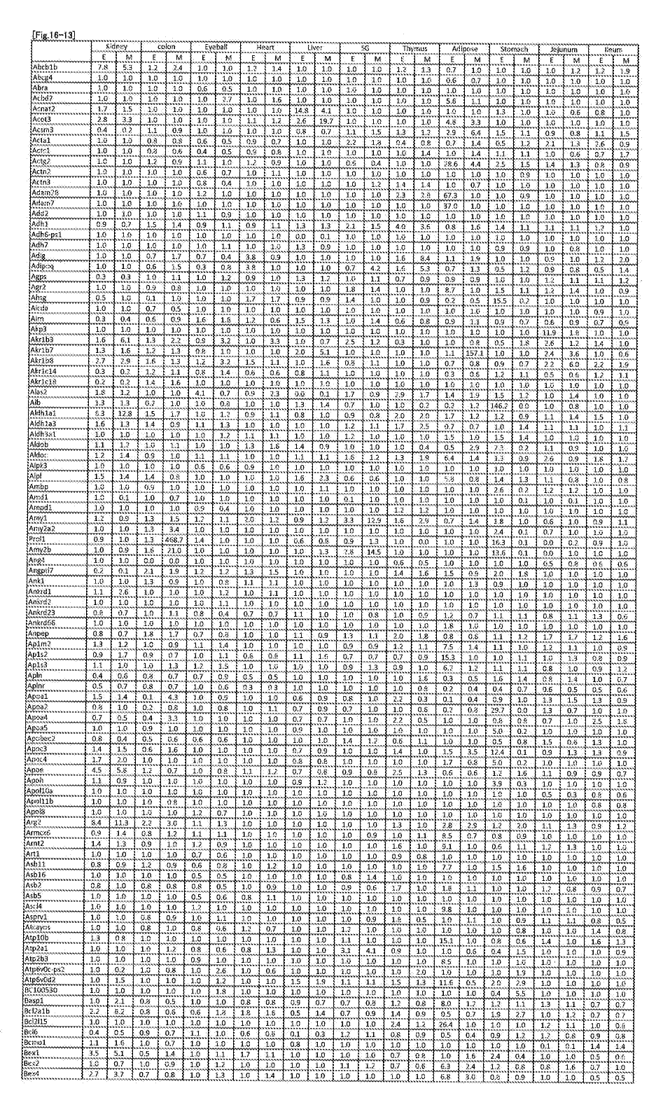

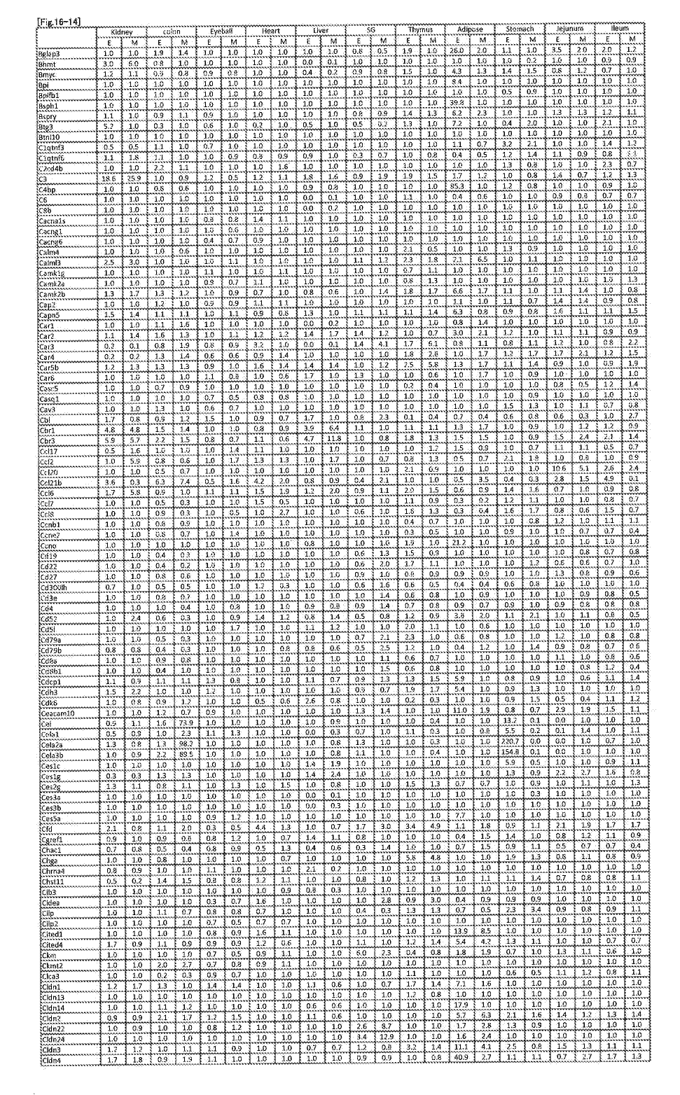

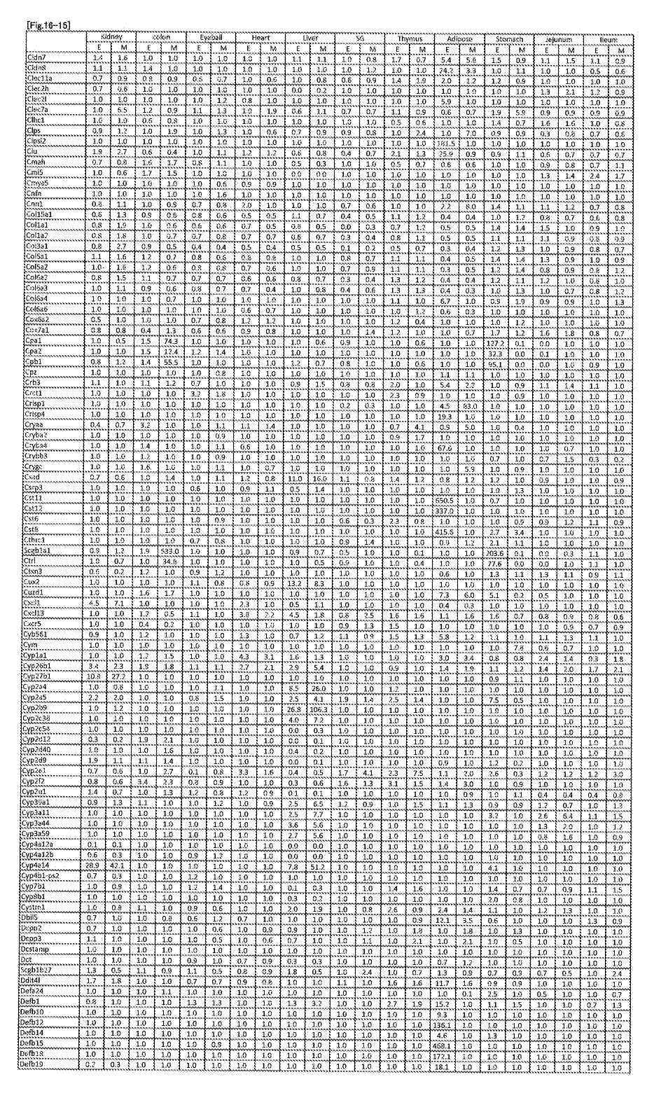

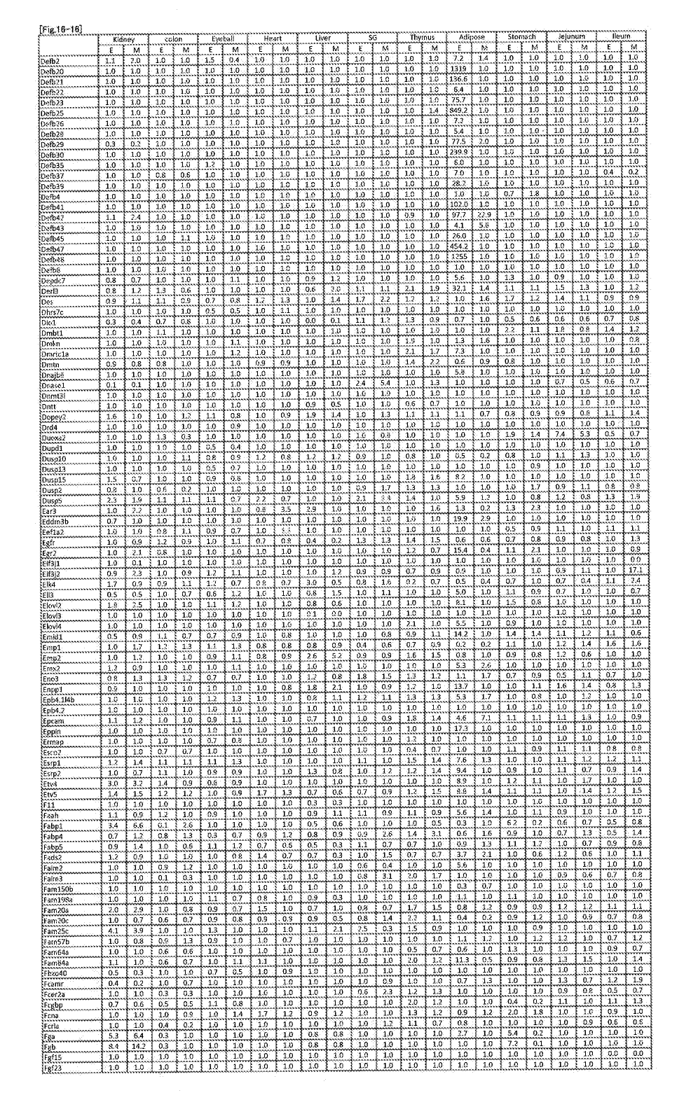

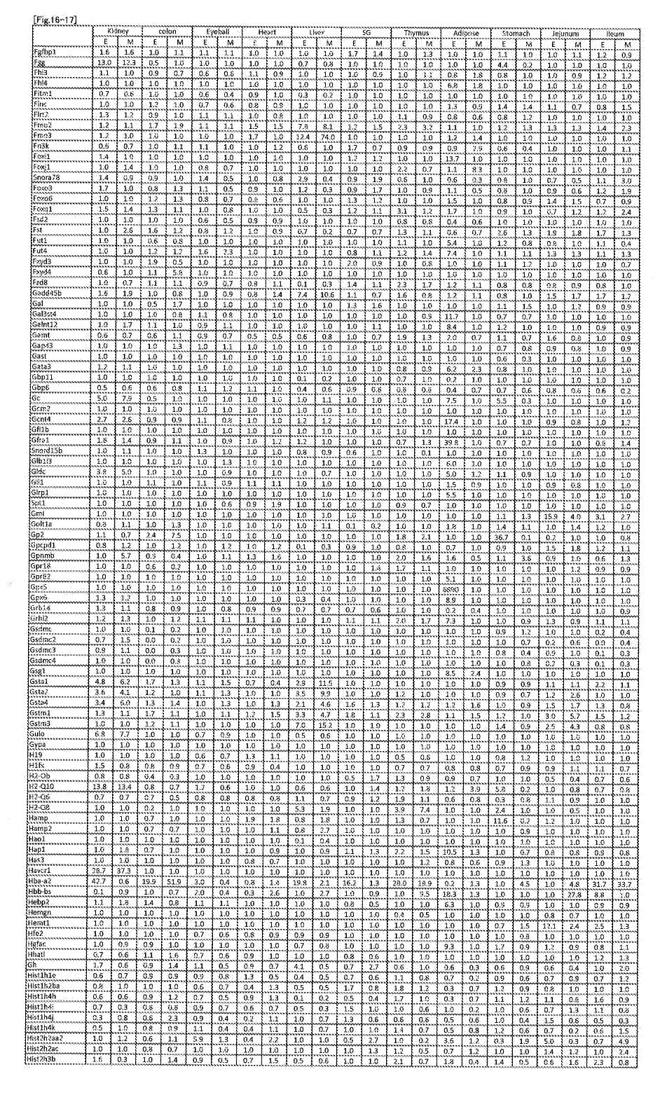

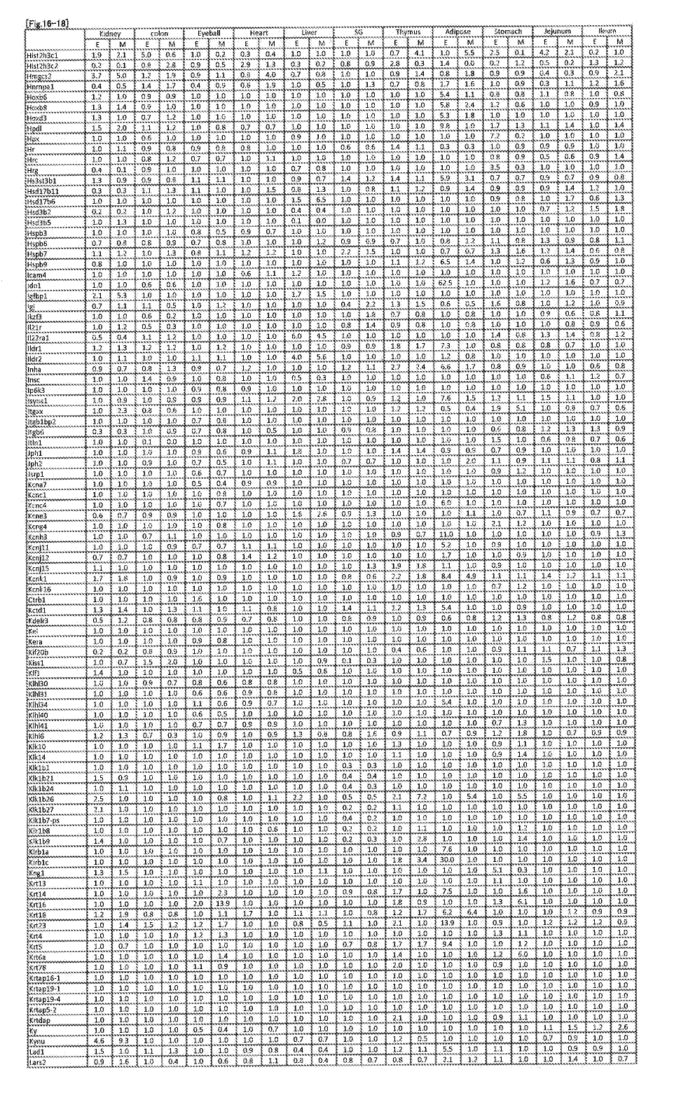

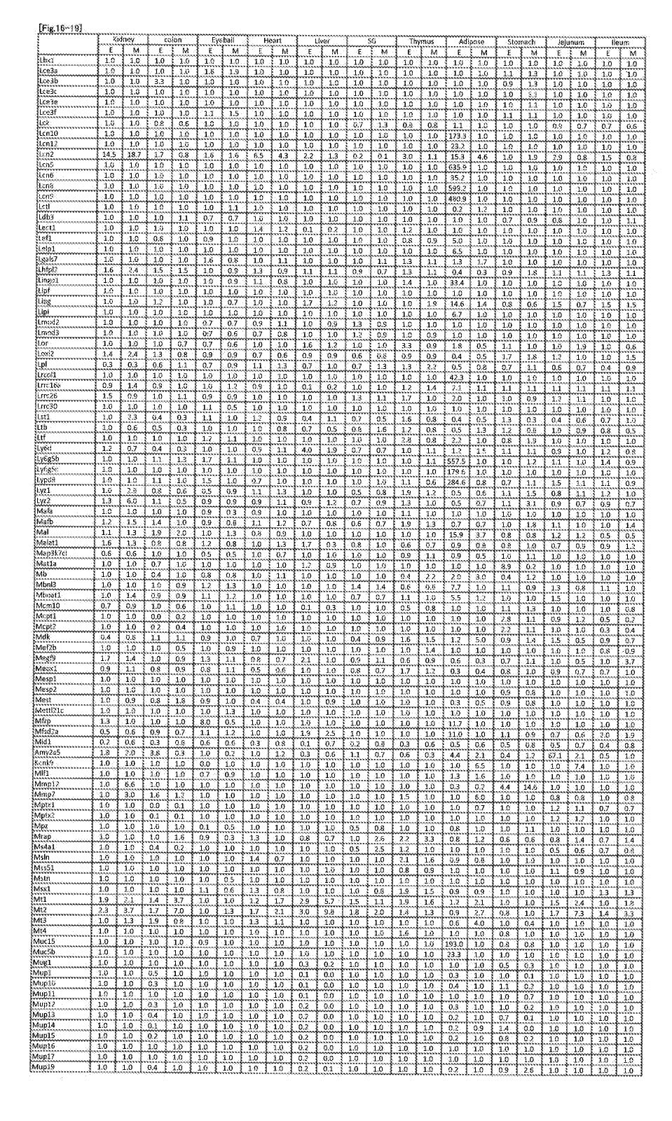

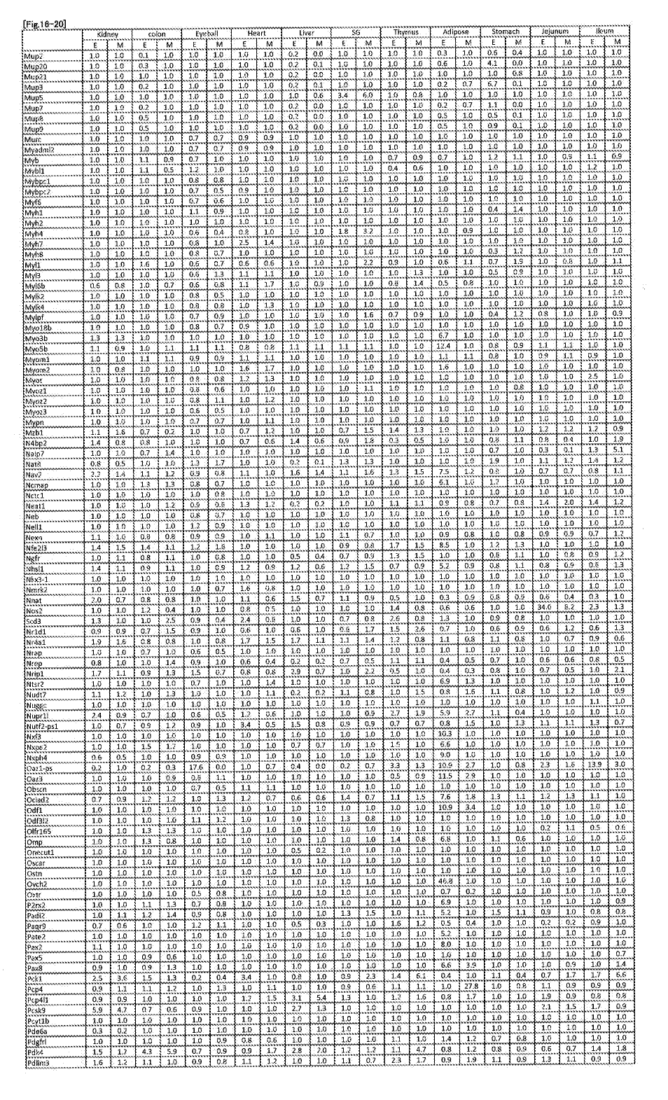

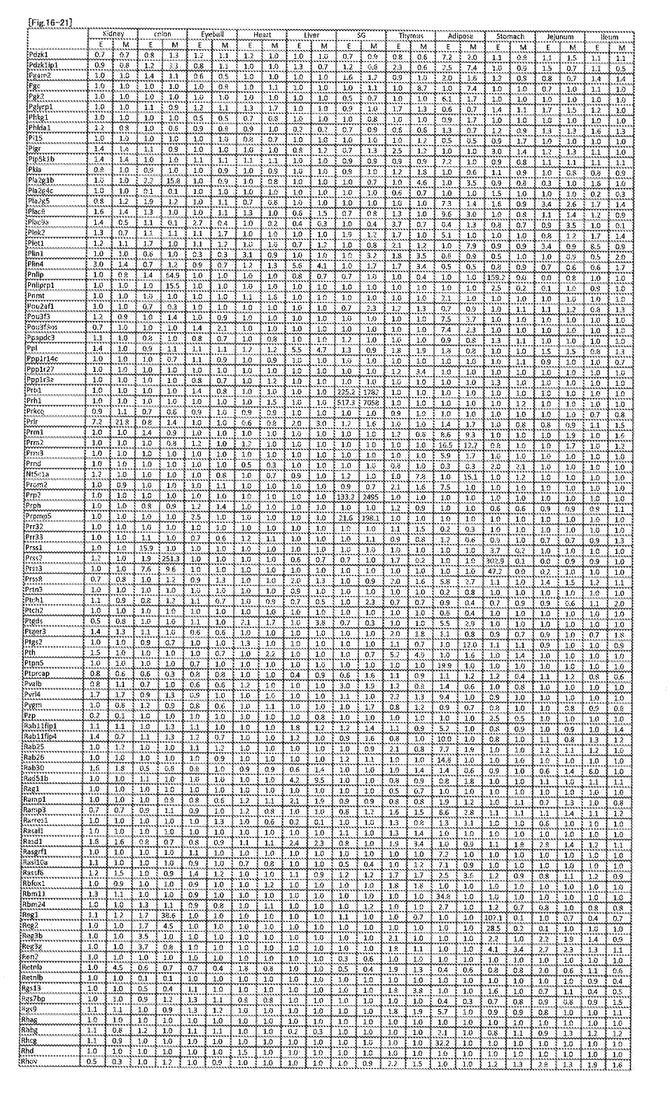

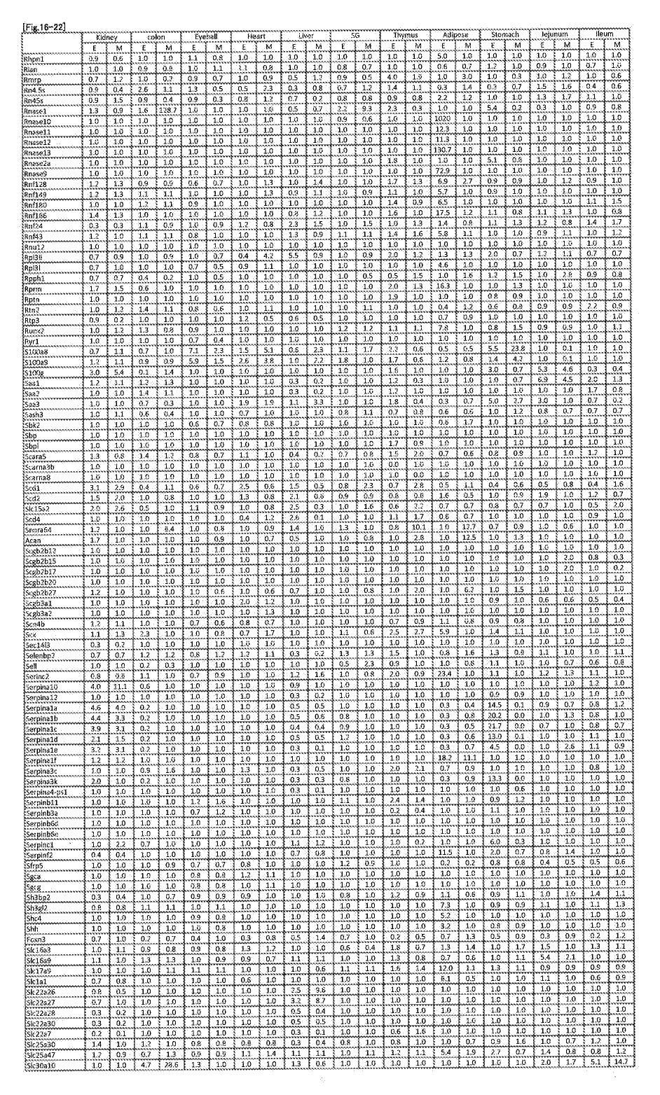

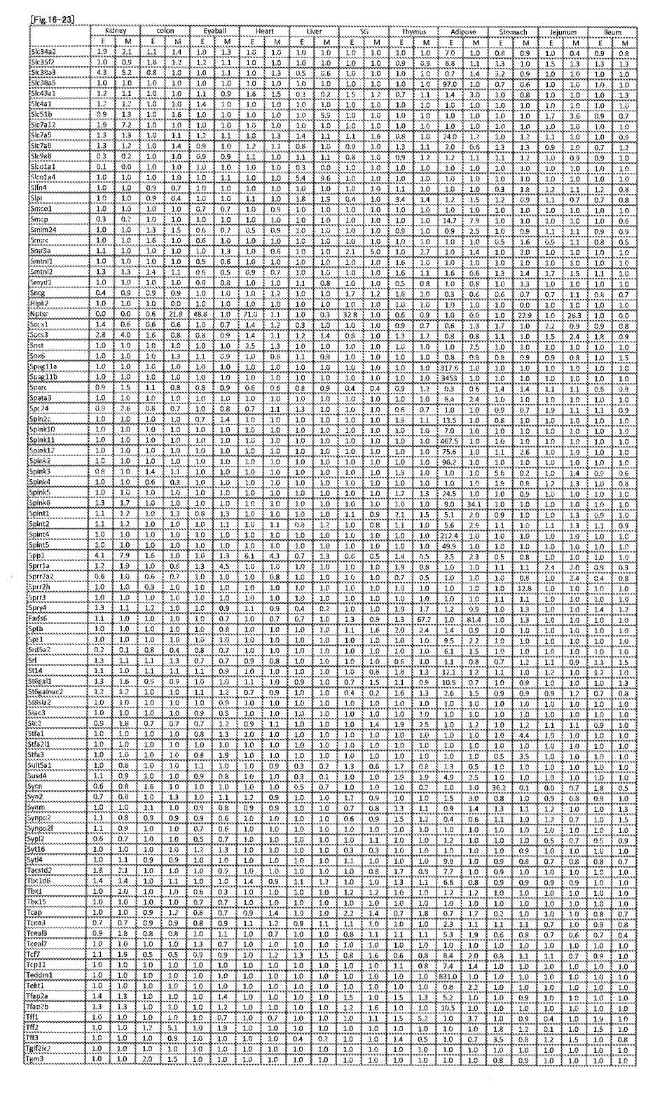

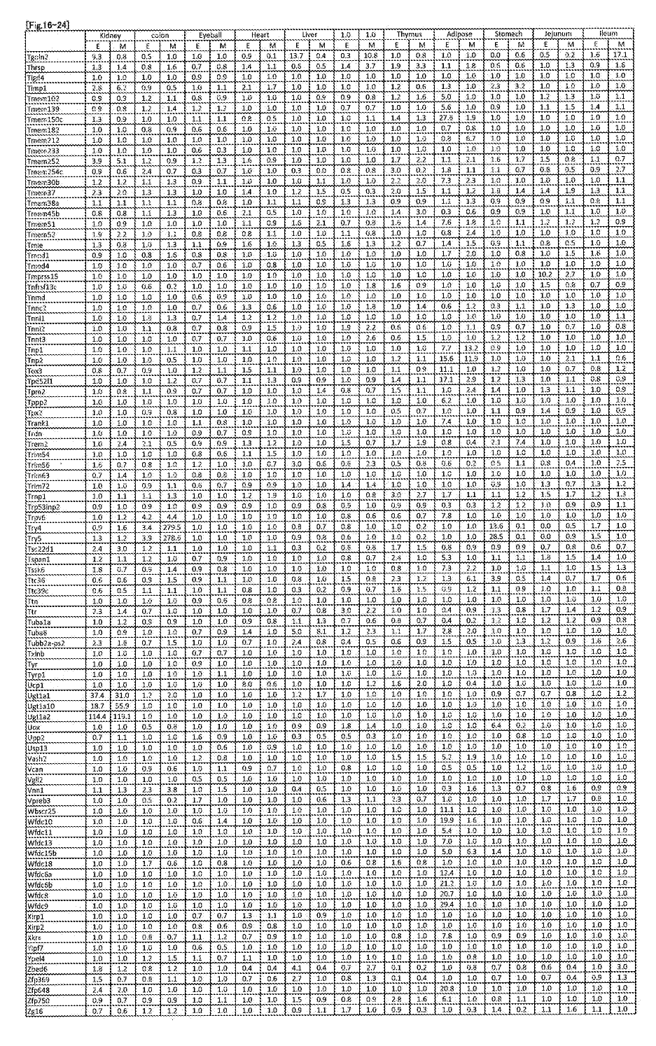

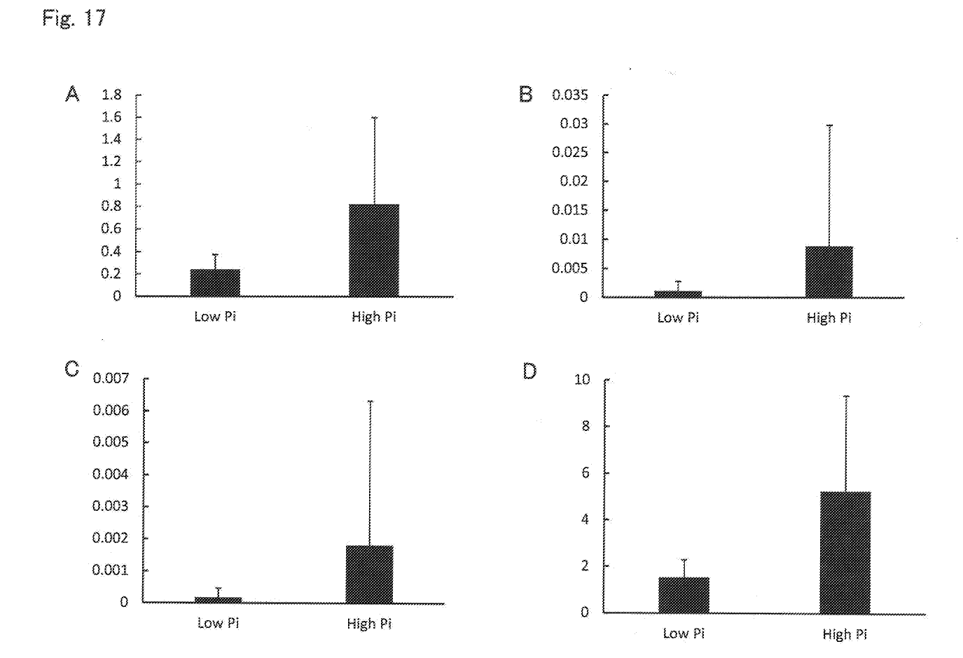

As shown in FIG. 17, the expression levels of Prb1 (FIG. 17A), Prh1 (FIG. 17B), Prp2 (FIG. 17C), and Prpmp5 (FIG. 17D) were increased in the parotid glands in the group fed the high-phosphorus diet (High Pi) than in the group fed the low-phosphorus diet (Low Pi).

### Example 3: Expression of PRPs in Subject Who Ingested High-Phosphorus Diet

### (1) Subject

Subjects were selected according to the following inclusion criteria.

i. Person who is able to fully understand this study plan, and is able to give consent by themselves
ii. Person aged 20 years or older at the time of obtaining consent

However, persons who meet any of the following criteria were excluded from the subjects.

i. Person with a history of kidney disease to date
ii. Person having a cardiovascular risk factor (obesity, high blood pressure, diabetes, smoking)
iii. Person who is deemed unsuitable as a subject by a researcher

### (2) High-Phosphorus Diet Ingestion

A high-phosphorus diet group (group A) was asked to ingest a high-phosphorus diet in addition to a normal diet. A normal diet group (group B) was a subject group that ingests a normal diet; the normal diet group was, however, asked to ingest as few phosphorus-rich foods as possible.

Both group A and group B were asked to refrain from ingesting phosphorus-rich foods (foods listed in Table 5) as much as possible from Day −7. Group A was asked to select one of the dietary patterns shown in Table 6, and ingest the diet from Day 1 for 7 days. Group B was asked to ingest a normal diet (refrain from ingesting phosphorus-rich foods as much as possible). Table 7 shows the diet and saliva collection schedule.

TABLE 5
Fish and shellfish
dried whitebait, dried small sardine, sardine, sand lance, splendid alfonsino, ayu (sweetfish), smelt,
eel, dried squid, dried shrimp, salmon roe, cod roe, sea urchin, etc.
Pulses
freeze-dried tofu, soybean, soybean flour, pea, fermented soybean, etc.
Dairy products
processed cheese, skim milk powder, milk, etc.
Flesh
egg yolk, beef jerky, liver, etc.
Nuts
sesame, pine nut, cashew nut, almond, pistachio, peanut, walnut, etc.
Processed foods rich in inorganic phosphorus as a food additive
pastry, instant noodles, Chinese noodles, confectionery (biscuit, cookie)/seasoning for sprinkling
over rice, rice ball containing solid ingredients, take-out meal in a box, savory bread, instant
Chinese noodles, ham/sausage, bacon, frozen food, hamburger steak, fish meat/fish-paste
product, food boiled in soy sauce, pastry, biscuit, cookie, carbonated drink, etc.
Processed foods in which the following are indicated as ingredients
lye water (potassium phosphate, sodium phosphate)
yeast food (phosphoric acid salt)
emulsifier etc.
leavening agent (calcium phosphate)
binding agent (phosphoric acid salt, potassium polyphosphate)
agent for quality improvement (sodium polyphosphate)

TABLE 6

| | | | | |
|---|---|---|---|---|
| Pattern A | Drink | Skim milk (Megmilk Snow Brand | 1 L (powder: 96 g) | 960 mg |
| | | Mainichi Honebuto*) | | |
| Pattern B | Side dish | 6P cheese (Megmilk Snow Brand*) | 18 g × 6 pieces | 800 mg |
| | Snack between meals | One of snacks a to c | | 200 mg |
| Pattern C | Staple food | Cup Noodles* (Nissin*) | 1 | 120 mg |
| | Drink | Skim milk (Megmilk Snow Brand | 500 ml (powder: 48 g) | 480 mg |
| | | Mainichi Honebuto*) | | |
| | Side dish | 6P cheese (Megmilk Snow Brand*) | 18 g × 3 pieces | 400 mg |
| Pattern D | Drink | Skim milk (Megmilk Snow Brand | 500 ml (powder: 48 g) | 480 mg |
| | | Mainichi Honebuto*) | | |
| | Side dish | 6P cheese (Megmilk Snow Brand*) | 18 g × 3 pieces | 400 mg |
| | Snack between meals | One of snacks a to c | | 200 mg |

Snack a: popcorn (Seven & i), one bag (90 g)
Snack b: chocolate bar (Meiji Milk Chocolate), two bars (116 g)
Snack c: Glico Pucchin Purin* (pudding), 67 g × 3 (201 g)
The asterisk (*) indicates a registered trademark.

### 3) Preparation of Saliva Sample

Saliva was collected with a Saliva Collection Aid (SCA) (Salimetrics LLC, Carlsbad, Calif.) on Days −1, 1, 3, 5, and 7. The collected saliva was stored in a freezer before measurement.

The saliva was stored in the freezer until measurement after collection from the subjects. At the time of measurement, the cryopreserved saliva was thawed, and part of the saliva was transferred to a 1.5-mL tube, followed by centrifugation at 1,000 g for 15 minutes at 4° C. After the centrifugation, the supernatant was collected, and PRPs in saliva samples obtained by diluting the supernatant 100- to 800-fold (hPRH2) or 10,000- to 80,000-fold (hPRB1, hPRB2) in phosphate buffer (PBS) were quantified by ELISA.

### (4) ELISA Protocol

The concentrations of human PRPs in each saliva sample were measured by using ELISA kits sold by Cloud-Clone Corp. (SED810Hu (for detection of hPRB1), SED809Hu (for detection of hPRB2), SED812Hu (for detection of hPRH2)).

100 μl of the saliva sample or a sample for a calibration curve containing a recombinant protein in a predetermined amount, which is supplied with each kit, was added to each well of an ELISA plate supplied with the kit. The plate containing the sample was sealed, followed by incubation at 37° C. for 2 hours. After the incubation, the sample in each well was aspirated and discarded, and 100 μl of Detection Reagent A containing a biotin-labeled antibody prepared according to the kit protocol was added to each well. The plate was sealed, followed by incubation at 37° C. for 1 hour. After the incubation, the solution in each well was aspirated and discarded, and each well was washed three times with a wash solution. After the wash solution was thoroughly removed, 100 μl of Detection Reagent B containing enzyme-labeled avidin prepared according to the kit protocol was added to each well. The plate was sealed, followed by incubation at 37° C. for 30 minutes. After the 30 minutes, the solution in each well was aspirated and discarded, and each well was washed five times with a wash solution. After the wash solution was thoroughly removed, 90 μl of substrate solution supplied with the kit was added to each well. The plate was sealed, followed by incubation at 37° C. for 20 minutes (hPRB1, hPRB2) or 40 minutes (PRH2). Thereafter, 50 μl of reaction stop solution supplied with the kit was added to each well, and absorbance at 450 nm was measured with an absorbance microplate reader (Multiskan GO, Thermo Fisher Scientific Inc.). The concentration of each protein, i.e., the concentration of each of PRPs in the saliva, was calculated by making a calibration curve using each recombinant protein supplied with the kit.

### (5) Results

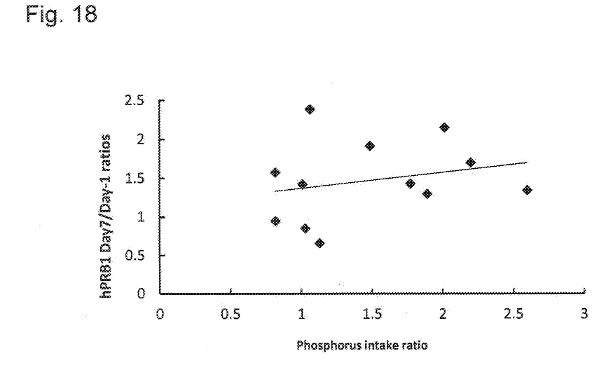

FIG. 18 shows the ratios of the concentrations of hPRB1 in saliva on the final day of the test (Day 7) divided by the concentrations of hPRB1 in saliva on one day before the start of the test (Day −1) in group A and group B (hPRB1 Day7/Day−1 ratios).

Phosphorus intake was calculated from the ingested diets. “Phosphorus intake ratio” in FIG. 18 is a ratio of the total phosphorus intake for 7 days after the start of the high-phosphorus diet (or normal diet) ingestion test divided by the total phosphorus intake for 7 days before the start of the high-phosphorus diet (or normal diet) ingestion test. In subjects showing a high phosphorus intake ratio (subjects who ingested the high-phosphorus diet), the amount of hPRB1 in saliva increased. This indicates that PRPs reflect kidney function and phosphorus intake.

### Example 4: Expression of PRPs in Patient with Kidney Disease

### (1) Subject

Subjects were selected according to the following inclusion criteria.

i. Patient diagnosed with chronic kidney disease or diabetic nephropathy
Regarding chronic kidney disease, patient in GFR category G3 to G5
Regarding diabetic nephropathy, patient in clinical stage 1 to 3
Patient with multiple myeloma and at risk for kidney disease Alternatively, healthy subject (including subject at risk for lifestyle-related disease)
   ii. Person who is able to fully understand this study plan, and is able to give consent by themselves
   iii. Person aged 20 years or older at the time of obtaining consent

However, persons who meet any of the following criteria were excluded from the subjects.

i. Person who is deemed unsuitable as a subject by a researcher
ii. HBs antigen-positive person, HCV antibody-positive person
iii. Subject undergoing dialysis

Table 8 shows clinical data of subjects diagnosed with chronic kidney disease or diabetic nephropathy.

TABLE 8

| Subject No. | | | | | | | |
|---|---|---|---|---|---|---|---|
| 1 | 2 | 3 | 4 | 5 | 6 | Reference value | Unit |

| | | | | | | |
|---|---|---|---|---|---|---|
| Underlying disease | CKD | MM | MM | CKD | CKD | CKD |
| Notes | MDS, ML | | | ML | MDS | MDS |
| Body height | 166.8 | 147.7 | 167.6 | 156.3 | 152.2 | 160.7 |
| Body weight | 61.6 | 55.2 | 65.4 | 55.2 | 53.2 | 60.8 |
| BMI | 22.14 | 25.3 | 23.28 | 22.6 | 26.67 | 23.16 |
| Age | 72 | 77 | 72 | 81 | 85 | 75 |
| Sex | M | M | M | M | M | M |
| Blood pressure | 106/52 | 122/64 | 120/82 | 125/80 | 138/75 | 113/59 |

Blood cell count

| | | | | | | | | |
|---|---|---|---|---|---|---|---|---|
| WBC | 1500 | 3100 | 5600 | 6300 | 2900 | 1300 | 3400-8600 | /μl |
| RBC | 262 | 242 | 302 | 351 | 328 | 237 | 429-571(M), 369-491(F) | ×10\hat{ }4/μl |
| Hb | 9 | 7.6 | 9.6 | 10.9 | 9 | 7.4 | 13.4-17.1(M), 11.4-15.1(F) | g/dl |
| Ht | 26.6 | 23.7 | 29.9 | 33.5 | 29.7 | 23.2 | 39.9-50.1(M), 34.9-45.1(F) | % |
| Plt | 0.4 | 0.8 | 21.7 | 22.8 | 3.1 | 1.8 | 14.9-35.1 | ×10\hat{ }4/μl |

Biochemical data

| | | | | | | | | |
|---|---|---|---|---|---|---|---|---|
| GOT | 16 | 10 | 19 | 20 | 23 | 19 | 13-33 | U/L |
| GPT | 13 | 15 | 32 | 24 | 12 | 14 | 6.0-27 | U/L |
| yGTP | 15 | 35 | 16 | 49 | 18 | 30 | 10-47 | U/L |
| Neutral fat | 186 | 96 | 157 | 165 | 101 | 199 | 30-149 | mg/dl |
| HDL-Chol | 39.5 | 50.8 | 52.4 | 50.4 | 47 | 24 | 40-96 | mg/dl |
| LDL-Chol | 93 | 63 | 70 | 104 | 90 | 86 | 70-139 | mg/dl |
| Creatinine | 2.05 | 0.85 | 1.19 | 1.27 | 1.31 | 1.12 | 0.4-0.7 | mg/dl |
| eGFR | 25.9 | 66.6 | 47 | 42.3 | 40.3 | 49.6 | | |
| Inorganic P | 3.8 | 3.5 | 4.5 | 3.1 | 3.6 | 2.6 | 2.5-4.7 | mg/dl |
| BUN | 23.9 | 23.1 | 22.3 | 19.7 | 37.6 | 12.3 | 8.0-22 | mg/dl |
| Albumin | 3.3 | 3.4 | 3 | 3.7 | 3.8 | 2.7 | 4.0-5.0 | mg/dl |
| FGF23 (serum) | 162 | 33 | 122 | 12 | 69 | 46 | ?? | pg/ml |
| Fasting blood sugar | 89 | 79 | 102 | 77 | 109 | 89 | 70-109 | mg/dl |
| HbA1c(NGSP) | 5.8 | 5.7 | 6.2 | 6 | 5.4 | 6.2 | 4.6-6.2 | % |
| Urinary protein | — | 2+ | — | — | — | — | | |
| Urinary sugar | — | — | — | — | — | — | | |
| L-FABP (urine) concentration | 9.26 | 106.82 | 1.77 | 4.42 | 2.84 | 9.76 | ?? | ng/ml |
| L-FABP in terms of creatinine | 13.35 | 177.41 | 2.37 | 7.93 | 4.94 | 19.06 | 8.4 or less | μg/gCr |
| hPrb1 in saliva | 145.9 | 472.8 | 1018.1 | 904.1 | 602.2 | 1013.6 | | μg/ml |

CKD: chronic kidney disease,
MM: multiple myeloma,
MDS: myelodysplastic syndrome,
ML: malignant lymphoma

(2) Measurement of hPRB1

The concentration of hPRB1 in saliva of each subject was measured according to the methods of Example 3 (3) and (4).

Statistical analysis was performed by using Student's t-test.

### (3) Results

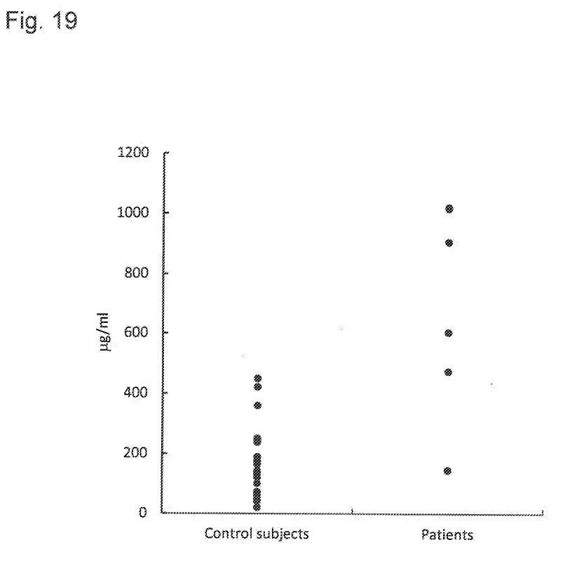

As shown in FIG. 19, the concentration of hPRB1 in saliva of the subjects diagnosed with chronic kidney disease or diagnosed as having multiple myeloma and being at risk for kidney disease (Patients) was higher than that in the healthy subjects (Control Subjects) (p=1.3×10⁻⁶). This indicates that PRPs can be used as kidney function prediction markers.

### Example 5: Expression of PRPs in Subject Who Ingested High-Phosphorus Diet (2)

The proteins in saliva collected from the subjects of Example 3(1) were decomposed with a proteolytic enzyme, and the concentration of proline was measured to examine whether the proline concentration correlates with the high phosphorus diet.

### (1) Decomposition of Protein in Saliva and Derivatization of Decomposition Product

After cryopreserved saliva was thawed, 700 μL of the saliva was transferred to a 1.5-mL tube and centrifuged at 1,000×g for 15 minutes at 4° C., and the supernatant was collected. 10.5 μL of the saliva supernatant was diluted 20-fold with 199.5 μL of Digestion Buffer (0.1 M Tris-HCl (pH 7.5), 0.5% SDS). 100 μL of the diluted saliva supernatant was transferred to a 1.5-mL tube. 20 μL of Pronase (10 μg/mL) was added to 100 μL of the diluted saliva supernatant, and the tube was wrapped with aluminum foil, followed by reacting at room temperature for 1 hour.

1.5 μL of 2-isopropylmalic acid (internal standard) was added per mL of chromatography grade methanol, and a requisite amount of the resulting solution was prepared. Subsequently, 500 μL of the methanol solution containing 2-isopropylmalic acid was added to the Pronase reaction mixture, and the mixture was stirred by vortexing for 30 seconds for spin-down. After the mixture was allowed to stand at room temperature for 5 minutes, 200 μL of ultrapure water was added, and the mixture was stirred by vortexing for 30 seconds and centrifuged at 4600×g for 5 minutes at 4° C. 400 μL of the first supernatant was transferred from the centrifuge tube to another 1.5-mL tube. 200 μL of ultrapure water was added to the first supernatant, and the mixture was stirred by vortexing for 30 seconds and centrifuged at 4600×g for 5 minutes at 4° C. 400 μL of the second supernatant was transferred from the centrifuge tube to an ultrafiltration unit cup (Hydrophilic PTFE membrane, 0.2 μm; Millipore) and centrifuged at 9100×g for 15 minutes at 4° C. The filtrate was dried under reduced pressure at 65° C. for one hour and 30 minutes. 50 μL of a pyridine solution containing 20 mg/mL methoxyamine hydrochloride was added to the residue after drying, and the mixture was shaken with a shaker at 37° C. for 90 minutes. Thereafter, 50 μL of N-methyl-N-trimethylsilyltrifluoroacetamide (MSTFA) was further added, and the mixture was shaken with a shaker at 37° C. for 30 minutes, and trimethylsilylated.

### (2) GCMS Measurement

GCMS-TQ8030 (Shimadzu Corporation) was used for GCMS, and DB-5 (30 m×0.25 mm (inner diameter)×1.00 um (film thickness)) (Agilent Technologies) was used as a capillary column for GC. GC was performed under the following temperature increase conditions: the temperature was increased at a rate of 4° C./min from 100° C. to 320° C. The injector port temperature was 280° C. Helium was used as a carrier gas, and made to flow at a rate of 39.0 an/sec. The energy of the electron ionization was 150 eV, the ion source temperature was 200° C., and proline-2TMS {142.10/73.0} and 2-isopropylmalic acid {216.10/147.10} were measured in MRM mode. 1 μL of the sample was injected, the splitless mode was used, and measurement was performed at a detector voltage of 1.50 kV.

### (3) Analysis of GCMS Data

Analysis was performed by using GCMS Solution Ver. 4.2 data analysis software and the GCMS Metabolites Database (Shimadzu Corporation). A dilution series of purified proline at the following six points: 0.02, 0.01, 0.005, 0.0005, 0.00005, and 0.000005 (nmol/μL) was prepared, and a calibration curve was prepared by using known concentrations of proline.

The concentration of proline was determined by dividing the peak area of proline by the peak area of the internal standard (2-isopropylmalic acid) to obtain a ratio, and applying the ratio to the calibration curve.

### (4) Quantification Results

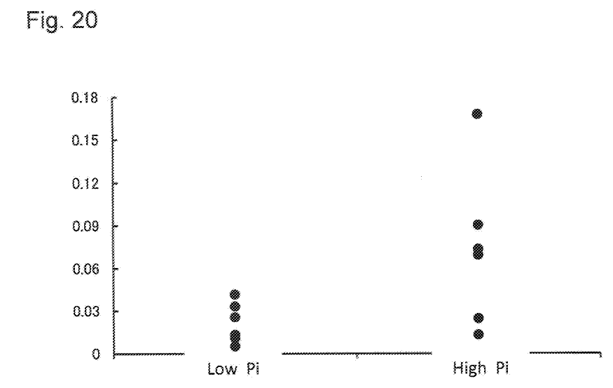

As shown in FIG. 20, the concentration of proline increased in the group that had ingested the high-phosphorus diet for 7 days (High Pi) compared with that in the group that had ingested the normal diet (Low Pi). This indicates that kidney function and phosphorus intake can be predicted by decomposing the proteins in saliva, and measuring the concentration of proline in the decomposition liquid.

### DESCRIPTION OF REFERENCE NUMERALS

1 to 5 Computing device
6 Input unit
7 Display unit
8 Measurement device
8a Measurement device
8b Measurement device
9 Test kit
11 Kidney disease marker value obtaining unit
12 Subject identification unit
13 Measurement value obtaining unit
14 Kidney function evaluation unit
21 Kidney disease marker value obtaining unit
22 Subject identification unit
23 Measurement value obtaining unit
24 Phosphorus intake estimation unit
25 Kidney function decrease risk prediction unit
31 Kidney disease marker value obtaining unit
32 Subject identification unit
33 Measurement value obtaining unit
34 Complication development prediction unit
41 Kidney disease marker value obtaining unit
42 Subject identification unit
43 First measurement value obtaining unit
44 Second measurement value obtaining unit
45 Complication development prediction unit
51 Kidney disease marker value obtaining unit
52 Subject identification unit
53 First measurement value obtaining unit
54 Second measurement value obtaining unit
55 Dietary therapy effect determination unit
82 Reaction/electrophoresis unit
83 Detection unit
84 Analysis unit
91a, 91b Container
92 Microplate
93 Attachment
94 Exterior box
100 System
101 CPU
102 Memory
103 Storage unit
104 Bus
105 Interface unit
109 Storage medium

1-97. (canceled)

98. A device for estimating phosphorus intake in a subject, the device comprising:
a memory for storing a program;
an interface; and
a processor, during execution of the program, configured to:
obtain, through the interface, a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and
estimate the phosphorus intake in the subject based on the measurement value,
the kidney function prediction markers being proline-rich proteins (PRPs).

99. The device according to claim 98, wherein in the case where expression of the kidney function prediction marker increases with an increase in phosphorus intake, the processor compares the measurement value with a predetermined threshold, and determines that the phosphorus intake in the subject is high when the measurement value is higher than the threshold in estimating the phosphorus intake in the subject, or
in the case where expression of the kidney function prediction marker decreases with an increase in phosphorus intake, the processor compares the measurement value with a predetermined threshold, and determines that the phosphorus intake in the subject is high when the measurement value is lower than the threshold in estimating the phosphorus intake in the subject.

100. The device according to claim 98, wherein the processor is further configured to determine that the subject is at risk of a future decrease in kidney function when the phosphorus intake in the subject is determined to be high.

101. The device according to claim 98, wherein the processor is further configured to
obtain a measurement value of at least one kidney disease marker in the subject, before the obtaining the measurement value; and
compare the measurement value of the kidney disease marker with a threshold of a corresponding kidney disease marker, and identifying a subject in which the measurement value of the kidney disease marker is within a range of the threshold of the kidney disease marker, and
estimate phosphorus intake in the identified subject.

102. The device according to claim 98, wherein the measurement value relating to at least one protein selected from the group consisting of the kidney function prediction markers is a measurement value of proline.

103. The device according to claim 98, wherein the specimen is at least one member selected from the group consisting of saliva and salivary glands.

104. The device according to claim 98, wherein, when the subject undergoes dietary therapy, the measurement value recited in claim 98 is the first measurement value, and the estimation recited in claim 98 is the first estimation,
the processor is further configured to:
obtain, through the interface, a second measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and
determine the effect of dietary therapy based on the first measurement value and the second measurement value.

105. The device according to claim 104, wherein in the case where expression of the kidney function prediction marker decreases with a decrease in phosphorus intake, the determination means compares the first measurement value with the second measurement value, and determines that the dietary therapy is effective when the second measurement value is lower than the first measurement value in estimating the phosphorus intake in the subject, or in the case where expression of the kidney function prediction marker increases with a decrease in phosphorus intake, the determination means compares the first measurement value with the second measurement value, and determines that the dietary therapy is effective when the second measurement value is higher than the first measurement value in estimating the phosphorus intake in the subject.

106. The device according to claim 104, wherein the subject is a subject diagnosed with chronic kidney disease GFR category G1 or chronic kidney disease GFR category G2.

107. A method for supporting the estimation of phosphorus intake in a subject, comprising the steps of:
obtaining a measurement value relating to at least one protein selected from the group consisting of kidney function prediction markers contained in a specimen collected from the subject, and/or a measurement value of at least one mRNA selected from the group consisting of kidney function prediction markers contained in a specimen collected from the subject; and
estimating the phosphorus intake in the subject based on the measurement value obtained in the obtaining step,
the kidney function prediction markers being PRPs.

108. The method according to claim 107, wherein in the estimation step, in the case where expression of the kidney function prediction marker increases with an increase in phosphorus intake, the measurement value is compared with a predetermined threshold in estimating the phosphorus intake in the subject, and it is determined that the phosphorus intake in the subject is high when the measurement value is higher than the threshold, or
in the case where expression of the kidney function prediction marker decreases with an increase in phosphorus intake, the measurement value is compared with a predetermined threshold, and it is determined that the phosphorus intake in the subject is high when the measurement value is lower than the threshold in estimating the phosphorus intake in the subject.

109. The method according to claim 107, further comprising a prediction step of determining that the subject is at risk of a future decrease in kidney function when the phosphorus intake in the subject is determined to be high.

110. The method according to claim 107, further comprising, before the obtaining step, the steps of:
obtaining a measurement value of at least one kidney disease marker in the subject; and
comparing the measurement value of the kidney disease marker obtained in the kidney disease marker value obtaining step with a threshold of a corresponding kidney disease marker, and identifying a subject in which the measurement value of the kidney disease marker is within a range of the threshold of the kidney disease marker.

111. The method according to claim 107, wherein the measurement value relating to at least one protein selected from the group consisting of the kidney function prediction markers is a measurement value of proline.

112. The method according to claim 107, wherein the specimen is at least one member selected from the group consisting of saliva and salivary glands.

113. The method according to claim 107, wherein, when the subject undergoes dietary therapy, the measurement value recited in claim 107 is the first measurement value, and the estimation recited in claim 107 is the first estimation,
the method further comprises:
a second obtaining step of obtaining a second measurement value relating to the at least one protein selected from the group consisting of kidney function prediction markers contained in a specimen collected later from the subject, and/or a second measurement value of the at least one mRNA selected from the group consisting of kidney function prediction markers contained in a specimen collected later from the subject; and
a step of determining the effect of dietary therapy based on the first measurement value and the second measurement value.

114. The method according to claim 113, wherein in the determination step, in the case where expression of the kidney function prediction marker decreases with a decrease in phosphorus intake, the first measurement value is compared with the second measurement value, and it is determined that the dietary therapy is effective when the second measurement value is lower than the first measurement value in estimating the phosphorus intake in the subject, or in the case where expression of the kidney function prediction marker increases with a decrease in phosphorus intake, the first measurement value is compared with the second measurement value, and it is determined that the dietary therapy is effective when the second measurement value is higher than the first measurement value in estimating the phosphorus intake in the subject.

115. The method according to claim 113, wherein the subject is a subject diagnosed with chronic kidney disease GFR category G1 or chronic kidney disease GFR category G2.
